(19) United States
(12) Patent Application Publication
(10) Pub. No.: US 2025/0222117 A1
(43) Pub. Date: Jul. 10, 2025

(54) COMPOUND COMPRISING RALTITREXED OR 5-MTHF LINKED TO A THERAPEUTIC AGENT, COMPOSITION, AND METHOD OF USE

(71) Applicant: PURDUE RESEARCH FOUNDATION, West Lafayette, IN (US)

(72) Inventors: Philip S. LOW, West Lafayette, IN (US); Rami A. ALFAR, West Lafayette, IN (US)

(21) Appl. No.: 18/728,431

(22) PCT Filed: Jan. 12, 2023
(86) PCT No.: PCT/US2023/060594

§ 371 Date: Jul. 11, 2024

Related U.S. Application Data
(60) Provisional application No. 63/299,261, filed on Jan. 13, 2022.
(60) Provisional application No. 63/298,793, filed on Jan. 12, 2022.

(51) Int. Cl. A61K 47/55; A61K 47/54; A61P 35/00

(52) U.S. Cl. CPC A61K 47/55; A61K 47/545; A61K 47/549; A61K 47/554; A61P 35/00

(57) ABSTRACT

Compounds comprising a radical of a therapeutic agent conjugated to a radical of raltitrexed, 5-methyltetrahydrofolate (5-MTHF), an analog of raltitrexed, or an analog of 5-MTHF via a linker; compositions comprising same; and a method of immunomodulating Tregs.

### PRIORITY

This patent application is related to and claims the priority benefit of U.S. Provisional Patent Application No. 63/298,793 filed Jan. 12, 2022, and U.S. Provisional Patent Application No. 63/299,261 filed Jan. 13, 2022. The contents of the foregoing applications are hereby incorporated by reference in their entireties into this disclosure.

### TECHNICAL FIELD

This disclosure relates to compounds comprising a therapeutic agent conjugated (such as via a linker) to raltitrexed or 5-methyltetrahydrofolate (5-MTHF), compositions comprising same, and methods of use to immunomodulate regulatory T cells (Tregs), such as in a patient with cancer or a fibrotic disease or disorder.

### BACKGROUND

This section introduces aspects that may help facilitate a better understanding of the disclosure. Accordingly, these statements are to be read in this light and are not to be construed as admissions about what is or is not prior art.

Tumors are not just masses of malignant cells, but instead can be a composite of many different constituents, some of which surround and directly influence the growth and malignant behavior of cancer cells, which leads to invasion and metastasis. Currently, most FDA-approved therapeutics focus on targeting and killing tumor cells. Stromal cell types within the tumor microenvironment (TME), however, are genetically more stable. The TME comprises multiple types of stromal cells, including immune cells, fibroblasts, and epithelial cells.

The successful targeting of stromal cells in the TME could enable a treatment for cancer with universal application; it would not be limited to a specific type of cancer. Thus, identifying ways of reprogramming stromal cells to become anti-tumorigenic has become a major priority in cancer research.

Tumor cells in the TME are known to recruit regulatory T cells (Tregs) via several cytokines and chemokines. The Tregs promote tumor growth and metastasis and inhibit anti-tumor immunity via complex and dynamic paracrine signaling through a network of cytokines, as well as contact-dependent and contact-independent mechanisms. These mechanisms include direct cytotoxicity and inhibitory receptors, mainly including the inhibition of CD8+ T cells. Tregs, however, can be reprogrammed from inhibitors to promoters of anti-tumor immunity.

In view of the above, it is an object of the present disclosure to provide compounds, compositions comprising same, and methods of use thereof to immunomodulate Tregs, such as in a patient with cancer or a fibrotic disease or disorder. This and other objects and advantages, as well as inventive features, will be apparent from the description provided herein.

### SUMMARY

Provided is a compound of Formula (I):

T-L-E (I)

or a pharmaceutically acceptable salt thereof, wherein T is a radical of raltitrexed, 5-methyltetrahydrofolate (5-MTHF), an analog of raltitrexed, or an analog of 5-MTHF; L is a linker; and E is a radical of a therapeutic agent.

In certain embodiments, T has the structure of Formula (II):

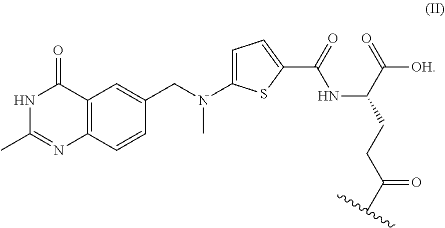 [SMILES: *C(=O)CC[C@H](NC(=O)c1ccc(N(C)Cc2ccc3nc(C)[nH]c(=O)c3c2)s1)C(=O)O]

In certain embodiments, T has the structure of Formula (III):

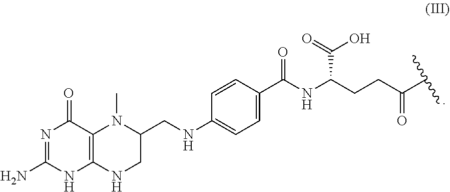 [SMILES: *C(=O)CC[C@H](NC(=O)c1ccc(NCC2CNc3[nH]c(N)nc(=O)c3N2C)cc1)C(=O)O]

The therapeutic agent can be selected from the group consisting of toll-like receptor 7 (TLR7) agonist, a phosphoinositide 3-kinase (PI3k) inhibitor, a steroid, a nucleotide-binding and oligomerization domain (NOD)-like receptor 2 (NLR2) agonist, a stimulatory of interferon gene (STING) agonist, an enhancer of zeste homolog 2 (EZH2) inhibitor, a NOD-, LRR- and pyrin domain-containing protein 3 (NLRP3) inhibitor, a Caspase I inhibitor, a retinoic acid-inducible gene I (RIG-I)-like receptor (RLR) agonist, an absent in melanoma 2 (AIM2)-like receptor agonist, and an agonist of a receptor for advanced glycation end products (RAGE).

The therapeutic agent can be a NLR2 agonist having the structure:

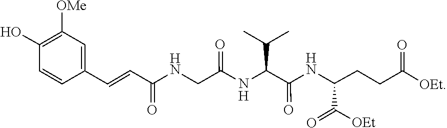 [SMILES: CCOC(=O)CC[C@@H](NC(=O)[C@@H](NC(=O)CNC(=O)/C=C/c1ccc(O)c(OC)c1)C(C)C)C(=O)OCC]

The therapeutic agent can be a STING agonist having the structure:

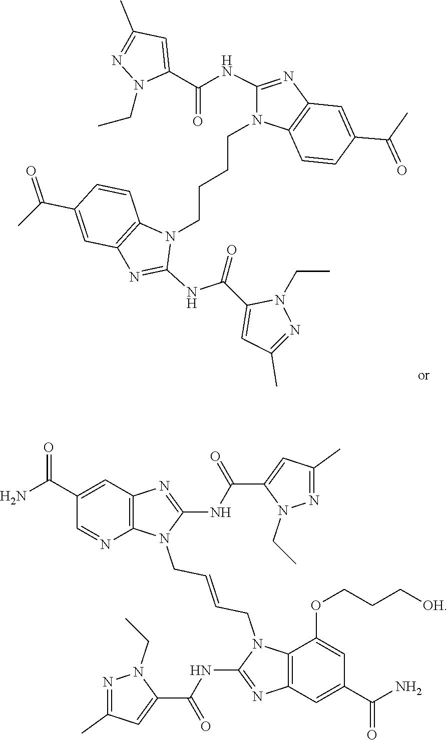 [SMILES: CCn1nc(C)cc1C(=O)Nc1nc2cc(C(C)=O)ccc2n1CCCCn1c(NC(=O)c2cc(C)nn2CC)nc2cc(C(C)=O)ccc21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO)c21]

In certain embodiments, the therapeutic agent is an EZH2 inhibitor. The EZH2 inhibitor can be

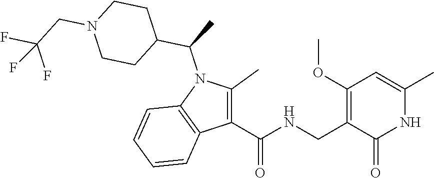 [SMILES: COc1cc(C)[nH]c(=O)c1CNC(=O)c1c(C)n([C@H](C)C2CCN(CC(F)(F)F)CC2)c2ccccc12]

or tazemetostat.

In certain embodiments, the therapeutic agent is a NLRP3 inhibitor having the structure:

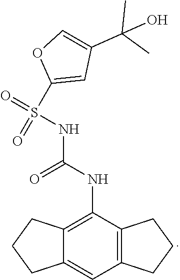 [SMILES: CC(C)(O)c1coc(S(=O)(=O)NC(=O)Nc2c3c(cc4c2CCC4)CCC3)c1]

In certain embodiments, the therapeutic agent is a Caspase I inhibitor having the structure:

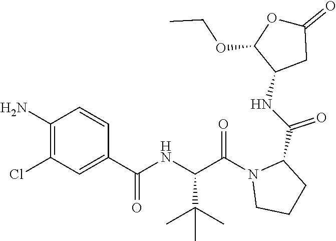 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C]

In certain embodiments, the therapeutic agent is a PI3 kinase inhibitor having the structure:

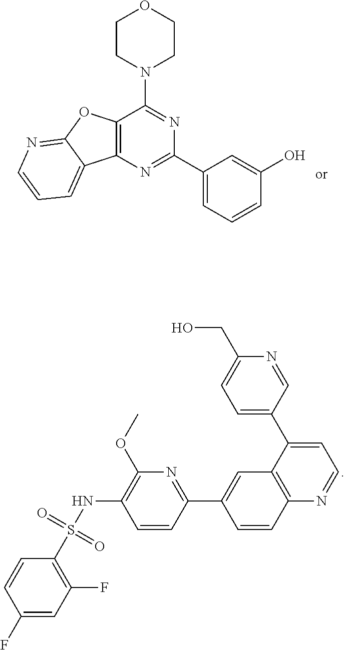 [SMILES: Oc1cccc(-c2nc(N3CCOCC3)c3oc4ncccc4c3n2)c1 ; COc1nc(-c2ccc3nccc(-c4ccc(CO)nc4)c3c2)ccc1NS(=O)(=O)c1ccc(F)cc1F]

In certain embodiments, the therapeutic agent is a RLR agonist having the structure:

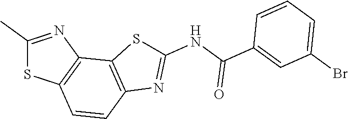 [SMILES: Cc1nc2c(ccc3nc(NC(=O)c4cccc(Br)c4)sc32)s1]

Also provided is a compound of the Formula (I):

T-L-E (I)

or a pharmaceutically acceptable salt thereof, wherein T is a radical of raltitrexed, 5-MTHF, an analog of raltitrexed, or an analog of 5-MTHF; L is a linker; and E is a radical of a TLR7 agonist represented by Formula (IV):

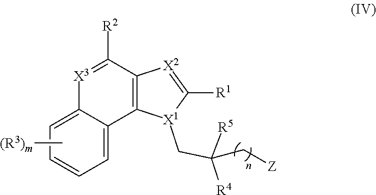

or a pharmaceutically acceptable salt thereof wherein:
R¹, R³, R⁴, R⁵ are each independently a hydrogen (H), alkyl, alkoxyl, alkenyl, alkynyl, cycloalkyl, aryl halo, heteroaryl, —COR^{2x},

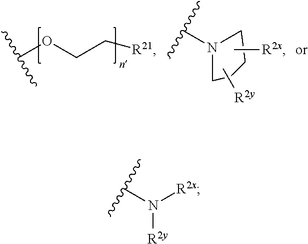

R² is a H, —OH, —NH₂, —NHR^{2x}, N₃, —NH—CH₂—NH₂, —CONH₂, —SO₂NH₂, —NH—CS—NH₂,

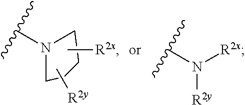

Z is a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

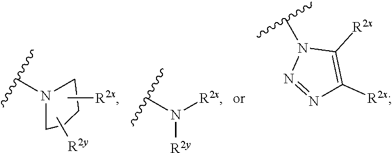

wherein:
   each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl; and
   each R^{2z} is independently selected from the group consisting of —NH₂, —NR^{2q}R^{2q′}, —O—R^{2q}, —SO—R^{2q}, and —COR^{2q},
   wherein each R^{2q} and R^{2q′} is independently alkyl or H,

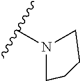 [SMILES: *N1CCCC1]

is a 3-10 membered N-containing non-aromatic, mono- or bicyclic heterocycle,
R²¹ is H or alkyl,
n′ is 0-30; and
wherein in Formula (IV), each of X¹, X², X³ is independently CR^{q} or N, wherein each R^{q} is independently H, halogen, or optionally substituted alkyl,
   n is 0-30,
   m is 0-4; and
   when n is 0, Y is not H, —OH, or —O—R^{2x}.

E can be a radical of a compound represented by Formula (IVA):

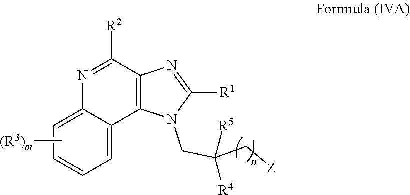

or a pharmaceutically acceptable salt thereof, wherein:
R¹ is an optionally substituted C₃-C₈ alkyl;
R² is H, —OR^{z}, —SO₂N(R^{z})₂, —NR^{2x}R^{2y}, or N₃, wherein:
   R^{2x} and R^{2y} are each independently H, —N(R^{z})₂, —CON(R^{z})₂, —C(R^{z})₂—N(R^{z})₂, —CS—N(R^{z})₂, or optionally substituted alkyl,
   each R^{z} is independently hydrogen, halogen, or optionally substituted alkyl, or
   R^{2x} and R^{2y} are taken together to form an optionally substituted heterocycloalkyl;
Z is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂,
each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein the alkyl, alkoxy, heteroalkyl, cycloalkyl, or heterocycloalkyl is optionally substituted;
R⁴ and R⁵ are each independently alkyl, alkoxy, halogen, or cycloalkyl, wherein each of the alkyl, alkoxy, and cycloalkyl is optionally substituted;
n is 1-6; and
m is 0-4.

In certain embodiments, the compound of Formula (I) is represented by Formula (IVB) or Formula (IVC):

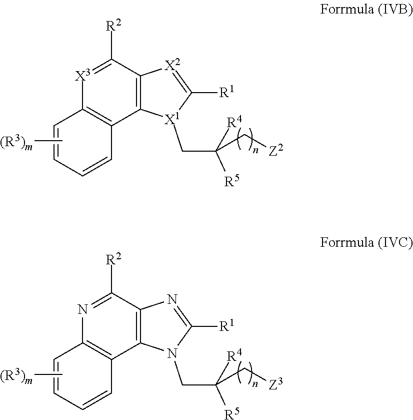

or a pharmaceutically acceptable salt thereof, wherein: each R¹ is independently an optionally substituted C₃-C₈ alkyl; each R² is independently H, —OR^{z}, —SO₂N(R^{z})₂, —NR^{2x}R^{2y}, or N₃; each R^{2x} and R^{2y} are independently H, —N(R^{z})₂, —CON(R^{z})₂, —C(R^{z})₂—N(R^{z})₂, —CS—N(R^{z})₂, or optionally substituted alkyl, each R^{z} is independently H, halogen, or an optionally substituted alkyl, or R^{2X} and R^{2y} are taken together to form an optionally substituted heterocycloalkyl; each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein each of the alkyl, alkoxy, heteroalkyl, cycloalkyl, or heterocycloalkyl is optionally substituted; each R⁴ and R⁵ are independently alkyl, alkoxy, halogen, or cycloalkyl, wherein each of the alkyl, alkoxy, and cycloalkyl, is optionally substituted; n is 1-6; m is 0-4; each Z² and Z³ is independently a group of the formula T-L-, T-L-O—, T-L-O-alkyl-, T-L-S¹—, T-SO₂—NH—, T-L-NR^{a}R^{b}—, T-L-S(O)ₓ-alkyl-, T-L-CO—, T-L-aryl-, T-L-NH—CO—NH—, T-L-NH—O—, T-L-NH—NH—, T-L-NH—CS—NH, T-L-C(O)-alkyl-, or T-L-SO₂—; R^{a} and R^{b} are each independently H, halo, hydroxy, alkoxy, aryl, amino, acyl or C(O)R^{c}, wherein R^{c} is alkyl, aryl, oxy or alkoxy; S¹ is a spacer; x is 0-3; n is 1-3 and m is 0-4.

In other embodiments, the compound of Formula (I) is represented by Formula (IVB):

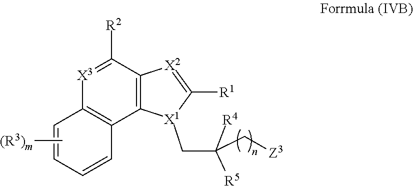

or a pharmaceutically acceptable salt thereof, wherein: R is an optionally substituted C₃-C₈ alkyl; R² is H, —OR^{z}, —SO₂N(R^{z})₂, —NR^{2x}R^{2y}, or N₃; R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl, each R^{z} is independently H, halogen, or an optionally substituted alkyl, or R^{2x} and R^{2y} are taken together to form an optionally substituted heterocycloalkyl; each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein each of the alkyl, alkoxy, heteroalkyl, cycloalkyl, or heterocycloalkyl is optionally substituted; R⁴ and R⁵ are each independently alkyl, alkoxy, halogen, or cycloalkyl, wherein each of the alkyl, alkoxy, and cycloalkyl, is optionally substituted; n is 1-6; m is 0-4; each of Z² is a group of the formula T-L-, T-L-O—, T-L-O-alkyl-, T-L-S¹—, T-SO₂—NH—, T-L-NR^{a}R^{b}—, T-L-S(O)ₓalkyl-, T-L-CO—, T-L-aryl-, T-L-NH—CO—NH—, T-L-NH—O—, T-L-NH—NH—, T-L-NH—CS—NH, T-L-C(O)-alkyl-, or T-L-SO₂—; R^{a} and R^{b} are each independently H, halo, hydroxy, alkoxy, aryl, amino, acyl or C(O)R^{c}, wherein R^{c} is alkyl, aryl, oxy or alkoxy; x is 0-3; n is 1-3; S¹ is a spacer and m is 0-4.

In other embodiments, the compound of Formula (I) is represented by Formula (IVC):

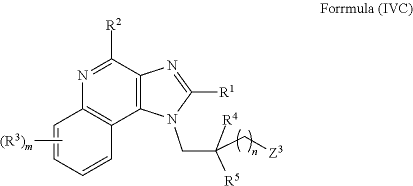

or a pharmaceutically acceptable salt thereof, wherein: R is an optionally substituted C₃-C₈ alkyl; R² is H, —OR^{z}, —SO₂N(R^{z})₂, —NR^{2x}R^{2y}, or N₃; R^{2x} and R^{2y} are each independently H, —N(R^{z})₂, —CON(R^{z})₂, —C(R^{z})₂—N(R^{z})₂, —CS—N(R^{z})₂, or optionally substituted alkyl, each R^{z} is independently H, halogen, or an optionally substituted alkyl, or R^{2x} and R^{2y} are taken together to form an optionally substituted heterocycloalkyl; each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein each of the alkyl, alkoxy, heteroalkyl, cycloalkyl, or heterocycloalkyl is optionally substituted; R⁴ and R⁵ are each independently alkyl, alkoxy, halogen, or cycloalkyl, wherein each of the alkyl, alkoxy, and cycloalkyl, is optionally substituted; n is 1-6; m is 0-4; each of Z³ is a group of the formula T-L-, T-L-O—, T-L-O-alkyl-, T-L-S¹—, T-SO₂—NH—, T-L-NR^{a}R^{b}—, T-L-S(O)ₓ-alkyl-, T-L-CO—, T-L-aryl-, T-L-NH—CO—NH—, T-L-NH—O—, T-L-NH—NH—, T-L-NH—CS—NH, T-L-C(O)-alkyl-, or T-L-SO₂—; R^{a} and R^{b} are each independently H, halo, hydroxy, alkoxy, aryl, amino, acyl or C(O)R^{c}, wherein R^{c} is alkyl, aryl, oxy or alkoxy; x is 0-3; S¹ is a spacer; n is 1-3 and m is 0-4.

In certain embodiments where the compound of Formula (I) is represented by Formula (IVB) or Formula (IVC), R¹ is a C₁-C₆ alkyl optionally substituted with 1-3 substituents, each substituent independently being halogen or C₁-C₆ alkoxy; R² is —NR^{2x}R^{2y}, where R^{2x} and R^{2y} are each independently a H or a C₁-C₆ alkyl; each R³ is independently a halogen, —CN, C₁-C₆ alkyl, C₁-C₆ heteroalkyl, C₃-C₇ cycloalkyl, C₁-C₆ alkoxy, amino, hydroxy, carboxyl, or thiol; R⁴ and R⁵ are each independently C₁-C₆ alkyl; each X¹, X², and X³ is N; each of Z² and Z³ is independently T-L-, or T-L-O—; n is 1; and m is 0-4. In certain embodiments where the compound of Formula (I) is represented by Formula (IVB) or Formula (IVC), each of Z² and Z³ is T-L-O—.

In certain embodiments of Formula (IV) or Formula (IVA), Z can be a group of the formula T-L-, T-L-O—, T-L-O-alkyl-, T-L-S¹—, T-SO₂—NH—, T-L-NR^{a}R^{b}—, T-L-S(O)ₓ-alkyl-, T-L-CO—, T-L-aryl-, T-L-NH—CO—NH—, T-L-NH—O—, T-L-NH—NH—, T-L-NH—CS—NH, T-L-C(O)-alkyl-, or T-L-SO₂—, wherein:

R^{a} and R^{b} are each independently H, halo, hydroxy, alkoxy, aryl, amino, acyl or C(O)R^{c}, wherein R^{c} is alkyl, aryl, oxy or alkoxy; S¹ is a spacer; and x is 0-3.

In certain embodiments of Formula (IVA), R¹ is a C₁-C₆ alkyl optionally substituted with 1-3 substituents, each substituent independently being halogen or C₁-C₆ alkoxy; R² is —NR^{2x}R^{2y}, where R^{2x} and R^{2y} are each independently aH or a C₁-C₆ alkyl; each R³ is independently a halogen, —CN, C₁-C₆ alkyl, C₁-C₆ heteroalkyl, C₃-C₇ cycloalkyl, C₁-C₆ alkoxy, amino, hydroxy, carboxyl, or thiol; R⁴ and R⁵ are each independently be C₁-C₆ alkyl; each X¹, X², and X³ is N; Z is T-L- or T-L-O—; n is 1; and m is 0. Z can be T-L-O—.

R¹ can be optionally substituted C₃-C₆ alkyl. R¹ can be an optionally substituted acyclic C₃-C₆ alkyl. R² can be —NR^{2x}R^{2y}. R² can be —NH₂.

The compound of Formula (IVA) can be one of the formulae:

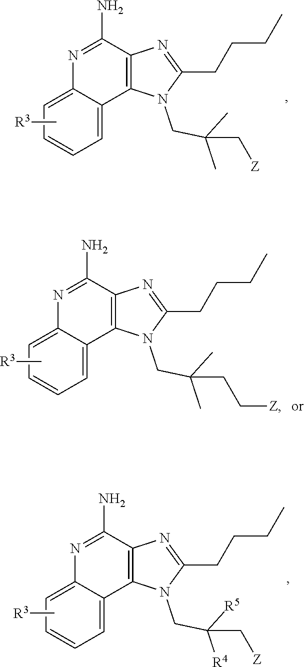

or a pharmaceutically acceptable salt thereof, wherein R³ is optionally absent. The compound of Formula (IV) can be one of the formulae:

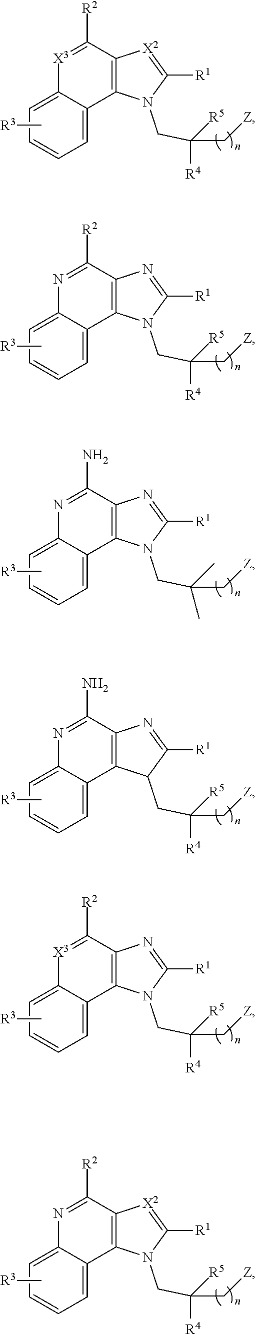

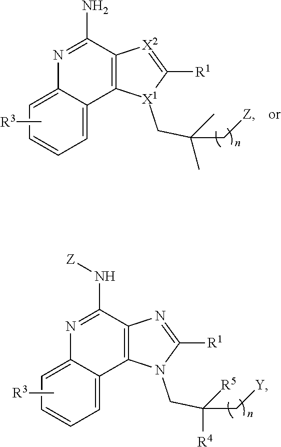

or a pharmaceutically acceptable salt of any of the foregoing formulae, wherein R³ is optionally absent.

R¹ can be a C₁-C₆ alkyl. R² can be —NH₂. R³ can be absent.

In certain embodiments. R¹ is a C₁-C₆ alkyl; R² is —NH₂; n is 1; and R³ is absent.

In certain embodiments, the compound of the Formula (I) is a compound represented by Formula (V):

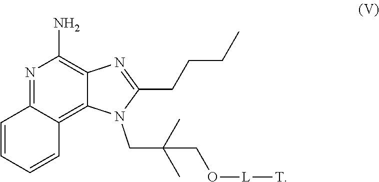

In certain embodiments, the radical of the TLR7 agonist (e.g, E) has the structure:

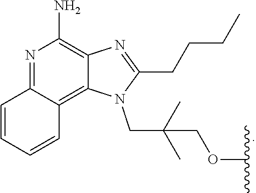

Further provided is a compound of the Formula (I):

T-L-E (I)

or a pharmaceutically acceptable salt thereof, wherein T is a radical of raltitrexed, 5-MTHF, an analog of raltitrexed, or an analog of 5-MTHF; L is a linker; and E is

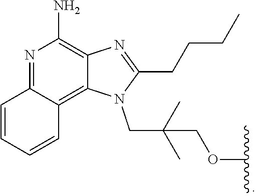

Still further provided is a compound of the Formula (I):

T-L-E (I)

or a pharmaceutically acceptable salt thereof, wherein T is a radical of raltitrexed, 5-MTHF, an analog of raltitrexed, or an analog of 5-MTHF; L is a linker; and E is a radical of the structure:

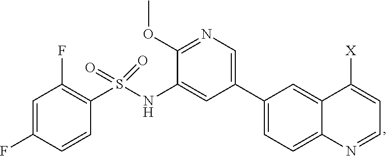

wherein X can be any of the following:

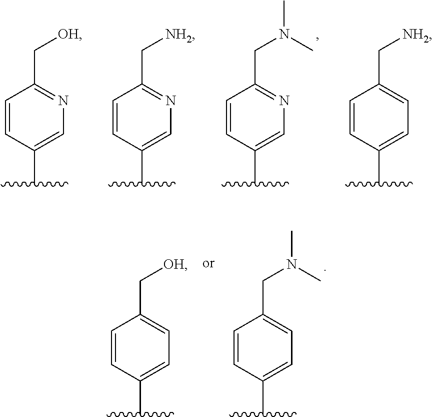

E can comprise a radical of the structure:

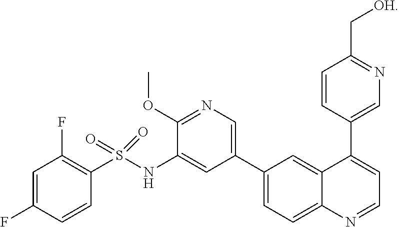

In certain embodiments, the compound is of Formula (I):

T-L-E (I)

or is a pharmaceutically acceptable salt thereof, wherein:
T is a radical of raltitrexed, 5-MTHF, an analog of raltitrexed, or an analog of 5-MTHF;
L is a linker; and
E is a radical of a corticosteroid.

The corticosteroid can be betamethasone, cortisone, cortivazol, difluprednate, hydrocortisone, prednisolone, methylprednisolone, prednisone, dexamethasone, hydrocortisone-17-valerate, budesonide, flumethazone, fluticasone propionate, fluorocortisone, fludrocortisone, paramethasone, eplerenone, or an ester of any of the foregoing.

L can be a releasable linker. L can be a non-releasable linker. L can comprise an optionally substituted heteroalkyl. In certain embodiments, the optionally substituted heteroalkyl is substituted with at least one substituent selected from the group consisting of alkyl, hydroxyl, acyl, polyethylene glycol (PEG), carboxylate, and halo. L can comprise a substituted heteroalkyl with at least one disulfide bond in the backbone thereof. L can comprise a peptide or a peptidoglycan with at least one disulfide bond in the backbone thereof. L can be a releasable linker that can be cleaved by enzymatic reaction, a reactive oxygen species (ROS), or reductive conditions. L can comprise the formula —NH—CH₂—CR⁶R⁷—S—S—CH₂—CH₂—O—CO—, wherein R⁶ and R⁷ are each, independently, H, alkyl, or heteroalkyl. L can be a group, or can comprise a group, of the formulae:

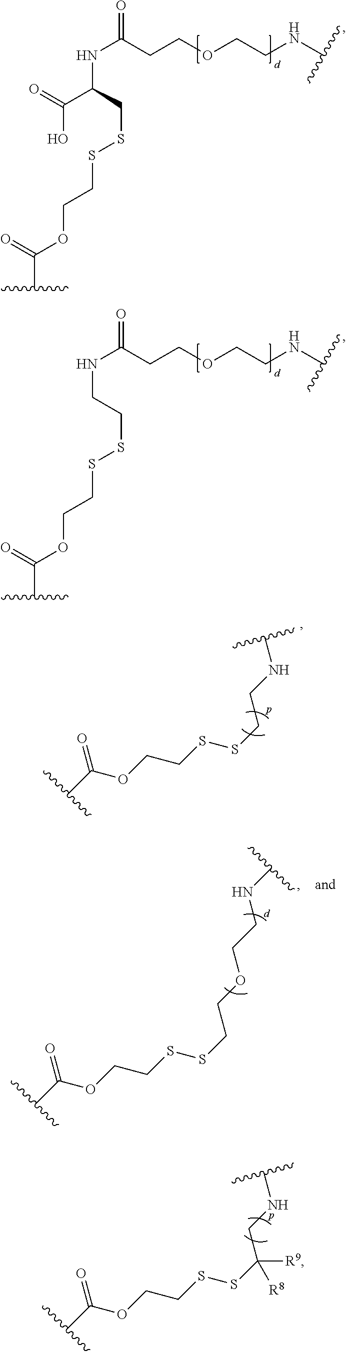

wherein p is an integer from 0 to 30; d is an integer from 1 to 40; and R⁸ and R⁹ are each, independently, H, alkyl, a heterocyclyl, a cycloalkyl, an aryl, or a heteroalkyl.

L can comprise one or more linker moieties, each of the one or more linker moieties independently selected from the group consisting of alkylene, heteroalkylene, —O— alkynylene, alkenylene, acyl, aryl, heteroaryl, amide, oxime, ether, ester, triazole, PEG, carboxylate, carbonate, carbamate, amino acid, peptide, and peptidoglycan. L can be, or can comprise, a peptide or a peptidoglycan. L can be, or can comprise, an amino acid. L can be, or can comprise, a PEG group. L can be, or can comprise, a polysaccharide. L can be, or can comprise, a group represented by the structure:

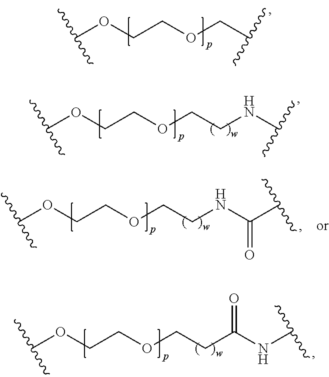

wherein w is 0-5 and p is 1-30. L can be, or can comprise, a linker moiety selected from the group consisting of

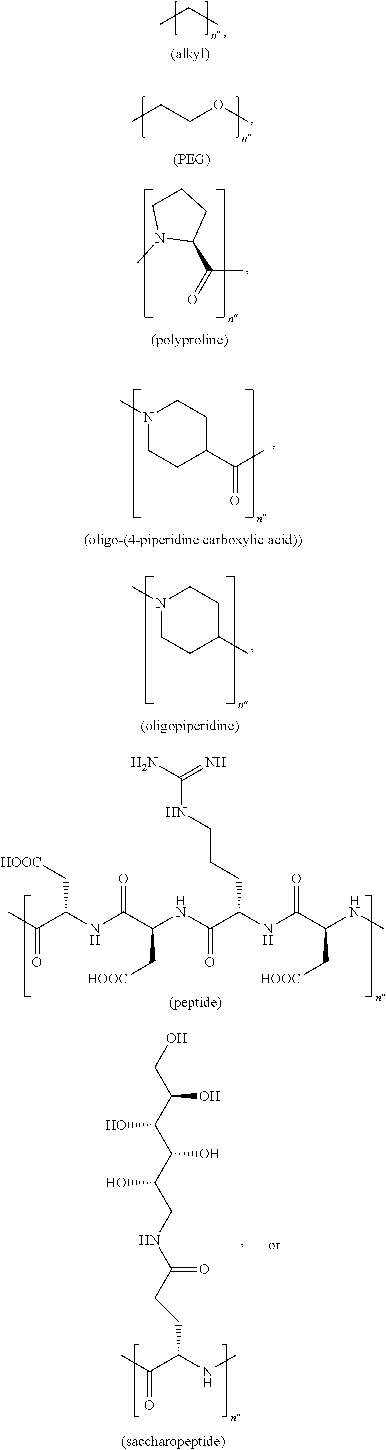

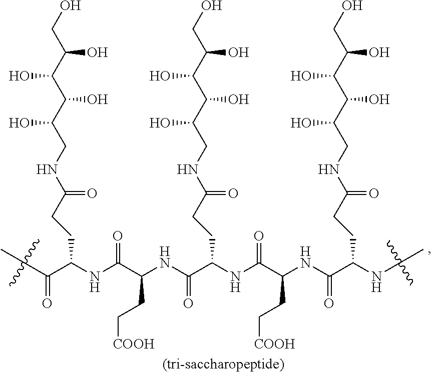

wherein n″ is 0-30. L can be a bivalent linker. L can be a trivalent linker.

Any of the above compounds can further comprise a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the linker.

The compound can further comprise an albumin binding group, e.g., an albumin binding group selected from the group consisting of

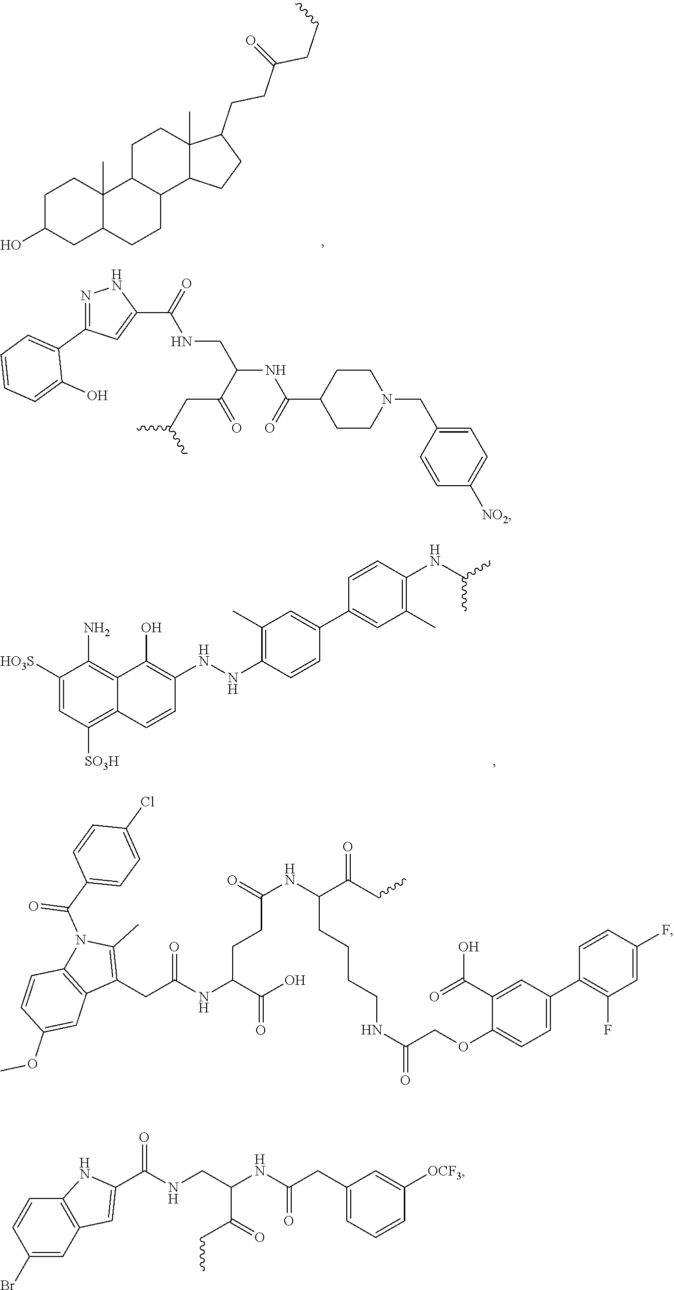

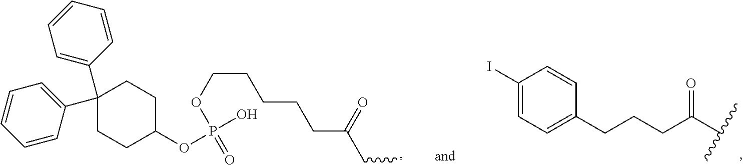

e.g., an albumin binding group selected from a group consisting of:

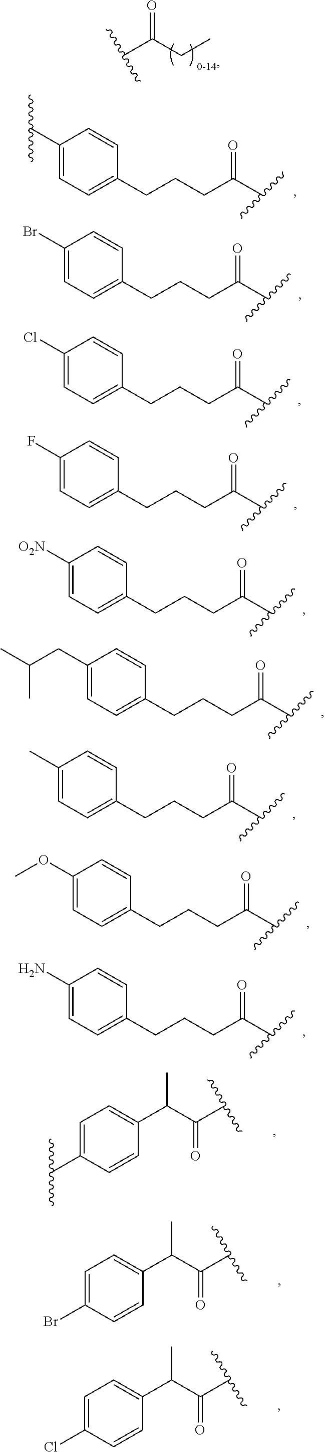

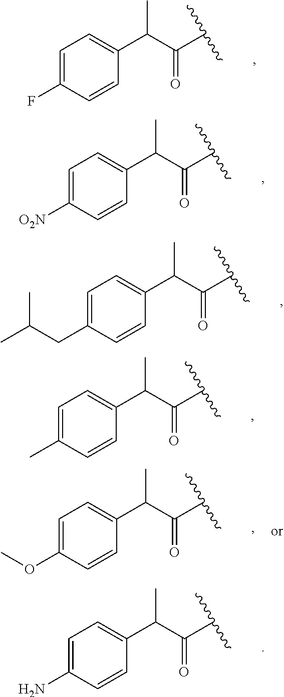

In certain embodiments, the compound comprises (e.g., consists of) one of the following structures.

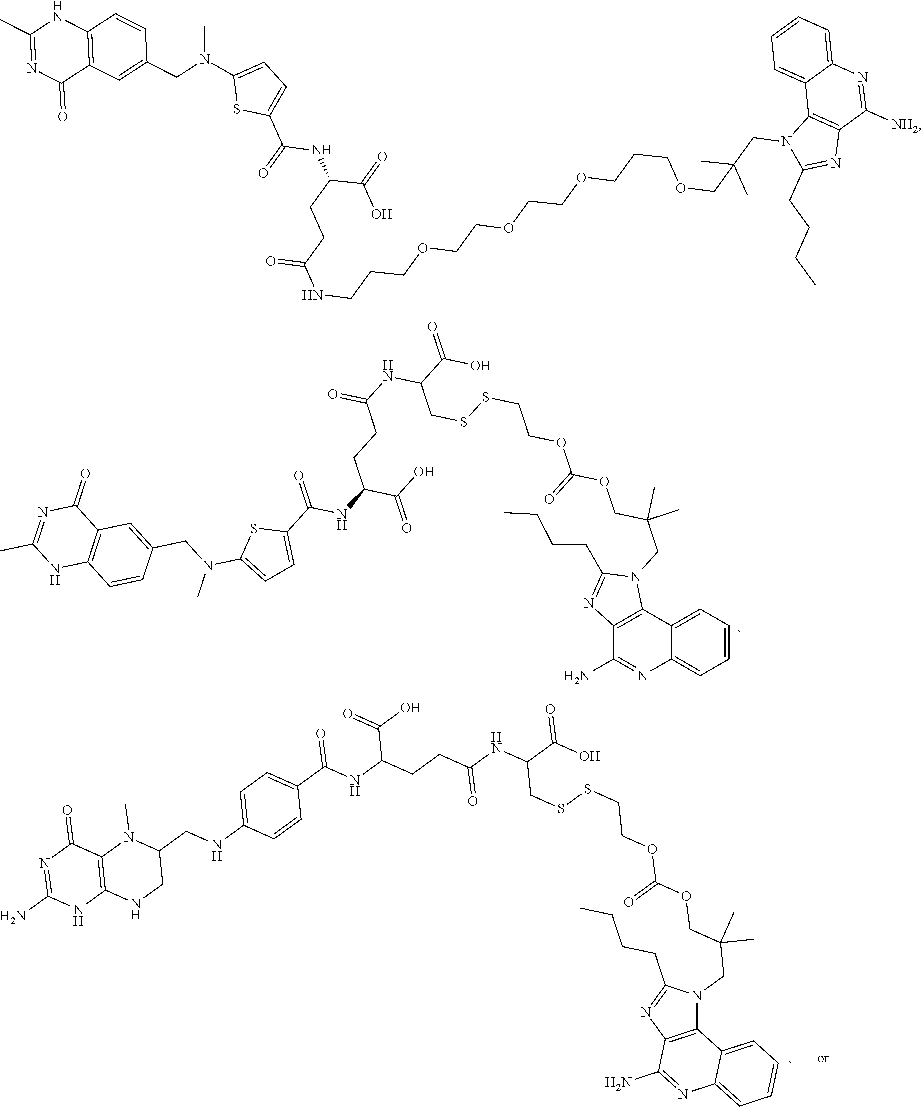 [SMILES: CCCCc1nc2c(N)nc3ccccc3c2n1CC(C)(C)COCCCOCCOCCOCCCNC(=O)CC[C@H](NC(=O)c1ccc(N(C)Cc2ccc3[nH]c(C)nc(=O)c3c2)s1)C(=O)O ; CCCCc1nc2c(N)nc3ccccc3c2n1CC(C)(C)COC(=O)OCCSSCC(NC(=O)CC[C@H](NC(=O)c1ccc(N(C)Cc2ccc3[nH]c(C)nc(=O)c3c2)s1)C(=O)O)C(=O)O ; CCCCc1nc2c(N)nc3ccccc3c2n1CC(C)(C)COC(=O)OCCSSCC(NC(=O)CCC(NC(=O)c1ccc(NCC2CNc3[nH]c(N)nc(=O)c3N2C)cc1)C(=O)O)C(=O)O]

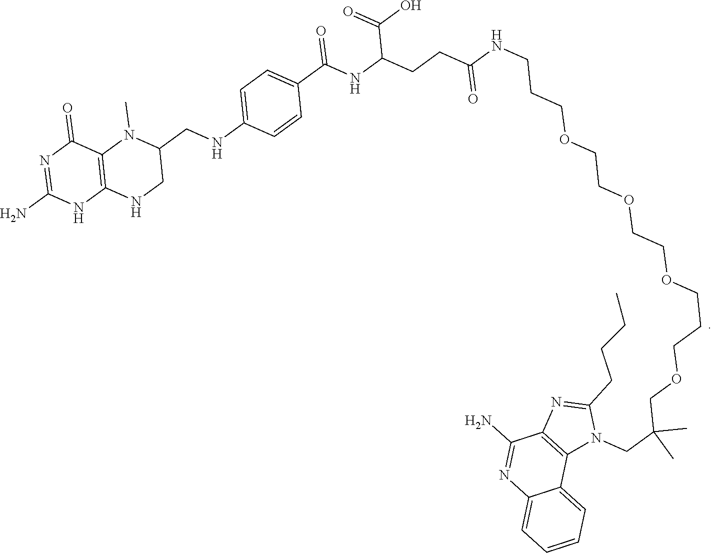 [SMILES: CCCCc1nc2c(N)nc3ccccc3c2n1CC(C)(C)COCCCOCCOCCOCCCNC(=O)CCC(NC(=O)c1ccc(NCC2CNc3[nH]c(N)nc(=O)c3N2C)cc1)C(=O)O]

In certain embodiments, the compound comprises (e.g., consists of) one of the following structures:

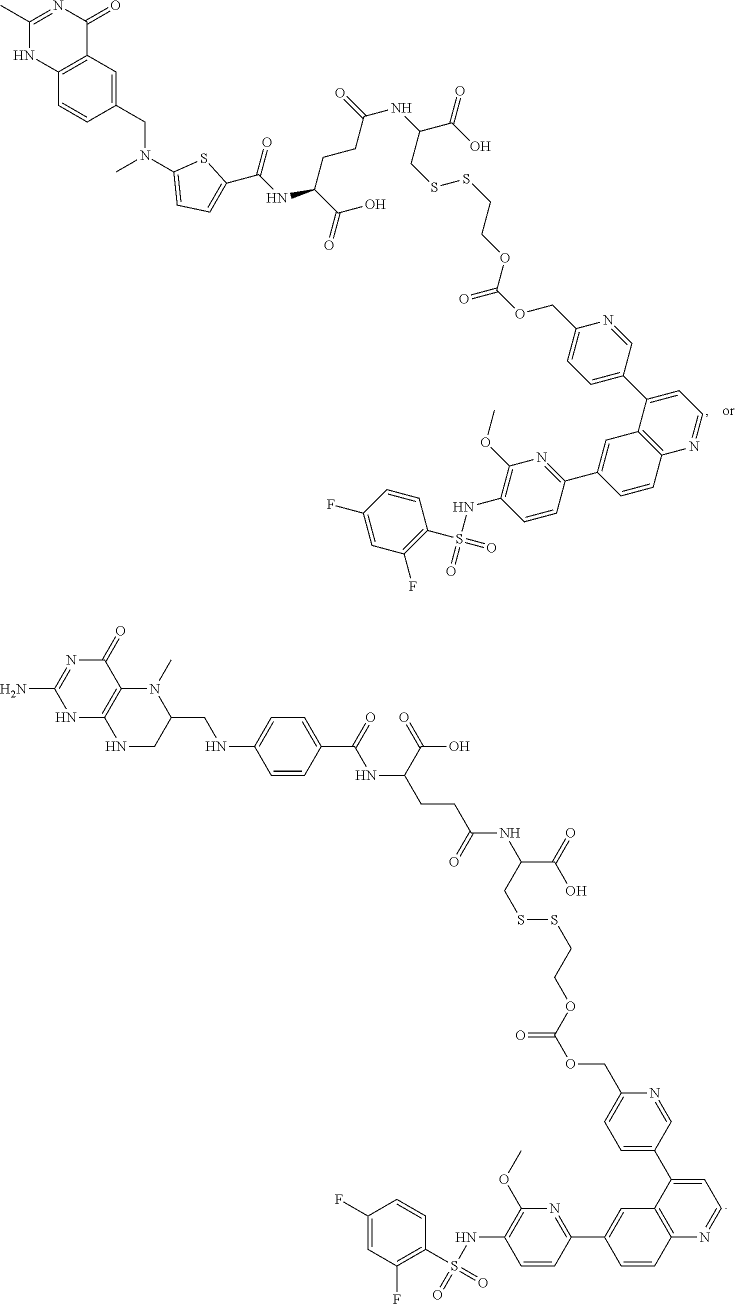 [SMILES: COc1nc(-c2ccc3nccc(-c4ccc(COC(=O)OCCSSCC(NC(=O)CC[C@H](NC(=O)c5ccc(N(C)Cc6ccc7[nH]c(C)nc(=O)c7c6)s5)C(=O)O)C(=O)O)nc4)c3c2)ccc1NS(=O)(=O)c1ccc(F)cc1F ; COc1nc(-c2ccc3nccc(-c4ccc(COC(=O)OCCSSCC(NC(=O)CCC(NC(=O)c5ccc(NCC6CNc7[nH]c(N)nc(=O)c7N6C)cc5)C(=O)O)C(=O)O)nc4)c3c2)ccc1NS(=O)(=O)c1ccc(F)cc1F]

In certain embodiments, the compound comprises (e.g., consists of) one of the following structures:

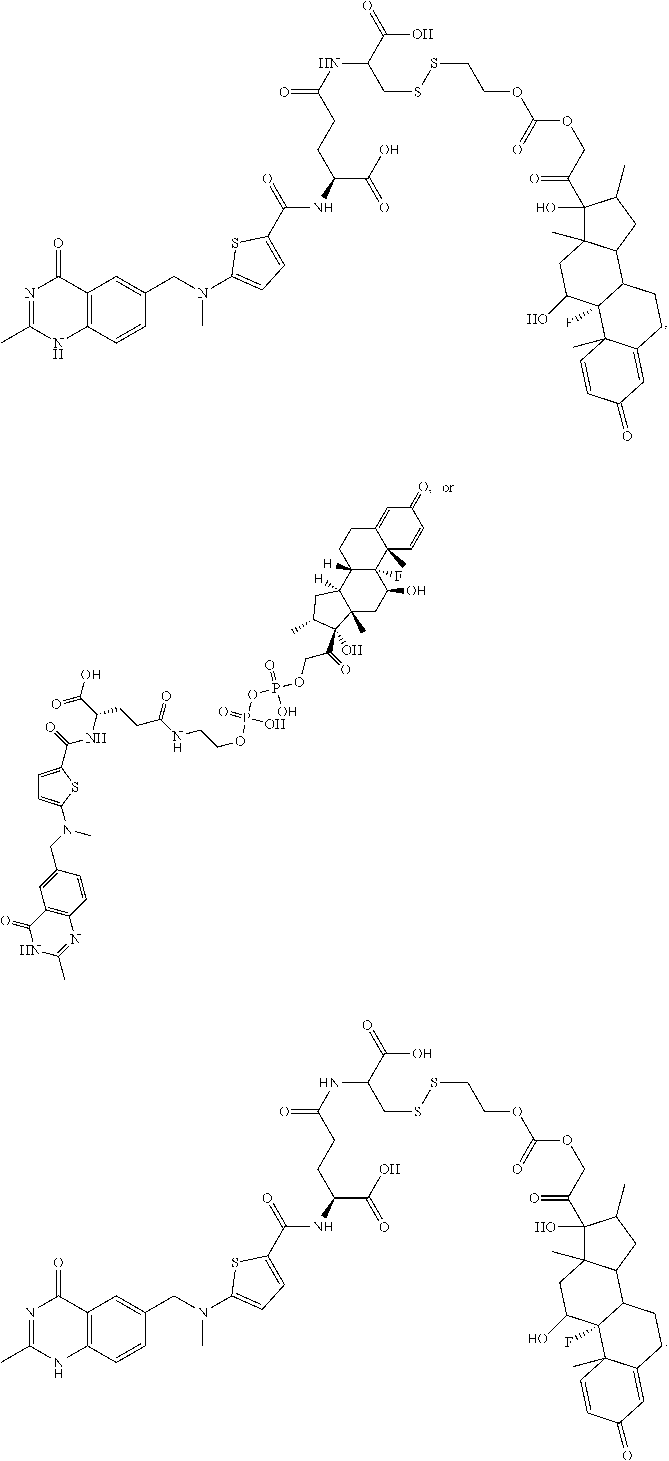 [SMILES: Cc1nc(=O)c2cc(CN(C)c3ccc(C(=O)N[C@@H](CCC(=O)NC(CSSCCOC(=O)OCC(=O)C4(O)C(C)CC5C6CCC7=CC(=O)C=CC7(C)[C@@]6(F)C(O)CC54C)C(=O)O)C(=O)O)s3)ccc2[nH]1 ; Cc1nc2ccc(CN(C)c3ccc(C(=O)N[C@@H](CCC(=O)NCCOP(=O)(O)OP(=O)(O)OCC(=O)[C@@]4(O)[C@H](C)C[C@H]5[C@@H]6CCC7=CC(=O)C=C[C@]7(C)[C@@]6(F)[C@@H](O)C[C@@]54C)C(=O)O)s3)cc2c(=O)[nH]1 ; Cc1nc(=O)c2cc(CN(C)c3ccc(C(=O)N[C@@H](CCC(=O)NC(CSSCCOC(=O)OCC(=O)C4(O)C(C)CC5C6CCC7=CC(=O)C=CC7(C)[C@@]6(F)C(O)CC54C)C(=O)O)C(=O)O)s3)ccc2[nH]1]

In certain embodiments, the compound comprises (e.g., consists of) one of the following structures:

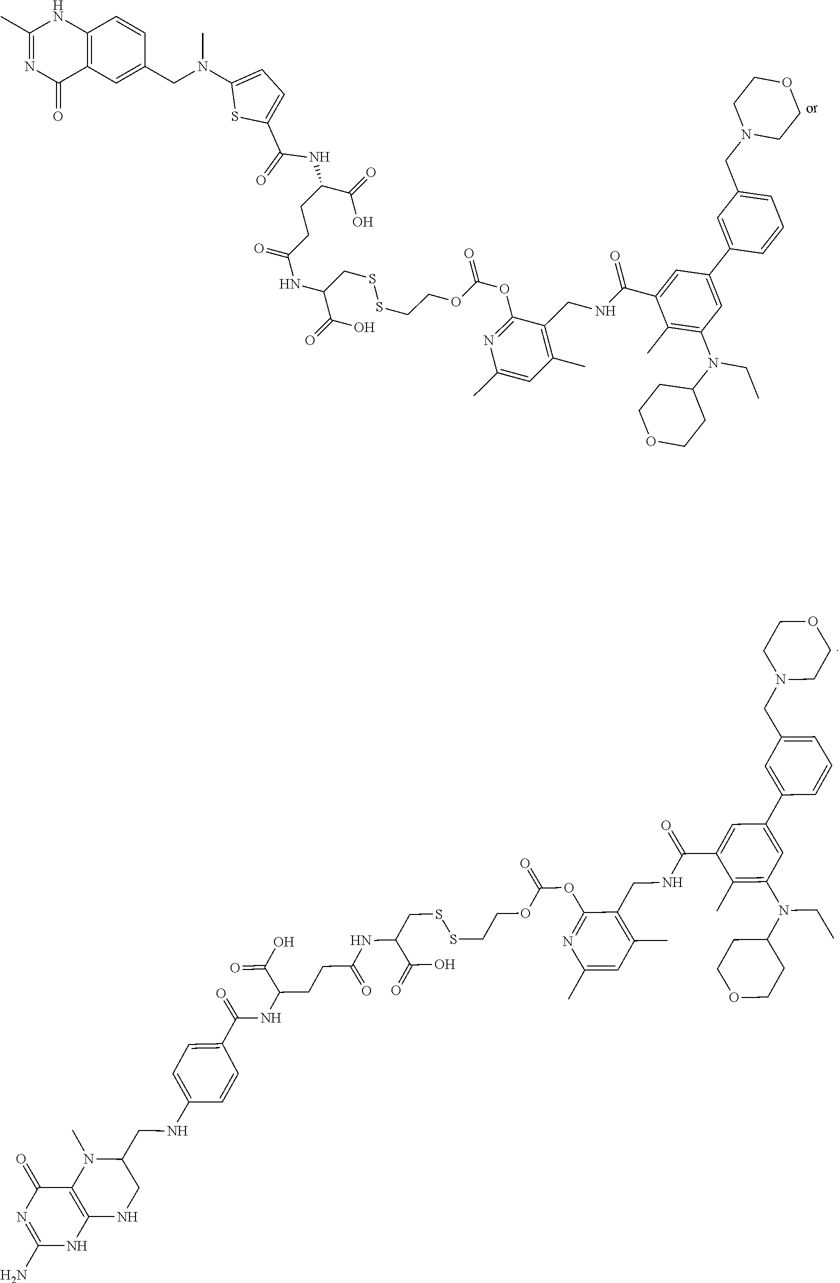 [SMILES: CCN(c1cc(-c2cccc(CN3CCOCC3)c2)cc(C(=O)NCc2c(C)cc(C)nc2OC(=O)OCCSSCC(NC(=O)CC[C@H](NC(=O)c2ccc(N(C)Cc3ccc4[nH]c(C)nc(=O)c4c3)s2)C(=O)O)C(=O)O)c1C)C1CCOCC1 ; CCN(c1cc(-c2cccc(CN3CCOCC3)c2)cc(C(=O)NCc2c(C)cc(C)nc2OC(=O)OCCSSCC(NC(=O)CCC(NC(=O)c2ccc(NCC3CNc4[nH]c(N)nc(=O)c4N3C)cc2)C(=O)O)C(=O)O)c1C)C1CCOCC1]

Any of the above compounds can further comprise a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the linker. The compound can further comprise an albumin binding group, e.g., an albumin binding group selected from the group consisting of:

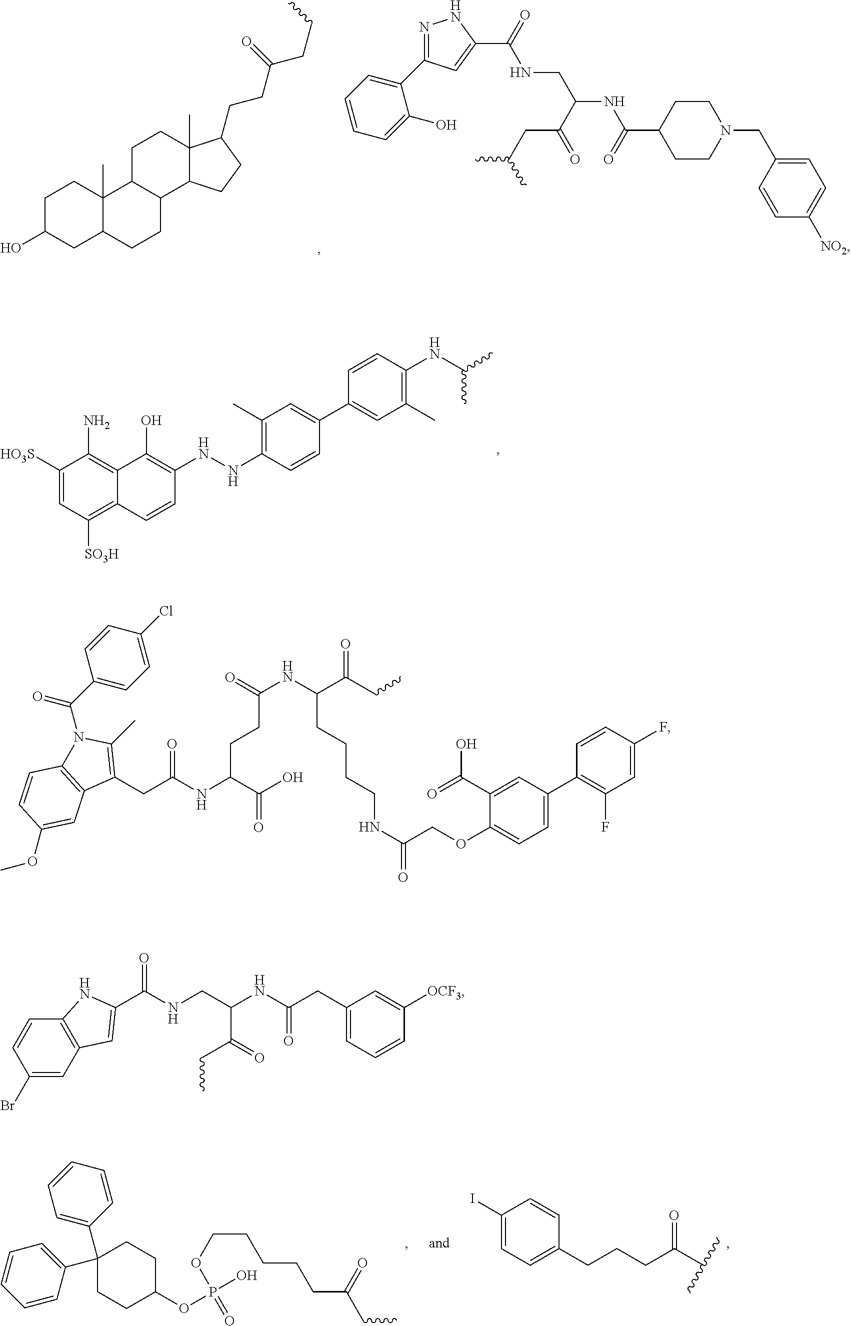

e.g, an albumin binding group selected from a group consisting of

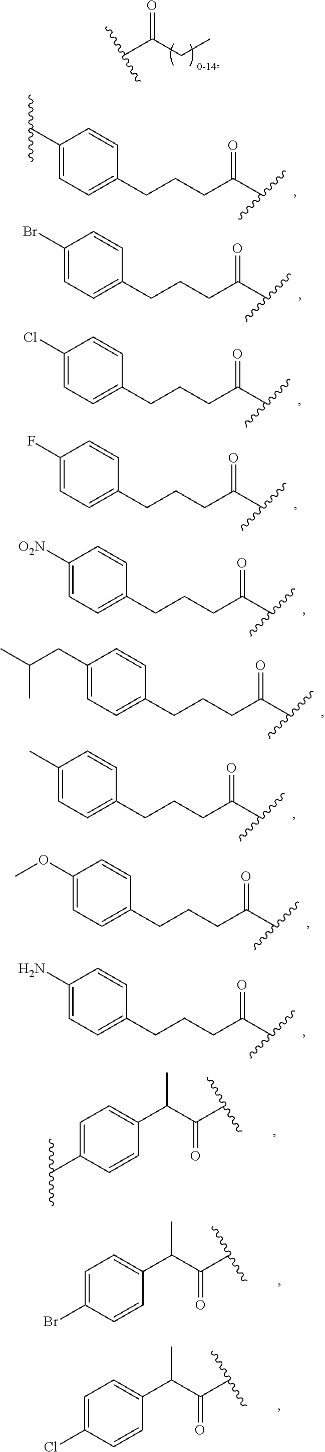

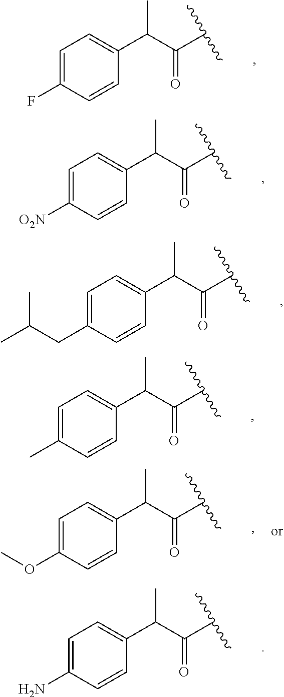

A pharmaceutical composition is also provided. In certain embodiments, provided herein are pharmaceutical compositions comprising a compound described herein (e.g., a compound of Formula (I)) and one or more pharmaceutically acceptable excipients. The pharmaceutical composition can further comprise a second compound of formula

F-L′-G,

or a pharmaceutically acceptable salt thereof, wherein F is a radical of folate or an analog thereof; L′ is a linker; and G is a radical of glucosamine.

A combination of pharmaceutical compositions is also provided. The combination can comprise (i) an aforementioned pharmaceutical composition (e.g., a pharmaceutical composition comprising a compound of the formula (I) or a pharmaceutically acceptable salt thereof), and (ii) a pharmaceutical composition comprising a compound of formula F-L′-G or a pharmaceutically acceptable salt thereof, wherein F is a radical of folate or an analog thereof; L′ is a linker; and G is a radical of glucosamine. In certain embodiments, (i) and (ii) can be administered by the same route. In certain embodiments, (i) and (ii) can be administered by different routes. Whether administered by the same route or different routes, the combination can be administered simultaneously or sequentially in either order.

Further provided is a method of immunomodulating regulatory T cells (Tregs) in a subject in need thereof. In certain embodiments, the method comprises administering to the subject an effective amount of a first compound (e.g., a compound of the formula (I) or a pharmaceutically acceptable salt thereof) or a pharmaceutical composition comprising the first compound. The method can further comprise administering a second compound of formula F-L′-G or a pharmaceutically acceptable salt thereof, wherein F is a radical of folate or an analog thereof; L′ is a linker; and G is a radical of glucosamine. Administering the second compound can be performed simultaneously or sequentially with the first compound or first pharmaceutical composition in either order, by the same or different routes.

In certain embodiments of the method, the subject has cancer, E (of the first compound) is a radical of a TLR7 agonist, a PI3k inhibitor, a NLR2 agonist, a STING agonist, an EZH2 inhibitor, an NLRP3 inhibitor, a Caspase I inhibitor, or an RLR agonist, and administration of an effective amount of the first compound or first pharmaceutical composition alters Tregs' promotion of tumor growth and metastasis and/or inhibition of anti-tumor immunity in the subject. The method can further comprise administering to the subject a third therapeutic agent, such as an anti-cancer agent. The anti-cancer agent can be, for example, a chemotherapeutic agent or a radiotherapeutic agent.

The method can further comprise administering to a subject a second compound of formula F-L′-G or a pharmaceutically acceptable salt thereof, wherein F is a radical of folate or an analog thereof; L′ is a linker; and G is a radical of glucosamine, wherein administering the second compound can be performed simultaneously or sequentially with administering the first compound, in either order and by the same or different routes.

In certain embodiments of the method, the subject has a fibrotic disease or disorder, and E of the first compound (e.g., a compound of the formula (I) or a pharmaceutically acceptable salt thereof) or the first pharmaceutical composition is a radical of a therapeutic agent selected from the group consisting of a TLR7 agonist, a PI3k inhibitor, a steroid, a NLR2 agonist, a STING agonist, an EZH2 inhibitor, a NLRP3 inhibitor, a Caspase I inhibitor, and a RLR agonist. The method can further comprise administering a second compound of formula F-L′-G or a pharmaceutically acceptable salt thereof, wherein F is a radical of folate or an analog thereof; L′ is a linker; and G is a radical of glucosamine, wherein administering the second compound can be simultaneously or sequentially with administering the first compound, in either order and by the same or different routes.

In yet another embodiment of the method, the subject has an inflammatory disease, and the method comprises administering an effective amount of the first compound (e.g., a compound of the formula (I) or a pharmaceutically acceptable salt thereof) or the first pharmaceutical composition, in which E is a radical of a steroid. The method can further comprise administering a second compound of formula F-L′-G or a pharmaceutically acceptable salt thereof, wherein F is a radical of folate or an analog thereof; L′ is a linker; and G is a radical of glucosamine, wherein administering the second compound can be performed simultaneously or sequentially with administration of the first compound, in either order and by the same or different routes.

Further provided herein is a method of treating cancer in a subject (e.g., a subject in need thereof), comprising administering to the subject a therapeutically effective amount of a first compound (e.g., a compound of the formula (I) or a pharmaceutically acceptable salt thereof).

Further provided herein is a method of treating a fibrotic disease or disorder in a subject in need thereof, comprising administering to the subject a therapeutically effective amount of a compound described herein (e.g., a compound of the formula (I)) or a pharmaceutically acceptable salt thereof.

Further provided herein is a method of treating an inflammatory disease in a subject (e.g., a subject in need thereof), comprising administering to the subject a therapeutically effective amount of a compound described herein (e.g., a compound of the formula (I)) or a pharmaceutically acceptable salt thereof.

### DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIGS. 1A-1D show data from an in silico analysis of the binding affinity to mouse and human folate receptor delta (FRM) of several folate analogs, wherein FIG. 1A shows structures of folic acid and raltitrexed; FIG. 1B shows predicted relative binding free energies (MMGBSA dGbind) and docking scores (XP GScore) for the binding of folic acid analogs to human FRδ (PDB: 5F4Q) and mouse FRδ (PDB: 5JYJ); FIG. 1C shows binding poses of raltitrexed on human FRδ; FIG. 1D shows binding poses of raltitrexed on mouse FRδ. The ribbon diagrams display the ligand-protein interactions while surface topographies reveal the electrostatic potential maps and binding orientations of the ligand in the FRδ binding cavity. In FIGS. 1C and 1D, areas labeled “blue” represent positively charged regions and areas labeled “red” represent negatively charged regions of the binding site.

FIGS. 2A and 2B show Raltitrexed-S0456 (Ral-S056) and Folate-S456 targeting a tumor environment in vivo, wherein FIG. 2A shows imaging of tumor-bearing mice taken four hours post-injection with 10 nmol of Raltitrexed-S0456 (R-S0456) with or without 200× of Raltitrexed-glucosamine (Ral-Glucosamine) and 10 nmol of Folate-S0456 with or without 200× of Folate-glucosamine. The glucosamine conjugates served as competing ligands and were compared to a negative control; and FIG. 2B shows imaging of the major organs taken from treated mice shown in FIG. 2A.

FIGS. 3A-3K show the gating strategy and the flow cytometric analysis of digested tumors and spleen from the treated mice, where FIG. 3A shows the flow cytometry scatter plot results for live cells; FIG. 3B shows the flow cytometry scatter plot results for cells labeled with anti-CD45 antibody; FIG. 3C shows the flow cytometry scatter plot results for cells labeled with anti-CD4 and anti-CD25 antibodies; FIG. 3D shows the flow cytometry scatter plot results for cells labeled with anti-CD127 and anti-FRδ antibodies; FIG. 3E shows the uptake of Ral-S0456 by CD45⁺CD4⁺CD25⁺CD127⁺ FRδ⁺ Tregs isolated from murine tumor and spleen, with or without 200λ Ral-Glucosamine competition, as compared to Folate-S0456 and an unstained negative control; FIG. 3F shows a comparison of the uptake of Ral-S0456 and Folate-S0456 by different white blood cell populations in the tumors; FIG. 3G shows results that support no binding of Ral-S0456 or Folate-S0456 to CD45− cells conjugates; FIG. 3H shows graphical data supporting no binding of Ral-S0456 or Folate-S0456 to CD45+CD8+ cytotoxic T cells; FIG. 3I shows graphical results supporting no binding of Ral-S0456 or Folate-S0456 to CD45⁺CD4⁺CD25⁻FRδ⁻ cells;

FIG. 3J shows graphical results supporting some binding of Ral-S0456, but significantly higher binding of Folate-S0456, to CD45⁺CD11b⁺F4/80 macrophages; and FIG. 3K shows graphical data supporting binding of Ral-S0456 to tumor Tregs in FRδ wild type mice vs binding to Tregs in FRδ knockout mice.

FIG. 4 shows a scheme for synthesis of Raltitrexed-S0456 that was utilized in the binding studies shown in FIG. 2A-3K.

FIG. 5 shows an alternative scheme for synthesis of an embodiment of Raltitrexed-S0456 conjugate described herein (ns=nonsignificant; *=significant and P<0.05; **=significant and P<0.01).

FIG. 6 shows a scheme for the synthesis of a toll-like receptor 7 (TLR7) agonist, referred to herein as TLR7-1A.

FIGS. 7A-7E show the immunomodulatory effect of TLR7-1A on murine CD45⁺CD4⁺CD25⁺ Tregs in vitro, with murine CD45⁺CD4⁺CD25⁻ effector T cells stained with carboxyfluorescein succinimidyl ester (CFSE) dye and cell divisions tracked with flow cytometry, where FIG. 7A shows graphical data representative of the parent CFSE-labeled CD4⁺CD25⁻ population without co-cultured Tregs, treatment, or CD3/CD28 beads activation; FIG. 7B shows graphical data representative of divided CFSE-labeled CD4⁺CD25⁻ population upon activation, without co-cultured Tregs or treatment; FIG. 7C shows graphical data representative of the effect of Tregs on the division of CFSE-labeled CD4⁺CD25⁻ effector T cells when co-cultured at 1:4 ratio; FIG. 7D shows graphical data representative of the effect of pre-treating Tregs with 10 nM TLR7-1A for 3 hours before co-culturing with CFSE-labeled CD4⁺CD25⁻ cells at 1:4 ratio; and FIG. 7E shows a bar graph representation of the data of FIGS. 7A-7D.

FIG. 8 shows graphical data representing the immunomodulatory effect of TLR7-1A on murine CD45⁺CD4⁺CD25⁺ Tregs in vitro, where Tregs were pre-treated with TLR7-1A for 3 hours before being co-cultured with murine CD45⁺CD4⁺CD25⁻ effector T cells for 48 hours, after which supernatant was analyzed by enzyme-linked immunoassay (ELISA) for interleukin-10 (IL-10) and transforming growth factor beta (TGF-β) release.

FIG. 9 shows a scheme for synthesis of a Raltitrexed-TLR7-1A (Ral-TLR7-1A) releasable conjugate.

FIGS. 10A-10C show data relating to the effect of Ral-TLR7-1A on tumor growth, body weight, and immune cell composition in murine breast 4T1 subcutaneous tumors in BALB/c mice, where FIG. 10A shows a graph of the 4T1 tumor volume change over the course of treatment; FIG. 10B shows a graph of body weight change of mice over the course of treatment; and FIG. 10C shows a bar graph of the analysis of the phenotypic markers of Tregs, CD8⁺ cytotoxic T cells and macrophages, with the relevant phenotypic markers listed on each y-axis and the treatment regimen indicated on each x-axis as 1 (untreated control), 2 (Ral-TLR7-1A), 3 (Ral-TLR7-1A+Ral-Gluc), or 4 (Raltitrexed).

FIG. 11 shows graphs of data resulting from the evaluation of the effect of Ral-TLR7-TA on phenotypic markers of splenic Tregs and CD8⁺ cytotoxic T cells isolated from the tumor-bearing mice of FIG. 10A, with the relevant phenotypic markers listed on each y-axis and the treatment regimen indicated on each x-axis as 1 (untreated control), 2 (Ral-TLR7-TA), 3 (Ral-TLR7-TA+Ral-Gluc), or 4 (Raltitrexed).

FIG. 12 shows graphs of data representing the effect of Ral-TLR7-TA on tumor growth and body weight in murine colorectal CT26 subcutaneous tumors in BALB/c mice.

FIGS. 13A and 13B show graphical data relating to the effect of Ral-TLR7-1A on tumor growth, body weight, and immune cells composition in murine bladder MB49 subcutaneous tumor in FRβ knockout C57BL/6 mice, with FIG. 13A showing plots of MB49 tumor volume change and mice body weight change over the course of the treatment; and FIG. 13B showing a graph of the analysis of the phenotypic markers of Tregs and CD8⁺ cytotoxic T cells.

FIG. 14 shows a scheme for synthesis of a Ral-dexamethasone described herein and used in Example 10.

FIGS. 15A and 15B show graphical data related to the effect of Ral-TLR7-TA or Ral-dexamethasone on tumor growth and immune cells composition in murine breast 4T1 subcutaneous tumor in BALB/c mice, with FIG. 15A showing 4T1 tumor volume change over the course of the treatment; and FIG. 15B showing an analysis of the phenotypic markers of Tregs and CD8⁺ cytotoxic T cells.

While the present disclosure is susceptible to various modifications and alternative forms, exemplary embodiments thereof are shown by way of example in the drawings and are herein described in detail.

### DETAILED DESCRIPTION

For the purposes of promoting an understanding of the principles hereof, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology is illustrated and described in one or more preferred embodiments, the compounds, compositions, and methods of use thereof can comprise many different configurations, forms, materials, and accessories.

### Compounds

The present disclosure is predicated, at least in part, on the design of conjugates comprising immunomodulatory small molecules conjugated to a suitable targeting ligand such as raltitrexed or 5-methyltetrahydrofolate (5-MTHF). The compounds (e.g., immunomodulatory small molecules) can alter regulatory T cells' (Tregs) promotion of tumor growth and metastasis and/or inhibition of anti-tumor immunity. The compounds can be internalized by the target cell upon ligand binding, thereby reducing, if not eliminating, off-target effects and toxicity.

T can be any suitable targeting ligand, such as a folate mimetic (i.e., a compound other than folic acid that mimics folic acid and can be bound by folate receptor beta (FRβ) or folate receptor delta (FRδ)). T can be an antifolate. An antifolate can specifically bind to FRδ with relative affinity of about 0.05 or greater compared to folic acid at a temperature above about 20° C./25° C./30° C./physiological temperature.

A non-limiting example of a suitable antifolate is raltitrexed. Analogs and derivatives of folic acid, such as 5-MTHF, also can be suitable targeting ligands. Other suitable targeting ligands can be identified, for example, by screening for binding to FRδ on isolated Tregs and determining IC₅₀ (see, e.g., Example 2). Examples of targeting ligands that can be screened for binding to FRδ include, but are not limited to, folate analogs such as folinic acid, pteropolyglutamic acid, folate receptor-binding pteridines such as tetrahydropterins, dihydrofolates, tetrahydrofolates, and their deaza and dideaza analogs. “Deaza” and “dideaza” analogs refer to the art-recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, the 1,5 dideaza, 5,10-dideaza, 8,10-dideaza, and 5,8-dideaza analogs. Other examples of targeting ligands that can be screened for binding to FRδ include, but are not limited to, pemetrexed, proguanil, pyrimethamine, trimethoprim, pralatrexate, aminopterin, amethopterin (methotrexate), N10-methylfolate, 2-deamino-hydroxyfolate, and deaza analogs thereof, such as 1-deazamethopterin or 3-deazamethopterin, and 3′,5′-dichloro-4-amino-4-deoxy-N10-methylpteroylglutamic acid (dichloromethotrexate).

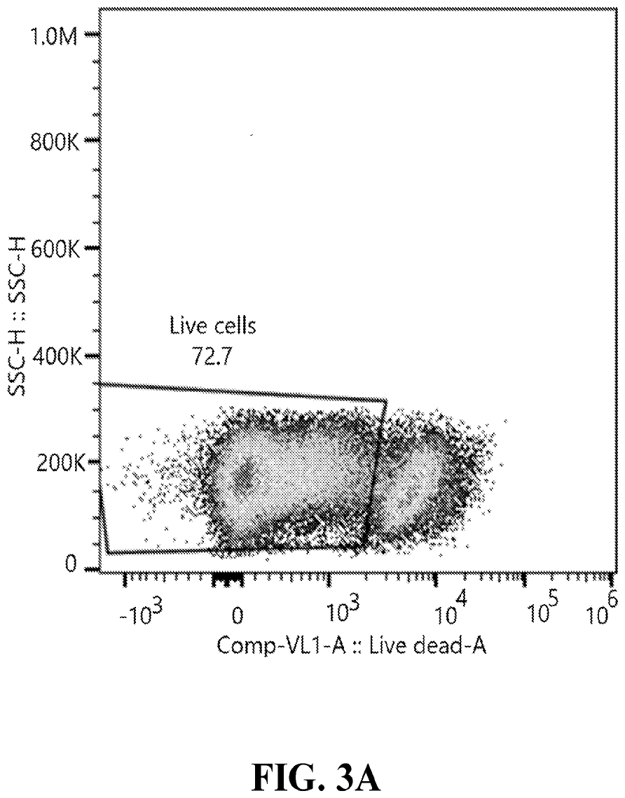

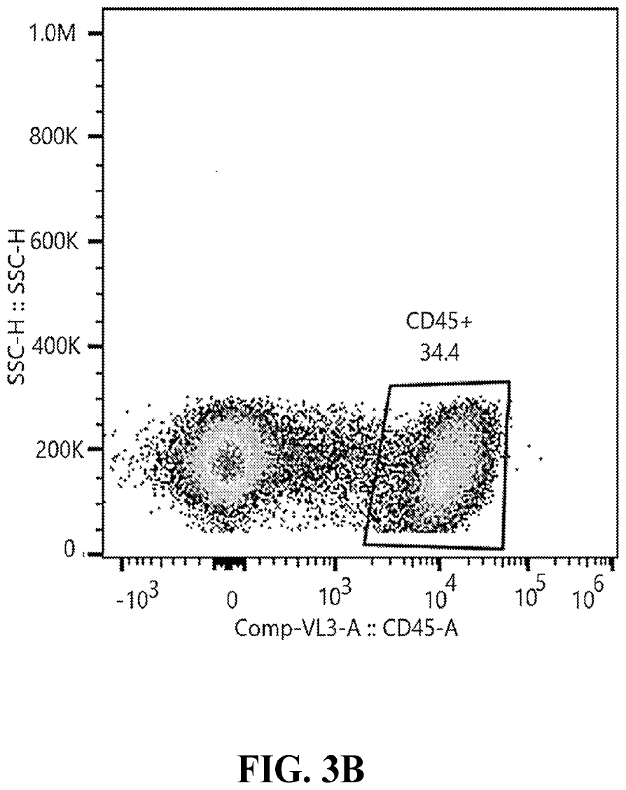

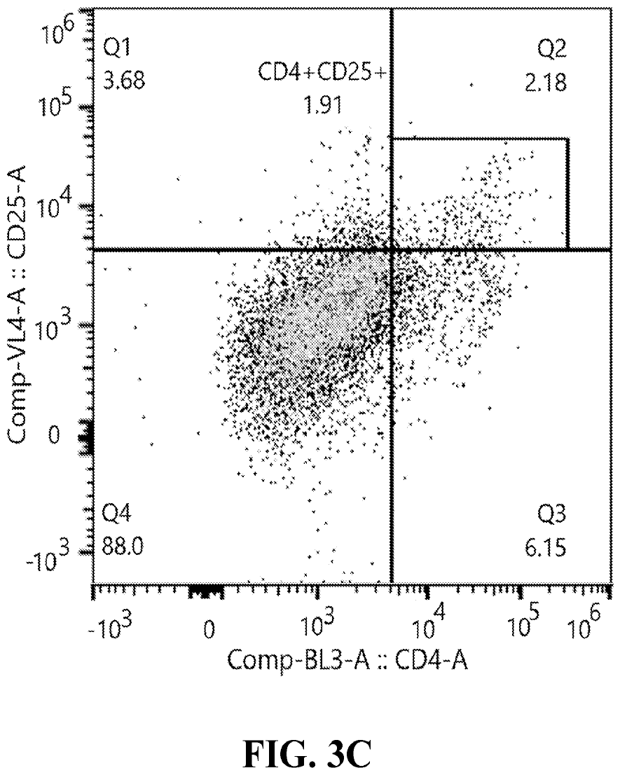

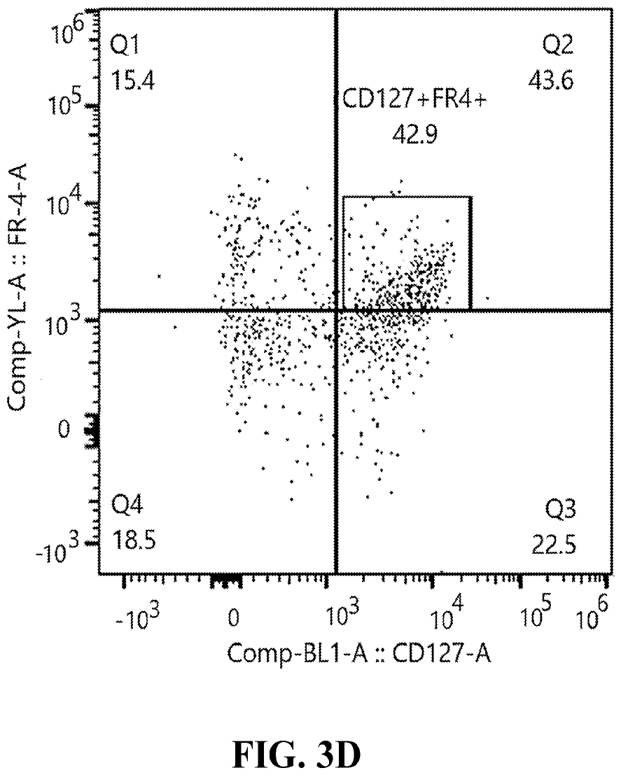

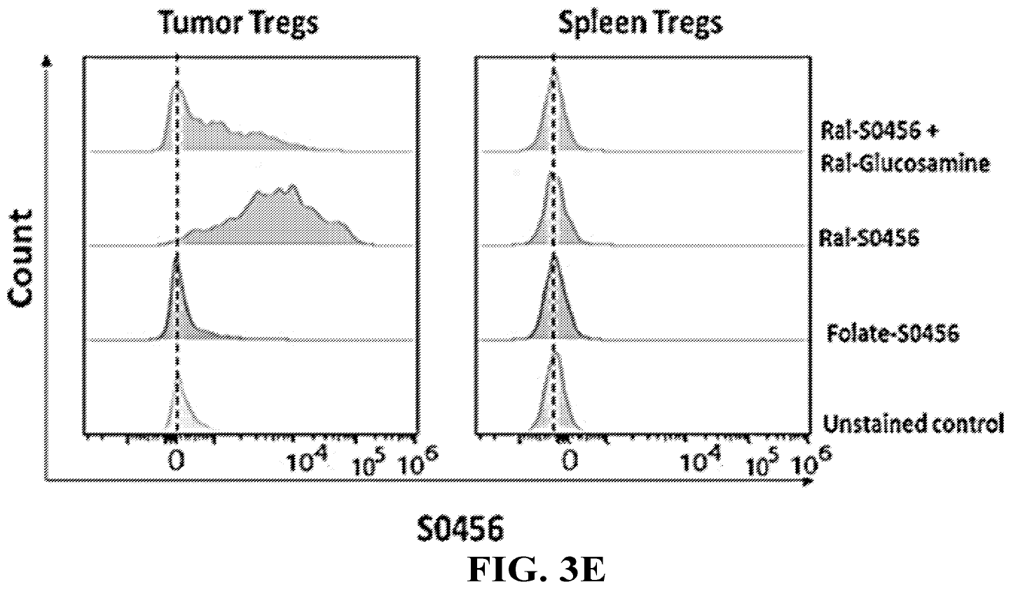

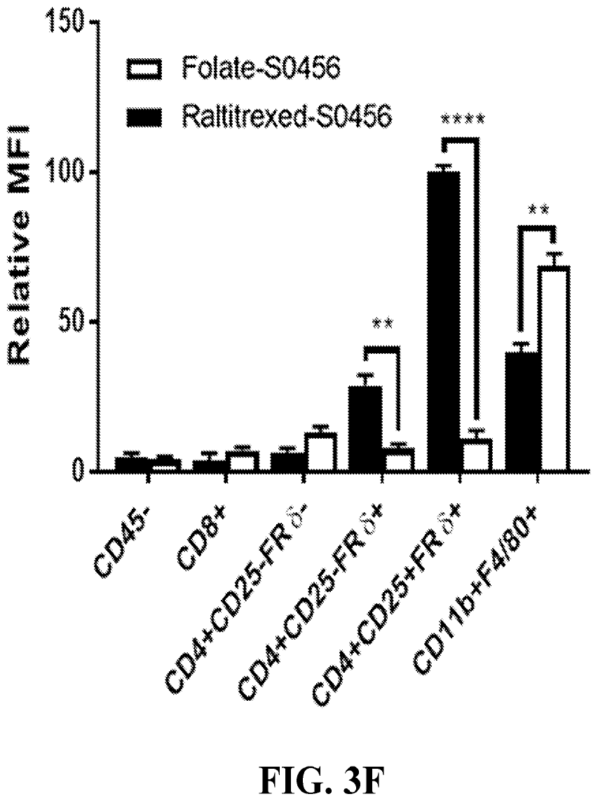

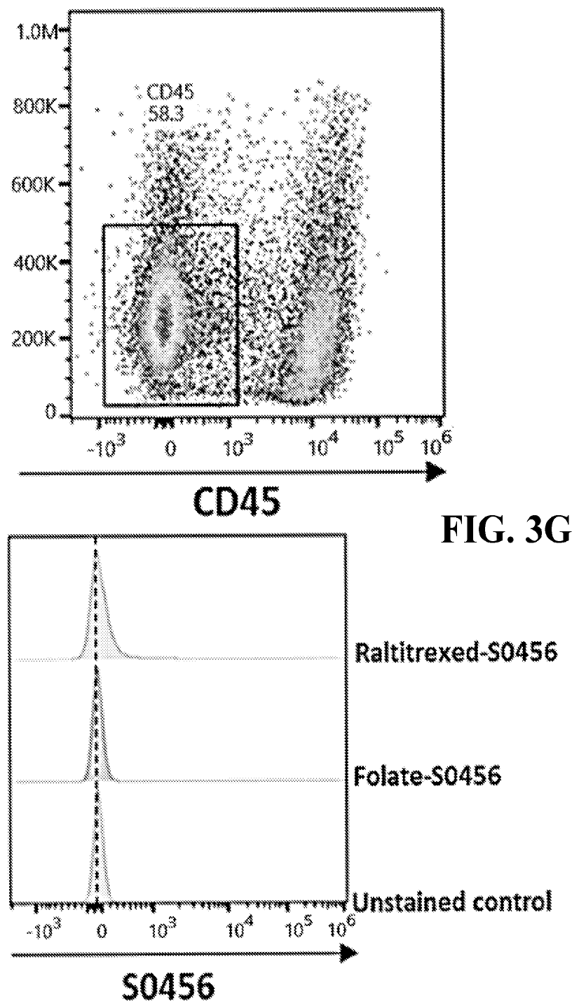

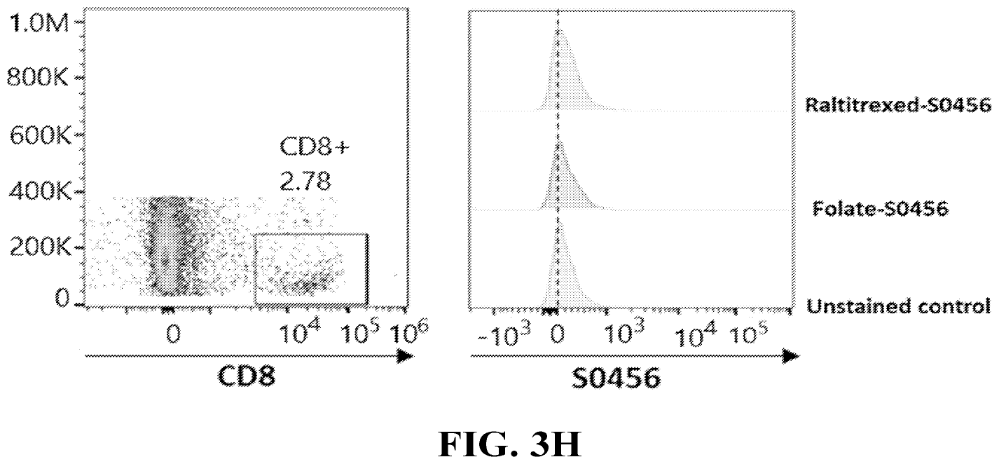

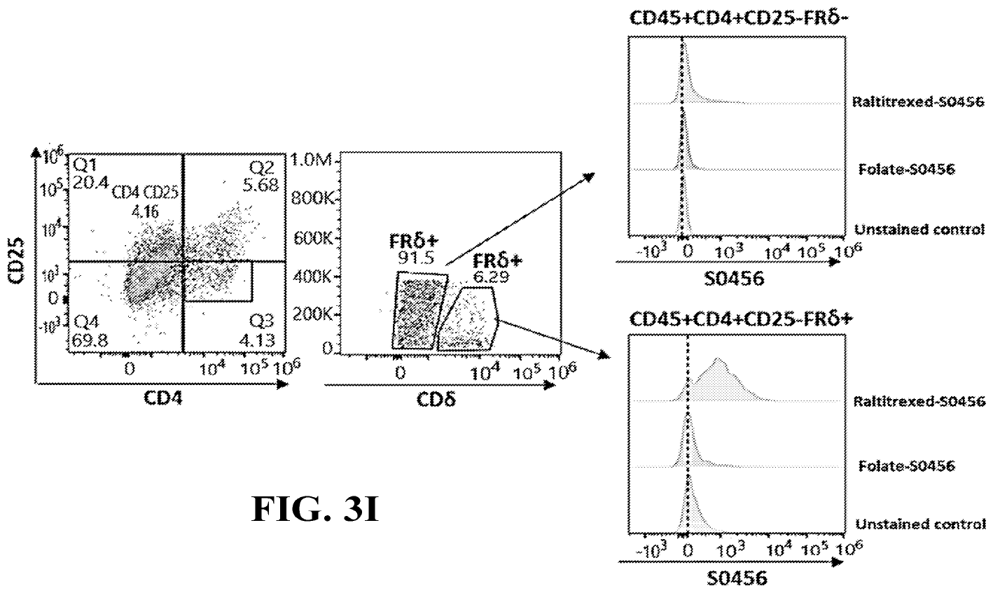

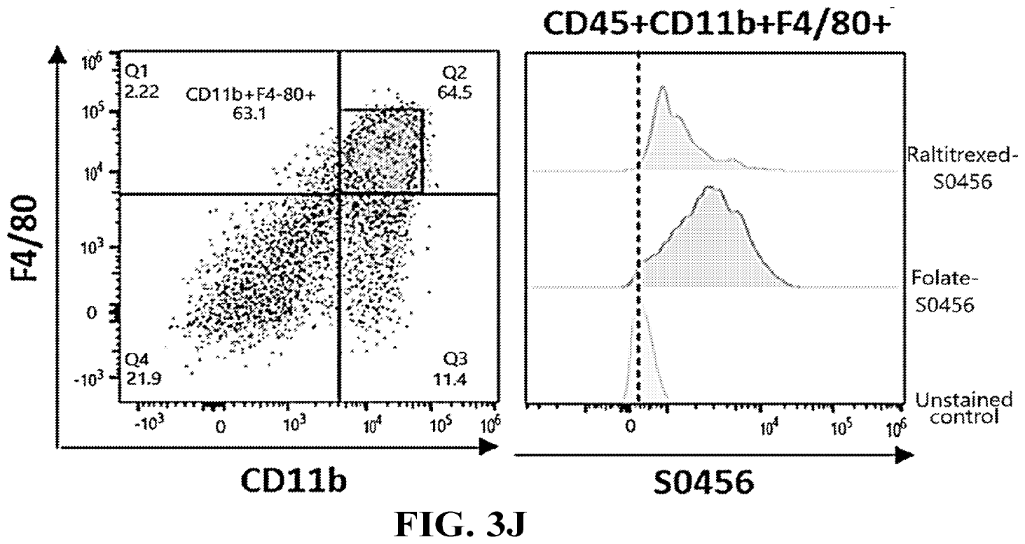

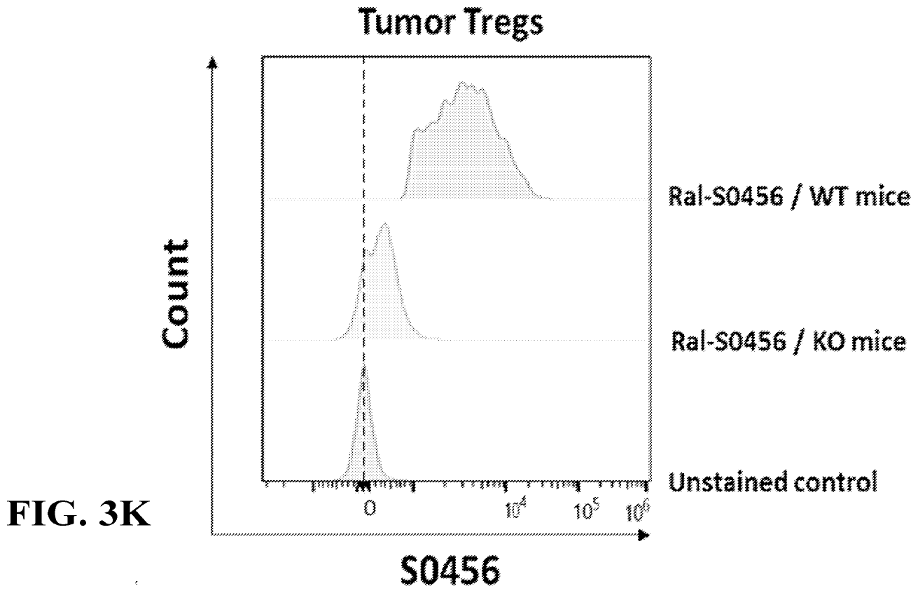

In certain embodiments, T could have a higher binding affinity for FRδ than FRβ (i.e., T could “preferentially” bind FRδ over FRβ). In certain embodiments, T could preferentially bind FRδ over FRβ at a ratio of about 1.5:1, about 2:1, or about 2.5:1 (see, e.g., the cell binding data shown in FIG. 3F, where the second to last column relates to uptake by Tregs (FRδ) and the last column relates to uptake by macrophages (FRβ), and wherein the ratio of uptake by Tregs to uptake by macrophages is approximately the same ratio as FRδ binding to FRβ binding).

Binding assays can be used to determine binding affinity and/or a ratio of binding affinity for a ligand hereof with respect to FRδ versus other folate isoforms (e.g. folate receptor alpha (FRα), FRβ, etc.). For example, and without limitation, comparative binding affinity can be determined in accordance with the methodologies exemplified in Example 3. In certain embodiments, comparative binding affinity can be determined by transfecting a cell line (e.g., a HEK293 cell line) with either mouse or human folate receptor isoforms (e.g., FRα, FRβ, or FRδ). When expression is confirmed, a ligand's binding affinity can be tested for each receptor of interest by incubating the cells with different, incremental concentrations of the different analogs linked to a dye (e.g., S0456 NIR dye) that can be detected and quantified (e.g., by flow cytometry). The relative binding of each ligand to each receptor isoform can then be calculated based on the quantified binding data to determine which ligand has a higher binding affinity (i.e. “preferentially binds”) for each receptor isoform as compared to the other ligands tested.

The present disclosure, in part, further provides a compound of the Formula (I):

T-L-E (I)

or a pharmaceutically acceptable salt thereof, wherein T is a radical of raltitrexed, 5-MTHF, or an analog thereof; L is a linker; and E is a radical of a therapeutic agent. In certain embodiments, T is a radical of raltitrexed. In other embodiments, T is a radical of 5-MTHF.

Further the present disclosure further provides a compound of the Formula (I′):

T′-L-E (I′)

or a pharmaceutically acceptable salt thereof, wherein T′ is a targeting moiety (e.g., a radical of raltitrexed, 5-MTHF, an analog of raltitrexed, or an analog of 5-MTHF, a compound of Formula I-VII, Formula II-VII, Formula III-VII, Formula IV-VII, Formula V-VII, Formula VI-VII, or Formula VII-VII, or a compound of Table I, II, or III, as described herein); L is a linker; and E is a radical of a therapeutic agent (e.g., a radical of a therapeutic agent described herein). In some embodiments, T binds to a receptor of a cell. In some embodiments, T binds to a pattern recognition receptor in a cell. In some embodiments, T binds to an immune cell receptor. In some embodiments, T selectively binds to a folate receptor. In some embodiments, T selectively binds to FRβ. In some embodiments, T selectively binds to FRδ or preferentially binds to FRδ as compared to FRβ.

In some embodiments, T is a radical that can have the structure of Formula I-VII:

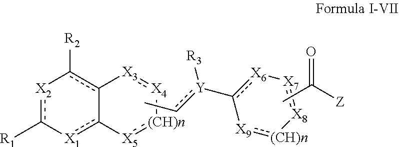

wherein:
X₁, X₂, X₃, X₄, X₅, X₆, X₇, X₈, and X₉ are each independently N, NH, CH, CH₂, O or S;
Y is C, CH, CH₂, N, NH, O, or S;
Z is glutamic acid, valine, or suberate;
R₁ and R₂ are each independently NH₂, OH, SH, CH₃, or H;
R₃ is H or an alkyl;
n is 0-1; and
[symbol] is a single C—C bond or a double C═C bond.

In some embodiments, T is a radical of a compound of Formula I-VII that can further have the structure of Formula II-VII or Formula III-VII:

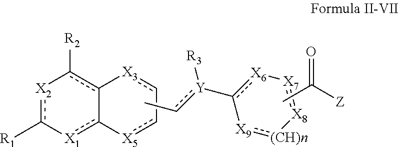

wherein:
X₁, X₂, X₃, X₅, X₆, X₇, X₈, and X₉ are each independently N, NH, CH, CH₂, O or S;
Y is C, CH, CH₂, N, NH, O, or S;
Z is glutamic acid, valine, or suberate;
R₁ and R₂ are each independently NH₂, OH, SH, CH₃, or H;
R₃ is H or an alkyl;
n is 0-1; and
[symbol] is a single C—C bond or a double C═C bond; or

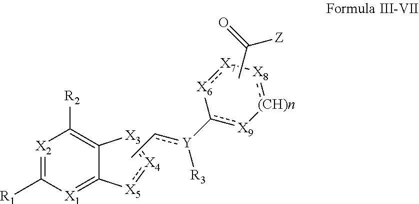

wherein:
X₁, X₂, X₃, X₄, X₅, X₆, X₇, X₈, and X₉ are each independently N, NH, CH, CH₂, O or S;
Y is C, CH, CH₂, N, NH, O, or S;
Z is glutamic acid, valine, or suberate;
R₁ and R₂ are each independently NH₂, OH, SH, CH₃, or H;
R₃ is H or an alkyl;
n is 0-1; and
[symbol] is a single C—C bond or a double C═C bond.

In some embodiments, T is a radical of a compound of Formula II-VII that can further have the structure of a Formula IV-VII or Formula V-VII:

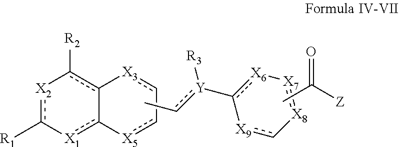

wherein:
X₁, X₂, X₃, X₅, X₆, X₇, X₈, and X₉ are each independently N, NH, CH, CH₂, O or S;
Y is C, CH, CH₂, N, NH, O, or S;
Z is glutamic acid, valine, or suberate;
R₁ and R₂ are each independently NH₂, OH, SH, CH₃, or H;
R₃ is H or an alkyl; and
[symbol] is a single C—C bond or a double C═C bond; or

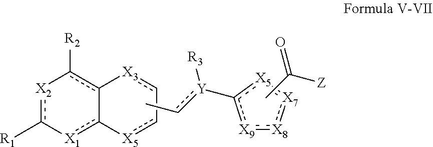

wherein:
X₁, X₂, X₃, X₅, X₆, X₇, X₈, and X₉ are each independently N, NH, CH, CH₂, O or S;
Y is C, CH, CH₂, N, NH, O, or S;
Z is glutamic acid, valine, or suberate;
R₁ and R₂ are each independently NH₂, OH, SH, CH₃, or H;
R₃ is H or an alkyl; and
[symbol] is a single C—C bond or a double C═C bond; or
In some embodiments, T is a radical of a compound of Formula III-VII that can further have the structure of a Formula VI-VI or Formula V-VII:

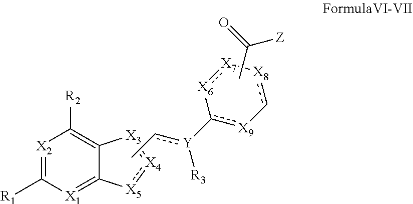

wherein:
X₁, X₂, X₃, X₄, X₅, X₆, X₇, X₈, and X₉ are each independently N, NH, CH, CH₂, O or S;
Y is C, CH, CH₂, N, NH, O, or S;
Z is glutamic acid, valine, or suberate;
R₁ and R₂ are each independently NH₂, OH, SH, CH₃, or H;
R₃ is H or an alkyl; and
[symbol] is a single C—C bond or a double C═C bond; or

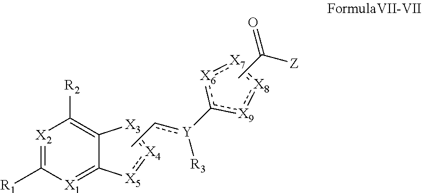

wherein:
X₁, X₂, X₃, X₄, X₅, X₆, X₇, X₈, and X₉ are each independently N, NH, CH, CH₂, O or S;
Y is C, CH, CH₂, N, NH, O, or S;
Z is glutamic acid, valine, or suberate;
R₁ and R₂ are each independently NH₂, OH, SH, CH₃, or H;
R₃ is H or an alkyl; and
[symbol] is a single C—C bond or a double C═C bond; or
In some embodiments, T is a radical of a compound of Formula IV-VII that can have a structure selected from Table I.

TABLE I

| | | |
|---|---|---|
| 1. | An FRδ-binding derivative of analog of folic acid, which has the structure | 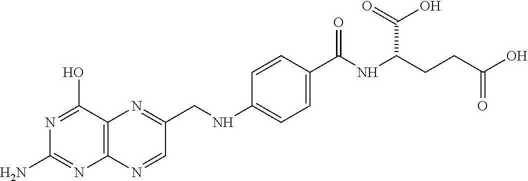 [SMILES: Nc1nc(O)c2nc(CNc3ccc(C(=O)N[C@@H](CCC(=O)O)C(=O)O)cc3)cnc2n1] |
| 2. | Methotrexate | 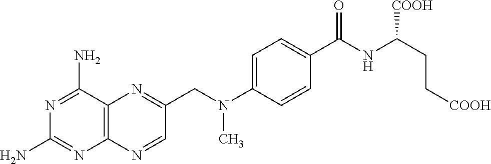 [SMILES: CN(Cc1cnc2nc(N)nc(N)c2n1)c1ccc(C(=O)N[C@@H](CCC(=O)O)C(=O)O)cc1] |
| 3. | Folinic acid | 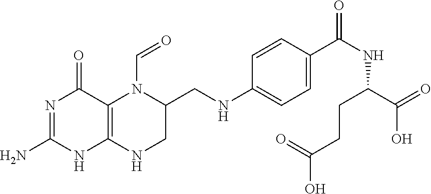 [SMILES: Nc1nc(=O)c2c([nH]1)NCC(CNc1ccc(C(=O)N[C@@H](CCC(=O)O)C(=O)O)cc1)N2C=O] |
| 4. | Pralatrexate | 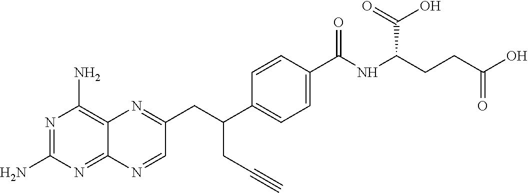 [SMILES: C#CCC(Cc1cnc2nc(N)nc(N)c2n1)c1ccc(C(=O)N[C@@H](CCC(=O)O)C(=O)O)cc1] |
| 5. | | 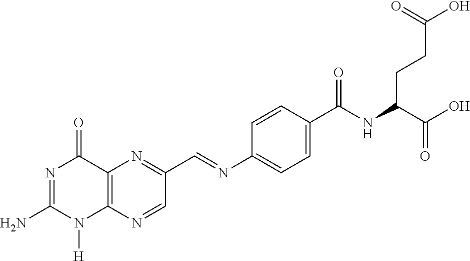 |
| 6. | Int'l Pat App Pub No. WO2010/0033733 | 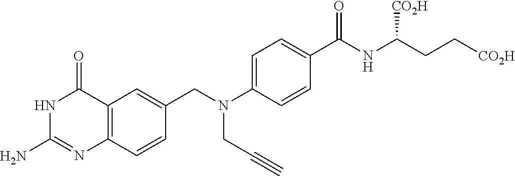 [SMILES: C#CCN(Cc1ccc2nc(N)[nH]c(=O)c2c1)c1ccc(C(=O)N[C@@H](CCC(=O)O)C(=O)O)cc1] |
| 7. | Int'l Pat App Pub No. WO2010/0033733 | 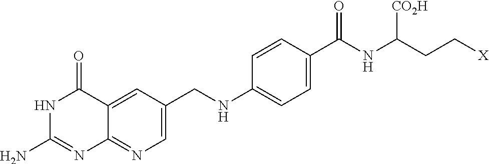 |
| 8. | Int'l Pat App Pub No. WO2010/0033733 | 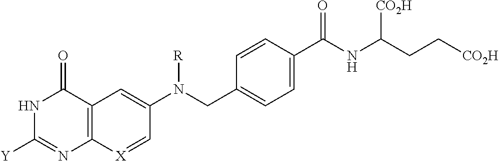 |
| 9. | Int'l Pat App Pub No. WO2010/0033733 | ` 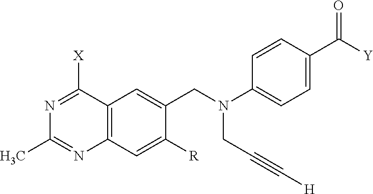 |

In some embodiments, T is a radical of a compound of Formula V-VII that can have a structure selected from Table II.

TABLE II

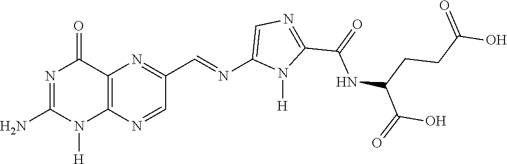

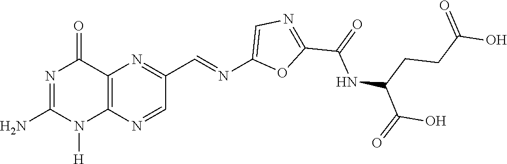

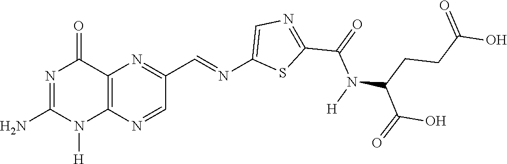

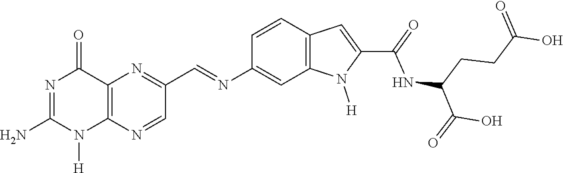

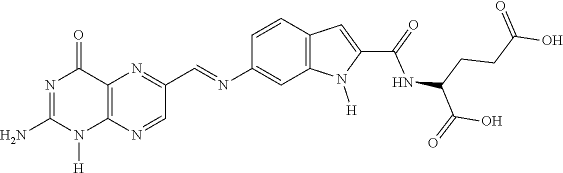

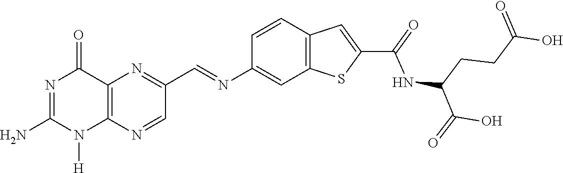

In some embodiments, T is a radical of a compound of Formula VI-VII that can have a structure selected from Table III.

TABLE III

| | | |
|---|---|---|
| 1. | Pemetrexed | 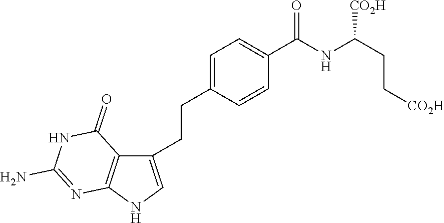 [SMILES: Nc1nc2[nH]cc(CCc3ccc(C(=O)N[C@@H](CCC(=O)O)C(=O)O)cc3)c2c(=O)[nH]1] |
| 2. | | 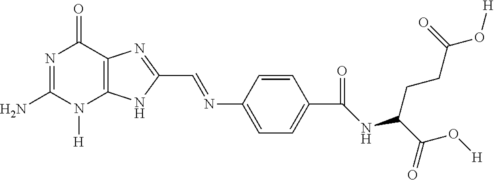 |
| 3. | | 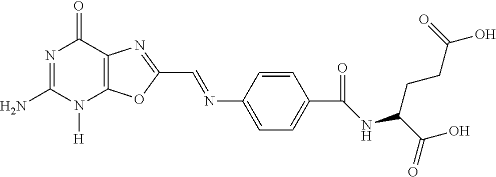 |
| 4. | | 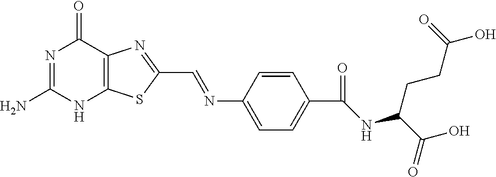 |

In certain embodiments, T is any suitable targeting ligand that can be bound by FRδ. In certain embodiments, T can be a radical of any of the following structures:

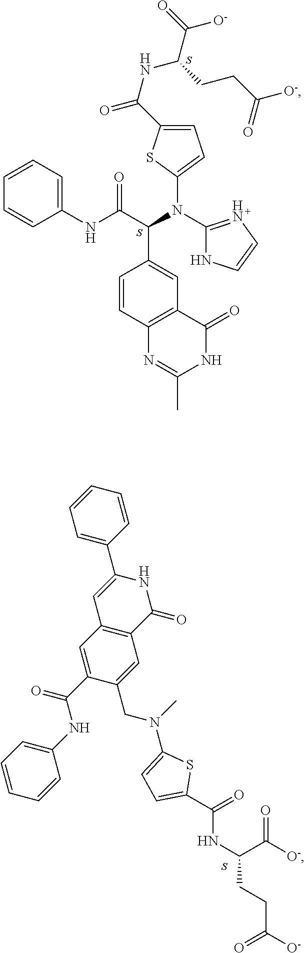

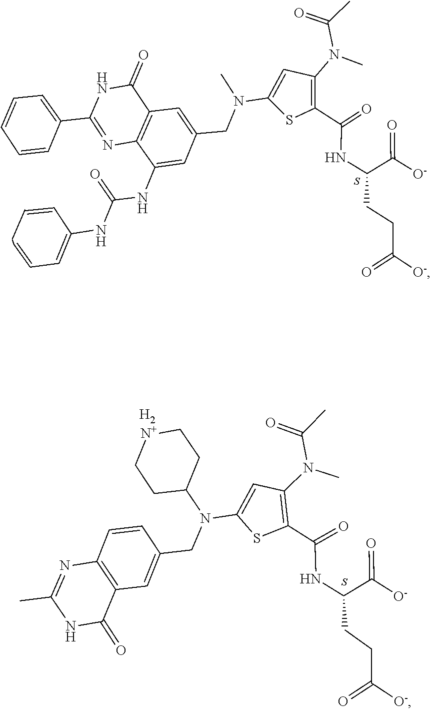

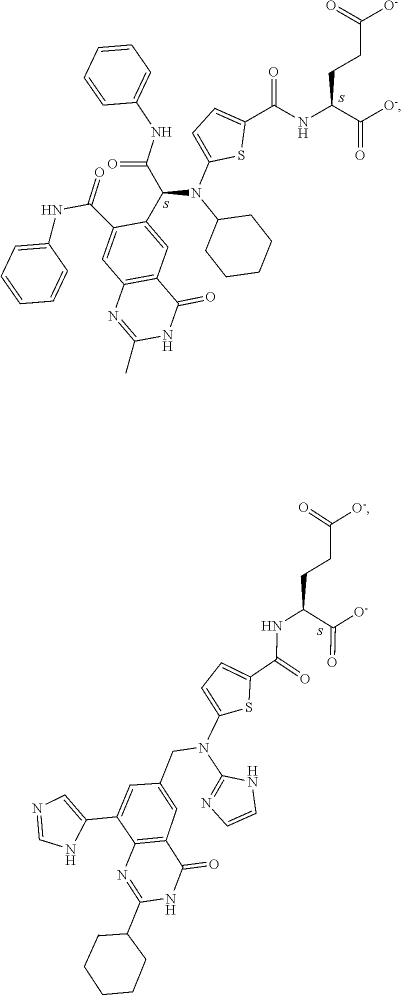

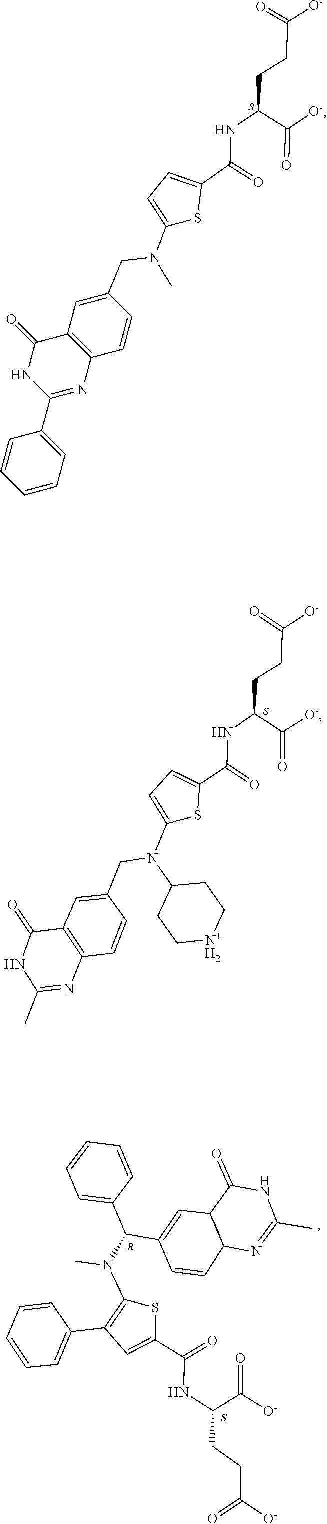

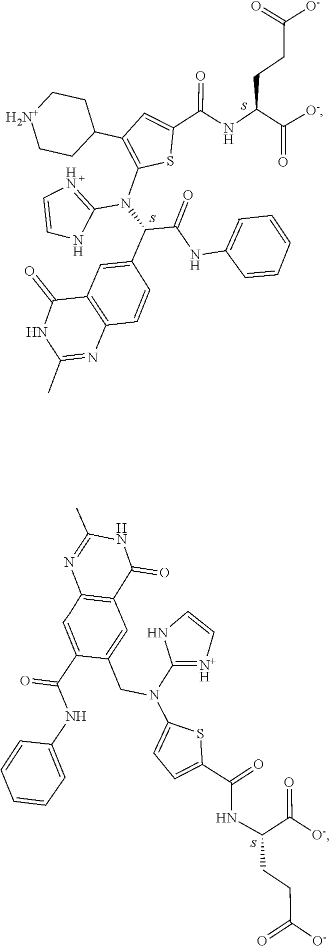

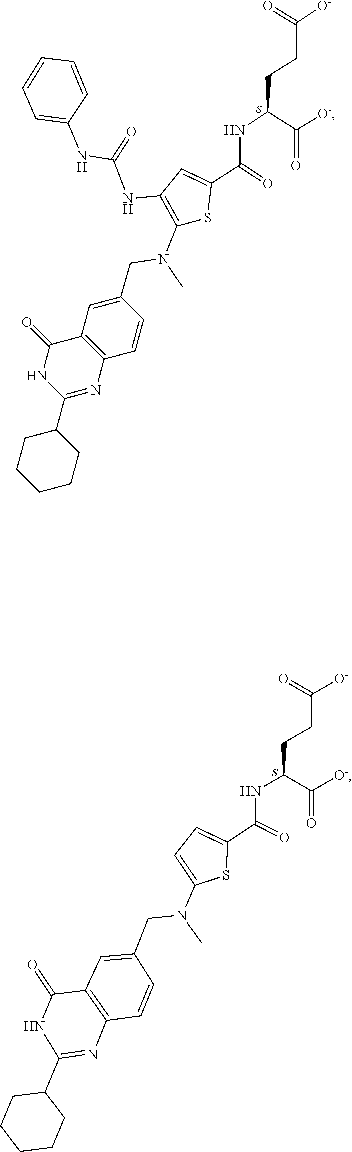

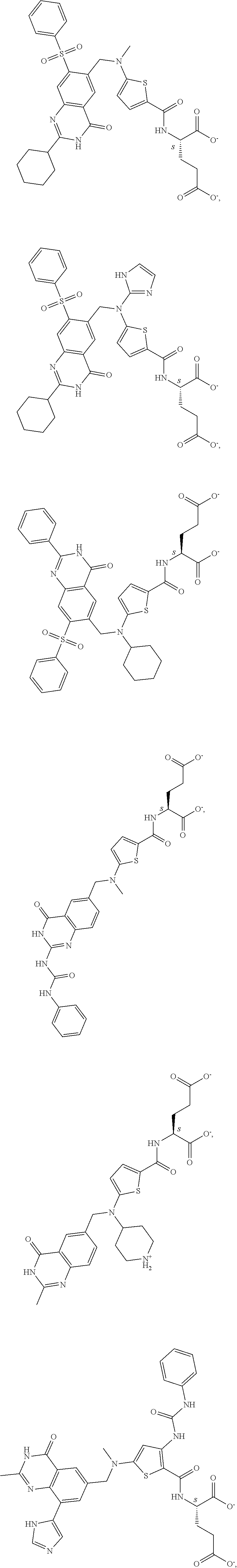

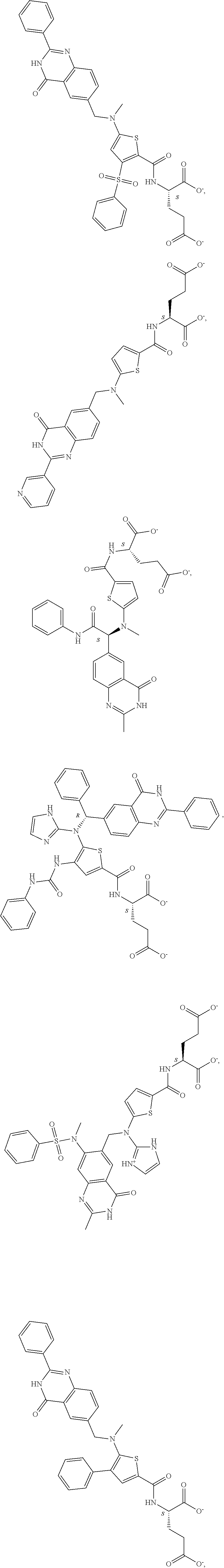

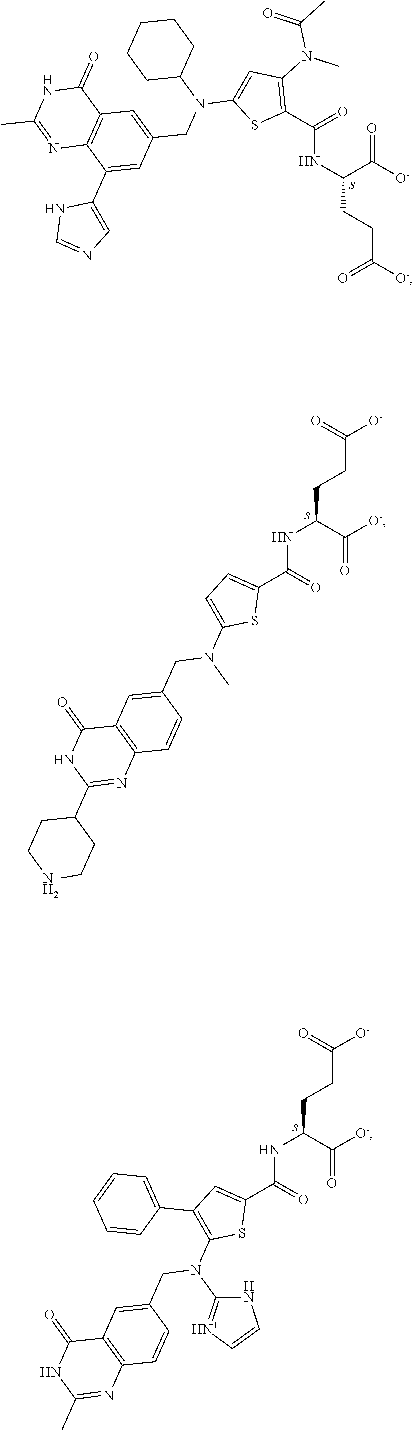

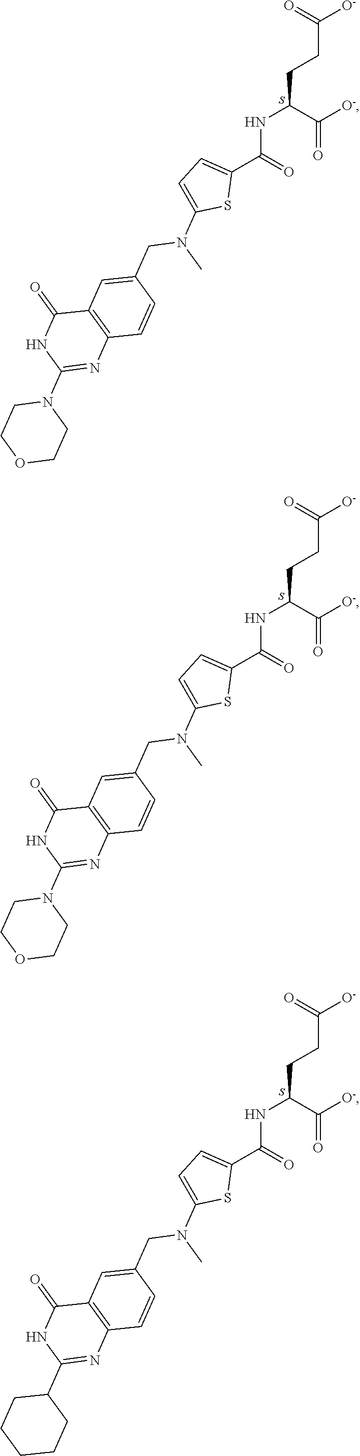

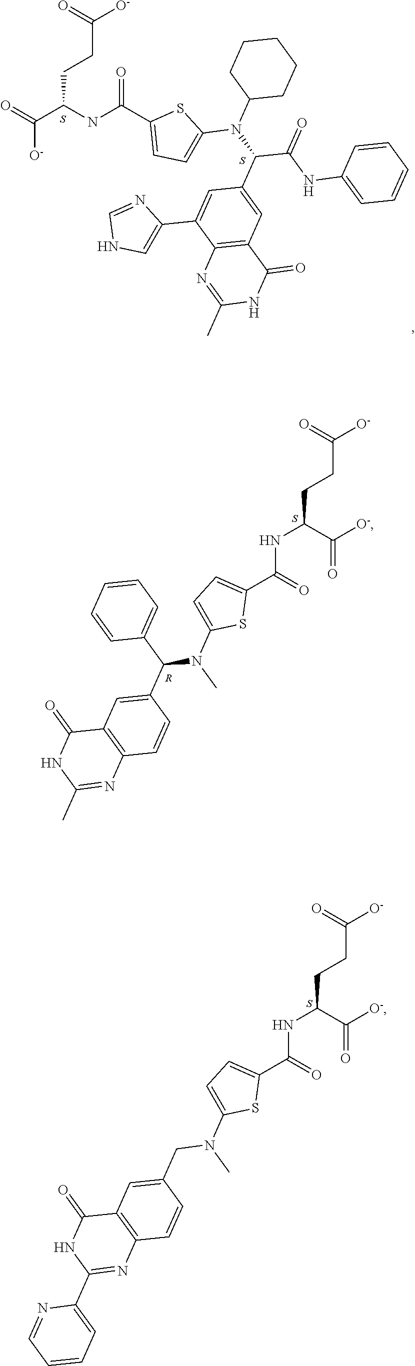

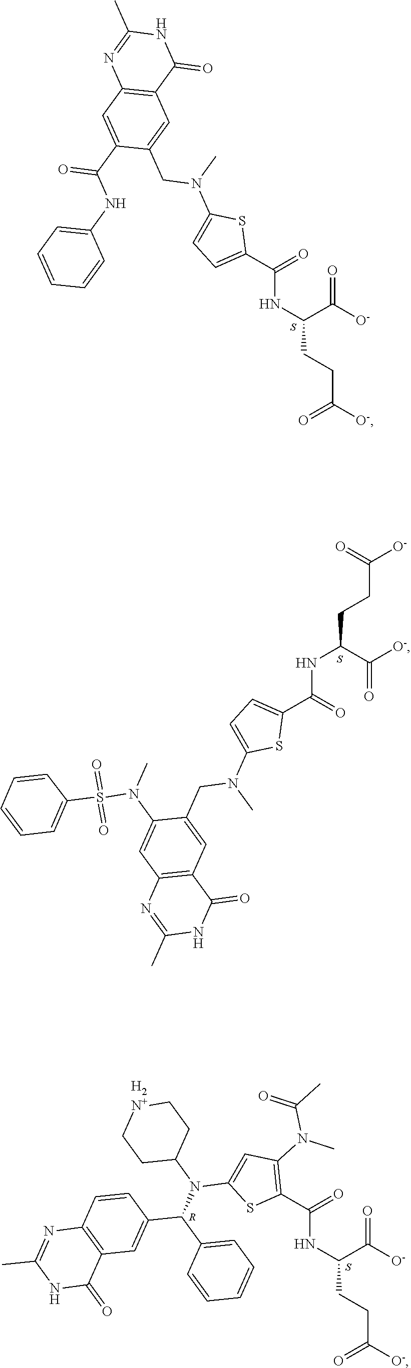

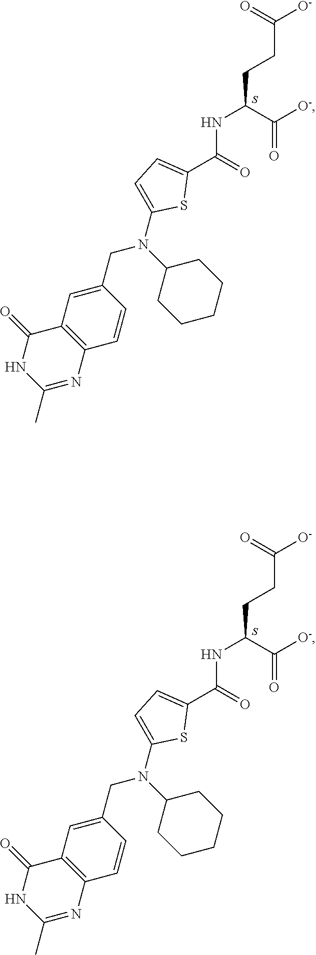

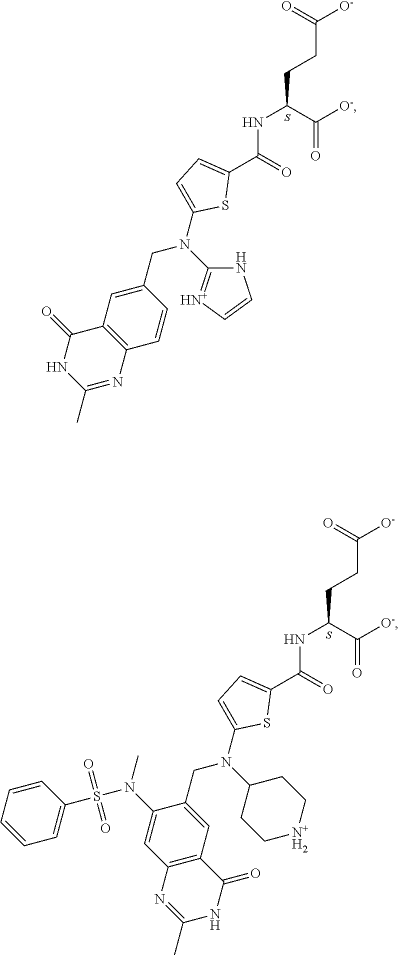

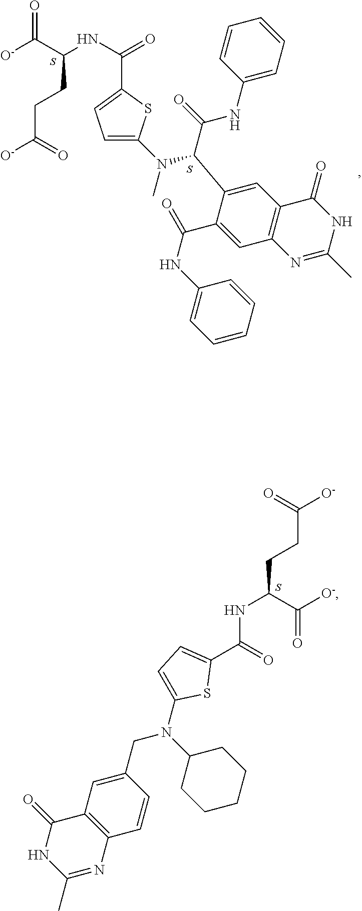

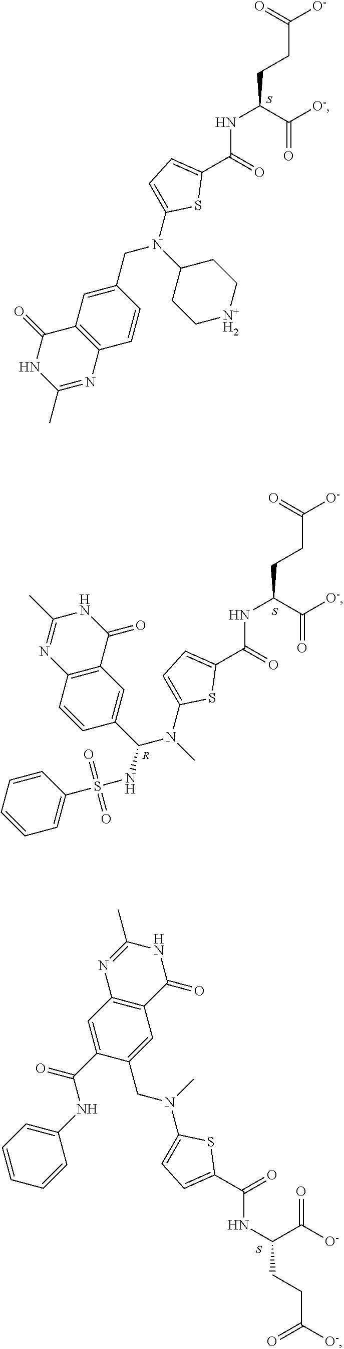

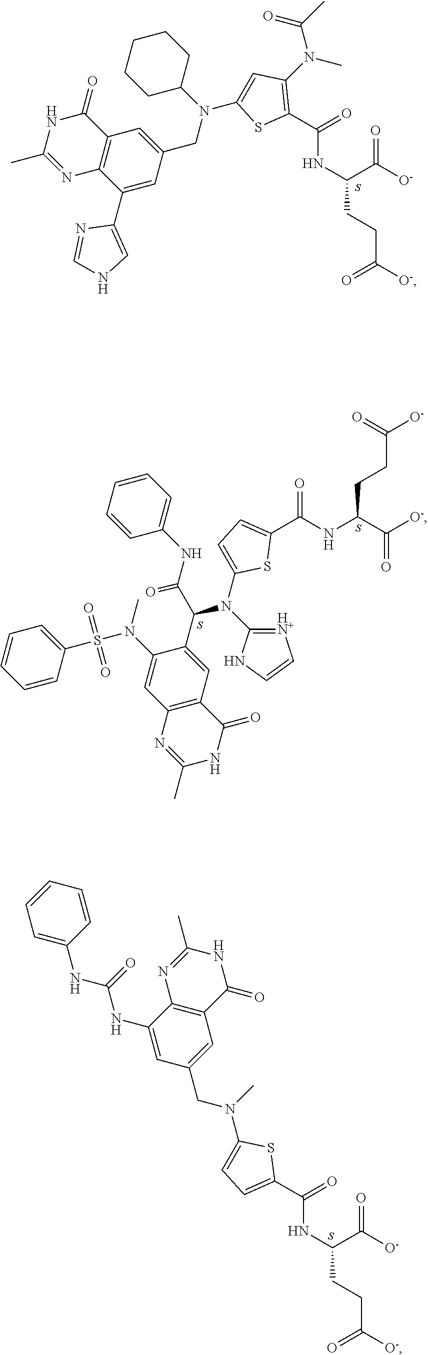

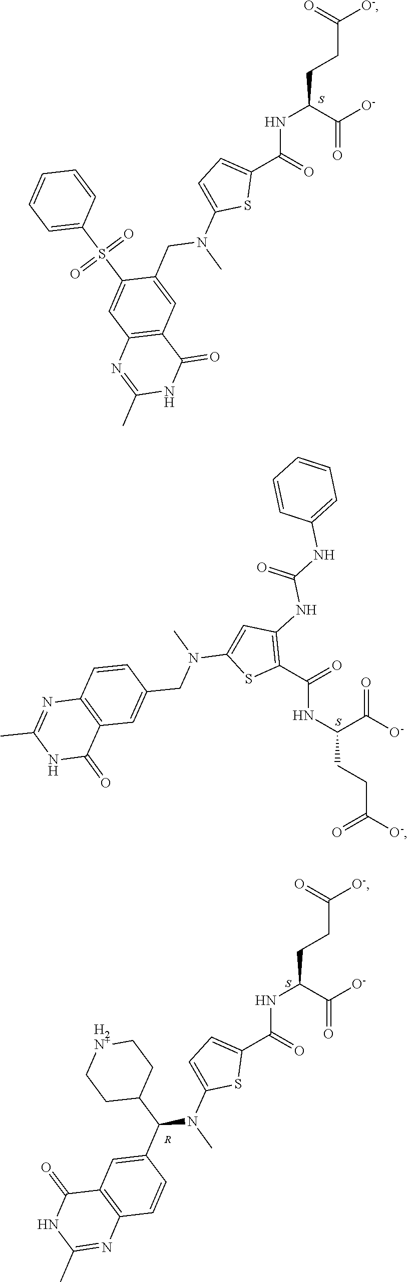

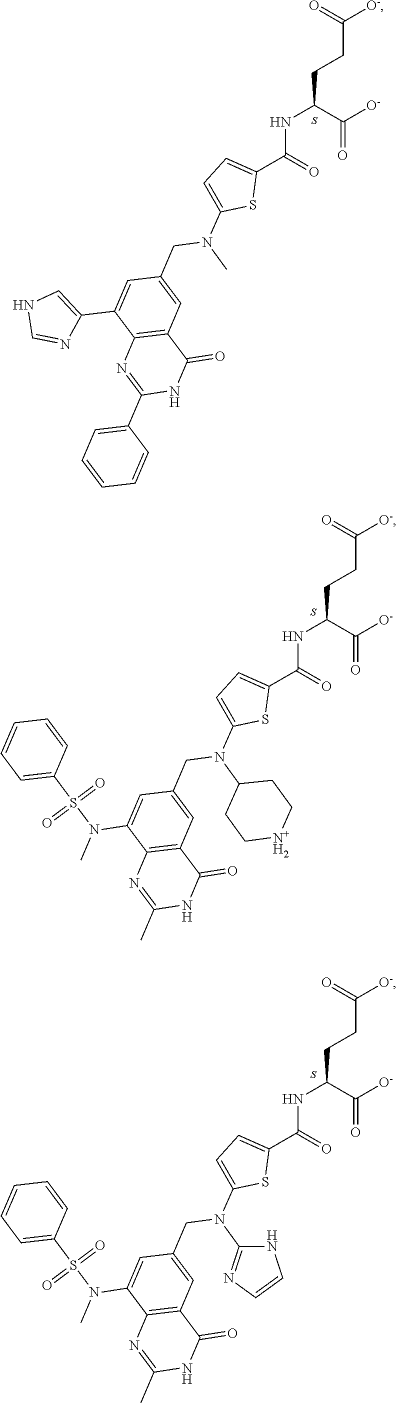

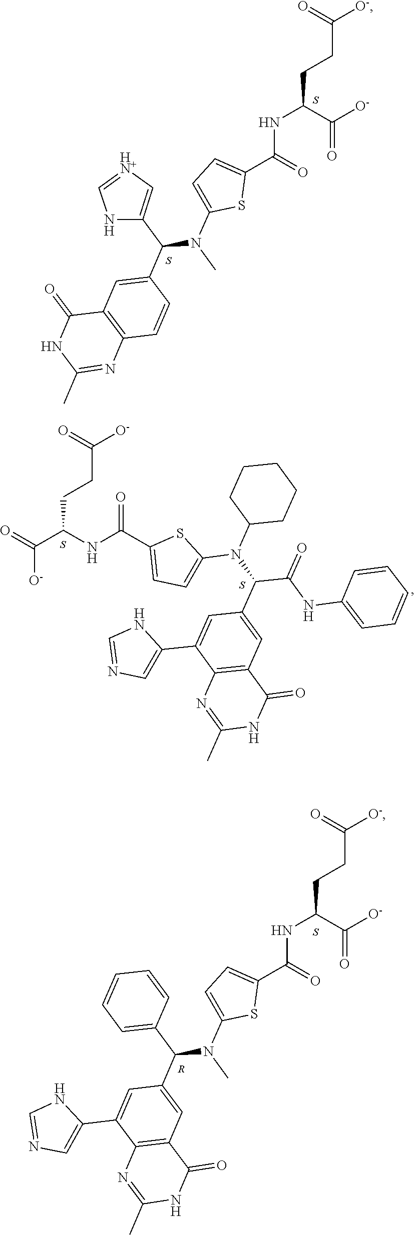

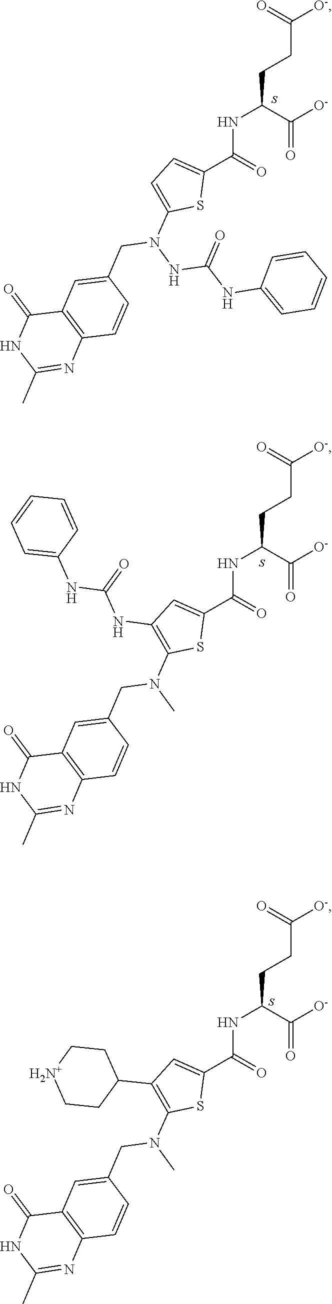

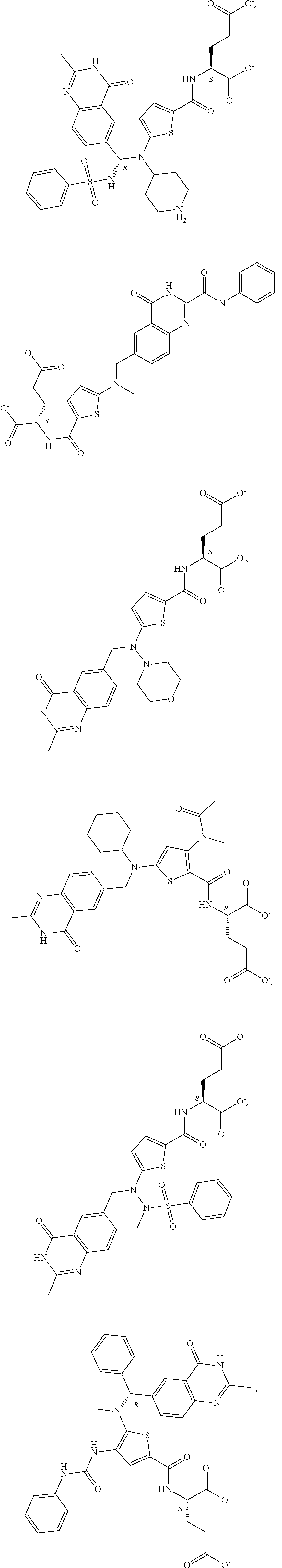

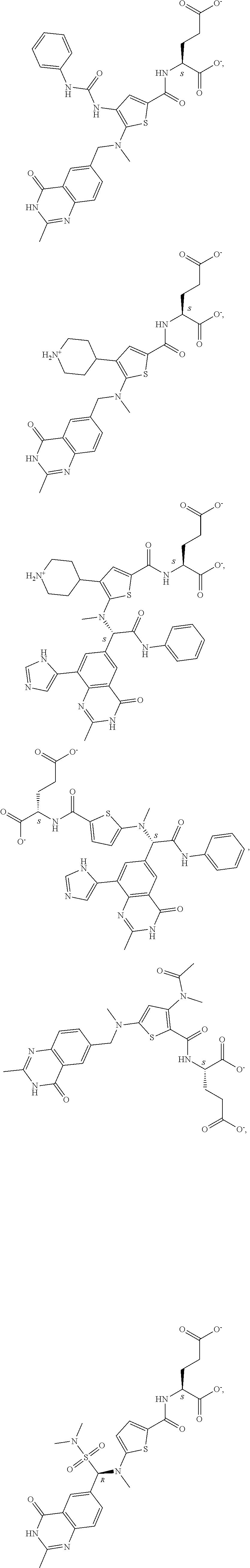

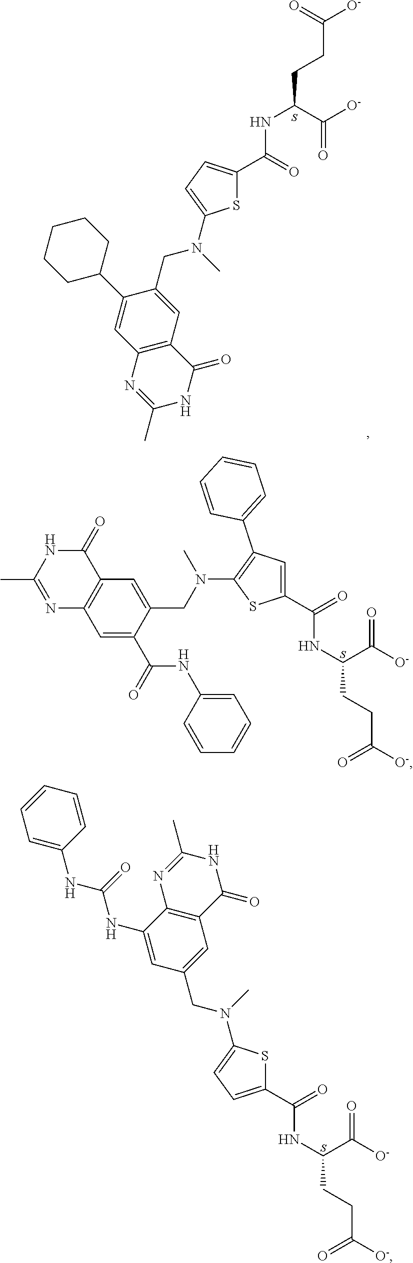

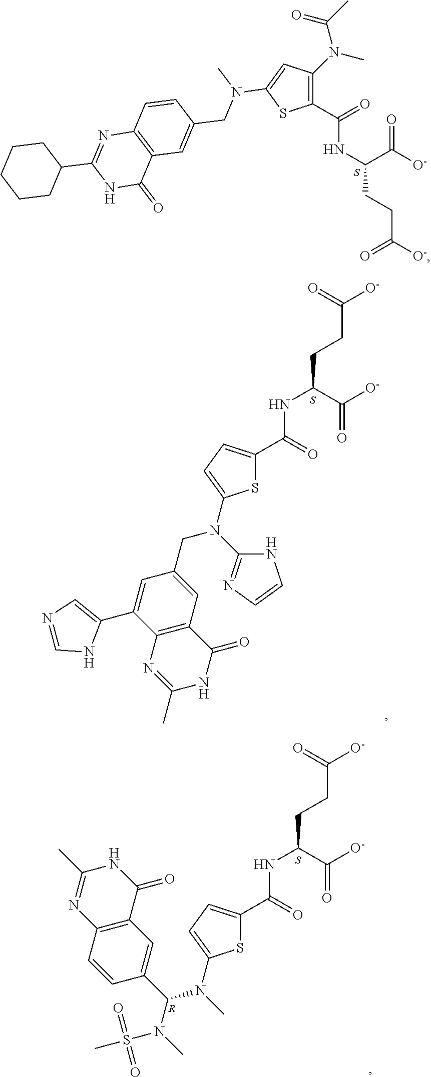

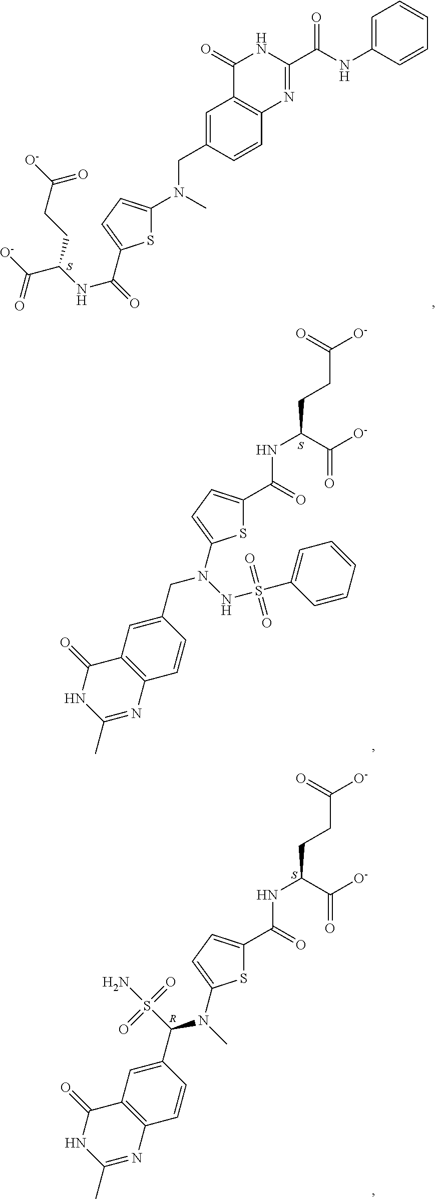

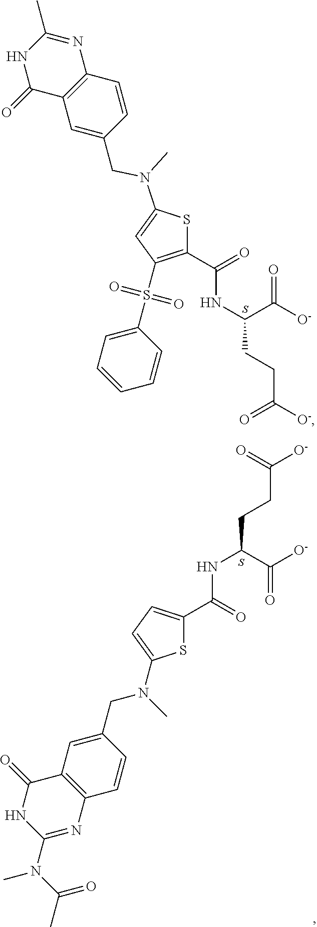

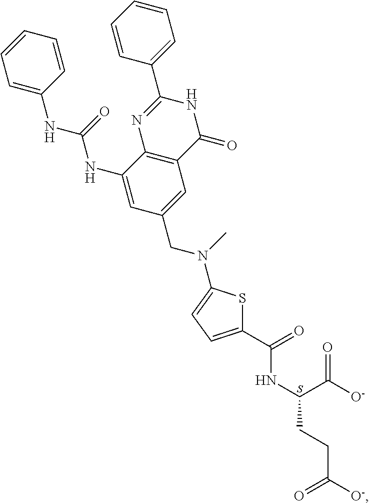

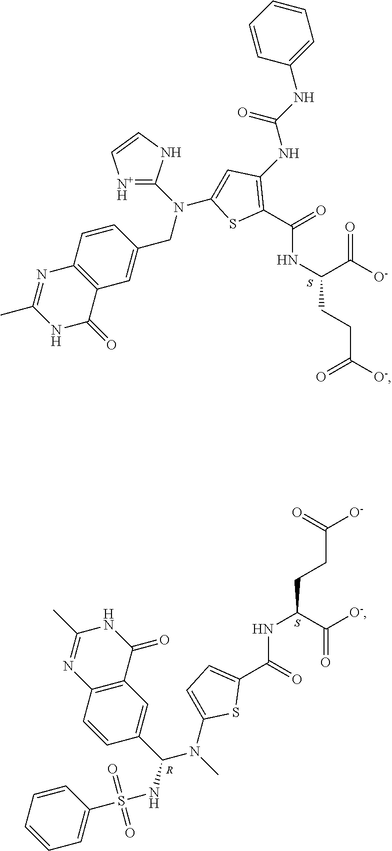

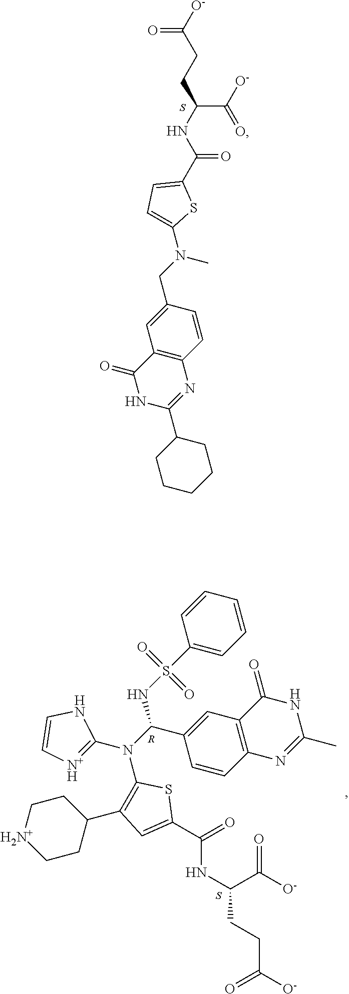

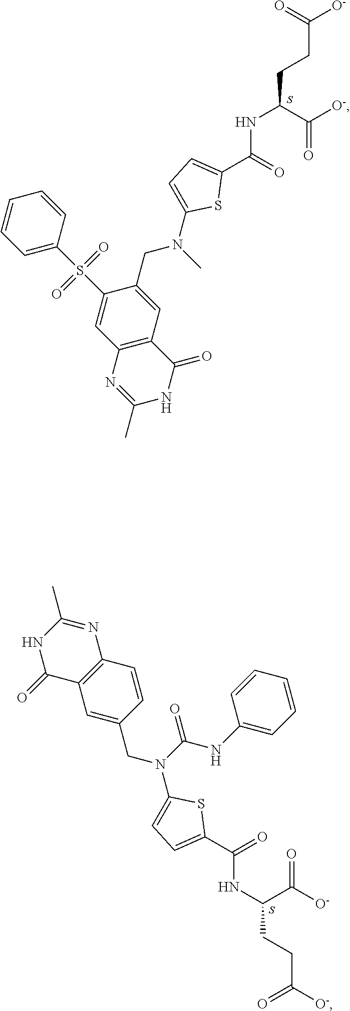

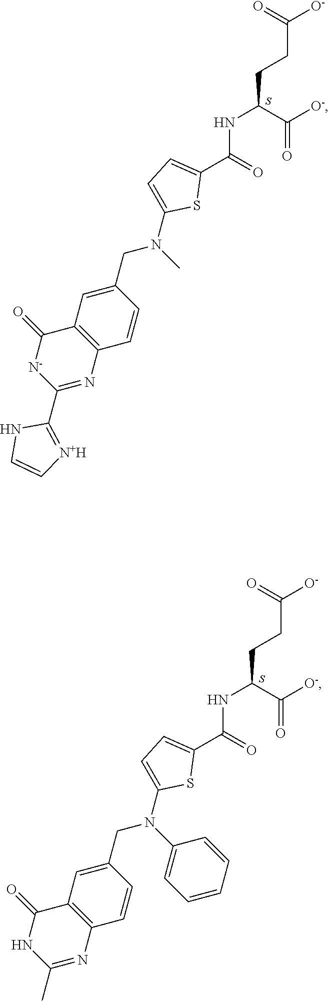

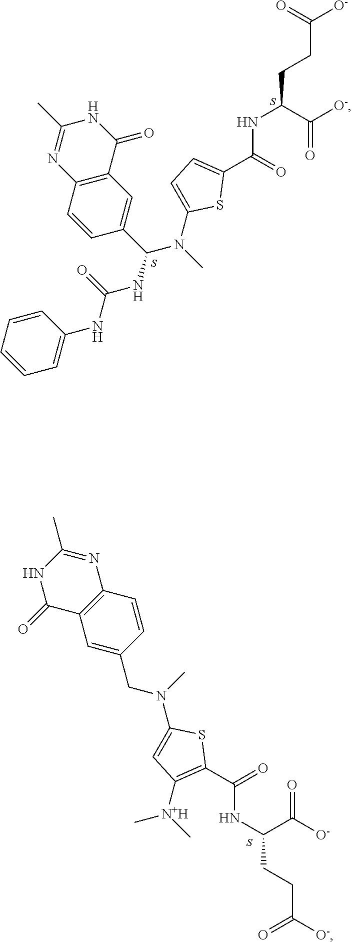

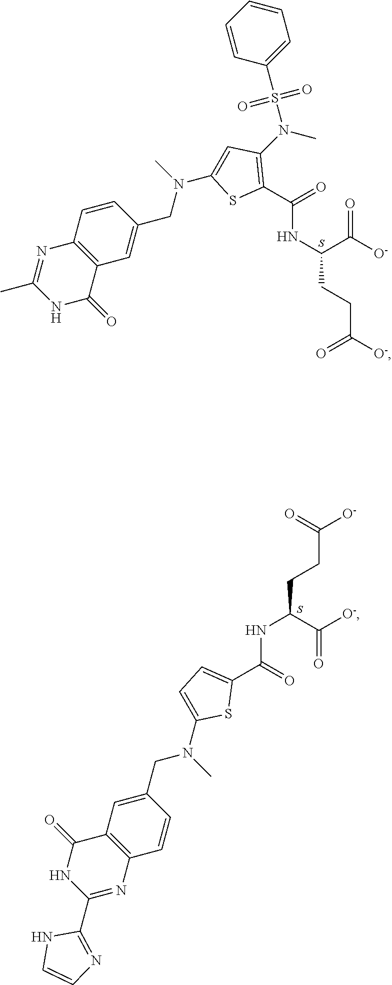

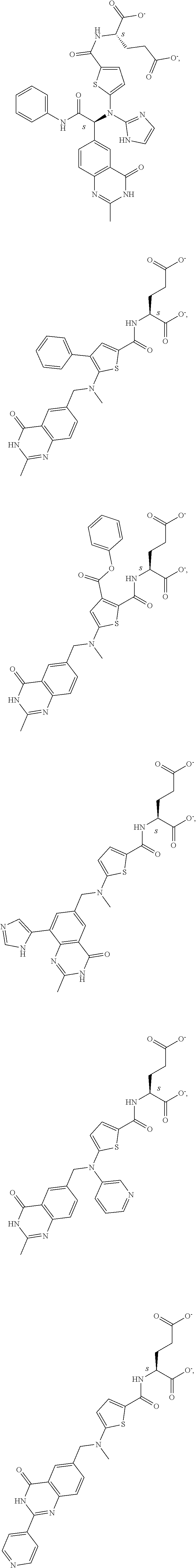

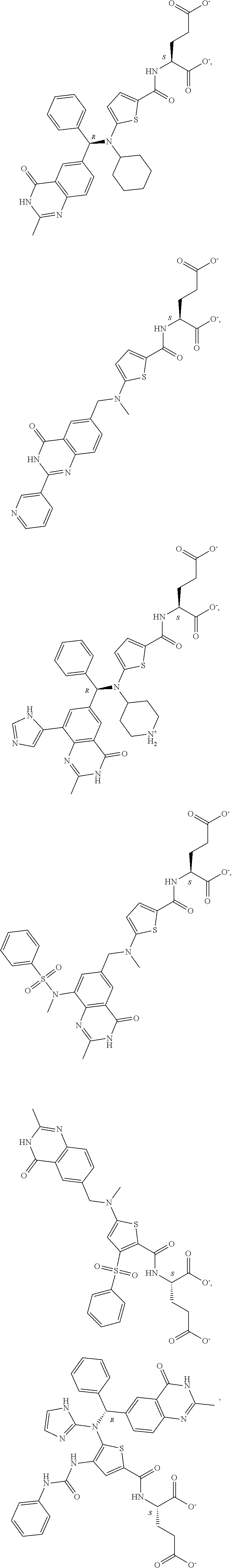

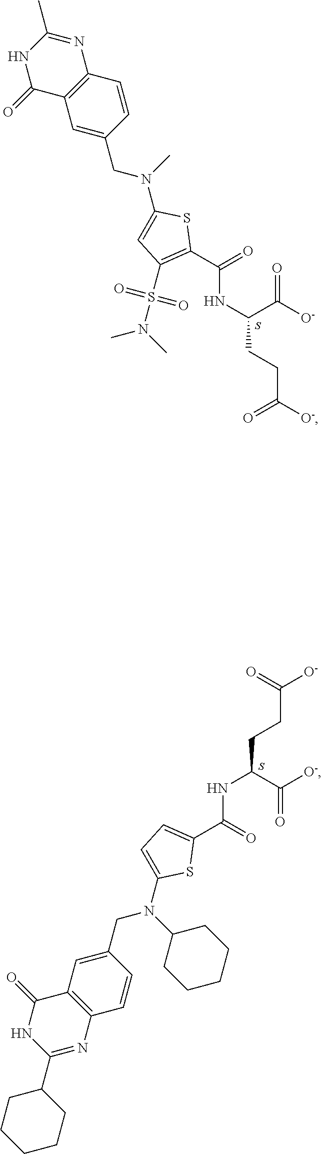

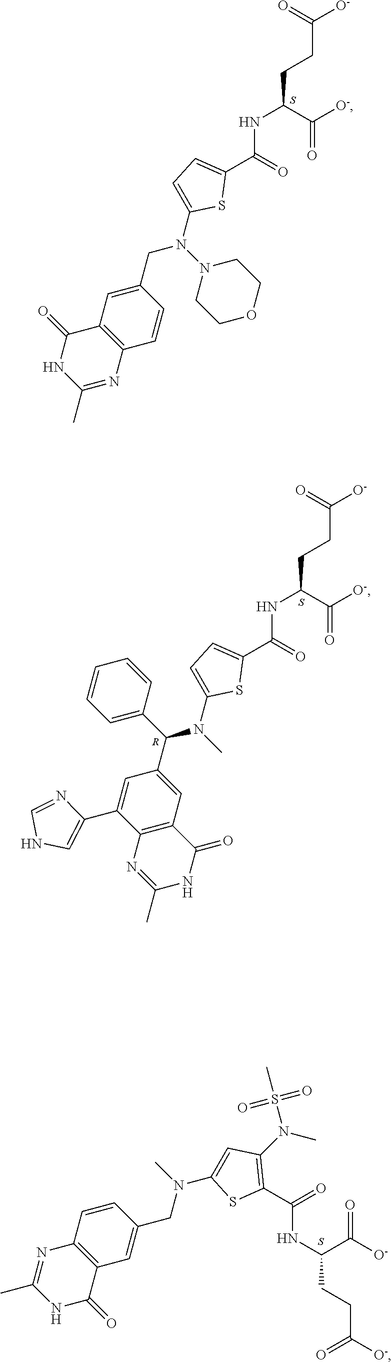

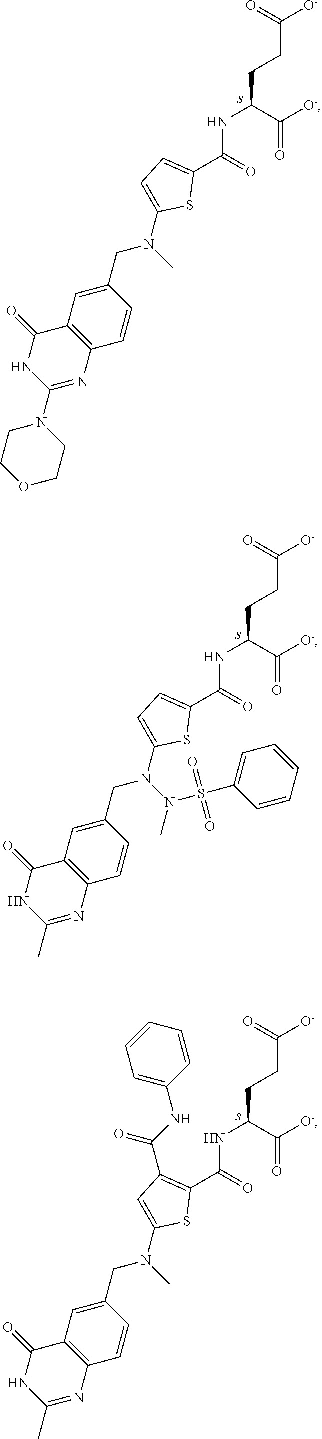

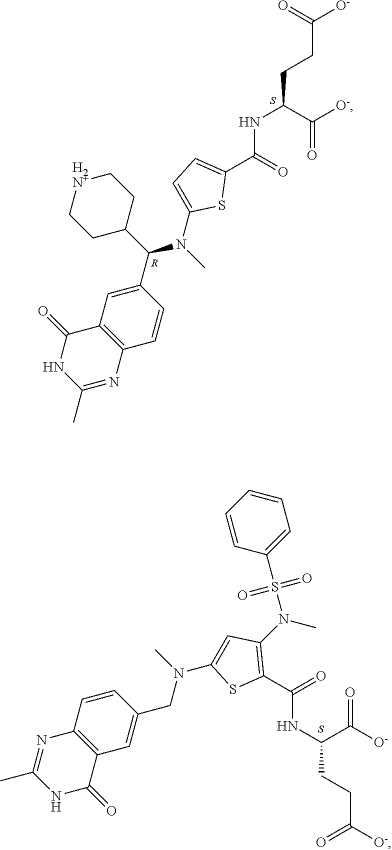

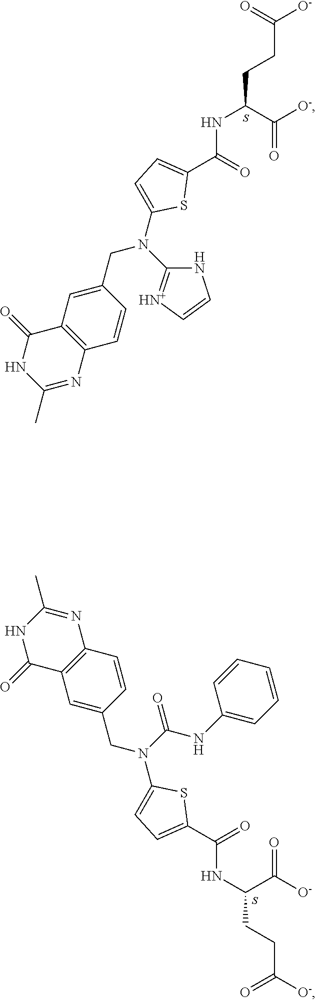

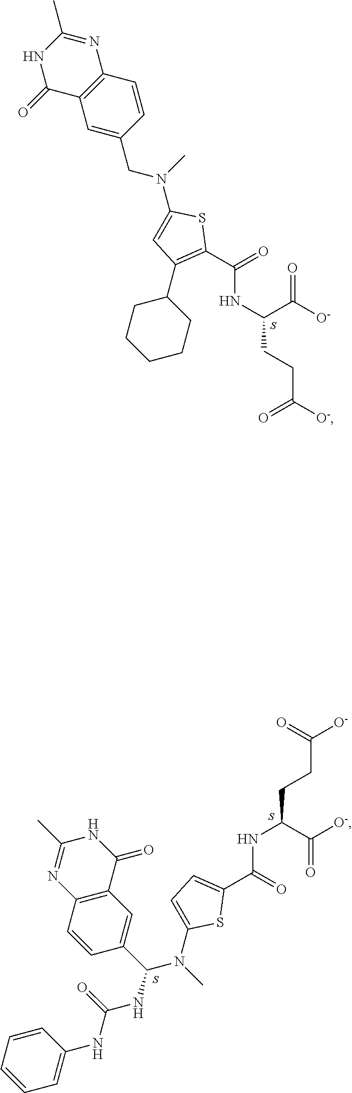

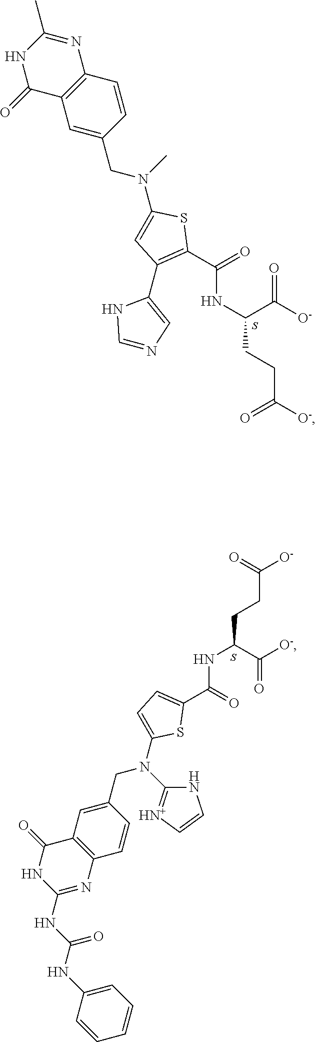

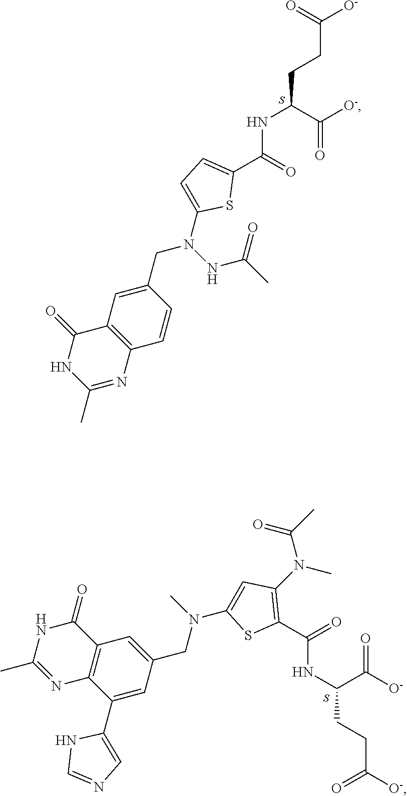

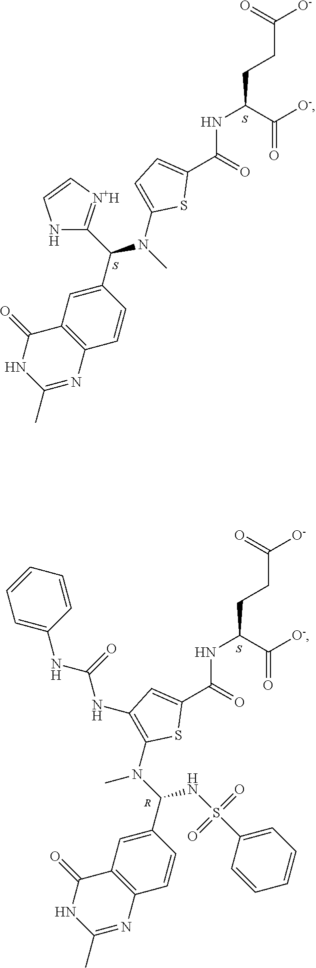

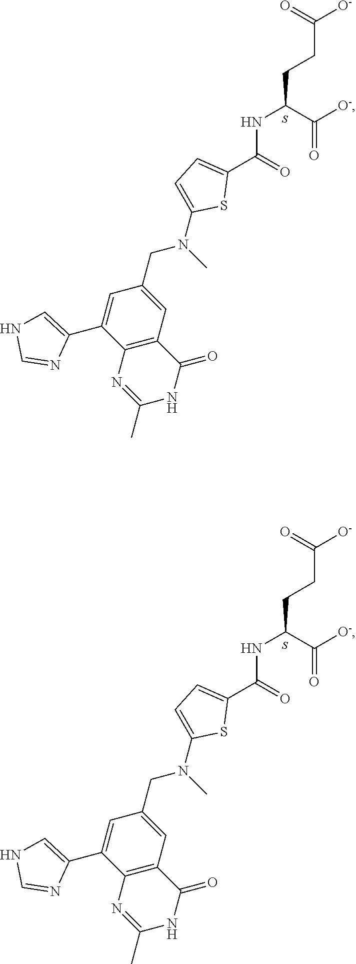

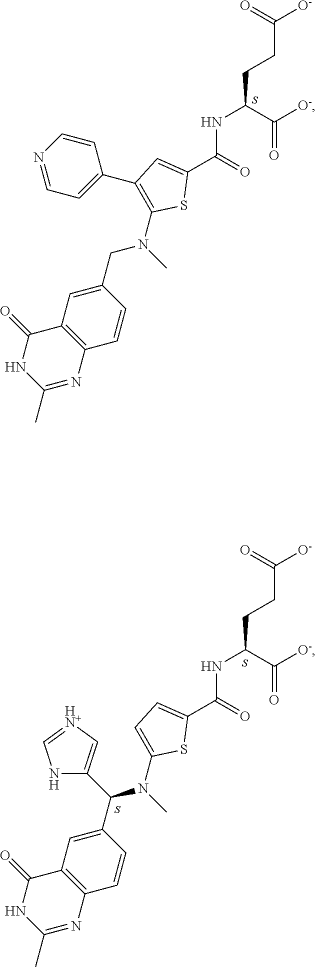

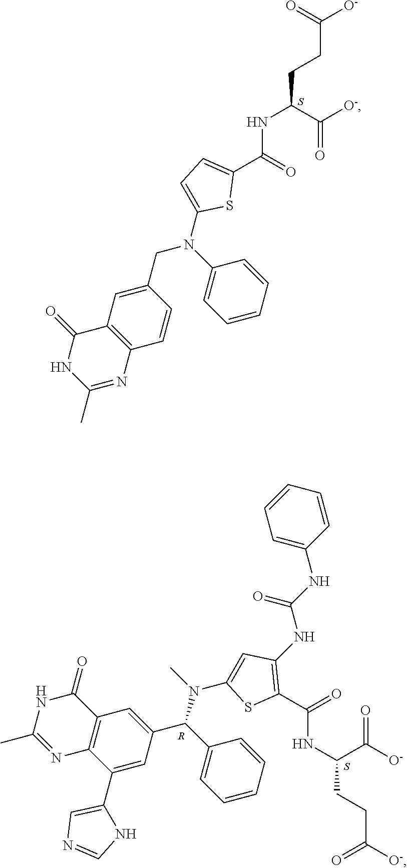

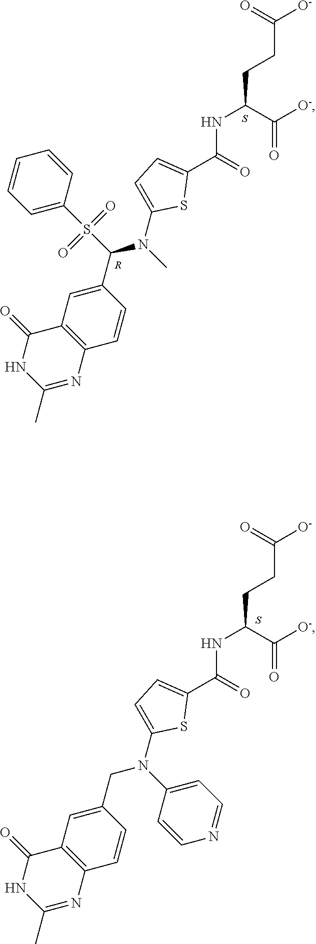

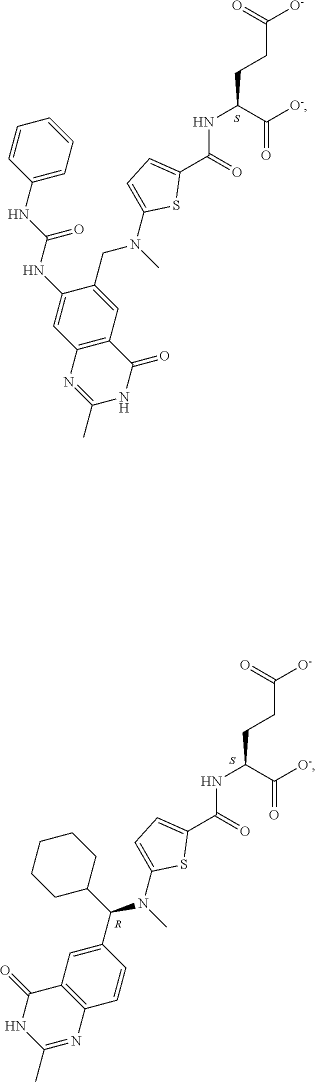

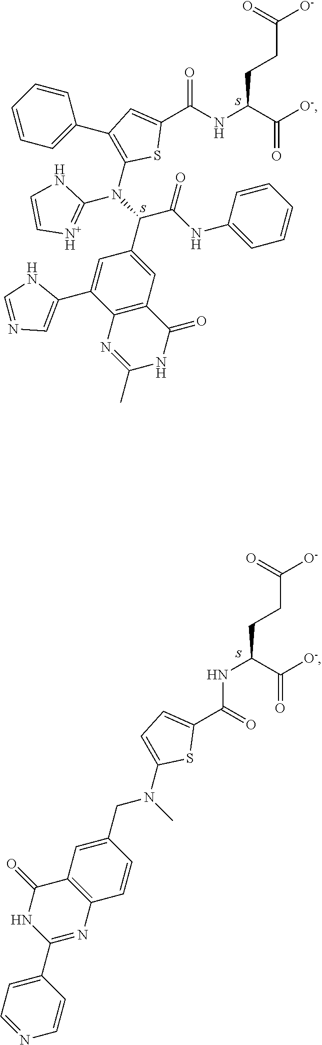

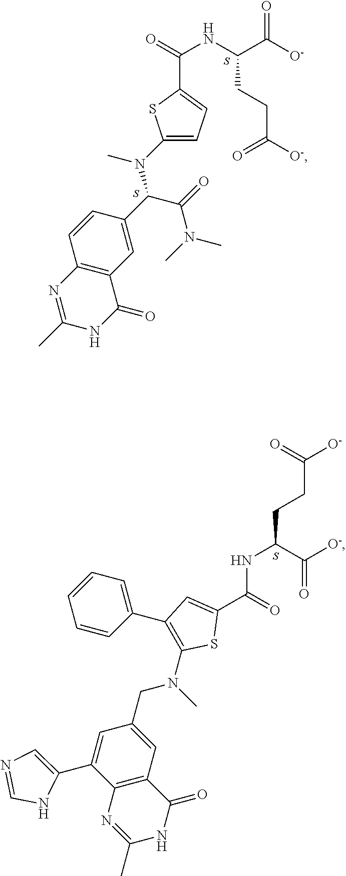

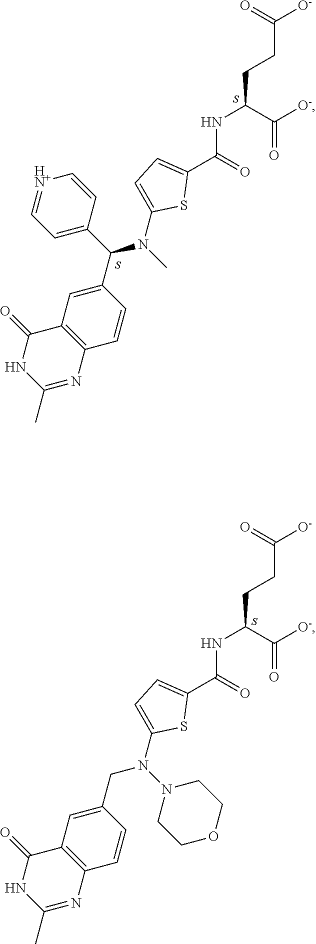

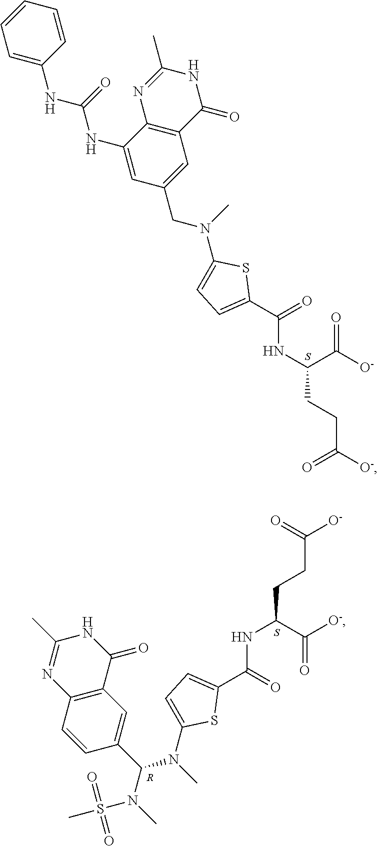

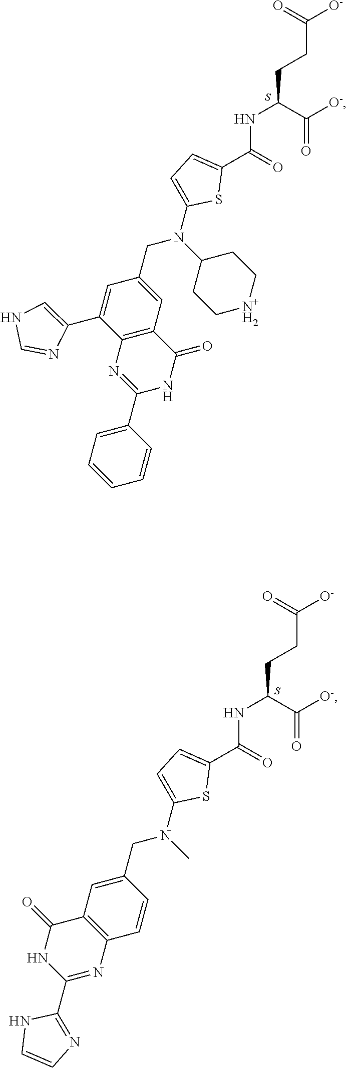

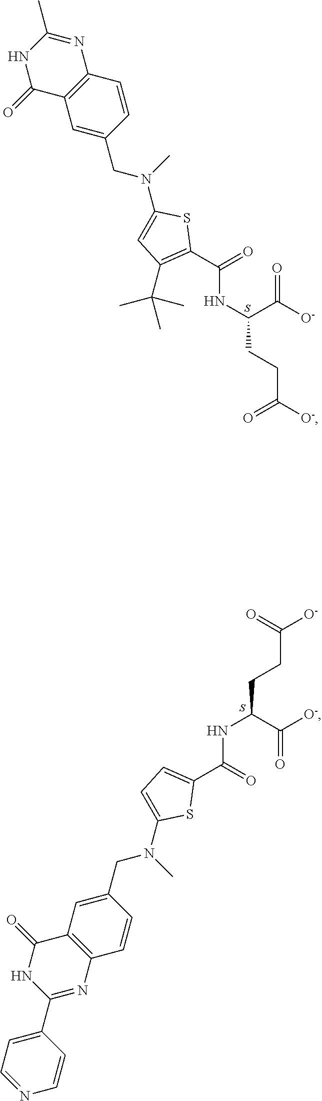

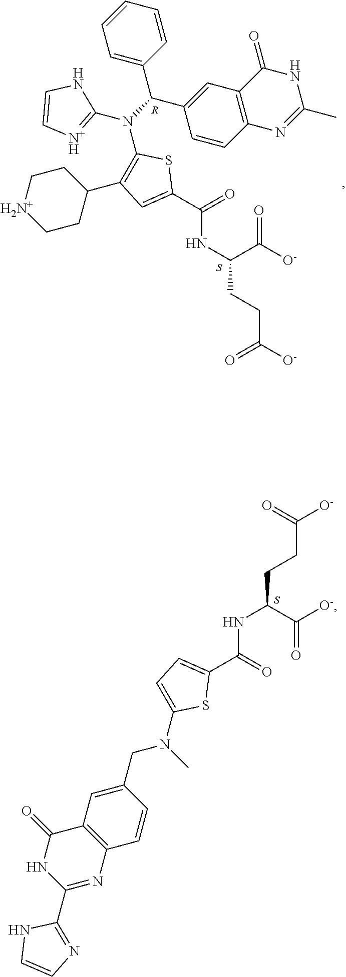

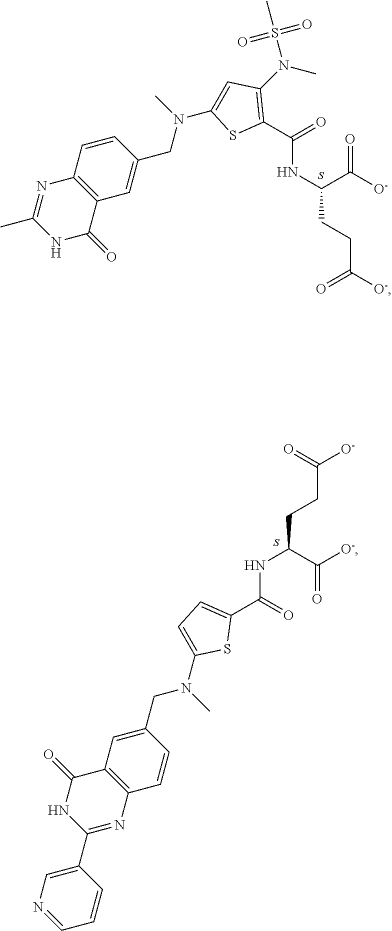

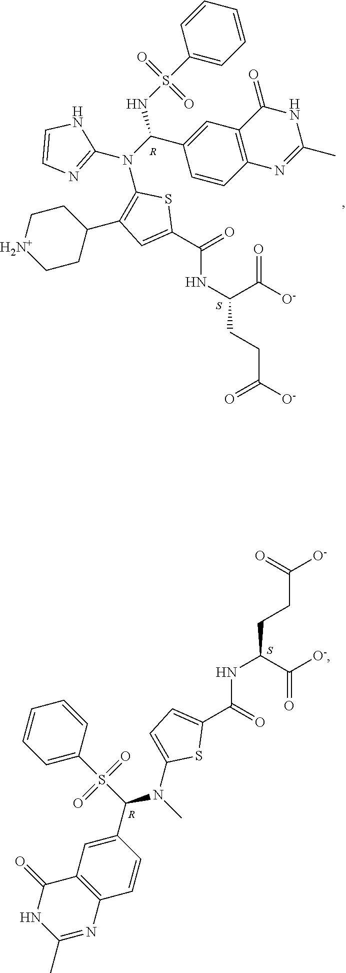

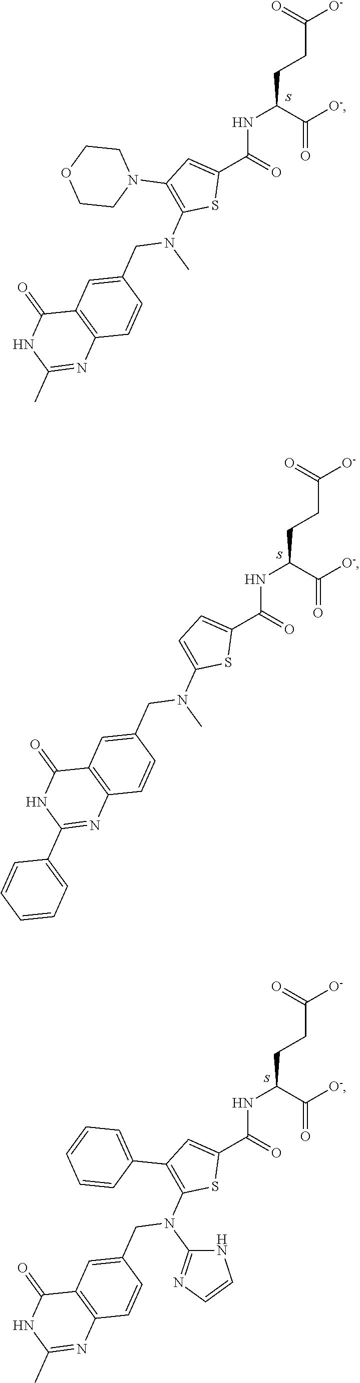

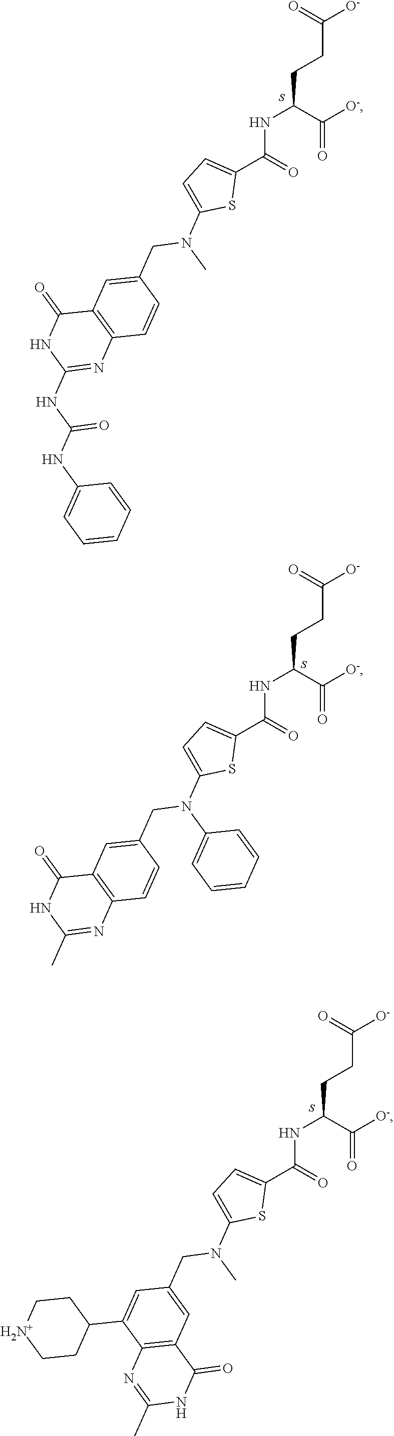

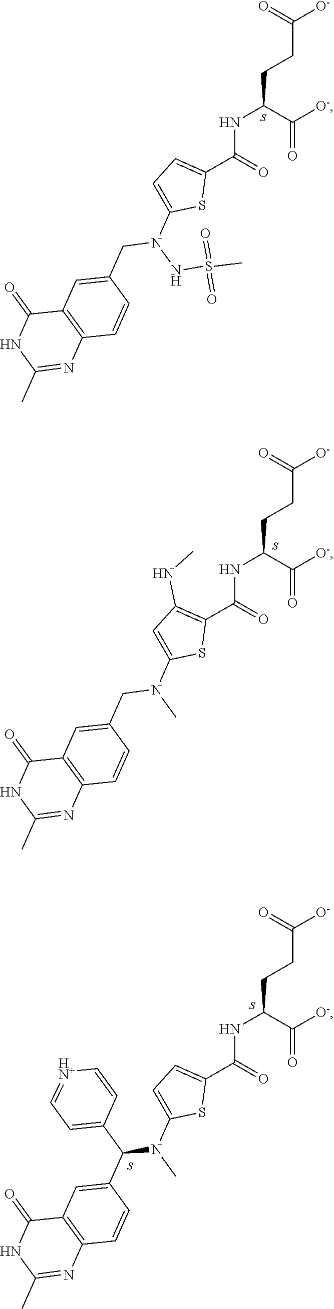

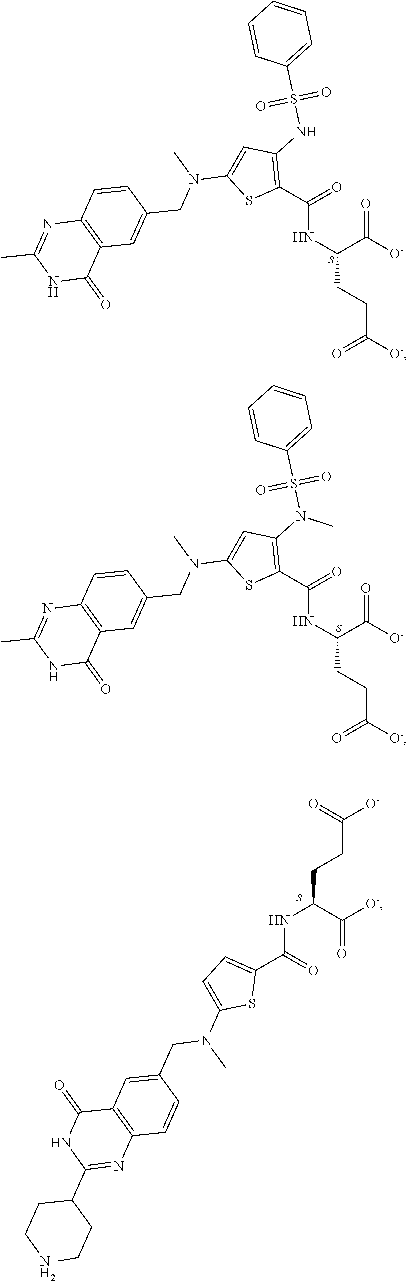

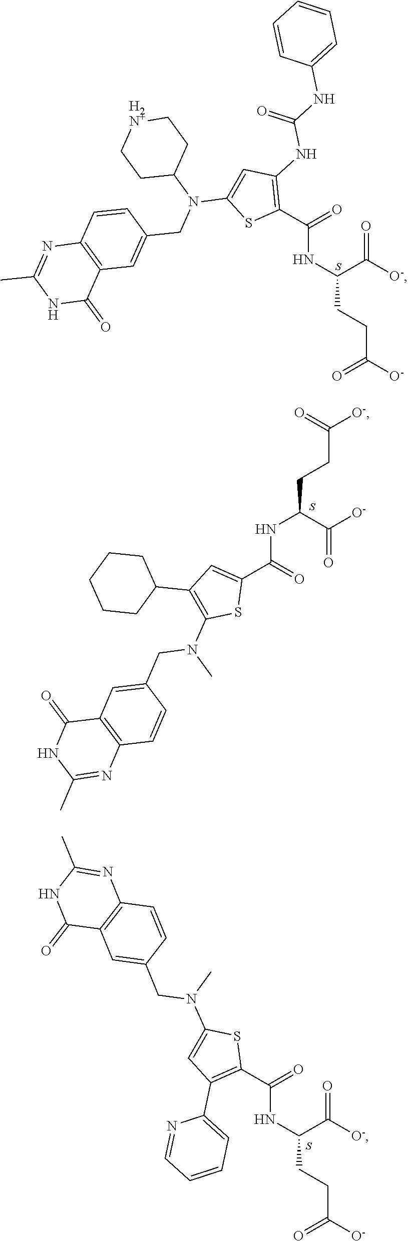

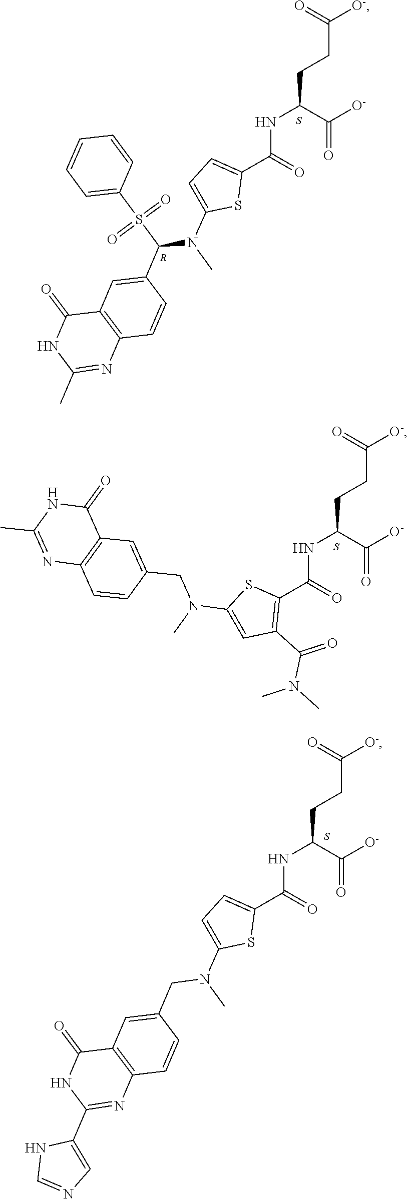

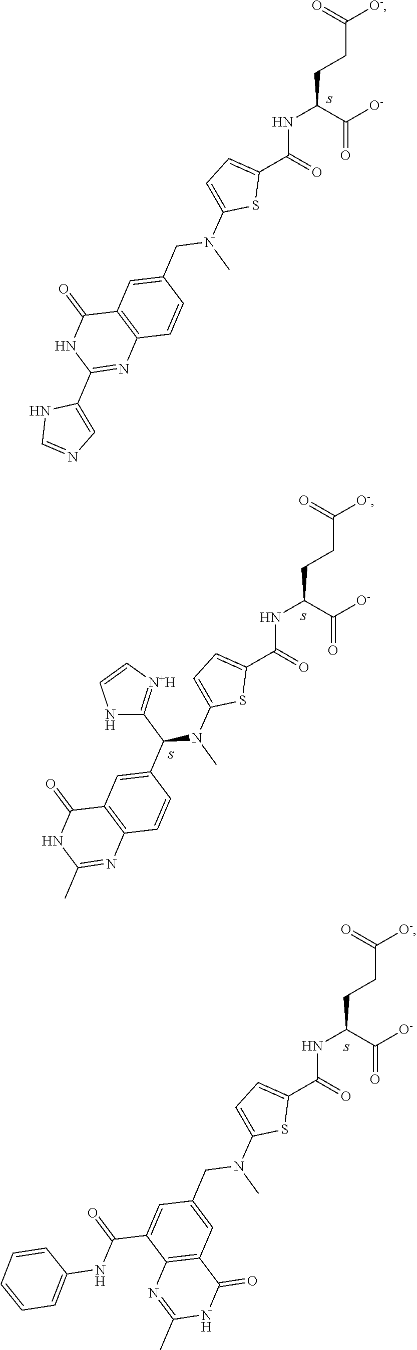

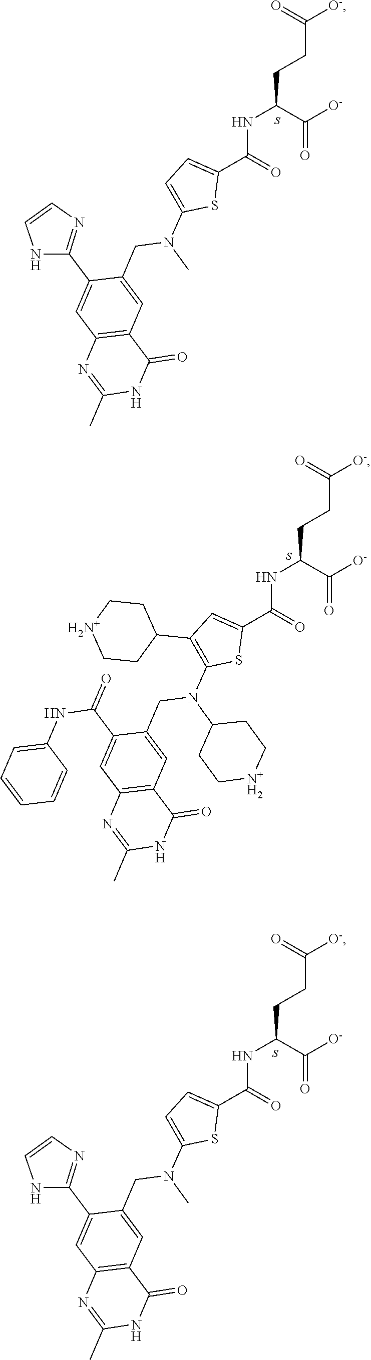

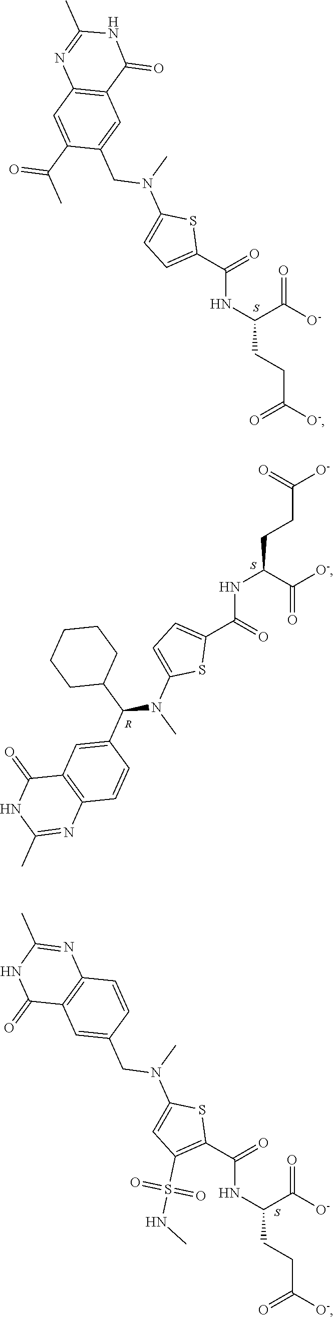

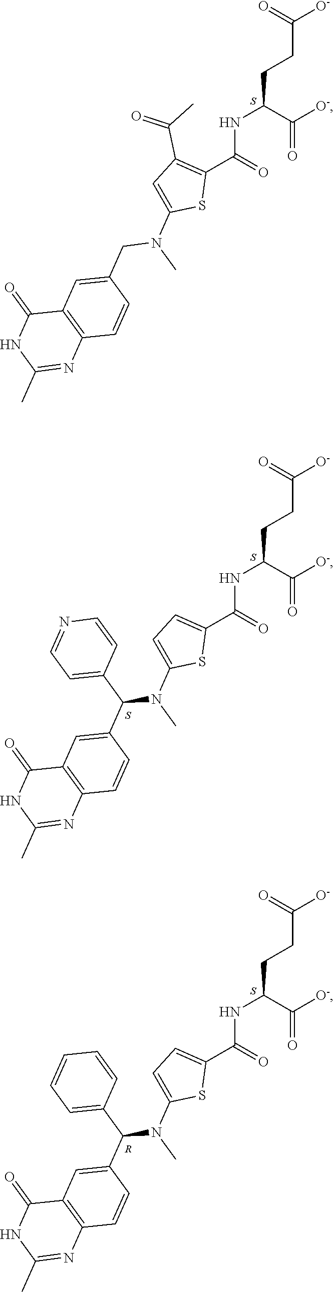

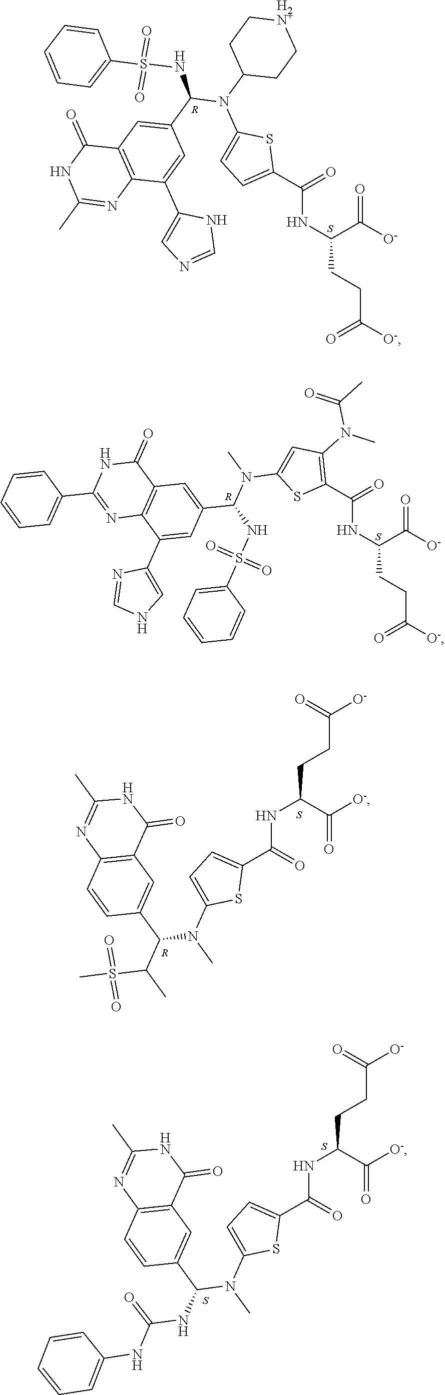

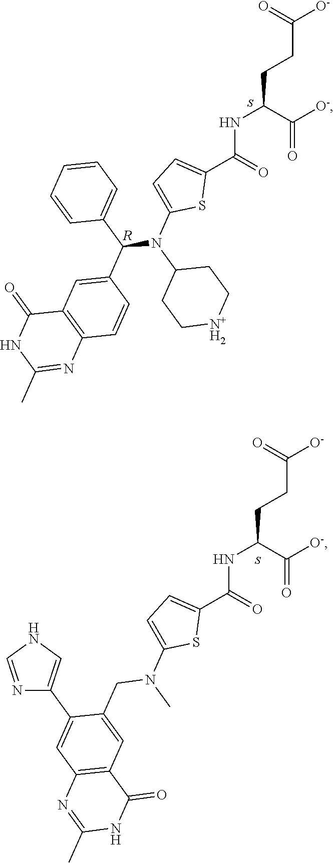

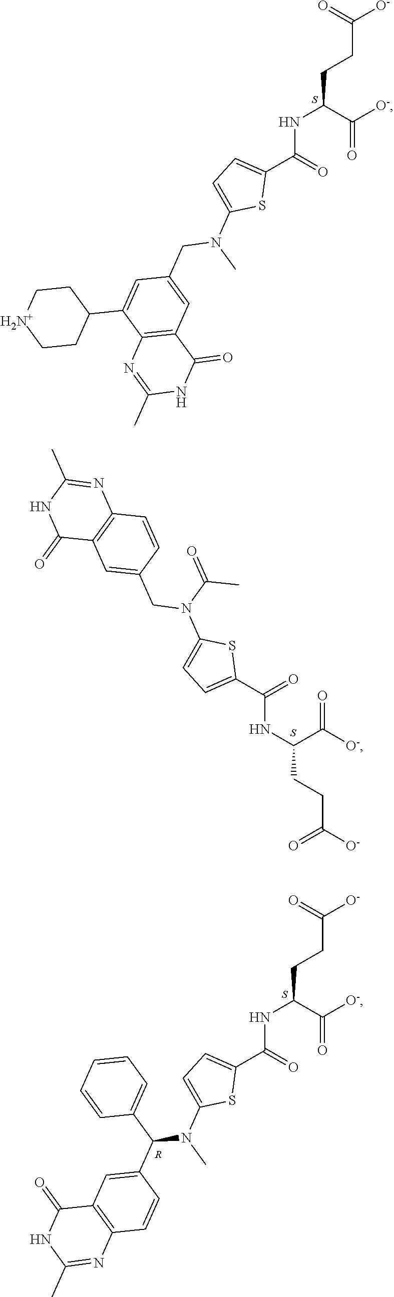

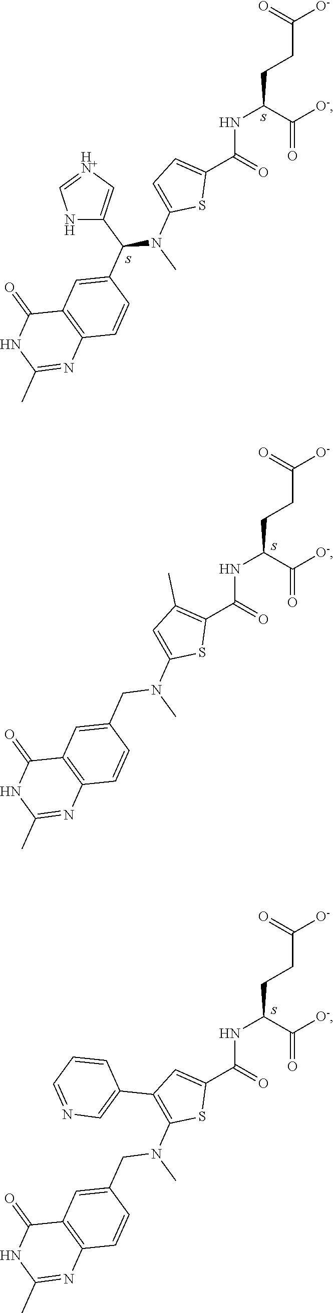

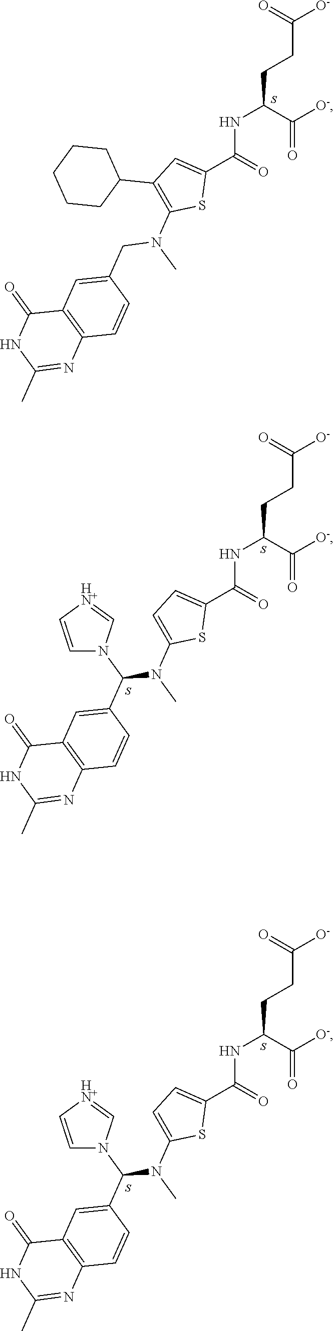

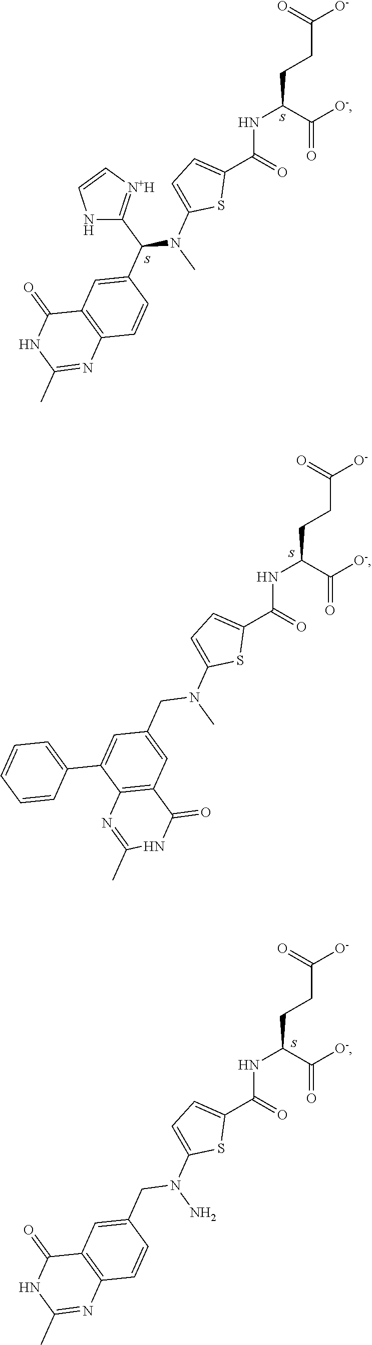

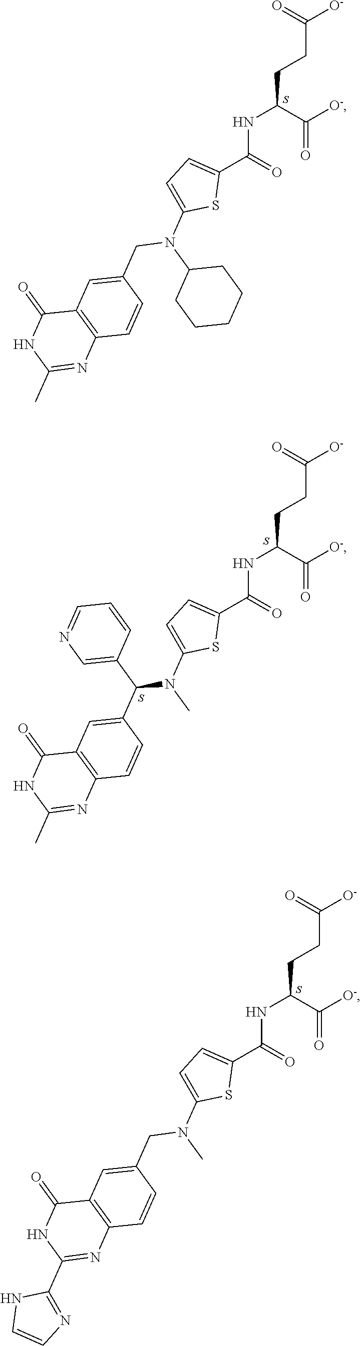

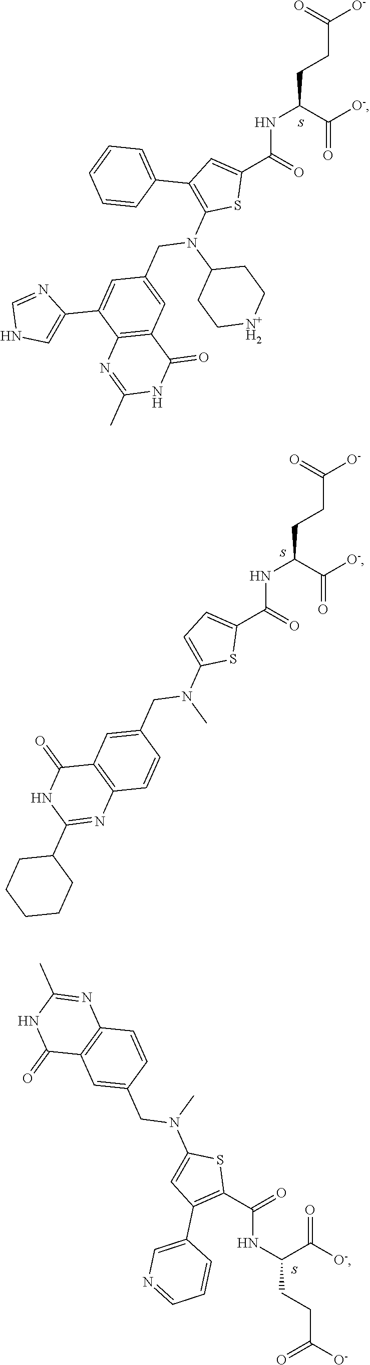

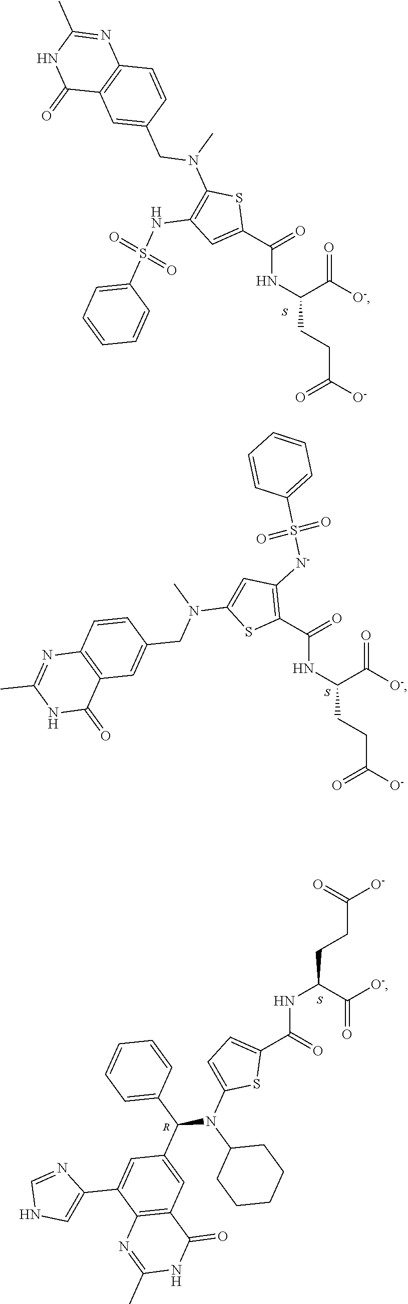

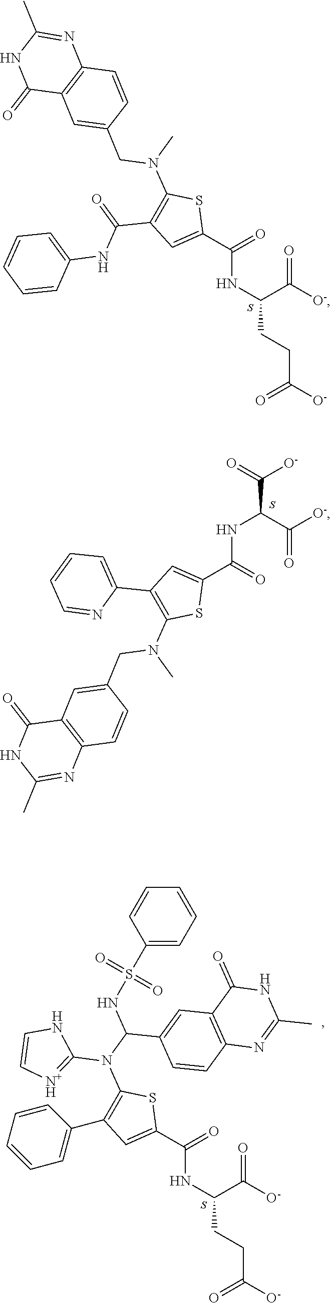

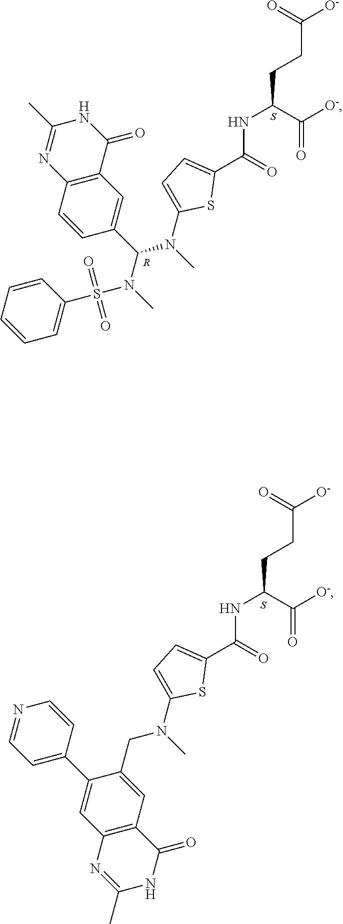

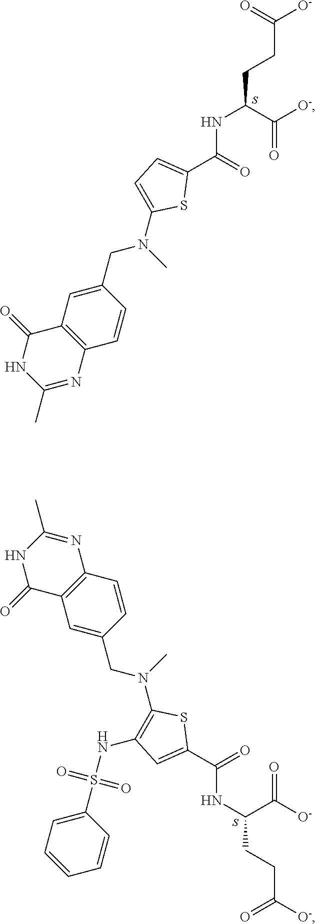

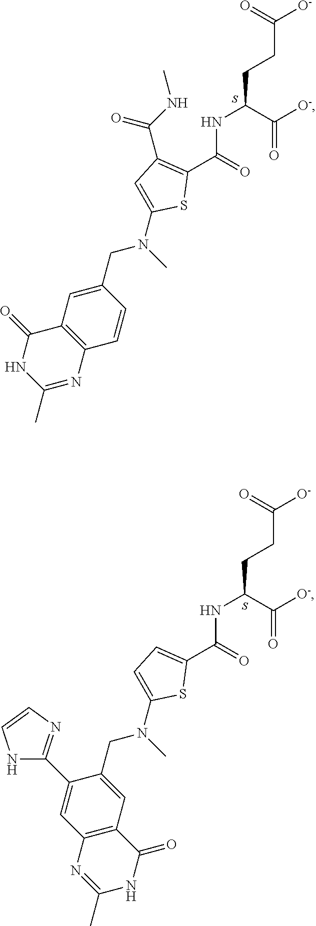

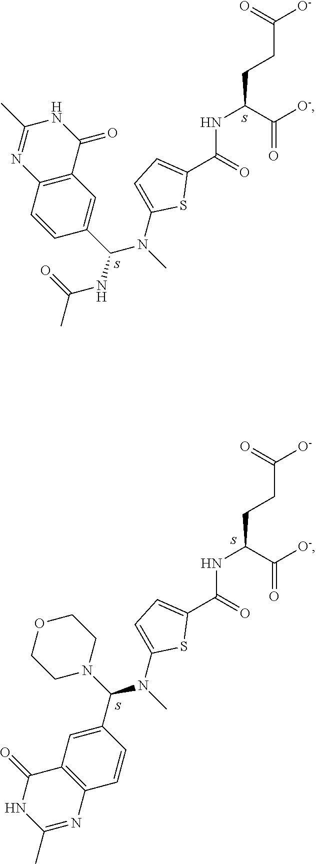

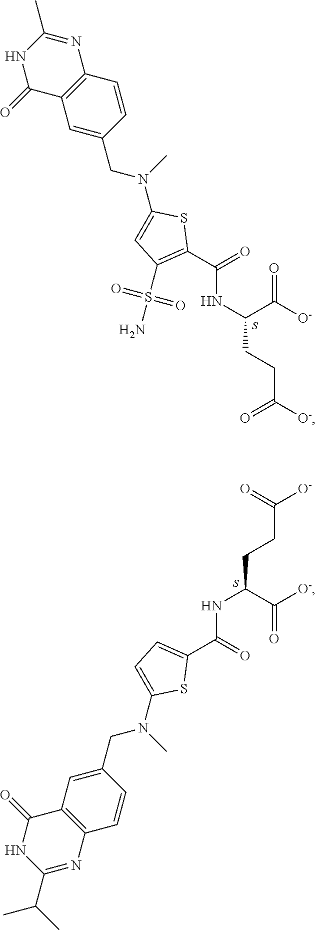

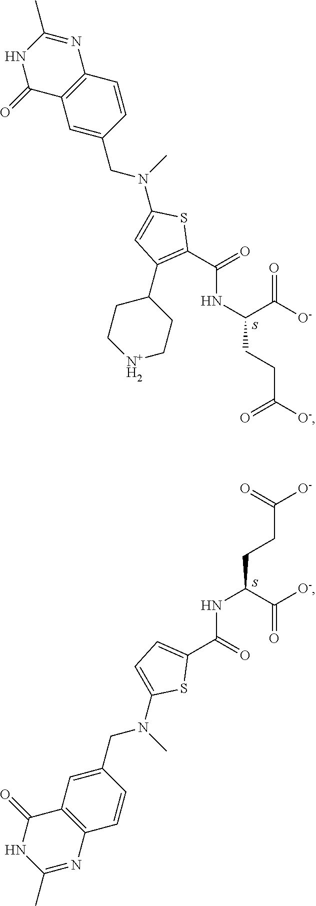

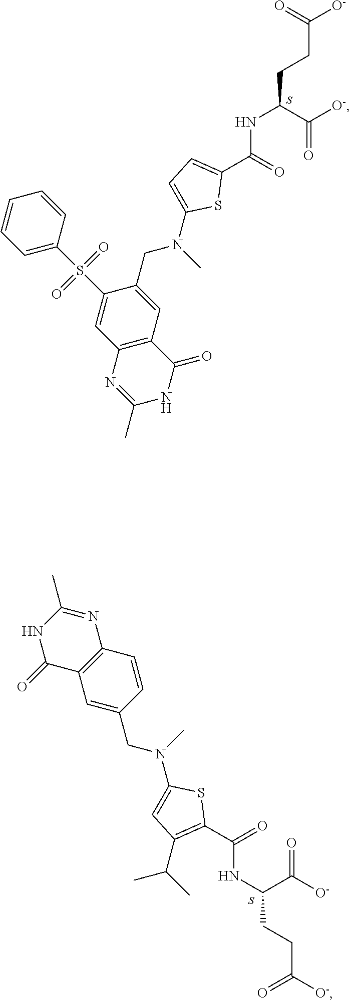

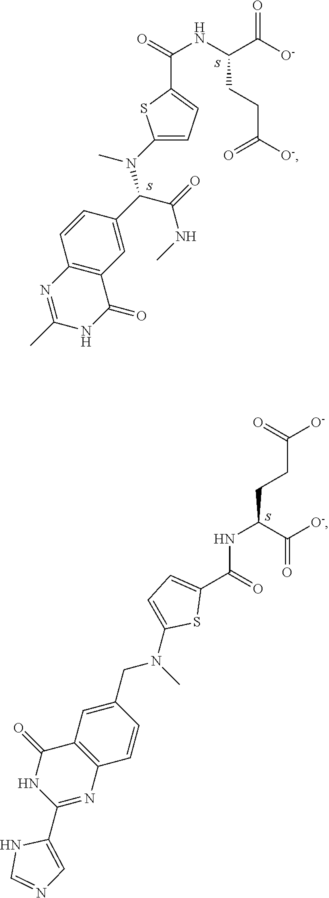

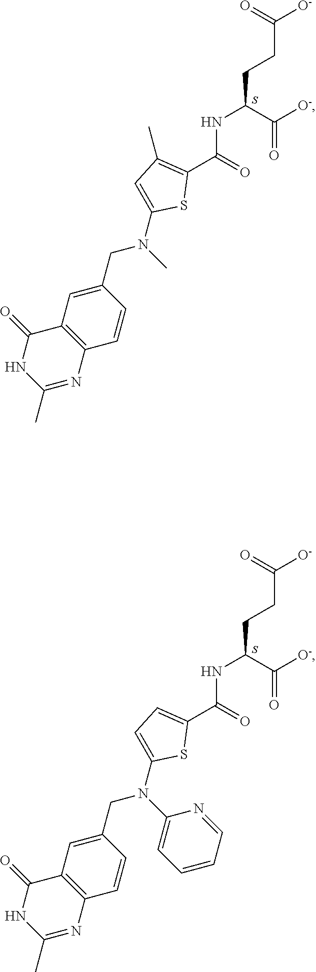

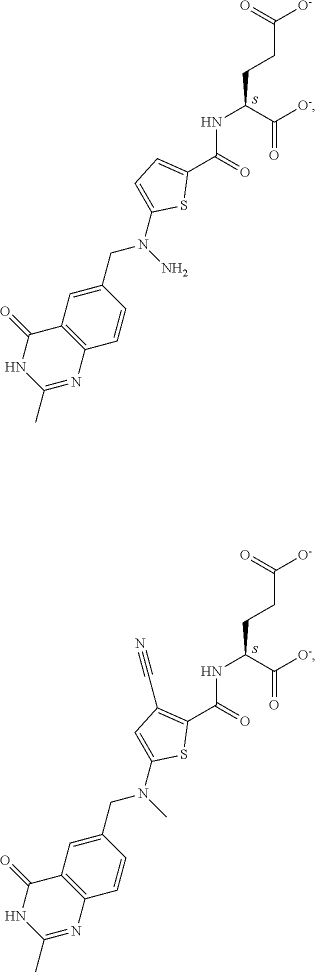

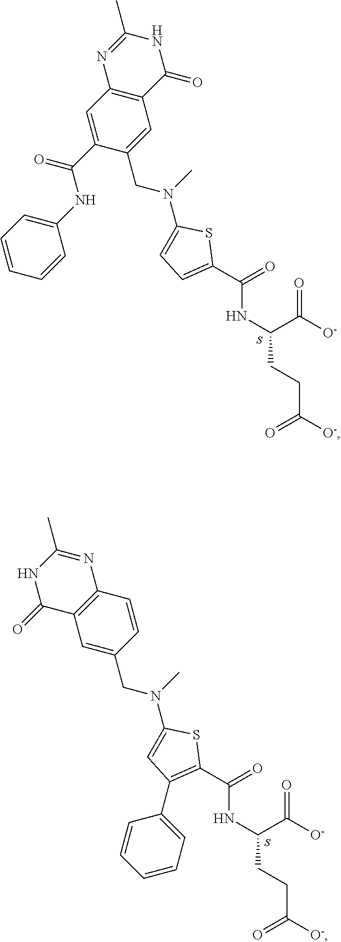

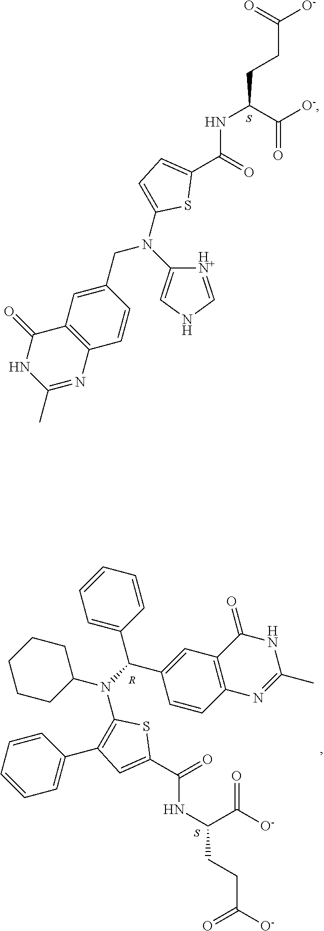

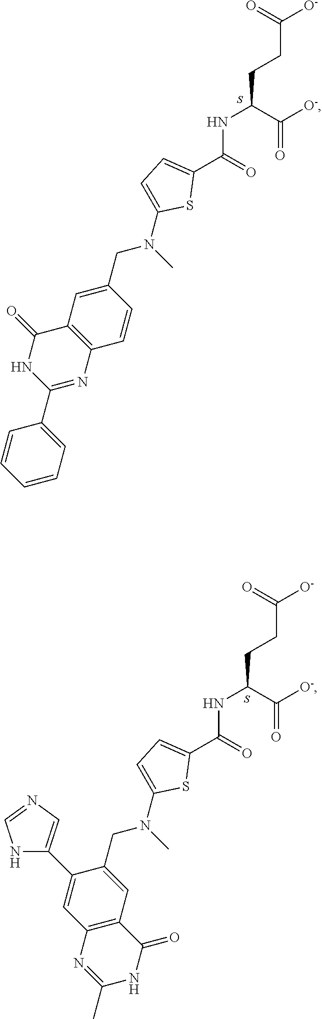

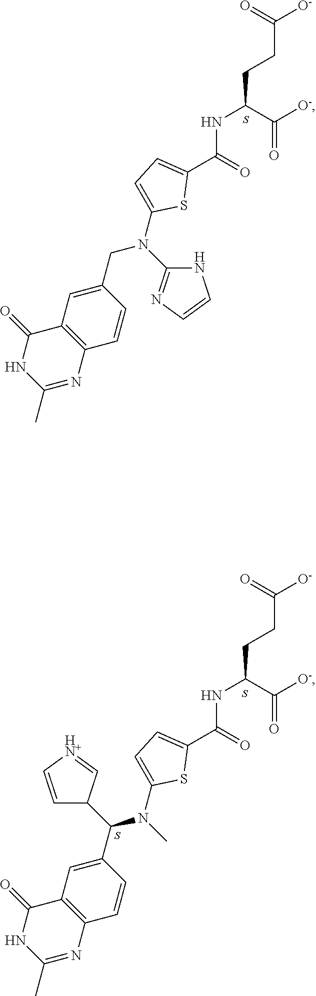

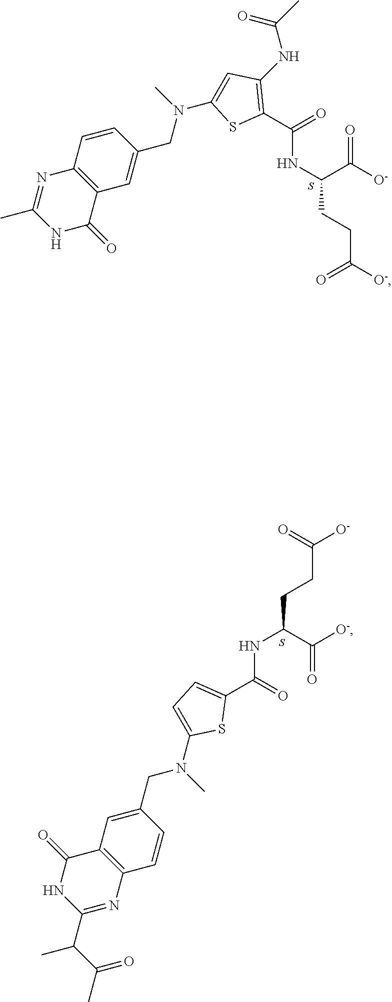

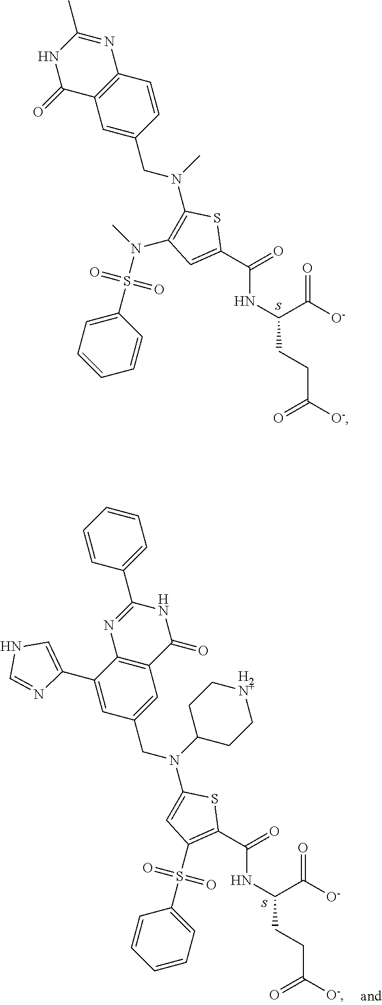

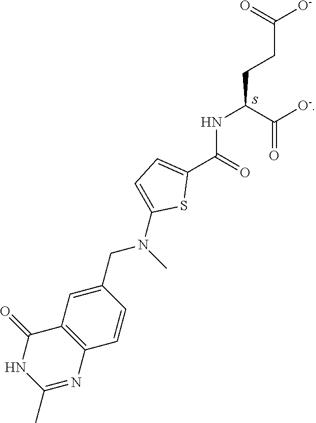

In certain embodiments, a compound described herein has a structure of Formula (I):

T-L-E (I)

or is a pharmaceutically acceptable salt thereof, wherein T is a radical of raltitrexed, 5-MTHF or an analog of either raltitrexed or 5-MTHF; L is a linker; and E is a radical of a therapeutic agent. In certain embodiments, L (i.e., the linker) can comprise a linker and a spacer (e.g., S¹).

T can have the structure of Formula (II):

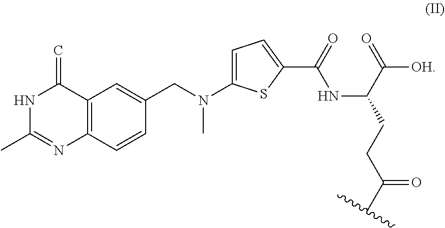

T can have the structure of Formula (III):

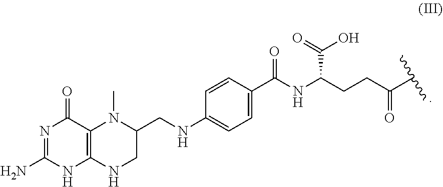 [SMILES: *C(=O)CC[C@H](NC(=O)c1ccc(NCC2CNc3[nH]c(N)nc(=O)c3N2C)cc1)C(=O)O]

T can be an analog of Formula (II) or Formula (III).

In certain embodiments, the compound has a structure of Formula (I):

T-L-E (I)

or is a pharmaceutically acceptable salt thereof, wherein T is a radical of raltitrexed, 5-MTHF, or an analog of raltitrexed or 5-MTHF; L is a linker; and E is a radical of a TLR7 agonist represented by Formula (IV):

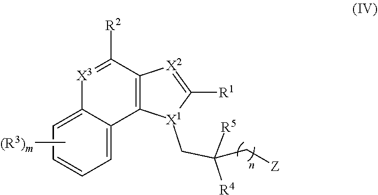

or a pharmaceutically acceptable salt thereof, wherein:
R¹, R³, R⁴, and R⁵ are each independently hydrogen (H), alkyl, alkoxyl, alkenyl, alkynyl, cycloalkyl, aryl halo, heteroaryl, —COR^{2x},

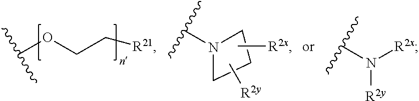

R² is a H, —OH, —NH₂, —NHR^{2x}, N₃, —NH—CH₂—NH₂, —CONH₂, —SO₂NH₂, —NH—CS—NH₂,

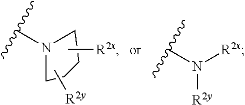

Z is a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

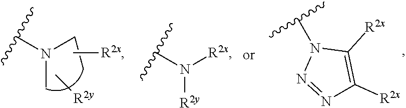

wherein:
   each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl,
   each R^{2z} is independently selected from the group consisting of —NH₂, —NR^{2q}R^{2q′}, —O—R^{2q}, —SO—R^{2q}, and —COR^{2q}; wherein each R^{2q} and R^{2q′} is independently alkyl or H,

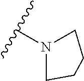 [SMILES: *N1CCCC1]

is a 3-10 membered N-containing non-aromatic, mono- or bicyclic heterocycle,
R²¹ is H or alkyl, and
n′ is 0-30; and
   wherein, in Formula (IV):
each of X¹, X², X³ is independently CR^{q} or N, wherein each R^{q} is independently H, halogen, or an optionally substituted alkyl;
n is 0-30;
m is 0-4; and
when n is 0, Y is not H, —OH, or —O—R^{2x}.

E of the compounds hereof (e.g., a radical of a therapeutic agent) can be any suitable therapeutic agent such as, for example, an immunomodulatory small molecule. Non-limiting examples of E include immunostimulants that stimulate the immune system by inducing activation or increasing activity of any of its components. The therapeutic agent can be selected from the group consisting of a toll-like receptor (TLR) agonist (e.g., a TLR7 agonist), a phosphoinositide 3-kinase (PI3K) inhibitor, a steroid, a nucleotide-binding and oligomerization domain (NOD)-like receptor 2 (NLR2) agonist, a stimulatory of interferon gene (STING) agonist, an enhancer of zeste homolog 2 (EZH2) inhibitor, a NOD-, LRR- and pyrin domain-containing protein 3 (NLRP3) inhibitor, a Caspase I inhibitor, a retinoic acid-inducible gene I (RIG-I)-like receptors (RLR) agonist, an absent in melanoma 2 (AIM2)-like receptor agonist, and an agonist of a receptor for advanced glycation end products (RAGE).

E can be a radical of any suitable immunomodulatory (e.g., immunoinhibitory) small molecule that binds to NLR2 (e.g., a NLR2 agonist). For example, the NLR2 agonist can be:

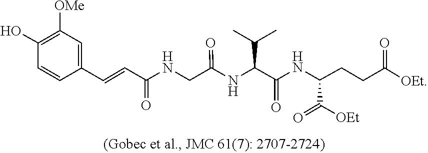

E can be a radical of any suitable immunomodulatory (e.g., immunoinhibitory) small molecule that binds to STING (e.g., a STING agonist). For example, the STING agonist can be:

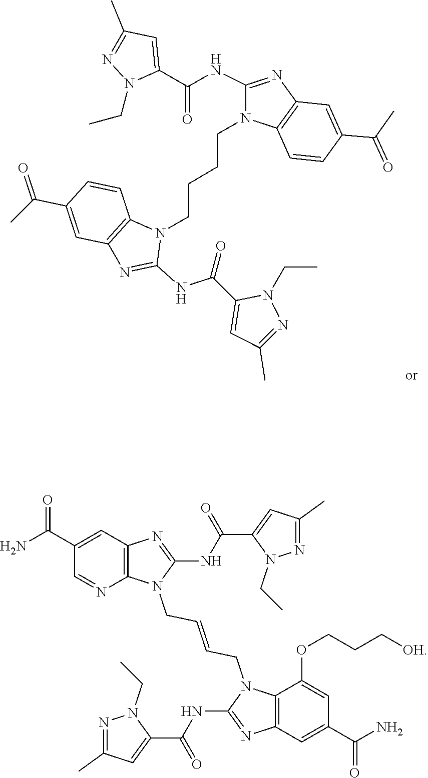

An additional non-limiting example of a STING agonist includes:

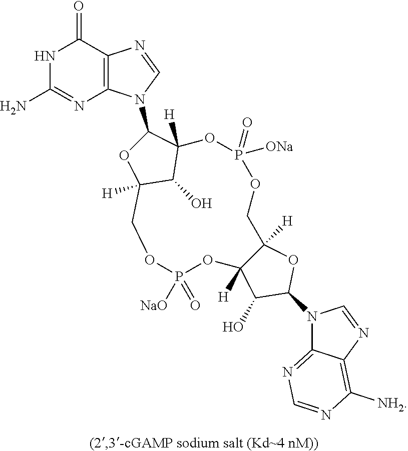

E can be a radical of any suitable immunomodulatory (e.g., immunoinhibitory) small molecule that binds to EZH2 (e.g., an EZH2 inhibitor). For example, and without limitation, the EZH2 inhibitor can be:

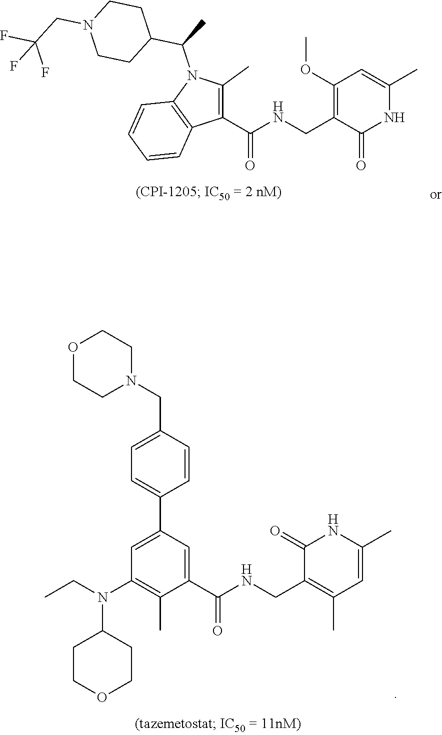

E can be a radical of any suitable immunomodulatory (e.g., immunoinhibitory) small molecule that binds to NLRP3 (e.g., a NLRP3 inhibitor). For example, the NLRP3 inhibitor can be

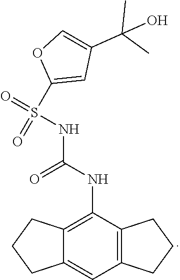

E can be a radical of any suitable immunomodulatory (e.g., immunoinhibitory) small molecule that binds to Caspase I (e.g., a Caspase I inhibitor). For example, the Caspase I inhibitor can be:

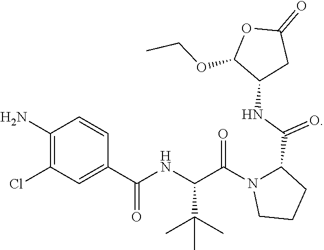

E can be a radical of any suitable immunomodulatory (e.g., immunoinhibitory) small molecule that binds to PI3K (e.g., a PI3K agonist). Non-limiting examples of PI3K agonists include, but are not limited to:

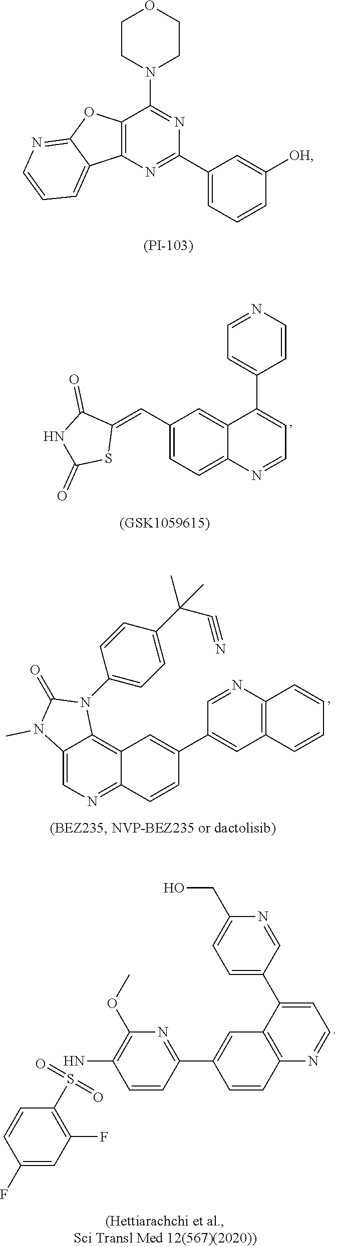

E can be a radical of any suitable immunomodulatory (e.g., immunoinhibitory) small molecule that binds to RLR (e.g., a RLR agonist). A non-limiting example of an RLR agonist is:

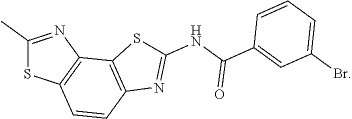

E can be a radical of any suitable immunomodulatory (e.g., immunoinhibitory) small molecule that binds to TLR (e.g., a TLR agonist). In certain embodiments, E is a radical of a TLR7 agonist.

In certain embodiments of the compound, E is a radical of a TLR7 agonist represented by Formula (IVA):

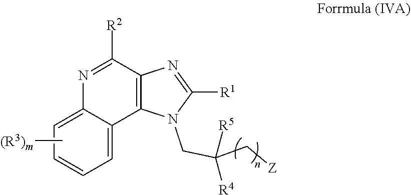

or is a pharmaceutically acceptable salt thereof, wherein:
R¹ is an optionally substituted C₃-C₈ alkyl;
R² is H, —OR^{z}, —SO₂N(R^{z})₂, —NR^{2x}R^{2y}, or N₃;
Z is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, wherein:
   R^{2x} and R^{2y} are each independently H, —N(R^{z})₂, —CON(R^{z})₂, —C(R^{z})₂—N(R^{z})₂, —CS—N(R^{z})₂, or an optionally substituted alkyl,
   each R^{z} is independently H, halogen, or optionally substituted alkyl, or
   R^{2x} and R^{2y} are taken together to form an optionally substituted heterocycloalkyl;
each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein the alkyl, alkoxy, heteroalkyl, cycloalkyl, or heterocycloalkyl and is optionally substituted;
R⁴ and R⁵ are each independently alkyl, alkoxy, halogen, or cycloalkyl, wherein the alkyl, alkoxy, and cycloalkyl is optionally substituted;
n is 1-6; and
m is 0-4.

In the compound having the radical of the structure of the TLR7 agonist of Formula (IVA), Z can be a group of the formula T-L-, T-L-O—, T-L-O-alkyl-, T-L-S¹—, T-SO₂—NH—, T-L-NR^{a}R^{b}—, T-L-S(O)ₓ-alkyl-, T-L-CO—, T-L-aryl-, T-L-NH—CO—NI—H—, T-L-NH—O—, T-L-NH—NH—, T-L-NH—CS—NH, T-L-C(O)-alkyl-, or T-L-SO₂—, wherein x is 0-3; wherein Si is a spacer; wherein R^{a} and Re are each independently H, halo, hydroxy, alkoxy, aryl, amino, acyl or C(O)R^{c}, wherein R^{c} is alkyl, aryl, oxy or alkoxy.

In certain embodiments of the compound having the structure of the TLR7 agonist of Formula (IVA), x is 1 or 2. In certain embodiments of the compound having the structure of the TLR7 agonist of Formula (IVA), n is 1-3. In certain embodiments of the compound having the structure of the TLR7 agonist of Formula (IVA), m is 0-4. In certain embodiments of the compound having the structure of the TLR7 agonist of Formula (IVA), x is 0-3; n is 1-3; and m is 0-4.

R¹ can be a C₁-C₆ alkyl optionally substituted with 1-3 substituents, each substituent independently being halogen or C₁-C₆ alkoxy; R² can be —NR^{2x}R^{2y}, where R^{2x} and R^{2y} are each independently a hydrogen or a C₁-C₆ alkyl. In addition, each R³ can be independently a halogen, —CN, C₁-C₆ alkyl, C₁-C₆ heteroalkyl, C₃-C₇ cycloalkyl, C₁-C₆ alkoxy, amino, hydroxy, carboxyl, or thiol; R⁴ and R⁵ can each independently be C₁-C₆ alkyl; each X¹, X², and X³ can be N; Z can be T-L- or T-L-O—; n can be 1; and m can be 0-Z can be T-L-O—.

R¹ can be optionally substituted C₃-C₆ alkyl. R¹ can be an optionally substituted acyclic C₃-C₆ alkyl. R² can be —NR^{2x}R^{2y} (as defined herein). R² can be —NH₂.

The TLR7 agonist of the compound can be a radical of one of the formulae:

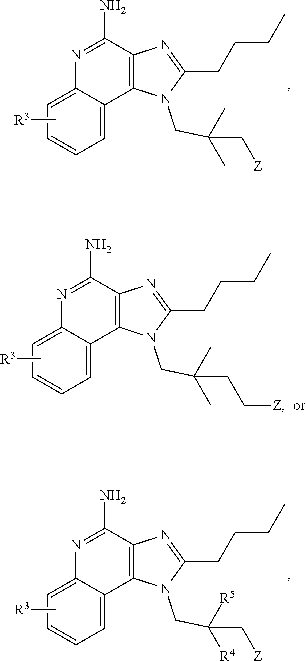

or a pharmaceutically acceptable salt of any of the foregoing formula, wherein R³ is optionally absent. The TLR7 agonist can be one of the formulae:

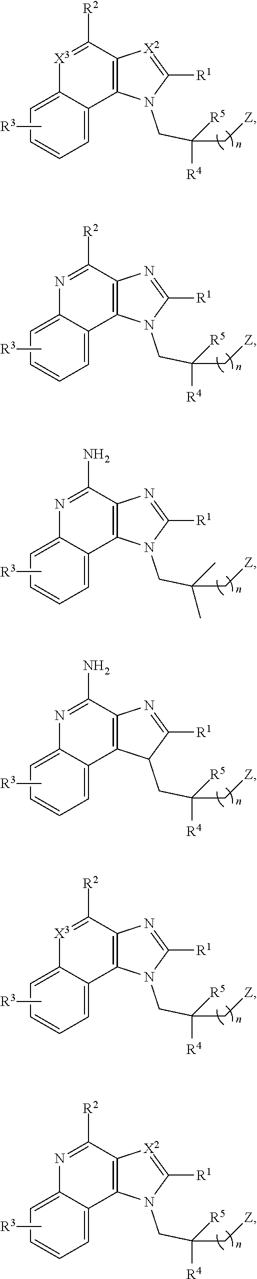

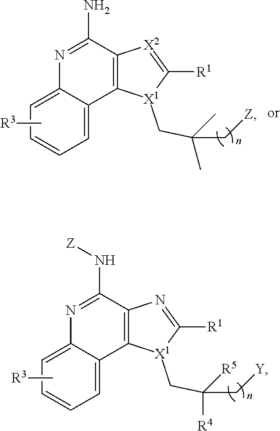

or a pharmaceutically acceptable salt of any of the foregoing formula, wherein R³ is optionally absent. R¹ can be a C₁-C₆ alkyl. R² can be —NH₂. In certain embodiments, R³ is absent. In certain embodiments, R¹ is a C₁-C₆ alkyl; R² is —NH₂; n is 1; and R³ is absent.

A compound comprising a radical of a TLR7 agonist of Formula (IVA) can be a compound of Formula (V):

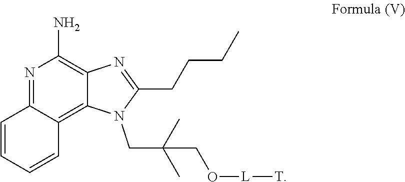

Further provided is a compound of the formula (I):

T-L-E (I)

or a pharmaceutically acceptable salt thereof, wherein T is a radical of raltitrexed, 5-MTHF, or an analog of raltitrexed or 5-MTHF; L is a linker; and E has the structure:

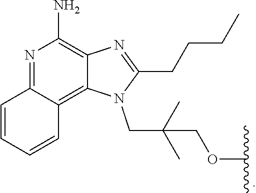

Still further provided is a compound of the formula (I):

T-L-E (I)

or a pharmaceutically acceptable salt thereof, wherein T is a radical of raltitrexed, 5-MTHF, an analog of raltitrexed, or an analog of 5-MTHF; L is a linker; and E is a radical of a compound that has the structure:

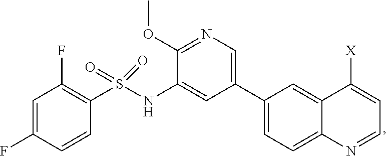

wherein X can be any of the following structures:

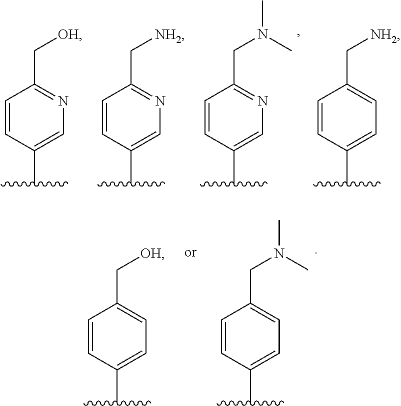

Non-limiting example of TLR agonists include a TLR 7 agonist, a TLR8 agonist, and a TLR7/8 agonist, such as:

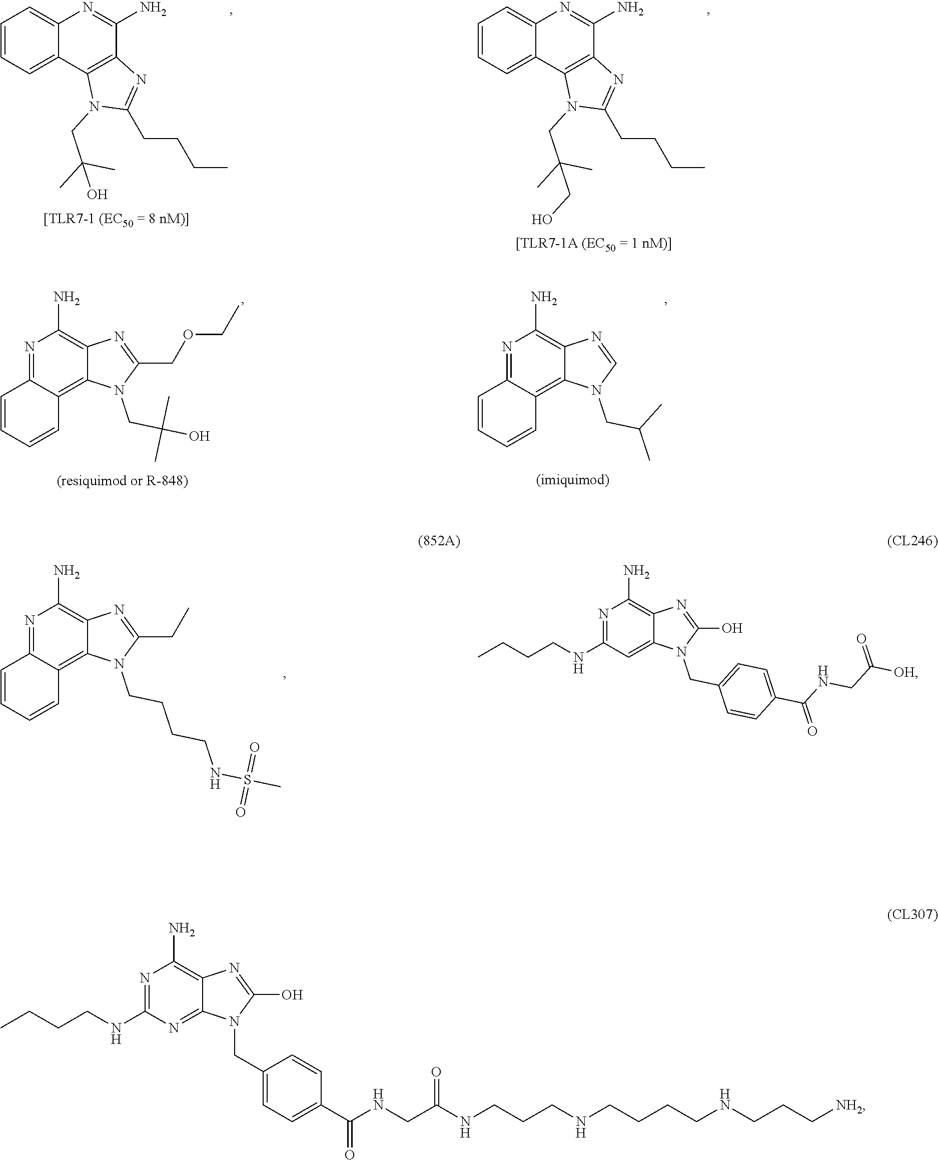

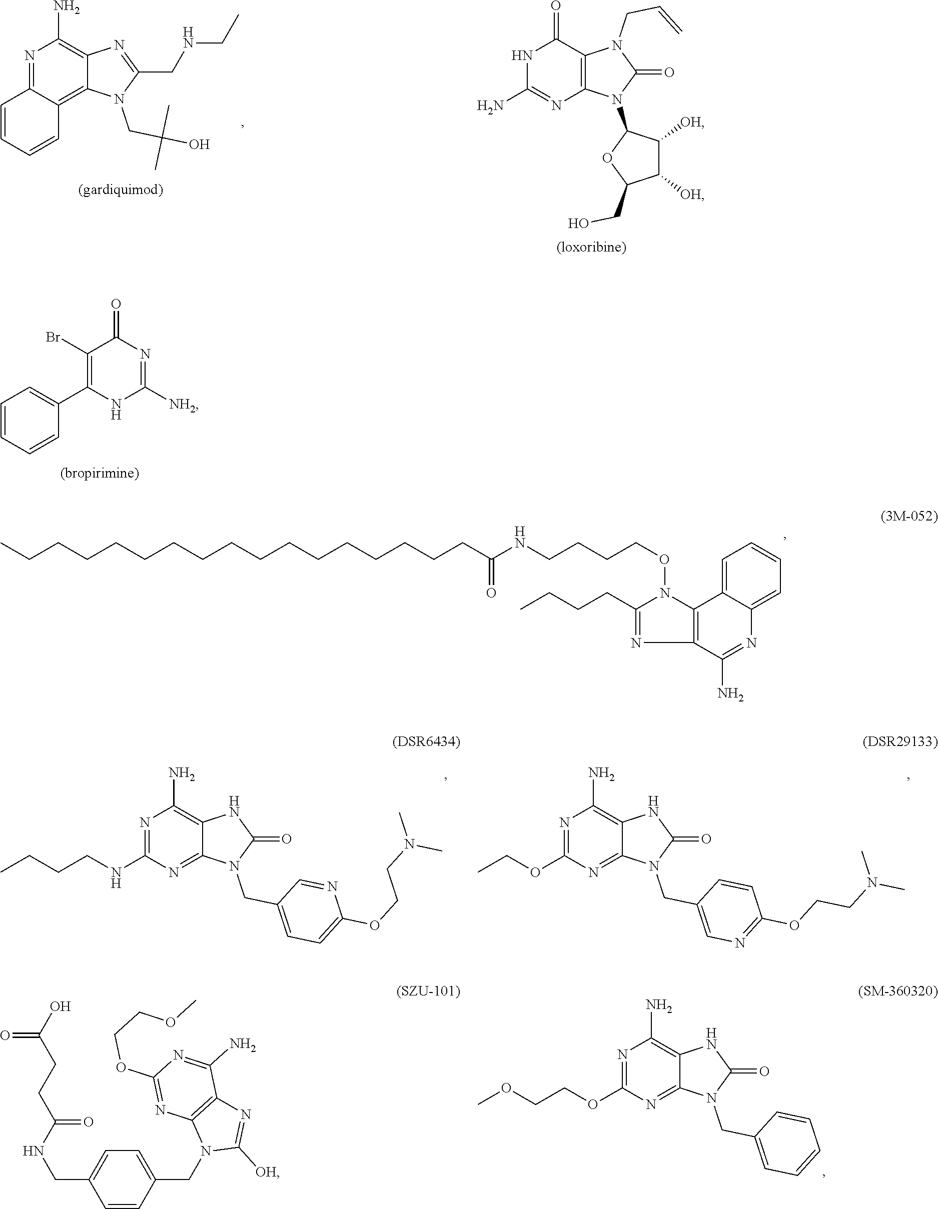

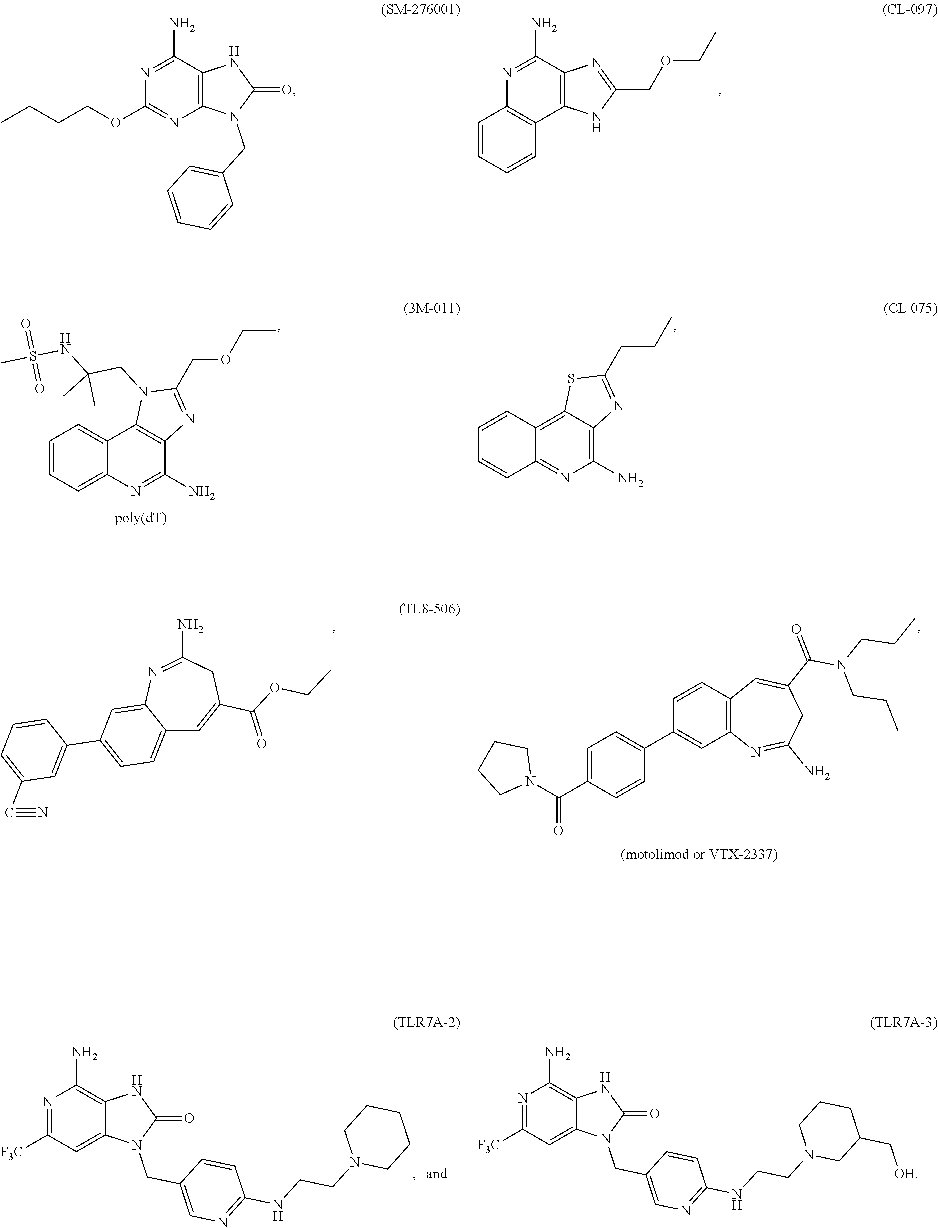

Poly deoxyhymine (poly dt) is a molecule made up of a string of deoxythymidines that are connected via 3′ to 5′ phosphodiester linkages. An oligonucleotide can be used for E. Examples of TLR9 agonists include, but are not limited to, CpG-ODN (short, synthetic ssDNA containing unmethylated CpG dinucleotide motifs within particular sequence contexts), IMO-2055 (synthetic oligonucleotide containing unmethylated CpG dinucleotides), and 1018 ISS (short, synthetic unmethylated CpG oligodeoxynucleotide (CpG ODN)). A nonlimiting example of a TLR3 agonist includes poly (I:C) (polyinosine homopolymer annealed to a strand of polycytidine homopolymer).

In some embodiments E can be an imaging agent, such as an optical or radioactive imaging agent. Nonlimiting examples of optical imaging agents include infrared, near infrared, and luminescent imaging agents. The optical imaging agent can be rhodamine or the indole-cyanine green-like dye S0456.

E can comprise a radical of the structure:

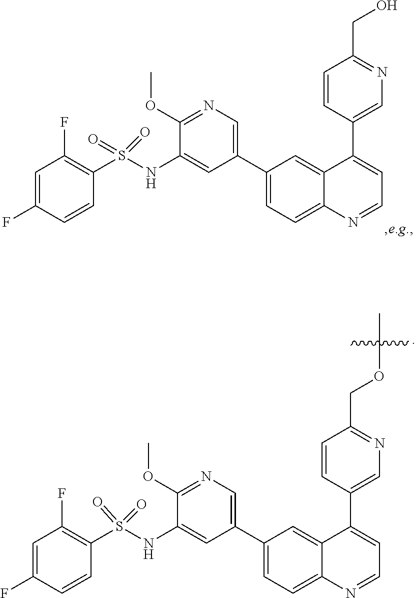

Another compound provided is one of formula (I):

T-L-E (I)

or a pharmaceutically acceptable salt thereof, wherein T is a radical of raltitrexed, 5-MTHF, an analog of raltitrexed, or an analog of 5-MTHF; L is a linker; and E is a radical of a corticosteroid. The corticosteroid can be betamethasone, cortisone, cortivazol, difluprednate, hydrocortisone, prednisolone, methylprednisolone, prednisone, dexamethasone, hydrocortisone-17-valerate, budesonide, flumethazone, fluticasone propionate, fluorocortisone, fludrocortisone, paramethasone, eplerenone, or an ester of any of the foregoing.

E (e.g., the radical of the therapeutic agent) of the compounds hereof can be conjugated to T via L (which may or may not additionally comprise a spacer (S¹)).

### Linkers (e.g., L and L′)

L of the compounds hereof can be a releasable linker. L of the compounds hereof can be a non-releasable linker. A releasable linker is a linker that includes at least one bond that can be broken under physiological conditions, such as reductive, acidic, basic, oxidative, metabolic, biochemical, enzymatic (e.g., cathepsin B-cleavable), or other conditions (e.g., p-aminobenzylic-based multivalent releasable bond (see, e.g., International Patent Application Publication Number WO 2017/0205661)). A non-releasable linker is a linker that includes an amide, an ester, an ether, an amine, or a thioether (e.g., thio-maleimide), for example.

L of the compounds hereof can comprise an optionally substituted heteroalkyl. The optionally substituted heteroalkyl can be substituted with at least one substituent selected from the group consisting of alkyl, hydroxyl, acyl, polyethylene glycol (PEG), carboxylate, and halo. L can comprise a substituted heteroalkyl with at least one disulfide bond in the backbone thereof. L can comprise a peptide or a peptidoglycan with at least one disulfide bond in the backbone thereof. In certain embodiments, L is a releasable linker that can be cleaved by enzymatic reaction, reaction oxygen species (ROS), or reductive conditions.

L can comprise the formula —NH—CH₂—CR⁶R⁷—S—S—CH₂—CH₂—O—CO—, wherein R⁶ and R⁷ are each, independently, H, alkyl, or heteroalkyl.

L can be a group or comprises a group of the formulae:

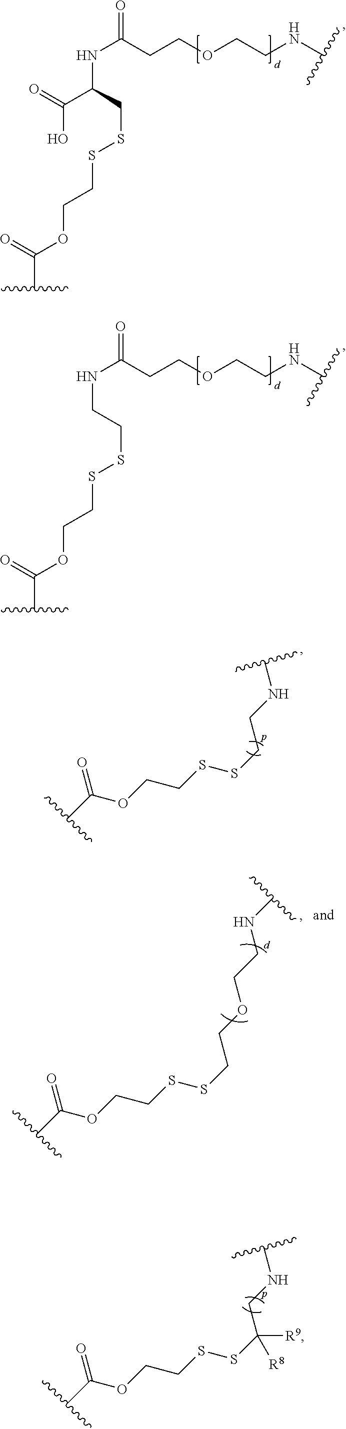

wherein p is an integer from 0 to 30; d is an integer from 1 to 40; and R⁸ and R⁹ are each, independently, H, alkyl, a heterocyclyl, a cycloalkyl, an aryl, or a heteroalkyl.

L can comprise one or more linker moieties independently selected from the group consisting of alkylene, heteroalkylene, —O— alkynylene, alkenylene, acyl, aryl, heteroaryl, amide, oxime, ether, ester, triazole, PEG, carboxylate, carbonate, carbamate, amino acid, peptide, and peptidoglycan. L can be or can comprise a peptide or a peptidoglycan. L can be or can comprise an amino acid. L can be or can comprise a PEG group. L can be or can comprise a polysaccharide.

L can be or can comprise a group represented by the structure:

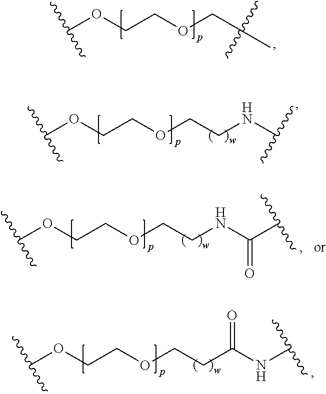

wherein w is 0-5 and p is 1-30. L can be or can comprise a linker moiety selected from the group consisting of

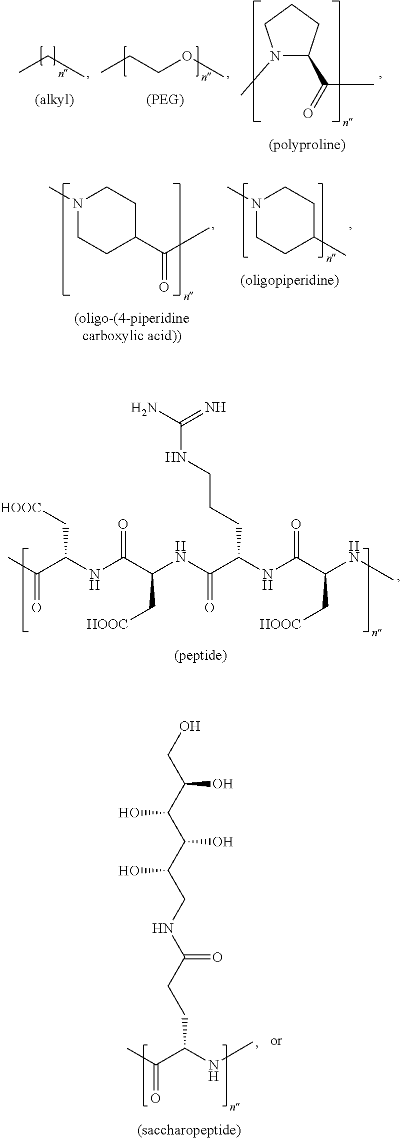

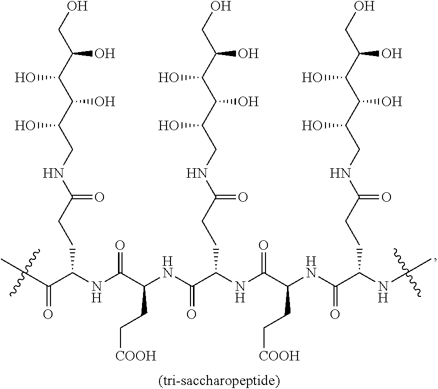

wherein n″ is 0-30. L can be a bivalent linker. L can be a trivalent linker.

In some embodiments, L is a pyrido[2,3-d]pyrimidine analog with the following structure:

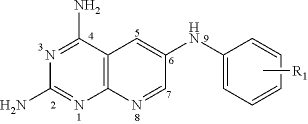

| | |
|---|---|
| 4 | R₁ = H |
| 5 | R₁ = 4′-OMe |
| 6 | R₁ = 2′-OMe |
| 7 | R₁ = 2′,5′-diOMe |
| 8 | R₁ = 3′,4′,5′-triOMe |
| 9 | R₁ = 4′-Me |
| 10 | R₁ = 4′-i-Pr |
| 11 | R₁ = 3′,4′-(C₄H₄) |
| 12 | R₁ = 4′-NO₂ |
| 13 | R₁ = 2′,3′-(C₄H₄) |
| 14 | R₁ = 2′,5′-diF |
| 15 | R₁ = 3′,4′,5′-triF |
| 16 | R₁ = H |
| 17 | R₁ = 4′-OMe |
| 18 | R₁ = 2′-OMe |
| 19 | R₁ = 2′,5′-diOMe |
| 20 | R₁ = 3′,4′,5′-triOMe |
| 21 | R₁ = 4′-Me |
| 22 | R₁ = 4′-i-Pr |
| 23 | R₁ = 4′-NO₂ |
| 24 | R₁ = 2′,3′-(C₄H₄) |
| 25 | R₁ = 2′,5′-diF |
| 26 | R₁ = 3′,4′,5-triF |

The linker can include a pharmacokinetic extender, such as an albumin binder or a hapten. Examples of albumin binders include, but are not limited to:

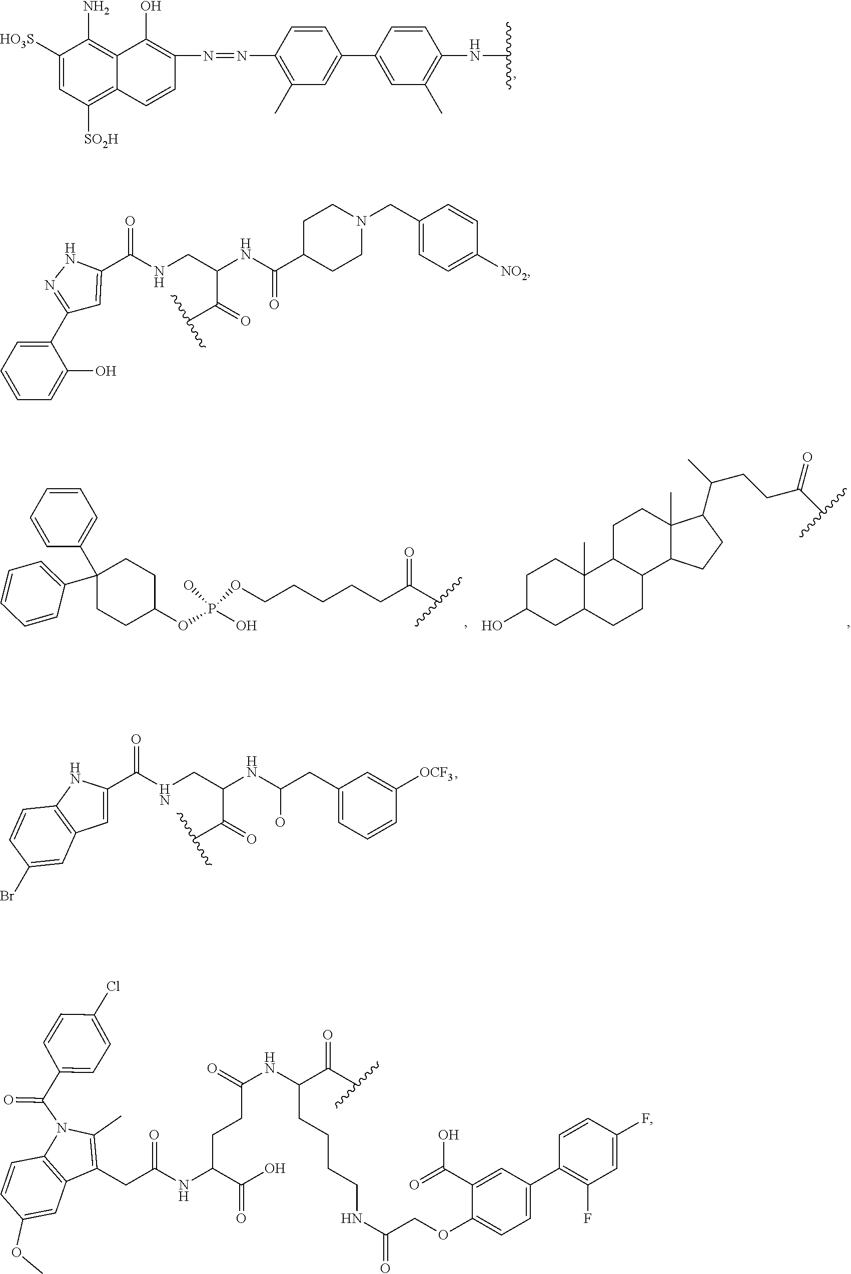

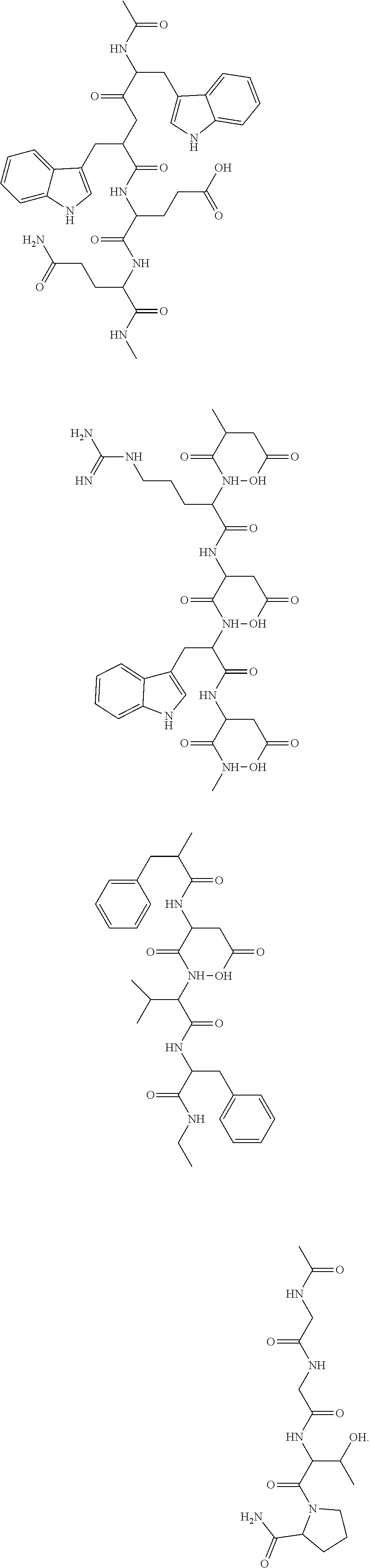 [SMILES: CNC(=O)C(CCC(N)=O)NC(=O)C(CCC(=O)O)NC(=O)C(CC(=O)C(Cc1c[nH]c2ccccc12)NC(C)=O)Cc1c[nH]c2ccccc12 ; CC1CC(=O)ON(C(CCCNC(=N)N)C(=O)NC2CC(=O)ON(C(Cc3c[nH]c4ccccc34)C(=O)NC3CC(=O)ON(C)C3=O)C2=O)C1=O ; CCNC(=O)C(Cc1ccccc1)NC(=O)C(C(C)C)N1OC(=O)CC(NC(=O)C(C)Cc2ccccc2)C1=O ; CC(=O)NCC(=O)NCC(=O)NC(C(=O)N1CCCC1C(N)=O)C(C)O]

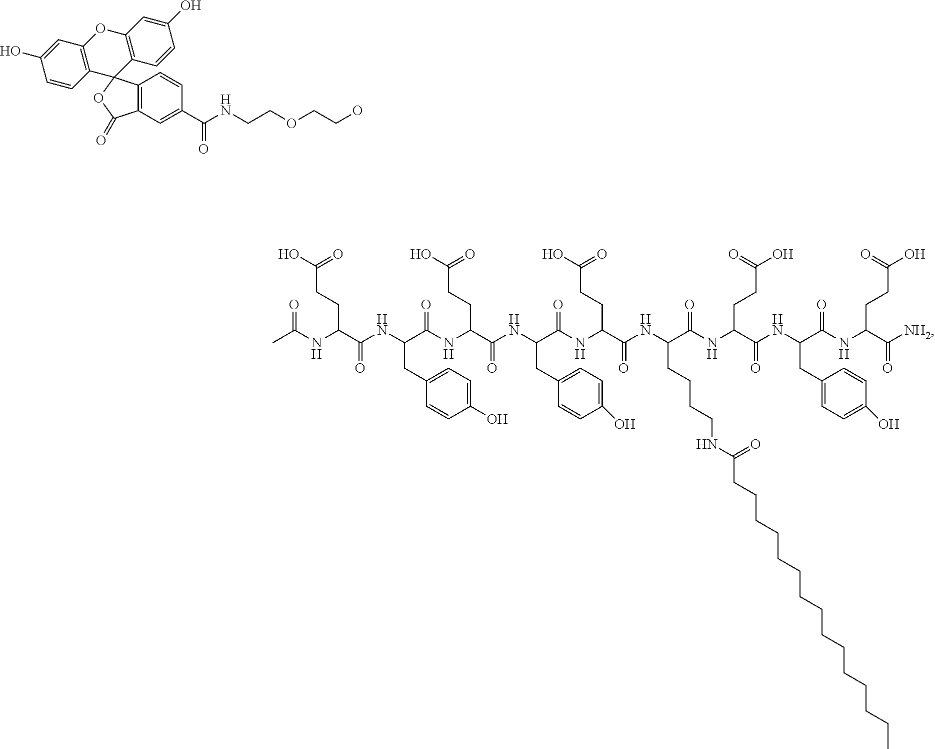 [SMILES: O=C(NCCOCCO)c1ccc2c(c1)C(=O)OC21c2ccc(O)cc2Oc2cc(O)ccc21 ; CCCCCCCCCCCCCCCC(=O)NCCCCC(NC(=O)C(CCC(=O)O)NC(=O)C(Cc1ccc(O)cc1)NC(=O)C(CCC(=O)O)NC(=O)C(Cc1ccc(O)cc1)NC(=O)C(CCC(=O)O)NC(C)=O)C(=O)NC(CCC(=O)O)C(=O)NC(Cc1ccc(O)cc1)C(=O)NC(CCC(=O)O)C(N)=O]

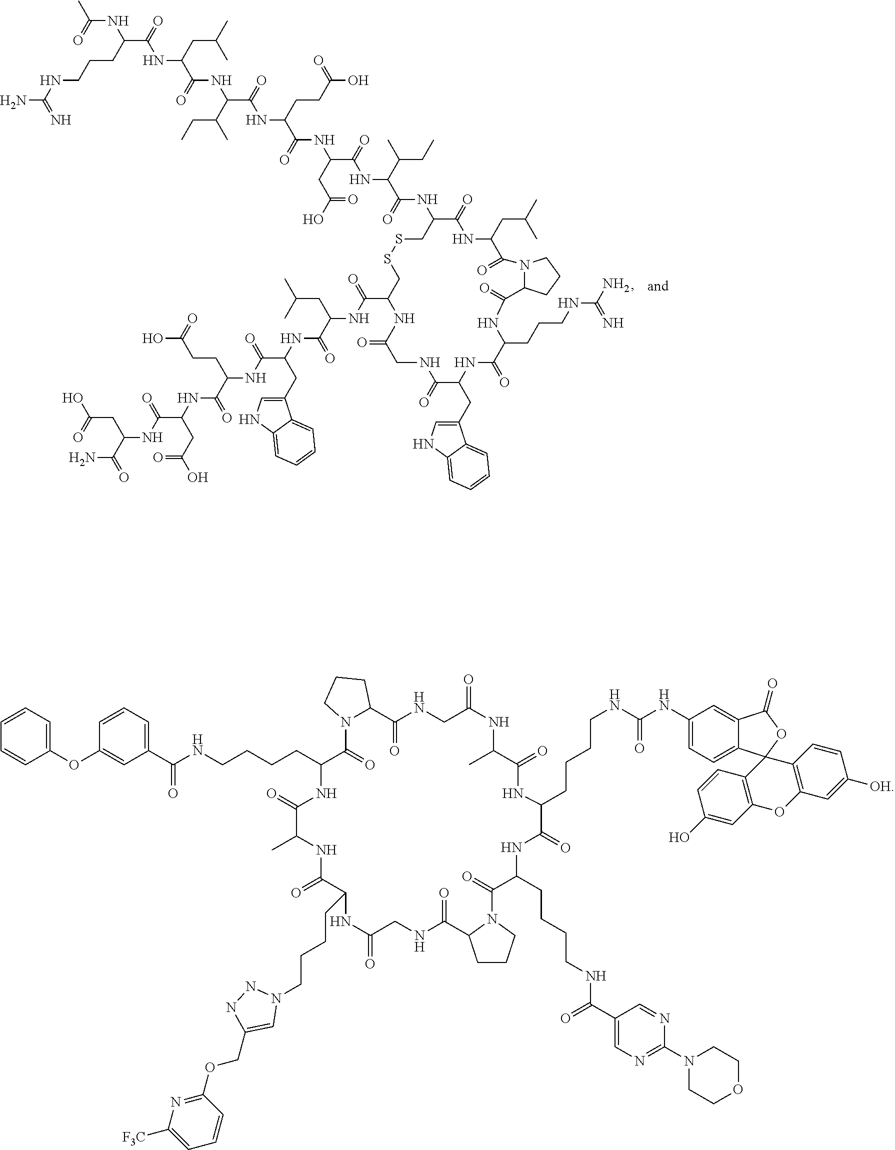

Examples of haptens include but are not limited to, 2,4-dinitrophenol (DNP) 2,4,6-trinitrophenol (TNP), rhamnose, galactose-α-1,3-galactose (α-Gal), or an antibody binder. Examples of antibody binders include, but are not limited to, a Fab, an scFv, a V_{H}, a V_{L}, a V_{H}H, a V-NAR, a monobody, an anticalin, an affibody, or a DARPin.

L of the compounds hereof can optionally be conjugated with and/or include a spacer (S¹). S¹ can be any suitable spacer. Examples of spacers include, but are not limited to, an alkyl chain with at least about 20 carbon atoms, e.g., at least 20 carbon atoms, in the chain, a PEG with at least about 20 units, e.g., at least 20 units, a sugar, a peptidoglycan, a clickable linker (e.g., a triazole), a rigid linker (e.g., a polyproline or a polypiperidine), or a combination of two or more of the foregoing.

Any of the compounds can further comprise S¹, which can include a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the L. The compound can further comprise an albumin binding group, e.g., an albumin binding group selected from the group consisting of

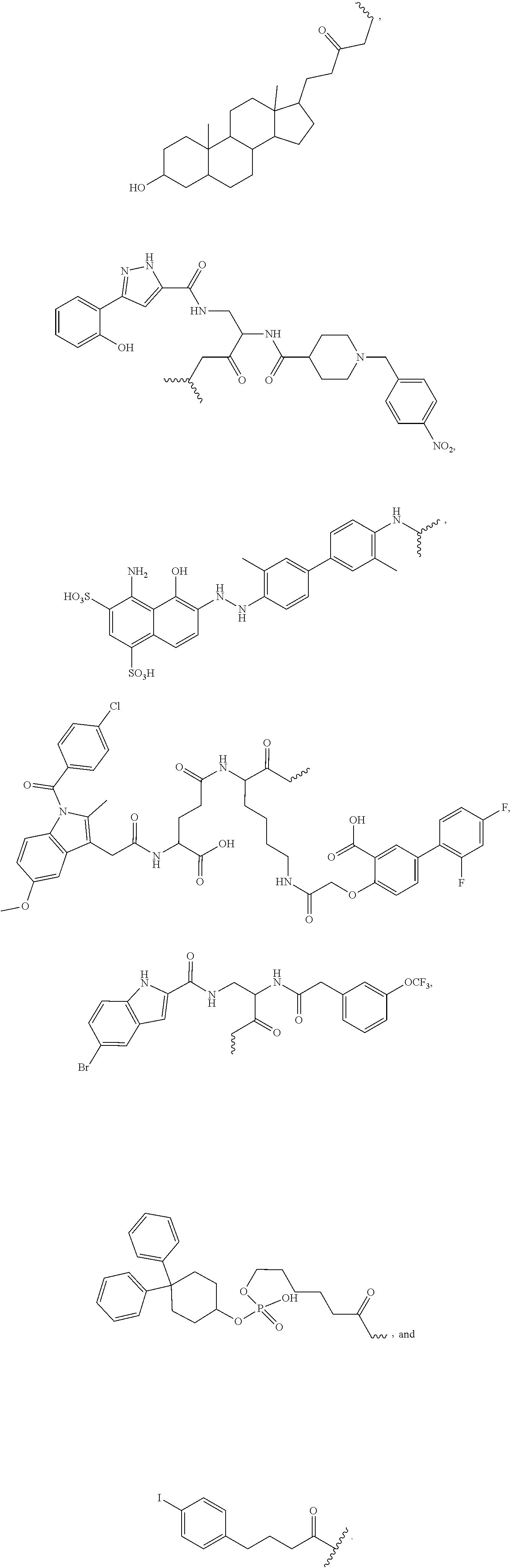

In certain embodiments, L comprises one or more releasable linkers that cleave under the conditions described herein by a chemical mechanism involving beta elimination. Such releasable linkers include beta-thio, beta-hydroxy, and beta-amino substituted carboxylic acids and derivatives thereof, such as esters, amides, carbonates, carbamates, and ureas. Such linkers also include 2- and 4-thioarylesters, carbamates, and carbonates.

An example of a releasable linker includes a linker of the formula:

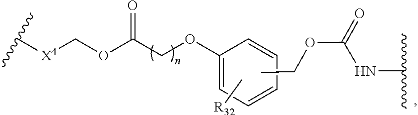

wherein X⁴ is NR, n is an integer selected from 0, 1, 2, and 3, and R₃₂ is H or a substituent, including a substituent capable of stabilizing a positive charge inductively or by resonance on the aryl ring, such as alkoxy, and the like. The releasable linker can be further substituted.

Assisted cleavage of releasable portions of L can include mechanisms involving benzylium intermediates, benzyne intermediates, lactone cyclization, oxonium intermediates, beta-elimination, and the like. In addition to fragmentation subsequent to cleavage of a releasable portion of L, the initial cleavage of the releasable linker can be facilitated by an anchimerically assisted mechanism. Thus, in the example of a releasable portion of L given above, the hydroxyalkanoic acid, which can cyclize, facilitates cleavage of the methylene bridge, by for example an oxonium ion, and facilitates bond cleavage or subsequent fragmentation after bond cleavage of the releasable linker. Alternatively, acid catalyzed oxonium ion-assisted cleavage of the methylene bridge can begin a cascade of fragmentation of this illustrative bivalent linker, or fragment thereof. Alternatively, acid-catalyzed hydrolysis of the carbamate can facilitate the beta elimination of the hydroxyalkanoic acid, which can cyclize, and facilitate cleavage of methylene bridge, by for example an oxonium ion. Other chemical mechanisms of bond cleavage under the metabolic, physiological, or cellular conditions can initiate such a cascade of fragmentation. Other chemical mechanisms of bond cleavage under the metabolic, physiological, or cellular conditions can initiate such a cascade of fragmentation.

Illustrative mechanisms for cleavage of the bivalent linkers described herein include the following 1,4 and 1,6 fragmentation mechanisms for carbonates and carbamates:

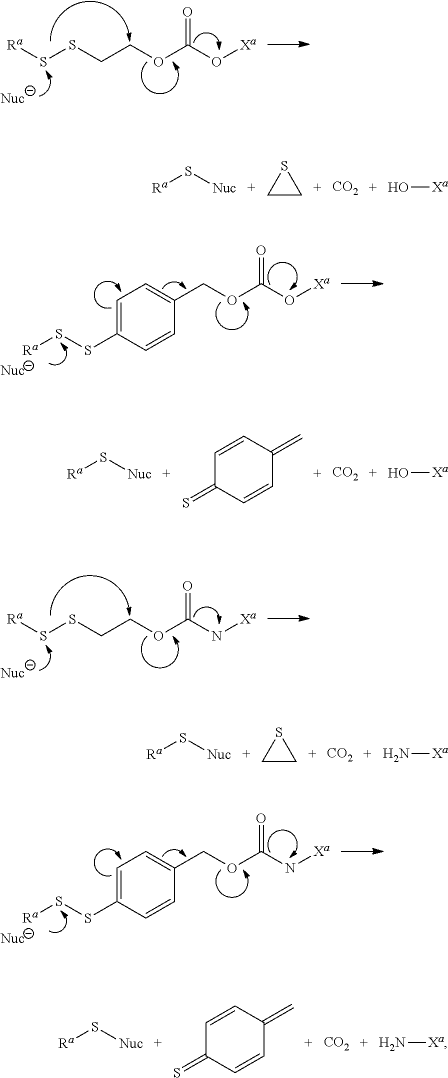

wherein Nuc⁻ is an exogenous or endogenous nucleophile, glutathione, or bioreducing agent, and the like, and R^{a} and X^{a} are connected through other portions of the bivalent linker. The location of R^{a} and X^{a} can be switched such that, e.g., the resulting products are X^{a}—S-Nuc and HO—R^{a} H2N—R^{a}.

Although the above fragmentation mechanisms are depicted as concerted mechanisms, any number of discrete steps can take place to affect the ultimate fragmentation of the bivalent linker to the final products shown. For example, the bond cleavage can also occur by acid catalyzed elimination of the carbamate moiety, which can be anchimerically assisted by the stabilization provided by either the aryl group of the beta sulfur or disulfide illustrated in the above examples. In those variations of this embodiment, the releasable linker is the carbamate moiety. Alternatively, the fragmentation can be initiated by a nucleophilic attack on the disulfide group, causing cleavage to form a thiolate. The thiolate can intermolecularly displace a carbonic acid or carbamic acid moiety and form the corresponding thiacyclopropane. In the case of the benzyl-containing bivalent linkers, following an illustrative cleavage of the disulfide bond, the resulting phenyl thiolate can further fragment to release a carbonic acid or carbamic acid moiety by forming a resonance-stabilized intermediate. In any of these cases, the releasable nature of the illustrative bivalent linkers described herein can be realized by whatever mechanism is relevant to the chemical, metabolic, physiological, or biological conditions present.

As described above, therefore, releasable linkers can comprise a disulfide group. Further examples of releasable linkers comprised in L include divalent radicals comprising alkyleneaziridin-1-yl, alkylenecarbonylaziridin-1-yl, carbonylalkylaziridin-1-yl, alkylenesulfoxylaziridin-1-yl, sulfoxylalkylaziridin-1-yl, sulfonylalkylaziridin-1-yl, or alkylenesulfonylaziridin-1-yl groups, wherein each of the releasable linkers is optionally substituted. Additional examples of releasable linkers comprised in L include divalent radicals comprising methylene, 1-alkoxyalkylene, 1-alkoxycycloalkylene, 1-alkoxyalkylenecarbonyl, 1-alkoxycycloalkylenecarbonyl, carbonylarylcarbonyl,carbonyl(carboxyaryl) carbonyl, carbonyl(biscarboxvaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio or carbonylalkylthio groups, wherein each of the releasable linkers is optionally substituted.

Additional examples of releasable linkers comprised in L can include an oxygen atom and methylene, 1-alkoxyalkylene, 1-alkoxycycloalkylene, 1-alkoxyalkylenecarbonyl or 1-alkoxycycloalkylenecarbonyl groups, wherein each of the releasable linkers is optionally substituted. Alternatively, in some embodiments the releasable linker includes an oxygen atom and a methylene group, wherein the methylene group is substituted with an optionally substituted aryl, and the releasable linker is bonded to the oxygen to form an acetal or ketal. Further, in some embodiments, the releasable linker includes an oxygen atom and a sulfonylalkyl group, and the releasable linker is bonded to the oxygen to form an alkylsulfonate.

Additional examples of releasable linkers comprised in L include a nitrogen and iminoalkylidenyl, carbonylalkylideniminyl, iminocycloalkylidenyl, and carbonylcycloalkylideniminyl groups, wherein each of the releasable linkers is optionally substituted and the releasable linker is bonded to the nitrogen to form a hydrazone. In some embodiments, the hydrazone is acylated with a carboxylic acid derivative, an orthoformate derivative, or a carbamoyl derivative to form various acylhydrazone releasable linkers.

Additional examples of releasable linkers comprised in L include an oxygen atom and alkylene(dialkylsilyl), alkylene(alkylarylsilyl), alkylene(diarylsilyl), (dialkylsilyl)aryl, (alkylarylsilyl)aryl or (diarylsilyl)aryl groups, wherein each of the releasable linkers is optionally substituted and the releasable linker is bonded to the oxygen to form a silanol.

Additional examples wherein L comprises a releasable linker include two independent nitrogens and carbonylarylcarbonyl, carbonyl(carboxyaryl)carbonyl, or carbonyl(biscarboxvaryl)carbonyl. In some embodiments the releasable linker is bonded to the heteroatom nitrogen to form an amide.

Additional examples wherein L comprises a releasable linker include an oxygen atom, a nitrogen, and a carbonylarylcarbonyl, carbonyl(carboxyaryl)carbonyl, or carbonyl(biscarboxvaryl)carbonyl. In some embodiments, the releasable linker forms an amide.

In some embodiments, L comprises an optionally substituted 1-alkylenesuccinimid-3-yl group and a releasable portion comprising methylene, 1-alkoxyalkylene, 1-alkoxycycloalkylene, 1-alkoxyalkylenecarbonyl or 1-alkoxycycloalkylenecarbonyl groups, each of which can be optionally substituted, to form a succinimid-1-ylalkyl acetal or ketal.

In some embodiments, L comprises carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl or 1-(carbonyltetrahydrofuranvl)succinimid-3-yl, each of which is optionally substituted. In some embodiments, L further comprises an additional nitrogen such that L comprises alkylenecarbonyl, cycloalkylenecarbonyl, carbonylalkylcarbonyl or 1-(carbonylalkyl)succinimid-3-yl groups, each of which is optionally substituted, bonded to the nitrogen to form an amide. In some embodiments, L further comprises a sulfur atom and alkylene or cycloalkylene groups, each of which is optionally substituted with carboxy, and is bonded to the sulfur to form a thiol. In some embodiments, L comprises a sulfur atom and 1-alkylenesuccinimid-3-yl and 1-(carbonylalkyl)succinimid-3-yl groups bonded to the sulfur to form a succinimid-3-ylthiol.

In some embodiments L comprises a nitrogen and a releasable portion comprising alkyleneaziridin-1-yl, carbonylalkylaziridin-1-yl, sulfoxylalkylaziridin-1-yl, or sulfonylalkylaziridin-1-yl, each of which is optionally substituted. In some embodiments, L comprises carbonyl, thionocarbonyl, alkylenecarbonyl, cycloalkylenecarbonyl, carbonylalkylcarbonyl, or 1-(carbonylalkyl)succinimid-3-yl, each of which can be optionally substituted and bonded to the releasable portion to form an aziridine amide.

L can comprise alkylene-amino-alkylenecarbonyl, alkylene-thio-(carbonylalkylsuccinimid-3-yl), and the like, as further illustrated by the following formulae:

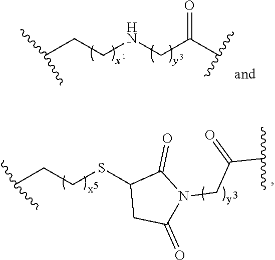

wherein x′ and y′ are each independently 1, 2, 3, 4, or 5.

L can have any suitable assortment of atoms in the chain, including C (e.g., —CH₂—, C(O)), N (e.g., NH, NR^{b}, wherein R^{b} is, e.g., H, alkyl, alkylaryl, and the like), O (e.g., —O—), P (e.g., —O— P(O)(OH)O—), and S (e.g., —S—). For example, the atoms used in forming L can be combined in all chemically relevant ways, such as chains of carbon atoms forming alkyl groups, chains of carbon and oxygen atoms forming polyoxyalkyl groups, chains of carbon and nitrogen atoms forming polyamines, and others, including rings, such as those that form aryl and heterocyclyl groups (e.g., triazoles, oxazoles, and the like). In addition, the bonds connecting atoms in the chain in L can be either saturated or unsaturated, such that for example, alkanes, alkenes, alkynes, cycloalkanes, arylenes, imides, and the like can be divalent radicals that are included in L. Further, the chain-forming L can be substituted or unsubstituted.

Additional examples of L groups include the groups 1-alkylsuccinimid-3-yl, carbonyl, thionocarbonyl, alkyl, cycloalkyl, alkylcycloalkyl, alkylcarbonyl, cycloalkylcarbonyl, carbonylalkylcarbonyl, 1-alkylsuccinimid-3-yl, 1-(carbonylalkyl)succinimid-3-yl, alkylsulfoxyl, sulfonylalkyl, alkylsulfoxylalkyl, alkylsulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranvl)succinimid-3-yl, wherein each group can be substituted or unsubstituted. Any of the aforementioned groups can be L or can be included as a portion of L. In some instances, any of the aforementioned groups can be used in combination (or more than once) (e.g., -alkyl-C(O)-alkyl) and can further comprise an additional nitrogen (e.g., alkyl-C(O)—NH—, —NH-alkyl-C(O)— or —NH-alkyl-), oxygen (e.g., -alkyl-O-alkyl-) or sulfur (e.g., -alkyl-S-alkyl-). Examples of such L groups are alkylcarbonyl, cycloalkylcarbonyl, carbonylalkylcarbonyl, 1-(carbonylalkyl)succinimid-3-yl, and succinimid-3-ylthiol, wherein each group can be substituted or unsubstituted.

In some embodiments, L is formed via click chemistry/click chemistry-derived. Those of skill in the art understand that the terms “click chemistry” and “click chemistry-derived” generally refer to a class of small molecule reactions commonly used in conjugation, allowing the joining of substrates of choice with specific molecules. Click chemistry is not a single specific reaction but describes a way of generating products that follow examples in nature, which also generates substances by joining small modular units. In many applications, click reactions join a biomolecule and a reporter molecule. Click chemistry is not limited to biological conditions: the concept of a “click” reaction has been used in pharmacological and various biomimetic applications. However, they have been made notably useful in the detection, localization and qualification of biomolecules.

Click reactions can occur in one pot, typically are not disturbed by water, can generate minimal byproducts, and are “spring-loaded”—characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (for in vivo systems).

Click chemistry examples include examples where L can be derived from copper-catalyzed azide-alkyne cycloaddition (CuAAC), strain-promoted azide-alkyne cycloaddition (SPAAC), inverse electron demand Diels-Alder reaction (IEDDA), and Staudinger ligation (SL). For example, X^{a} and R^{a} can be linked to each other as shown in Schemes 1-5:

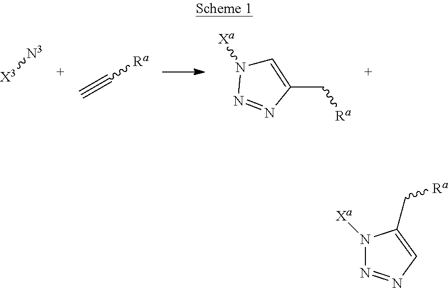

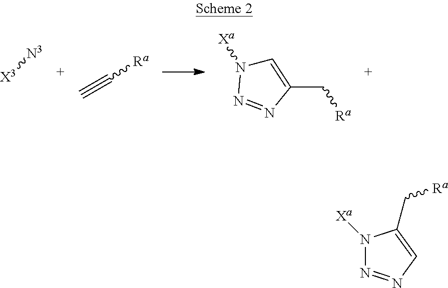

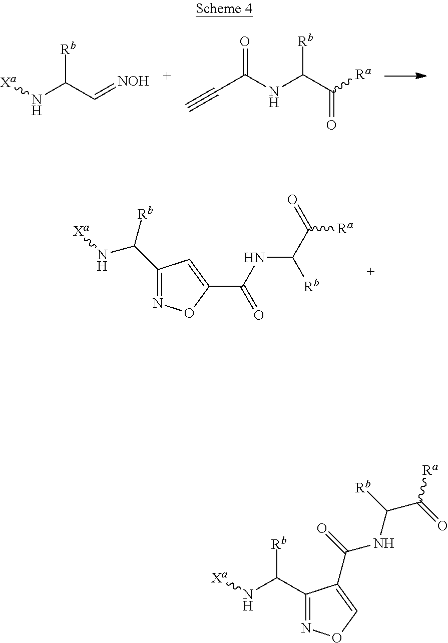

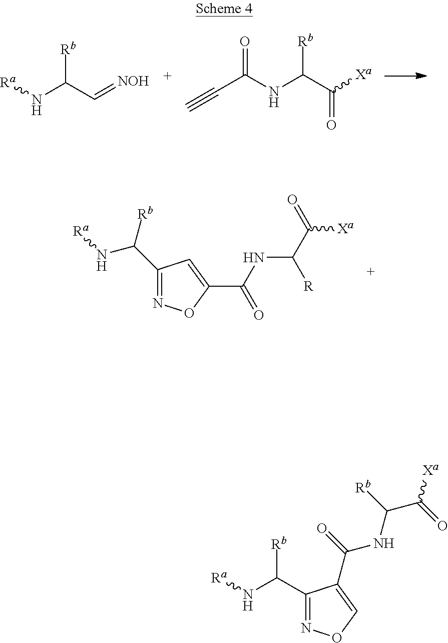

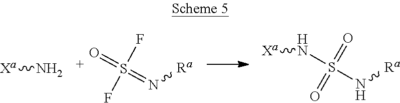

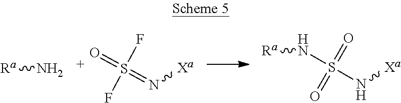

wherein each R^{b} is independently H, alkyl, arylalkyl, -alkyl-S-alkyl or arylalkyl or the side-chain of any naturally- or non-naturally occurring amino acid and the like. In Schemes 1-5, the wavy line connected to X^{a} and R^{a} represents a linkage between X^{a} and R^{a} and the groups to which they are attached. It should be appreciated that in Schemes 1-5, the triazole, oxazole, and the —NH—SO2-NH— group would be considered to be part of L.

In some embodiments, L is a linker selected from the group consisting of pegylated-, alkyl-, sugar-, and peptide-based dual linker; L is either a non-releasable linker or a releasable linker bivalently covalently attached to the folate ligand (or, in other embodiments, folate analogue or antifolate) and the steroid.

In some embodiments, L is:

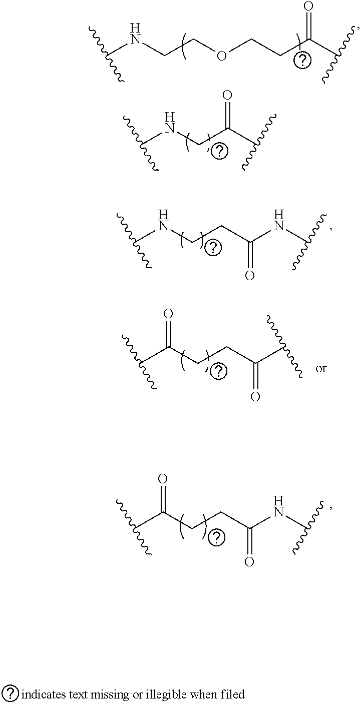

wherein
x″ is an integer from 0 to 10, and
y″ is an integer from 3 to 100.

In some aspects, x″ is an integer from 3 to 10.

In some embodiments, L is:

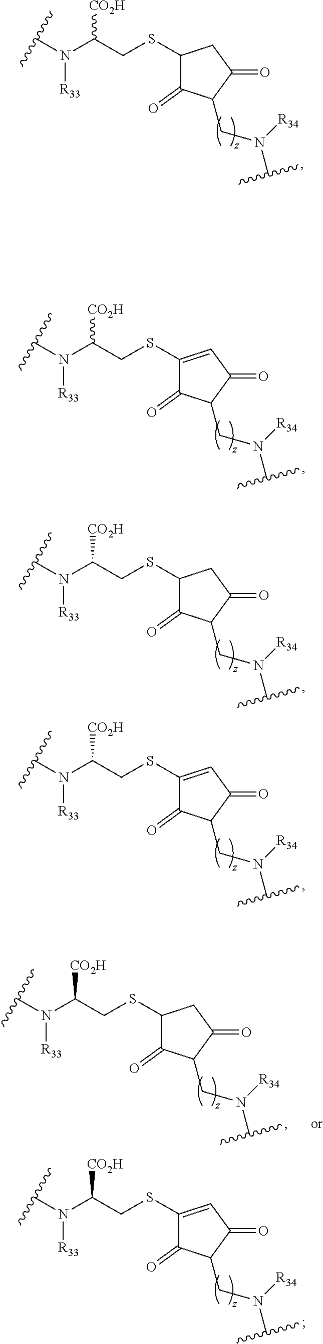

wherein each of R₃₃ and R₃₄ is independently H or C₁-C₆ alkyl;
and z is an integer from 1 to 8.

In some embodiments, L is:

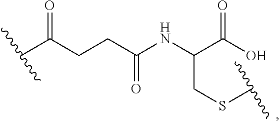 [SMILES: *SCC(NC(=O)CCC(*)=O)C(=O)O]

In some embodiments, L is:

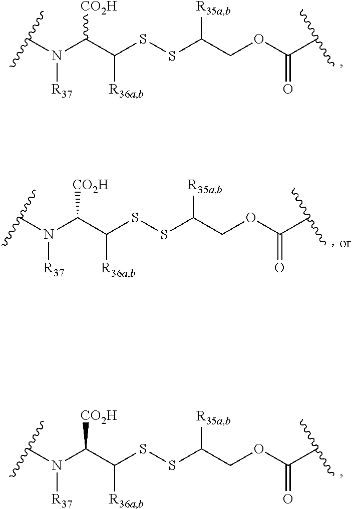

wherein R₃₇ is H or C₁-C₆ alkyl; R₃₅ₐ, R_{35b}, R₃₆ₐ, and R_{36b} each is independently H or C₁-C₆ alkyl.

In some embodiments, L comprises an amino acid. In some embodiments, L comprises an amino acid selected from the group consisting of Lys, Asn, Thr, Ser, Ile, Met, Pro, His, Gln, Arg, Gly, Asp, Glu, Ala, Val, Phe, Leu, Tyr, Cys, and Trp. In some embodiments, L comprises at least two amino acids independently selected from the group consisting of Glu and Cys. In some embodiments, L comprises Glu-Glu, wherein the glutamic acids are covalently bonded to each other through the carboxylic acid side chains.

In some embodiments, L comprises one or more hydrophilic spacer linkers comprising a plurality of hydroxyl functional groups.

In some embodiments, L comprises at least one 2,3-diaminopropionic acid group, at least one glutamic acid group (e.g., unnatural amino acid D-Glutamic acid), and at least one cysteine group. One example of such a linker is one having the non-natural amino acid, such as a linker having the repeating unit of the formula:

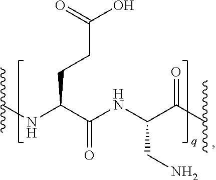

wherein q is an integer from 1 to 10 (e.g. 1 to 3 and 2 to 5). In some embodiments, L comprises the general formula:

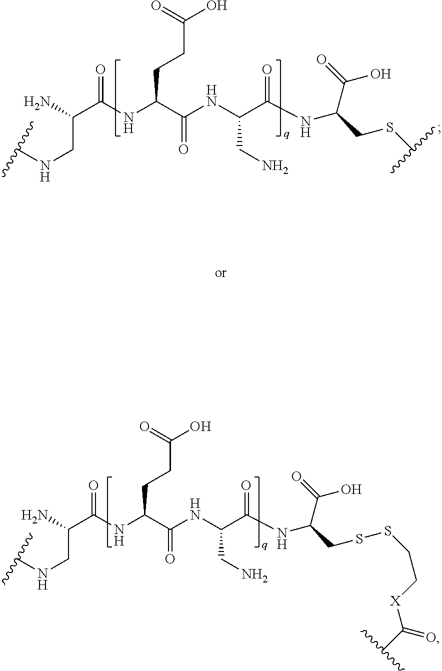

wherein X can be O, NH, NR or S, and q is an integer from 1 to 10. In some embodiments, L comprises the formula:

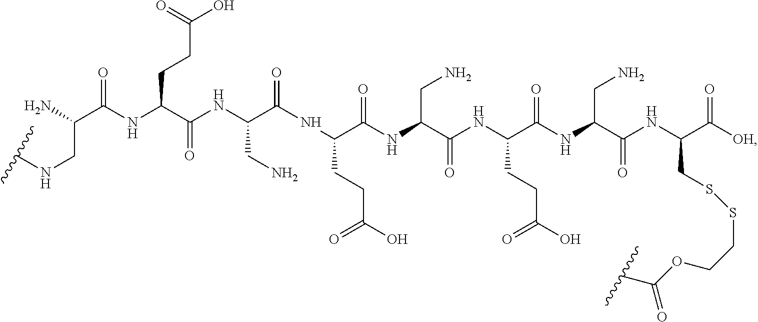 [SMILES: *NC[C@H](N)C(=O)N[C@@H](CCC(=O)O)C(=O)N[C@@H](CN)C(=O)N[C@@H](CCC(=O)O)C(=O)N[C@@H](CN)C(=O)N[C@@H](CCC(=O)O)C(=O)N[C@@H](CN)C(=O)N[C@H](CSSCCOC(*)=O)C(=O)O]

wherein the disulfide group is a part of a self-immolative group that can be generically described as a group of the formula —CH₂—S—S—CH₂—.

In some embodiments, the compounds described herein include linkages that cause the steroids described herein to be released by any suitable mechanism, including a release mechanism involving reduction, oxidation, or hydrolysis. An example of a reduction mechanism includes reduction of a disulfide group into two separate sulfyhydryl groups. Thus, for example, a group of the formula —CH₂—S—S—CH₂— would be reduced to two separate groups of the formula —CH₂—SH, such that if the linker were of the formula:

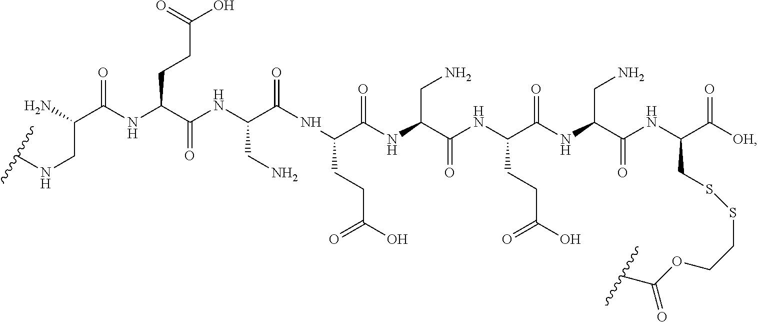 [SMILES: *NC[C@H](N)C(=O)N[C@@H](CCC(=O)O)C(=O)N[C@@H](CN)C(=O)N[C@@H](CCC(=O)O)C(=O)N[C@@H](CN)C(=O)N[C@@H](CCC(=O)O)C(=O)N[C@@H](CN)C(=O)N[C@H](CSSCCOC(*)=O)C(=O)O]

the reduction product would be of the formula:

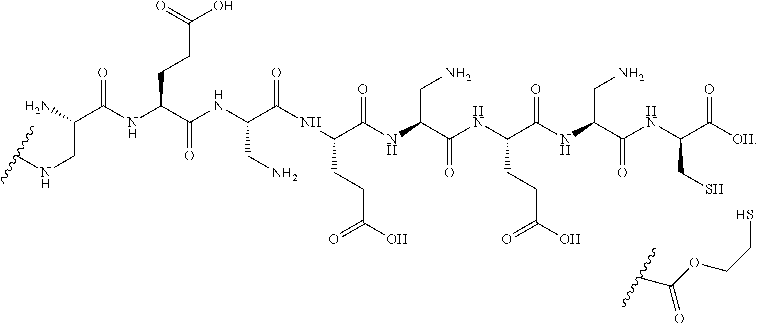 [SMILES: *NC[C@H](N)C(=O)N[C@@H](CCC(=O)O)C(=O)N[C@@H](CN)C(=O)N[C@@H](CCC(=O)O)C(=O)N[C@@H](CN)C(=O)N[C@@H](CCC(=O)O)C(=O)N[C@@H](CN)C(=O)N[C@H](CS)C(=O)O ; *C(=O)OCCS]

In this embodiment, the steroid is attached to the linker via a self-immolative moiety (e.g., a disulfide group).

An example of a self-immolative disulfide also includes a sterically protected disulfide bond. The steroid can be attached to the linker via any other suitable self-immolative bond, including via a self-immolative cathepsin cleavable amino acid sequence; via a self-immolative furin cleavable amino acid sequence; via a self-immolative β-glucuronidase cleavable moiety; via a self-immolative phosphatase cleavable moiety; or via a self-immolative sulfatase cleavable moiety. Multiple self-immolative linkages are also contemplated herein.

In some embodiments, the linker comprises a self-immolative moiety. In some embodiments, the linker comprises a self-immolative disulfide and or sterically protected disulfide bond. In some embodiments, the linker comprises a self-immolative cathepsin-cleavable amino acid sequence. In some embodiments, the linker comprises a self-immolative furin-cleavable amino acid sequence. In some embodiments, the linker comprises a self-immolative β-glucuronidase-cleavable moiety. In some embodiments, the linker comprises a self-immolative phosphatase-cleavable moiety. In some embodiments, the linker comprises a self-immolative sulfatase-cleavable moiety,

In some embodiments, the linker comprises a phosphate or pyrophosphate group. In some embodiments, the linker comprises a cathepsin B cleavable group. In some embodiments, the cathepsin B cleavable group is Valine-Citrulline. In some embodiments, the linker comprises a carbamate moiety. In some embodiments, the linker comprises a β-glucuronide.

In some embodiments, the compounds include linkages where the steroid is attached to the linker via an ester, phosphate, oxime, acetal, pyrophosphate, polyphosphate, disulfide, sulfate, hydrazide, imine, carbonate, carbamate or enzyme-cleavable amino acid sequence.

In some embodiments, the linker comprises an ester, phosphate, oxime, acetal, pyrophosphate, polyphosphate, disulfide, sulfate, hydrazide, imine, carbonate, carbamate or enzyme-cleavable amino acid sequence.

In some embodiments, L comprises one or more spacer linkers (e.g., S¹). Spacer linkers can be hydrophilic spacer linkers comprising a plurality of hydroxyl functional groups. A spacer can comprise any stable arrangement of atoms. Each spacer is independently selected from the group consisting an amide, ester, urea, carbonate, carbamate, disulfide, amino acid, amine, ether, alkyl, alkene, alkyne, heteroalkyl (e.g., polyethylene glycol), cycloakyl, aryl, heterocycloalkyl, heteroaryl, carbohydrate, glycan, peptidoglycan, polypeptide, or any combination thereof. In some embodiments, a spacer comprises any one or more of the following units: an amide, ester, urea, carbonate, carbamate, disulfide, amino acid, amine, ether, alkyl, alkene, alkyne, heteroalkyl (e.g., PEG), cycloakyl, aryl, heterocycloalkyl, heteroaryl, carbohydrate, glycan, peptidoglycan, polypeptide, or any combination thereof. In some embodiments, a spacer comprises a solubility enhancer or PK/PD modulator W. In some embodiments, a spacer comprises a glycosylated amino acid. In some embodiments, a spacer comprises one or more monosaccharide, disaccharide, polysaccharide, glycan, or peptidoglycan. In some embodiments, a spacer comprises a releasable moiety (e.g., a disulfide bond, an ester, or other moieties that can be cleaved in vivo). In some embodiments, a spacer comprises one or more units such as ethylene (e.g., polyethylene), ethylene glycol (e.g., PEG), ethanolamine, ethylenediamine, and the like (e.g., propylene glycol, propanolamine, propylenediamine). In some embodiments, a spacer comprises an oligoethylene, PEG, alkyl chain, oligopeptide, polypeptide, rigid functionality, peptidoglycan, oligoproline, oligopiperidine, or any combination thereof. In some embodiments, a spacer comprises an oligoethylene glycol or a PEG. A spacer can comprise an oligoethylene glycol. In some embodiments, a spacer comprises a PEG. In some embodiments, a spacer comprises an oligopeptide or polypeptide. In some embodiments, a spacer comprises an oligopeptide. In some embodiments, a spacer comprises a polypeptide. In some embodiments, a spacer comprises a peptidoglycan. In some embodiments, a spacer does not comprise a glycan. In some embodiments, a spacer does not comprise a sugar. In some embodiments, a rigid functionality is an oligoproline or oligopiperidine. In some embodiments, a rigid functionality is an oligoproline. In some embodiments, a rigid functionality is an oligopiperidine. In some embodiments, a rigid functionality is an oligophenyl. In some embodiments, a rigid functionality is an oligoalkyne. In some embodiments, an oligoproline or oligopiperidine has about two up to and including about fifty, about two to about forty, about two to about thirty, about two to about twenty, about two to about fifteen, about two to about ten, or about two to about six repeating units (e.g., prolines or piperidines).

In some embodiments, L comprises a solubility enhancer or PK/PD modulator. In some embodiments, L comprises PEG, sugar, peptide, or peptidoglycan. In some embodiments, L comprises a PEG, sugar, peptide, or peptidoglycan for achieving better solubility and PK/PD properties. In some embodiments, L comprises one or more monosaccharide, disaccharide, peptide, peptidoglycan, and/or serum albumin. In some embodiments, L comprises one or more PEG, peptide, peptidoglycan, or serum albumin. In some embodiments, W does not comprise a sugar. In some embodiments, W does not comprise a monosaccharide, disaccharide, or polysaccharide. In some embodiments, W does not comprise a glycan. In some embodiments, L comprises a glycosylated amino acid. In some embodiments, L comprises a glycosylate cysteine. In some embodiments, L comprises a free carboxylic acid. In some embodiments, L comprises a PEG.

In some embodiments, L comprises one or more monosaccharide, disaccharide, oligosaccharide, polysaccharide, peptide, peptidoglycan, serum albumin, solubility enhancer, PK/PD modulator, or a combination thereof. In some embodiments, L modulates a pharmacological, pharmacokinetic, pharmacodynamic, or physicochemical property. In some embodiments, L facilitates internalization. In some embodiments, L improves aqueous solubility. In some embodiments, L increases plasma protein binding. In some embodiments, W modulates (e.g., reduces) the compound's excretion, elimination, metabolism, stability (e.g., enzymatic stability, plasma stability), distribution, toxicity, or a combination thereof.

In some embodiments, a monosaccharide such as found in W exists in an equilibrium between its linear and cyclic form. In some embodiments, a monosaccharide is linear. In some embodiments, a monosaccharide is cyclic. In some embodiments, a monosaccharide exists as a D isomer. In some embodiments, a monosaccharide exists as an L isomer. As non-limiting examples, in some embodiments, L comprises one or more monosaccharides selected from the following: ribose, galactose, mannose, glucosefructose, N-acetylglucosamine, N-acetylmuramic acid or derivatives thereof (e.g., cyclic or linear forms, methylated derivatives, acetylated derivatives, phosphorylated derivatives, aminated derivatives, oxidized or reduced derivatives, D or L isomers, isotopes, stereoisomers, regioisomers, tautomers, or combinations thereof).

In some embodiments, a disaccharide, oligosaccharide, or polysaccharide, as can be disposed within W, contains an O-linkage, an N-linkage, a C-linkage, or a combination thereof. In some embodiments, a disaccharide, oligosaccharide, or polysaccharide contains a glycosidic linkage in either an alpha- or beta-orientation. In some embodiments, L comprises an oligosaccharide, a polysaccharide, or a glycan (e.g., a glycoprotein, glycopeptide, glycolipid, glycogen, proteoglycan, peptidoglycan, and the like).

In some embodiments, L comprises an amino acid, a peptide, a polypeptide, or a protein. In some embodiments, the amino acid is a natural amino acid (e.g., alanine (Ala), arginine (Arg), asparagine (Asn), aspartic acid (Asp), cysteine (Cys), glutamic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val)). Alternatively, in some embodiments, the amino acid is an unnatural or modified amino acid. L can comprise a sugar or sugar derivative covalently attached to the side chain of an amino acid (e.g., a glutamic acid, an aspartic acid).

In some embodiments, L comprises a glycosylated amino acid such as:

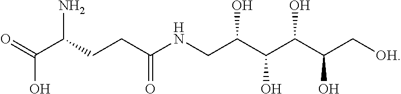 [SMILES: N[C@H](CCC(=O)NC[C@H](O)[C@@H](O)[C@H](O)[C@H](O)CO)C(=O)O]

In some embodiments, a peptide or polypeptide comprises a plurality of amino acids, natural and/or unnatural. In some embodiments, a peptide (or peptidoglycan) has about two and about twenty amino acids. In some embodiments, an amino acid, a peptide, a polypeptide, or a protein has a pharmacological or physicochemical effect that enhances one or more properties of the compound (e.g., modulating solubility, solubility, size, permeability, protein binding, target binding, excretion, metabolism, toxicity, distribution, half-life, and/or duration of action). In some embodiments, L comprises a pharmacokinetic modulator. In some embodiments, the pharmacokinetic modulator is a peptide or protein that can modulate (e.g., enhance) protein binding. In some embodiments, the pharmacokinetic modulator enhances plasma protein binding. In some embodiments, the pharmacokinetic modulator reduces the rate of elimination, excretion, or metabolism. In some embodiments, the pharmacokinetic modulator increases the duration of action of the compound.

In some embodiments, the linker comprises an albumin ligand. In some embodiments, the albumin ligand comprises

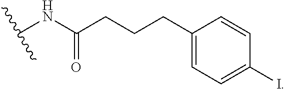 [SMILES: *NC(=O)CCCc1ccc(I)cc1]

In some embodiments, L comprises the following structure:

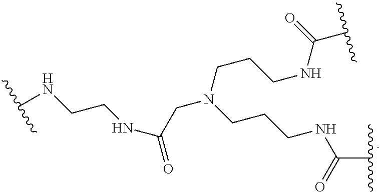 [SMILES: *NCCNC(=O)CN(CCCNC(*)=O)CCCNC(*)=O]

In some embodiments, L comprises a template (e.g., a multivalent template) that connects multiple arms of the compound and comprises a template (e.g., a repeating unit) of the following structure:

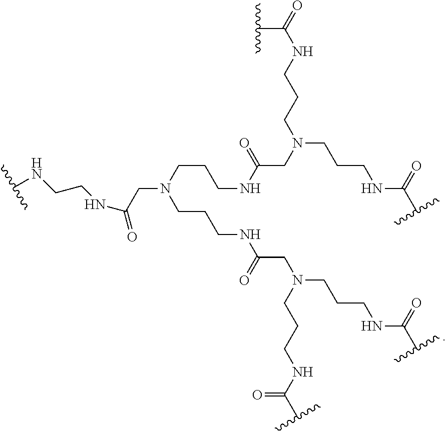

In some embodiments, L comprises a template that connects multiple arms of the compound that has a citric acid-based template. In some embodiments, L comprises a template (e.g., a multivalent template) that connects multiple arms of the compound and has a (e.g., citric acid-based) template of the following structure:

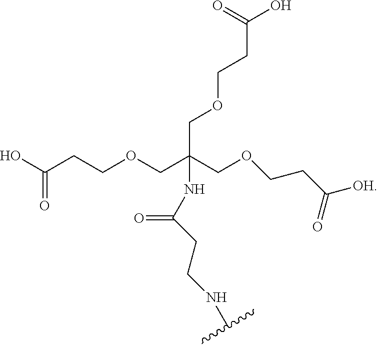 [SMILES: *NCCC(=O)NC(COCCC(=O)O)(COCCC(=O)O)COCCC(=O)O]

In some embodiments, L comprises a template (e.g., a multivalent template) that connects multiple arms of the compound and has a (e.g., citric acid-based) template of the following structure:

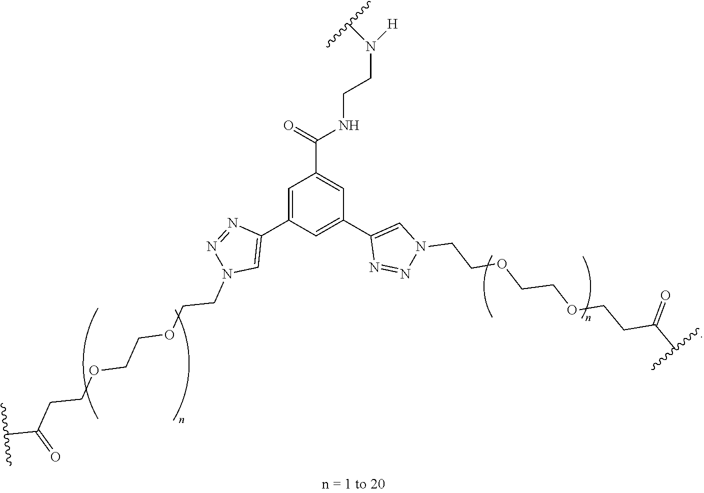

In some embodiments, L comprises a template (e.g., a multivalent template) that connects multiple arms of the compound and has a (e.g., citric acid-based) template of the following structure:

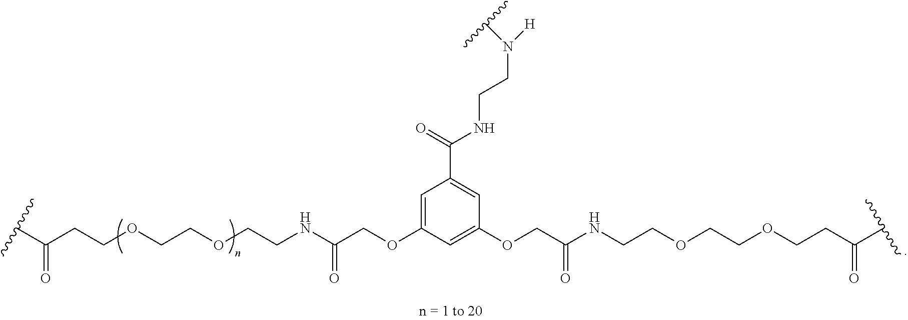

In some embodiments, the linker comprises a dimethylcysteine group. In some embodiments, the dimethylcysteine group is linked to a succinimide to form:

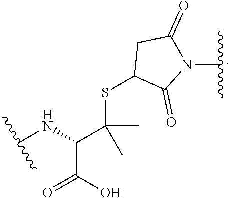 [SMILES: *N[C@@H](C(=O)O)C(C)(C)SC1CC(=O)N(*)C1=O]

L′ of the compounds hereof can comprise an optionally substituted heteroalkyl. The optionally substituted heteroalkyL′ can be substituted with at least one substituent selected from the group consisting of alkyl, hydroxyl, acyl, polyethylene glycol (PEG), carboxylate, and halo. L′ can comprise a substituted heteroalkyl with at least one disulfide bond in the backbone thereof. L′ can comprise a peptide or a peptidoglycan with at least one disulfide bond in the backbone thereof. L′ can be a releasable linker that can be cleaved by enzymatic reaction, reaction oxygen species (ROS), or reductive conditions.

L′ can comprise the formula —NH—CH₂—CR⁶R⁷—S—S—CH₂—CH₂—O—CO—, wherein R⁶ and R⁷ are each, independently, H, alkyl, or heteroalkyl.

L′ can be a group or comprises a group of the formulae:

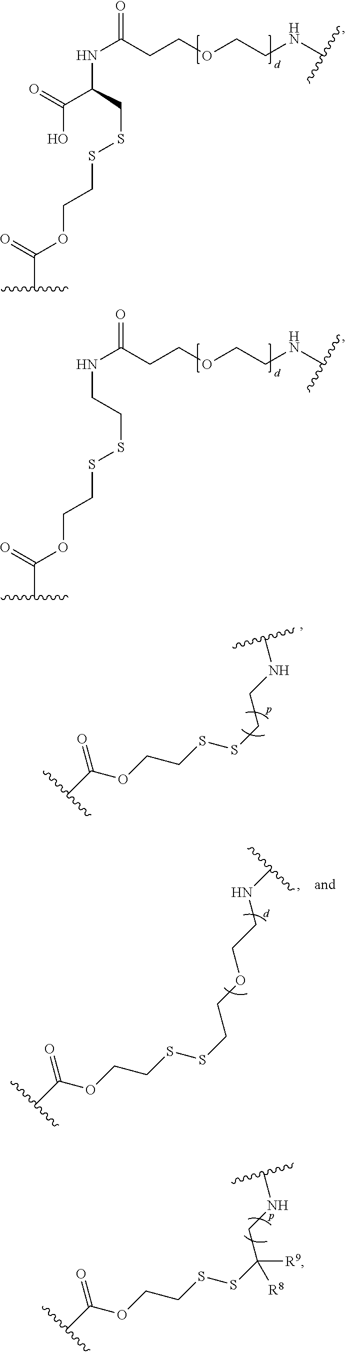

wherein p is an integer from 0 to 30; d is an integer from 1 to 40; and R⁸ and R⁹ are each, independently, H, alkyl, a heterocyclyl, a cycloalkyl, an aryl, or a heteroalkyl.

L′ can comprise one or more linker moieties independently selected from the group consisting of alkylene, heteroalkylene, —O— alkynylene, alkenylene, acyl, aryl, heteroaryl, amide, oxime, ether, ester, triazole, PEG, carboxylate, carbonate, carbamate, amino acid, peptide, and peptidoglycan. L′ can be or can comprise a peptide or a peptidoglycan. L′ can be or can comprise an amino acid. L′ can be or can comprise a PEG group. L′ can be or can comprise a polysaccharide. L′ can be or can comprise a group represented by the structure:

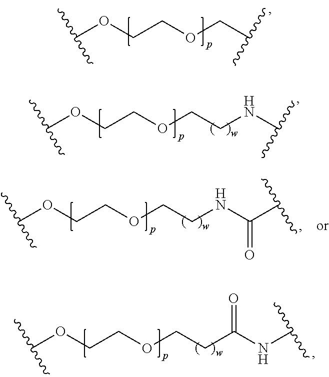

wherein w is 0-5 and p is 1-30. L′ can be or can comprise a linker moiety selected from the group consisting of:

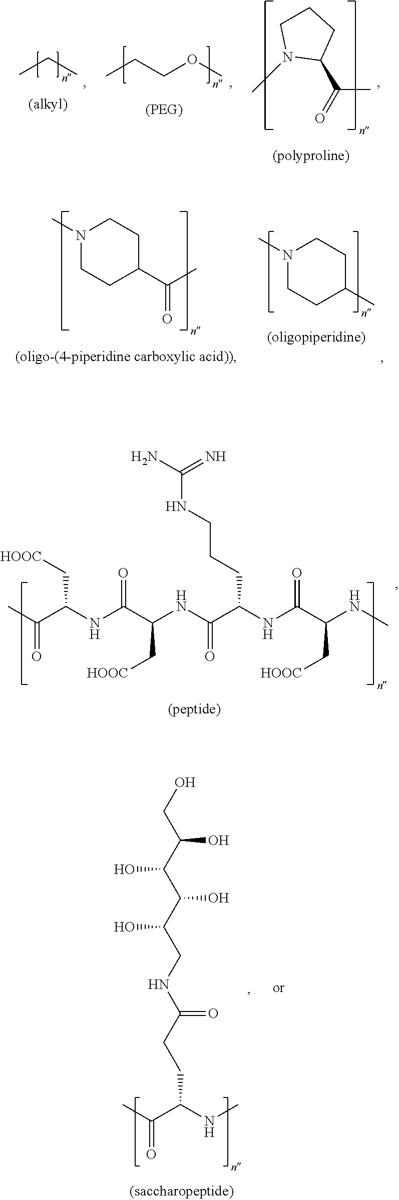

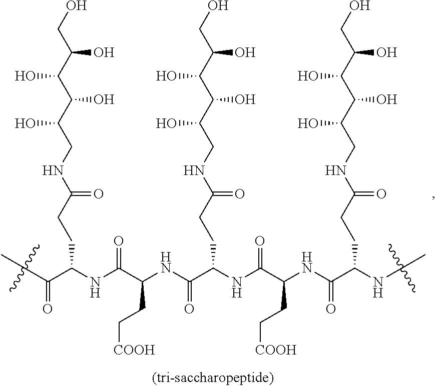

wherein n″ is 0-30.

L′ can be a bivalent linker. L′ can be a trivalent linker.

In some embodiments, L′ is a pyrido[2,3-d]pyrimidine analog with the following structure:

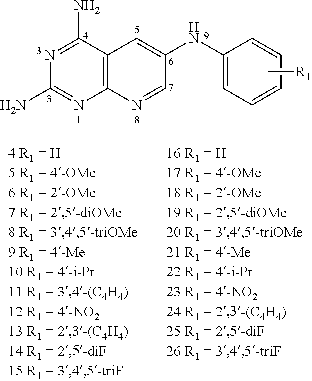

The linker can include a pharmacokinetic extender, such as an albumin binder or a hapten. Examples of albumin binders include, but are not limited to:

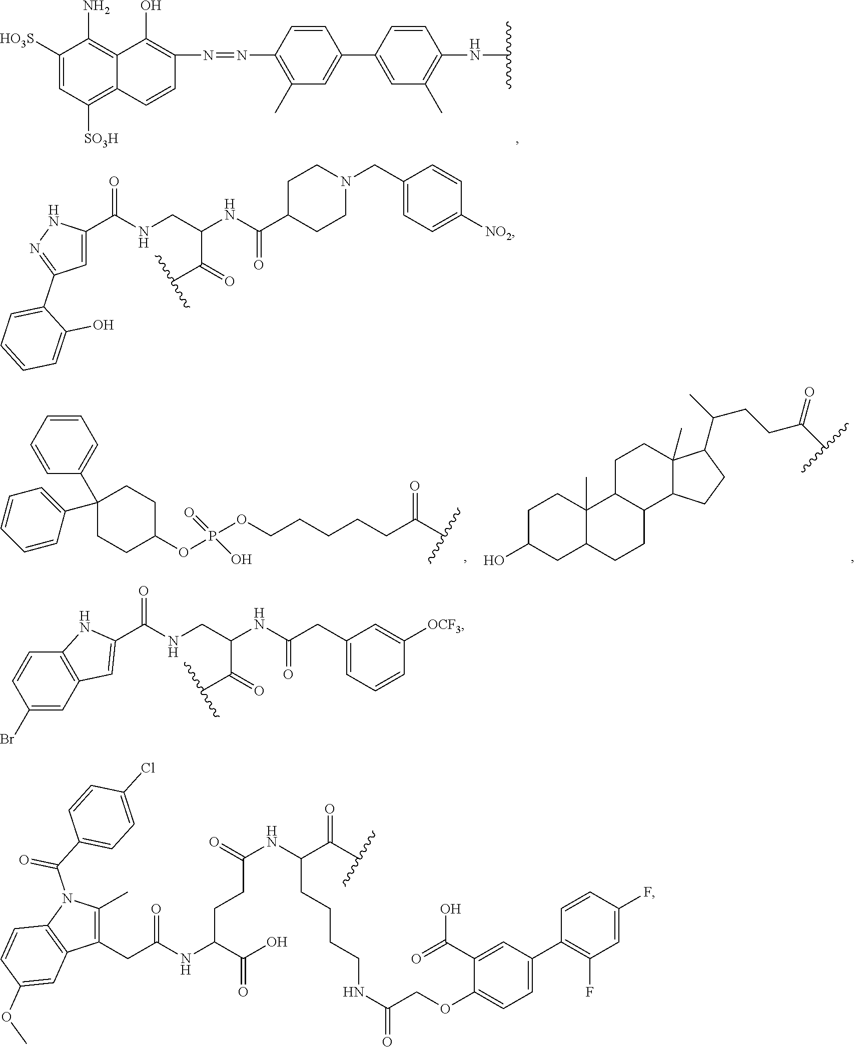

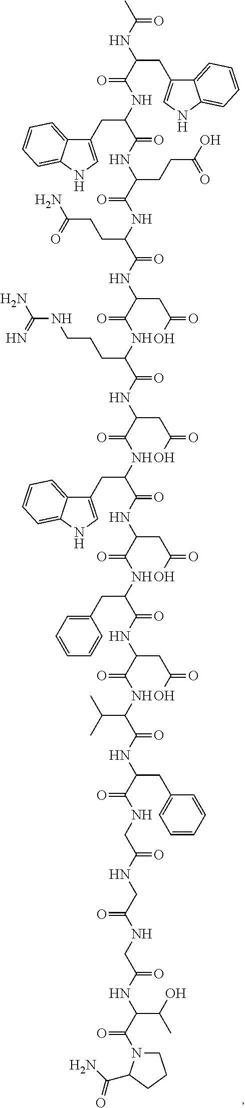 [SMILES: CC(=O)NC(Cc1c[nH]c2ccccc12)C(=O)NC(Cc1c[nH]c2ccccc12)C(=O)NC(CCC(=O)O)C(=O)NC(CCC(N)=O)C(=O)NC(CC(=O)O)C(=O)NC(CCCNC(=N)N)C(=O)NC(CC(=O)O)C(=O)NC(Cc1c[nH]c2ccccc12)C(=O)NC(CC(=O)O)C(=O)NC(Cc1ccccc1)C(=O)NC(CC(=O)O)C(=O)NC(C(=O)NC(Cc1ccccc1)C(=O)NCC(=O)NCC(=O)NCC(=O)NC(C(=O)N1CCCC1C(N)=O)C(C)O)C(C)C]

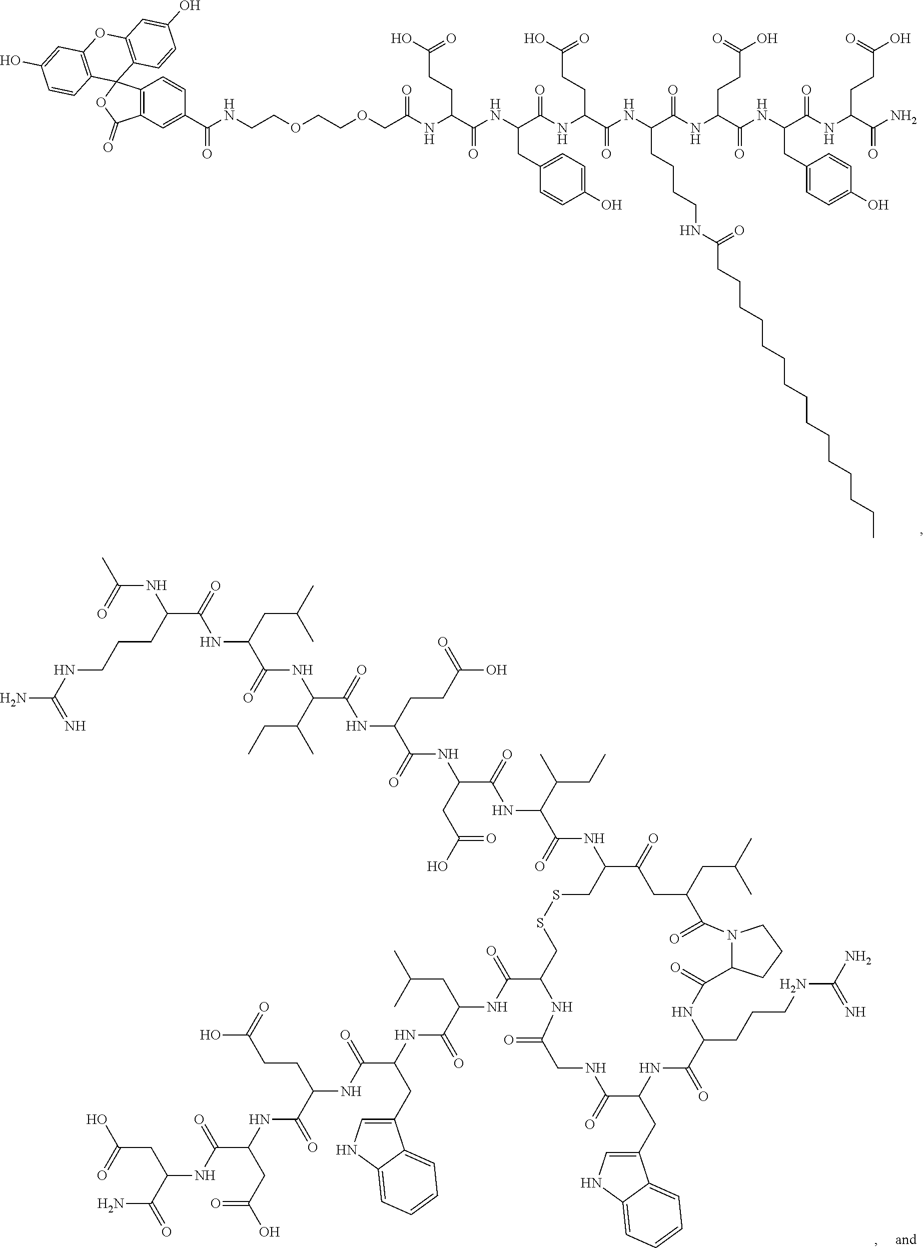

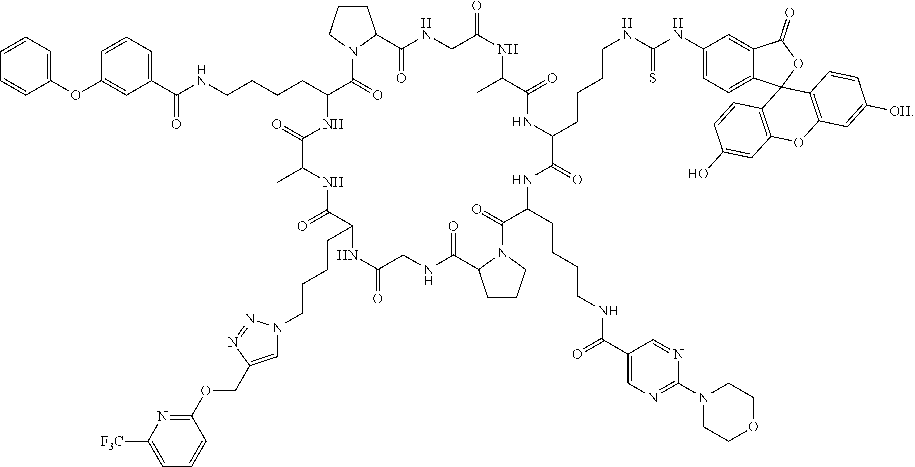 [SMILES: CC1NC(=O)CNC(=O)C2CCCN2C(=O)C(CCCCNC(=O)c2cccc(Oc3ccccc3)c2)NC(=O)C(C)NC(=O)C(CCCCn2cc(COc3cccc(C(F)(F)F)n3)nn2)NC(=O)CNC(=O)C2CCCN2C(=O)C(CCCCNC(=O)c2cnc(N3CCOCC3)nc2)NC(=O)C(CCCCNC(=S)Nc2ccc3c(c2)C(=O)OC32c3ccc(O)cc3Oc3cc(O)ccc32)NC1=O]

Examples of haptens include, but are not limited to, 2,4-dinitrophenol (DNP), 2,4,6-trinitrophenol (TNP), rhamnose, galactose-α-1,3-galactose (α-Gal), or an antibody binder. Examples of antibody binders include, but are not limited to, a Fab, an scFv, a V_{H}, a V_{L}, a V_{H}H, a V-NAR, a monobody, an anticalin, an affibody, or a DARPin.

L′ of the compounds hereof can optionally be conjugated with and/or include a spacer (S¹). S¹ can be any suitable spacer. Examples of spacers include, but are not limited to, an alkyl chain with at least about 20 carbon atoms, e.g., at least 20 carbon atoms, in the chain, a PEG with at least about 20 units, e.g., at least 20 units, a sugar, a peptidoglycan, a clickable linker (e.g., a triazole), a rigid linker (e.g., a polyproline or a polypiperidine), or a combination of two or more of the foregoing.

Any of the compounds can further comprise S¹, which can include a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the L. The compound can further comprise an albumin binding group, e.g., an albumin binding group selected from the group consisting of

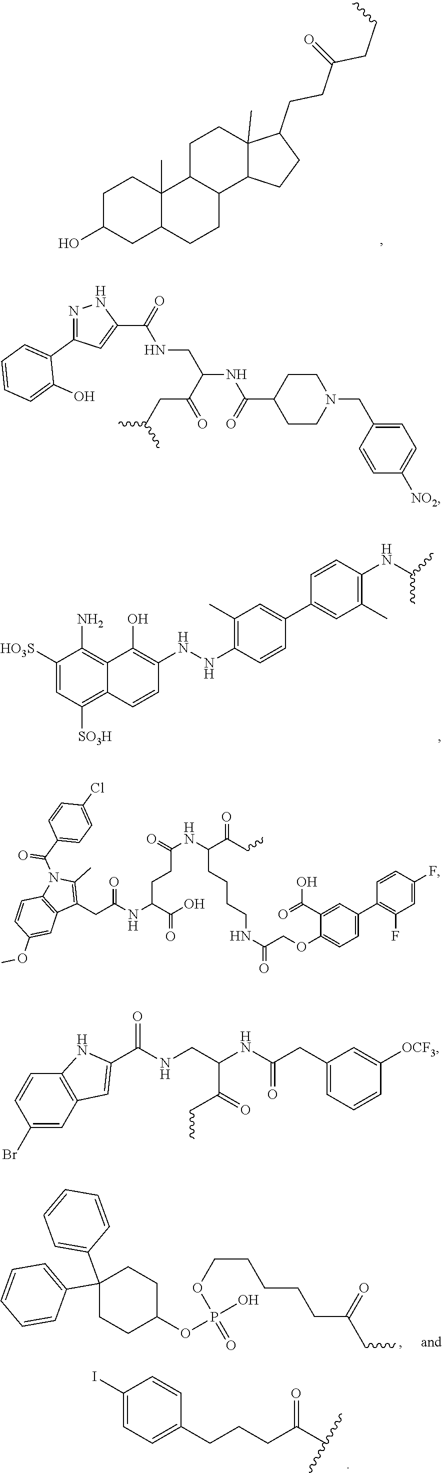

L′ can comprise one or more releasable linkers that cleave under the conditions described herein by a chemical mechanism involving beta elimination. Such releasable linkers include beta-thio, beta-hydroxy, and beta-amino substituted carboxylic acids and derivatives thereof, such as esters, amides, carbonates, carbamates, and ureas. Such linkers also include 2- and 4-thioarylesters, carbamates, and carbonates.

An example of a releasable linker includes a linker of the formula:

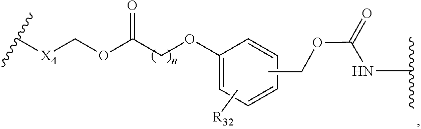

wherein X⁴ is NR, n is an integer selected from 0, 1, 2, and 3, R₃₂ is hydrogen, or a substituent, including a substituent capable of stabilizing a positive charge inductively or by resonance on the aryl ring, such as alkoxy, and the like. The releasable linker can be further substituted.

Assisted cleavage of releasable portions of L′ can include mechanisms involving benzylium intermediates, benzyne intermediates, lactone cyclization, oxonium intermediates, beta-elimination, and the like. In addition to fragmentation subsequent to cleavage of a releasable portion of L, the initial cleavage of the releasable linker can be facilitated by an anchimerically assisted mechanism. Thus, in the example of a releasable portion of L′ given above, the hydroxyalkanoic acid, which can cyclize, facilitates cleavage of the methylene bridge, by for example an oxonium ion, and facilitates bond cleavage or subsequent fragmentation after bond cleavage of the releasable linker. Alternatively, acid catalyzed oxonium ion-assisted cleavage of the methylene bridge can begin a cascade of fragmentation of this illustrative bivalent linker, or fragment thereof. Alternatively, acid-catalyzed hydrolysis of the carbamate can facilitate the beta elimination of the hydroxyalkanoic acid, which can cyclize, and facilitate cleavage of methylene bridge, by for example an oxonium ion. Other chemical mechanisms of bond cleavage under the metabolic, physiological, or cellular conditions can initiate such a cascade of fragmentation. Other chemical mechanisms of bond cleavage under the metabolic, physiological, or cellular conditions can initiate such a cascade of fragmentation.

Illustrative mechanisms for cleavage of the bivalent linkers described herein include the following 1,4 and 1,6 fragmentation mechanisms for carbonates and carbamates:

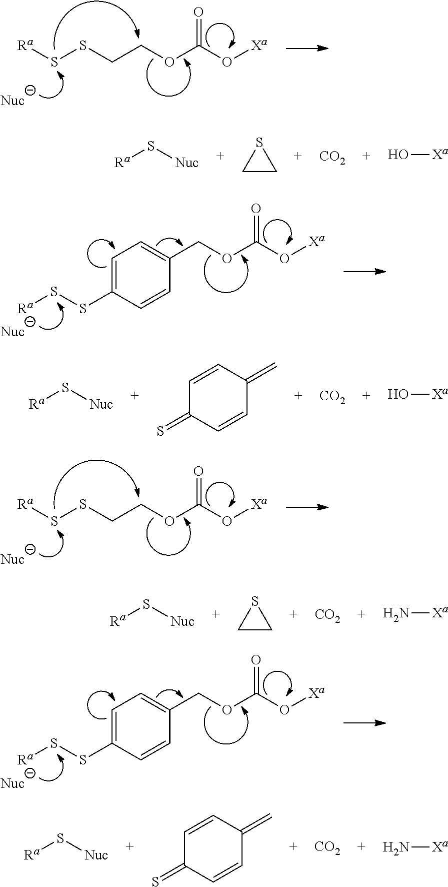

wherein Nuc⁻ is an exogenous or endogenous nucleophile, glutathione, or bioreducing agent, and the like, and R^{a} and X^{a} are connected through other portions of the bivalent linker. The location of R^{a} and X^{a} can be switched such that, e.g., the resulting products are X^{a}—S-Nuc and HO—R^{a} H2N—R^{a}.

Although the above fragmentation mechanisms are depicted as concerted mechanisms, any number of discrete steps can take place to affect the ultimate fragmentation of the bivalent linker to the final products shown. For example, the bond cleavage can also occur by acid catalyzed elimination of the carbamate moiety, which can be anchimerically assisted by the stabilization provided by either the aryl group of the beta sulfur or disulfide illustrated in the above examples. In those variations of this embodiment, the releasable linker is the carbamate moiety. Alternatively, the fragmentation can be initiated by a nucleophilic attack on the disulfide group, causing cleavage to form a thiolate. The thiolate can intermolecularly displace a carbonic acid or carbamic acid moiety and form the corresponding thiacyclopropane. In the case of the benzyl-containing bivalent linkers, following an illustrative cleavage of the disulfide bond, the resulting phenyl thiolate can further fragment to release a carbonic acid or carbamic acid moiety by forming a resonance-stabilized intermediate. In any of these cases, the releasable nature of the illustrative bivalent linkers described herein can be realized by whatever mechanism can be relevant to the chemical, metabolic, physiological, or biological conditions present.

As described above, therefore, releasable linkers can comprise a disulfide group. Further examples of releasable linkers comprised in L′ include divalent radicals comprising alkyleneaziridin-1-yl, alkylenecarbonylaziridin-1-yl, carbonylalkylaziridin-1-yl, alkylenesulfoxylaziridin-1-yl, sulfoxylalkylaziridin-1-yl, sulfonylalkylaziridin-1-yl, or alkylenesulfonylaziridin-1-yl groups, wherein each of the releasable linkers is optionally substituted. Additional examples of releasable linkers comprised in L′ include divalent radicals comprising methylene, 1-alkoxyalkylene, 1-alkoxycycloalkylene, 1-alkoxyalkylenecarbonyl, 1-alkoxycycloalkylenecarbonyl, carbonylarylcarbonyl,carbonyl(carboxyaryl) carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio or carbonylalkylthio groups, wherein each of the releasable linkers is optionally substituted.

Additional examples of releasable linkers comprised in L′ include an oxygen atom and methylene, 1-alkoxyalkylene, 1-alkoxycycloalkylene, 1-alkoxyalkylenecarbonyl or 1-alkoxycycloalkylenecarbonyl groups, wherein each of the releasable linkers is optionally substituted. Alternatively, in some embodiments the releasable linker includes an oxygen atom and a methylene group, wherein the methylene group is substituted with an optionally substituted aryl, and the releasable linker is bonded to the oxygen to form an acetal or ketal. Further, in some embodiments the releasable linker includes an oxygen atom and a sulfonylalkyl group, and the releasable linker is bonded to the oxygen to form an alkylsulfonate.

Additional examples of releasable linkers comprised in L′ include a nitrogen and iminoalkylidenyl, carbonylalkylideniminyl, iminocycloalkylidenyl, and carbonylcycloalkylideniminyl groups, wherein each of the releasable linkers is optionally substituted and the releasable linker is bonded to the nitrogen to form a hydrazone. In some embodiments, the hydrazone is acylated with a carboxylic acid derivative, an orthoformate derivative, or a carbamoyl derivative to form various acylhydrazone releasable linkers.

Additional examples of releasable linkers comprised in L′ include an oxygen atom and alkylene(dialkylsilyl), alkylene(alkylarylsilyl), alkylene(diarylsilyl), (dialkylsilyl)aryl, (alkylarvlsilyl)aryl or (diarylsilyl)aryl groups, wherein each of the releasable linkers is optionally substituted and the releasable linker is bonded to the oxygen to form a silanol.

Additional examples of releasable linkers comprised in L′ include two independent nitrogens and carbonylarylcarbonyl, carbonyl(carboxyaryl)carbonyl, or carbonyl(biscarboxvaryl)carbonyl. In some embodiments the releasable linker is bonded to the heteroatom nitrogen to form an amide.

Additional examples of releasable linkers comprised in L′ include an oxygen atom, a nitrogen, and a carbonylarylcarbonyl, carbonyl(carboxyaryl)carbonyl, or carbonyl(biscarboxvaryl)carbonyl. In some embodiments, the releasable linker forms an amide.

In some embodiments, L′ comprises an optionally substituted 1-alkylenesuccinimid-3-yl group and a releasable portion comprising methylene, 1-alkoxyalkylene, 1-alkoxycycloalkylene, 1-alkoxyalkylenecarbonyl or 1-alkoxycycloalkylenecarbonyl groups, each of which can be optionally substituted, to form a succinimid-1-ylalkyl acetal or ketal.

In some embodiments, L′ comprises carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl or 1-(carbonyltetrahydrofuranvl)succinimid-3-yl, each of which is optionally substituted. In some embodiments, L′ further comprises an additional nitrogen such that L′ comprises alkylenecarbonyl, cycloalkylenecarbonyl, carbonylalkylcarbonyl or 1-(carbonylalkyl)succinimid-3-yl groups, each of which is optionally substituted, bonded to the nitrogen to form an amide. In some embodiments, L′ further comprises a sulfur atom and alkylene or cycloalkylene groups, each of which is optionally substituted with carboxy, and is bonded to the sulfur to form a thiol. In some embodiments, L′ comprises a sulfur atom and 1-alkylenesuccinimid-3-yl and 1-(carbonylalkyl)succinimid-3-yl groups bonded to the sulfur to form a succinimid-3-ylthiol.

In some embodiments L′ comprises a nitrogen and a releasable portion comprising alkyleneaziridin-1-yl, carbonylalkylaziridin-1-yl, sulfoxylalkylaziridin-1-yl, or sulfonylalkylaziridin-1-yl, each of which is optionally substituted. In some embodiments, L′ comprises carbonyl, thionocarbonyl, alkylenecarbonyl, cycloalkylenecarbonyl, carbonylalkylcarbonyl, or 1-(carbonylalkyl)succinimid-3-yl, each of which is optionally substituted, and bonded to the releasable portion to form an aziridine amide.

Examples of L′ include alkylene-amino-alkylenecarbonyl, alkylene-thio-(carbonylalkylsuccinimid-3-yl), and the like, as further illustrated by the following formulae:

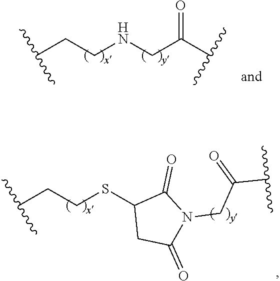

wherein x′ and y′ are each independently 1, 2, 3, 4, or 5.

L can have any suitable assortment of atoms in the chain, including C (e.g., —CH₂—, C(O)), N (e.g., NH, NR^{b}, wherein R^{b} is, e.g., H, alkyl, alkylaryl, and the like), O (e.g., —O—), P (e.g., —O— P(O)(OH)O—), and S (e.g., —S—). For example, the atoms used in forming L′ can be combined in all chemically relevant ways, such as chains of carbon atoms forming alkyl groups, chains of carbon and oxygen atoms forming polyoxyalkyl groups, chains of carbon and nitrogen atoms forming polyamines, and others, including rings, such as those that form aryl and heterocyclyl groups (e.g., triazoles, oxazoles, and the like). In addition, the bonds connecting atoms in the chain in L′ can be either saturated or unsaturated, such that for example, alkanes, alkenes, alkynes, cycloalkanes, arylenes, imides, and the like can be divalent radicals that are included in L. Further, the chain-forming L′ can be substituted or unsubstituted.

Additional examples of L′ groups include the groups 1-alkylsuccinimid-3-yl, carbonyl, thionocarbonyl, alkyl, cycloalkyl, alkylcycloalkyl, alkylcarbonyl, cycloalkylcarbonyl, carbonylalkylcarbonyl, 1-alkylsuccinimid-3-yl, 1-(carbonylalkyl)succinimid-3-yl, alkylsulfoxyl, sulfonylalkyl, alkylsulfoxylalkyl, alkylsulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranvl)succinimid-3-yl, wherein each group can be substituted or unsubstituted. Any of the aforementioned groups can be L′ or can be included as a portion of L. In some instances, any of the aforementioned groups can be used in combination (or more than once) (e.g., -alkyl-C(O)-alkyl) and can further comprise an additional nitrogen (e.g., alkyl-C(O)—NH—, —NH-alkyl-C(O)— or —NH-alkyl-), oxygen (e.g., -alkyl-O-alkyl-) or sulfur (e.g., -alkyl-S-alkyl-). Examples of such L′ groups are alkylcarbonyl, cycloalkylcarbonyl, carbonylalkylcarbonyl, 1-(carbonylalkyl)succinimid-3-yl, and succinimid-3-ylthiol, wherein each group can be substituted or unsubstituted.

In some embodiments, L′ is formed via click chemistry/click chemistry-derived. Those of skill in the art understand that the terms “click chemistry” and “click chemistry-derived” generally refer to a class of small molecule reactions commonly used in conjugation, allowing the joining of substrates of choice with specific molecules. Click chemistry is not a single specific reaction but describes a way of generating products that follow examples in nature, which also generates substances by joining small modular units. In many applications, click reactions join a biomolecule and a reporter molecule. Click chemistry is not limited to biological conditions: the concept of a “click” reaction has been used in pharmacological and various biomimetic applications. However, they have been made notably useful in the detection, localization and qualification of biomolecules.

Click reactions can occur in one pot, typically are not disturbed by water, can generate minimal byproducts, and are “spring-loaded”—characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (for in vivo systems).

Click chemistry examples include examples where L′ can be derived from copper-catalyzed azide-alkyne cycloaddition (CuAAC), strain-promoted azide-alkyne cycloaddition (SPAAC), inverse electron demand Diels-Alder reaction (IEDDA), and Staudinger ligation (SL). For example, X^{a} and R^{a} can be linked to each other as shown in Schemes 1-5:

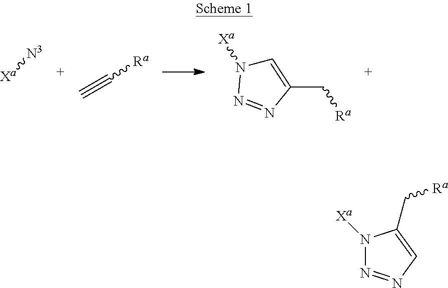

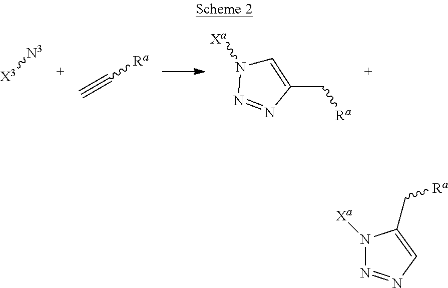

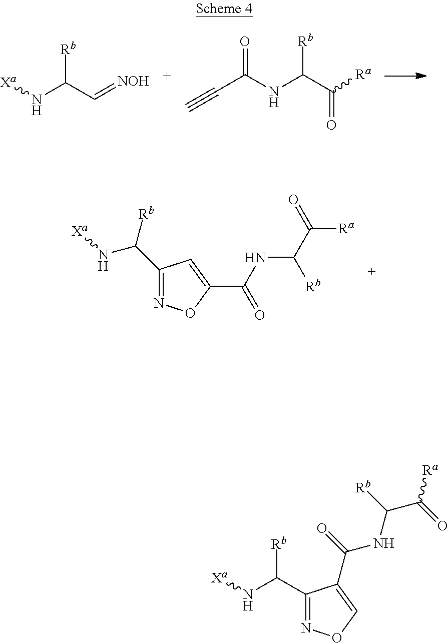

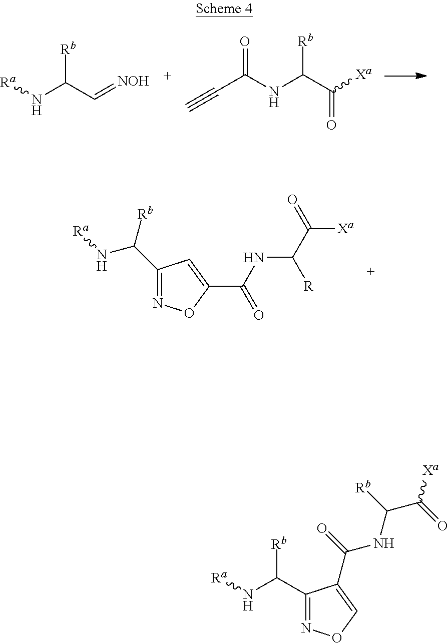

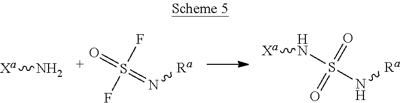

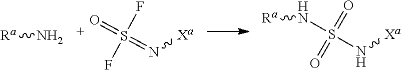

wherein each R^{b} is independently H, alkyl, arylalkyl, -alkyl-S-alkyl or arylalkyl or the side-chain of any naturally- or non-naturally occurring amino acid and the like. In Schemes 1-5, the wavy line connected to X^{a} and R^{a} represents a linkage between X^{a} and R^{a} and the groups to which they are attached. It should be appreciated that in Schemes 1-5, the triazole, oxazole, and the —NH—SO2-NH— group would be considered to be part of L.

In some embodiments, L′ is a linker selected from the group consisting of pegylated-, alkyl-, sugar-, and peptide-based dual linker; L′ is either a non-releasable linker or a releasable linker bivalently covalently attached to the folate ligand (or, in other embodiments, folate analogue or antifolate) and the steroid.

In some embodiments, L′ is:

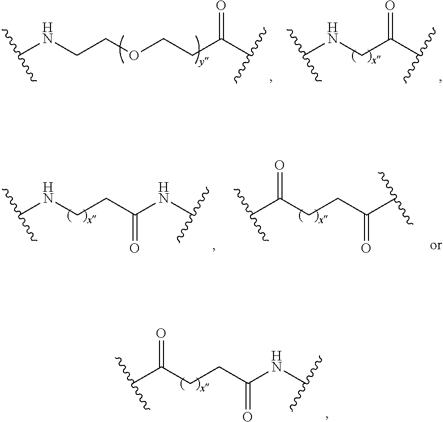

wherein
x″ is an integer from 0 to 10, and
y″ is an integer from 3 to 100.

In some aspects, x″ is an integer from 3 to 10.

In some embodiments, L′ is:

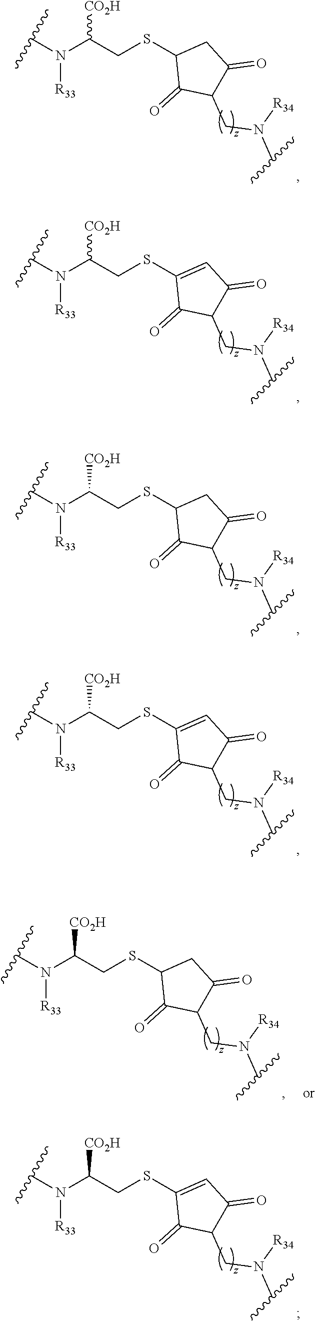

wherein each of R₃₃ and R₃₄ is independently H or C₁-C₆ alkyl;
and z is an integer from 1 to 8.

In some embodiments, L is:

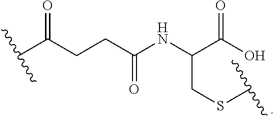 [SMILES: *SCC(NC(=O)CCC(*)=O)C(=O)O]

In some embodiments, L is:

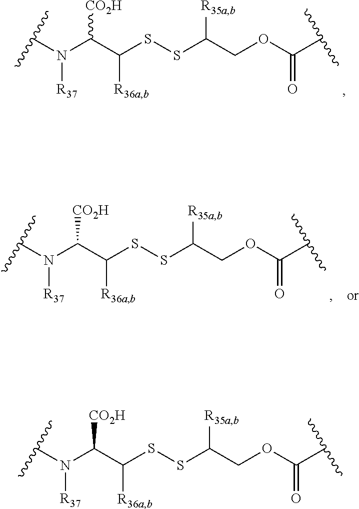

wherein R₃₇ is H or C₁-C₆ alkyl; R₃₅ₐ, R_{35b}, R₃₆ₐ, and R_{36b} each is independently H or C₁-C₆ alkyl.

In some embodiments, L′ comprises an amino acid. In some embodiments, L′ comprises an amino acid selected from the group consisting of Lys, Asn, Thr, Ser, Ile, Met, Pro, His, Gln, Arg, Gly, Asp, Glu, Ala, Val, Phe, Leu, Tyr, Cys, and Trp. In some embodiments, L′ comprises at least two amino acids independently selected from the group consisting of Glu and Cys. In some embodiments, L′ comprises Glu-Glu, wherein the glutamic acids are covalently bonded to each other through the carboxylic acid side chains.

In some embodiments, L′ comprises one or more hydrophilic spacer linkers comprising a plurality of hydroxyl functional groups.

In some embodiments, L′ comprises at least one 2,3-diaminopropionic acid group, at least one glutamic acid group (e.g., unnatural amino acid D-Glutamic acid), and at least one cysteine group. One example of such a linker is one having the non-natural amino acid, such as a linker having the repeating unit of the formula:

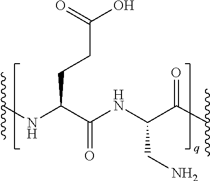

wherein q is an integer from 1 to 10 (e.g., 1 to 3 and 2 to 5). In some embodiments, L′ comprises the general formula:

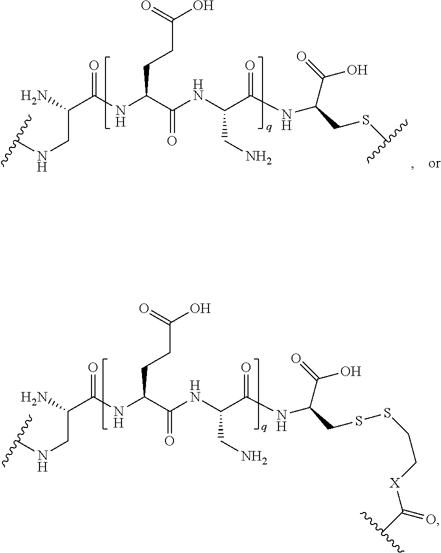

wherein X can be O, NH, NR, or S, and q is an integer from 1 to 10. In some embodiments, L′ comprises the formula:

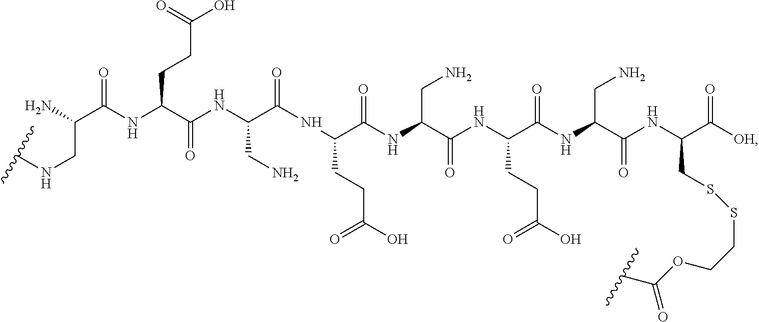 [SMILES: *NC[C@H](N)C(=O)N[C@@H](CCC(=O)O)C(=O)N[C@@H](CN)C(=O)N[C@@H](CCC(=O)O)C(=O)N[C@@H](CN)C(=O)N[C@@H](CCC(=O)O)C(=O)N[C@@H](CN)C(=O)N[C@H](CSSCCOC(*)=O)C(=O)O]

wherein the disulfide group is a part of a self-immolative group that can be generically described as a group of the formula —CH₂—S—S—CH₂—.

In some embodiments, the compounds described herein include linkages that cause the steroids described herein to be released by any suitable mechanism, including a release mechanism involving reduction, oxidation, or hydrolysis. An example of a reduction mechanism includes reduction of a disulfide group into two separate sulfyhydryl groups. Thus, for example, a group of the formula —CH₂—S—S—CH— would be reduced to two separate groups of the formula —CH₂—SH, such that if the linker were of the formula:

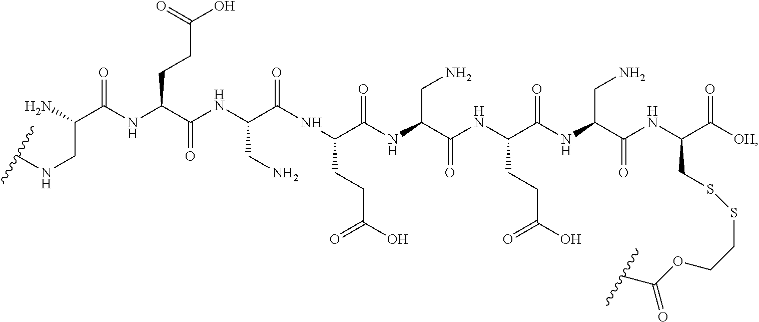 [SMILES: *NC[C@H](N)C(=O)N[C@@H](CCC(=O)O)C(=O)N[C@@H](CN)C(=O)N[C@@H](CCC(=O)O)C(=O)N[C@@H](CN)C(=O)N[C@@H](CCC(=O)O)C(=O)N[C@@H](CN)C(=O)N[C@H](CSSCCOC(*)=O)C(=O)O]

the reduction product would be of the formula.

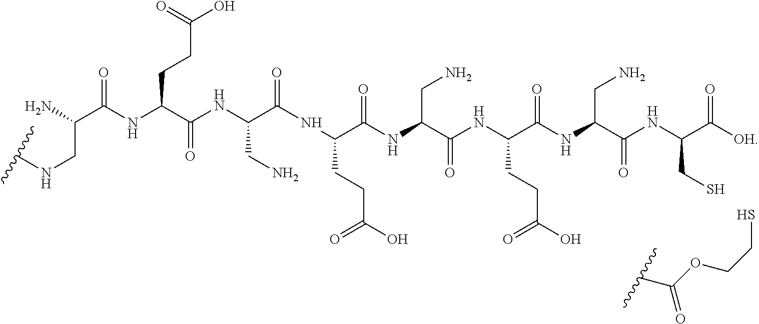 [SMILES: *NC[C@H](N)C(=O)N[C@@H](CCC(=O)O)C(=O)N[C@@H](CN)C(=O)N[C@@H](CCC(=O)O)C(=O)N[C@@H](CN)C(=O)N[C@@H](CCC(=O)O)C(=O)N[C@@H](CN)C(=O)N[C@H](CS)C(=O)O ; *C(=O)OCCS]

In this example, the steroid is attached to the linker via a self-immolative moiety (e.g., a disulfide group).

An example of a self-immolative disulfide also includes a sterically protected disulfide bond. The steroid can be attached to the linker via any other suitable self-immolative bond, including via a self-immolative cathepsin cleavable amino acid sequence; via a self-immolative furin cleavable amino acid sequence; via a self-immolative β-glucuronidase cleavable moiety; via a self-immolative phosphatase cleavable moiety; or via a self-immolative sulfatase cleavable moiety. Multiple self-immolative linkages are also contemplated herein.

In some embodiments, the linker comprises a self-immolative moiety. In some embodiments, the linker comprises a self-immolative disulfide and or sterically protected disulfide bond. In some embodiments, the linker comprises a self-immolative cathepsin-cleavable amino acid sequence. In some embodiments, the linker comprises a self-immolative furin-cleavable amino acid sequence. In some embodiments, the linker comprises a self-immolative D-glucuronidase-cleavable moiety. In some embodiments, the linker comprises a self-immolative phosphatase-cleavable moiety. In some embodiments, the linker comprises a self-immolative sulfatase-cleavable moiety.

In some embodiments, the linker comprises a phosphate or pyrophosphate group. In some embodiments, the linker comprises a cathepsin B cleavable group. In some embodiments, the cathepsin B cleavable group is Valine-Citrulline. In some embodiments, the linker comprises a carbamate moiety. In some embodiments, the linker comprises a β-glucuronide.

In some embodiments, the compounds described herein include linkages where the steroid is attached to the linker via an ester, phosphate, oxime, acetal, pyrophosphate, polyphosphate, disulfide, sulfate, hydrazide, imine, carbonate, carbamate or enzyme-cleavable amino acid sequence.

In some embodiments, the linker comprises an ester, phosphate, oxime, acetal, pyrophosphate, polyphosphate, disulfide, sulfate, hydrazide, imine, carbonate, carbamate or enzyme-cleavable amino acid sequence.

In some embodiments, L′ comprises one or more spacer linkers. Spacer linkers can be hydrophilic spacer linkers comprising a plurality of hydroxyl functional groups. A spacer can comprise any stable arrangement of atoms. Each spacer is independently selected from the group consisting an amide, ester, urea, carbonate, carbamate, disulfide, amino acid, amine, ether, alkyl, alkene, alkyne, heteroalkyl (e.g., polyethylene glycol), cycloakyl, aryl, heterocycloalkyl, heteroaryl, carbohydrate, glycan, peptidoglycan, polypeptide, or any combination thereof. In some embodiments, a spacer comprises any one or more of the following units: an amide, ester, urea, carbonate, carbamate, disulfide, amino acid, amine, ether, alkyl, alkene, alkyne, heteroalkyl (e.g., PEG), cycloakyl, aryl, heterocycloalkyl, heteroaryl, carbohydrate, glycan, peptidoglycan, polypeptide, or any combination thereof. In some embodiments, a spacer comprises a solubility enhancer or PK/PD modulator W. In some embodiments, a spacer comprises a glycosylated amino acid. In some embodiments, a spacer comprises one or more monosaccharide, disaccharide, polysaccharide, glycan, or peptidoglycan. In some embodiments, a spacer comprises a releasable moiety (e.g., a disulfide bond, an ester, or other moieties that can be cleaved in vivo). In some embodiments, a spacer comprises one or more units such as ethylene (e.g., polyethylene), ethylene glycol (e.g., PEG), ethanolamine, ethylenediamine, and the like (e.g., propylene glycol, propanolamine, propylenediamine). In some embodiments, a spacer comprises an oligoethylene, PEG, alkyl chain, oligopeptide, polypeptide, rigid functionality, peptidoglycan, oligoproline, oligopiperidine, or any combination thereof. In some embodiments, a spacer comprises an oligoethylene glycol or a PEG. A spacer can comprise an oligoethylene glycol. In some embodiments, a spacer comprises a PEG. In some embodiments, a spacer comprises an oligopeptide or polypeptide. In some embodiments, a spacer comprises an oligopeptide. In some embodiments, a spacer comprises a polypeptide. In some embodiments, a spacer comprises a peptidoglycan. In some embodiments, a spacer does not comprise a glycan. In some embodiments, a spacer does not comprise a sugar. In some embodiments, a rigid functionality is an oligoproline or oligopiperidine. In some embodiments, a rigid functionality is an oligoproline. In some embodiments, a rigid functionality is an oligopiperidine. In some embodiments, a rigid functionality is an oligophenyl. In some embodiments, a rigid functionality is an oligoalkyne. In some embodiments, an oligoproline or oligopiperidine has about two up to and including about fifty, about two to about forty, about two to about thirty, about two to about twenty, about two to about fifteen, about two to about ten, or about two to about six repeating units (e.g., prolines or piperidines).

n some embodiments, L′ comprises a solubility enhancer or PK/PD modulator. In some embodiments, L′ comprises polyethylene glycol (PEG), sugar, peptide, or peptidoglycan. In some embodiments, L′ comprises a PEG, sugar, peptide, or peptidoglycan for achieving better solubility and PK/PD properties. In some embodiments, L′ comprises one or more monosaccharide, disaccharide, peptide, peptidoglycan, and/or serum albumin. In some embodiments, L′ comprises one or more PEG, peptide, peptidoglycan, or serum albumin. In some embodiments, W does not comprise a sugar. In some embodiments, W does not comprise a monosaccharide, disaccharide, or polysaccharide. In some embodiments, W does not comprise a glycan. In some embodiments, L′ comprises a glycosylated amino acid. In some embodiments, L′ comprises a glycosylate cysteine. In some embodiments, L′ comprises a free carboxylic acid. In some embodiments, L′ comprises a PEG.

In some embodiments, L′ comprises one or more monosaccharide, disaccharide, oligosaccharide, polysaccharide, peptide, peptidoglycan, serum albumin, solubility enhancer, PK/PD modulator, or a combination thereof. In some embodiments, L′ modulates a pharmacological, pharmacokinetic, pharmacodynamic, or physicochemical property. In some embodiments, L′ facilitates internalization. In some embodiments, L′ improves aqueous solubility. In some embodiments, L′ increases plasma protein binding. In some embodiments, W modulates (e.g., reduces) the compound's excretion, elimination, metabolism, stability (e.g., enzymatic stability, plasma stability), distribution, toxicity, or a combination thereof.

In some embodiments, a monosaccharide such as found in W exists in an equilibrium between its linear and cyclic form. In some embodiments, a monosaccharide is linear. In some embodiments, a monosaccharide is cyclic. In some embodiments, a monosaccharide exists as a D isomer. In some embodiments, a monosaccharide exists as an L′ isomer. As non-limiting examples, in some embodiments, L′ comprises one or more monosaccharides selected from the following: ribose, galactose, mannose, glucosefructose, N-acetylglucosamine, N-acetylmuramic acid or derivatives thereof (e.g., cyclic or linear forms, methylated derivatives, acetylated derivatives, phosphorylated derivatives, aminated derivatives, oxidized or reduced derivatives, D or L′ isomers, isotopes, stereoisomers, regioisomers, tautomers, or combinations thereof).

In some embodiments, a disaccharide, oligosaccharide, or polysaccharide, as can be disposed within W, contains an O-linkage, an N-linkage, a C-linkage, or a combination thereof. In some embodiments, a disaccharide, oligosaccharide, or polysaccharide contains a glycosidic linkage in either an alpha- or beta-orientation. In some embodiments, L′ comprises an oligosaccharide, a polysaccharide, or a glycan (e.g., a glycoprotein, glycopeptide, glycolipid, glycogen, proteoglycan, peptidoglycan, and the like).

In some embodiments, L′ comprises an amino acid, a peptide, a polypeptide, or a protein. In some embodiments, the amino acid is a natural amino acid (e.g., alanine (Ala), arginine (Arg), asparagine (Asn), aspartic acid (Asp), cysteine (Cys), glutamic acid (Glu), glutamine (Gln), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val)). Alternatively, in some embodiments, the amino acid is an unnatural or modified amino acid. L′ can comprise a sugar or sugar derivative covalently attached to the side chain of an amino acid (e.g., a glutamic acid, an aspartic acid).

In some embodiments, L′ comprises a glycosylated amino acid such as:

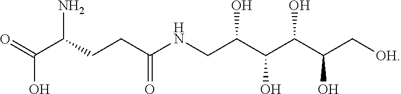 [SMILES: N[C@H](CCC(=O)NC[C@H](O)[C@@H](O)[C@H](O)[C@H](O)CO)C(=O)O]

In some embodiments, a peptide or polypeptide comprises a plurality of amino acids, natural and/or unnatural. In some embodiments, a peptide (or peptidoglycan) has about two and about twenty amino acids. In some embodiments, an amino acid, a peptide, a polypeptide, or a protein has a pharmacological or physicochemical effect that enhances one or more properties of the compound (e.g., modulating solubility, solubility, size, permeability, protein binding, target binding, excretion, metabolism, toxicity, distribution, half-life, and/or duration of action). In some embodiments, L′ comprises a pharmacokinetic modulator. In some embodiments, the pharmacokinetic modulator is a peptide or protein that can modulate (e.g., enhance) protein binding. In some embodiments, the pharmacokinetic modulator enhances plasma protein binding. In some embodiments, the pharmacokinetic modulator reduces the rate of elimination, excretion, or metabolism. In some embodiments, the pharmacokinetic modulator increases the duration of action of the compound.

In some embodiments, the linker comprises an albumin ligand. In some embodiments, the albumin ligand comprises

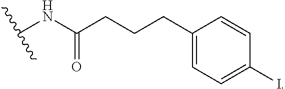 [SMILES: *NC(=O)CCCc1ccc(I)cc1]

In some embodiments, the linker comprises a dimethylcysteine group. In some embodiments, the dimethylcysteine group is linked to a succinimide to form:

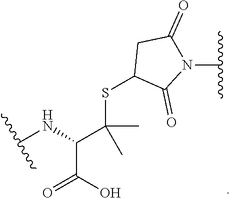 [SMILES: *N[C@@H](C(=O)O)C(C)(C)SC1CC(=O)N(*)C1=O]

A compound hereof can have, or can comprise, the following structure:

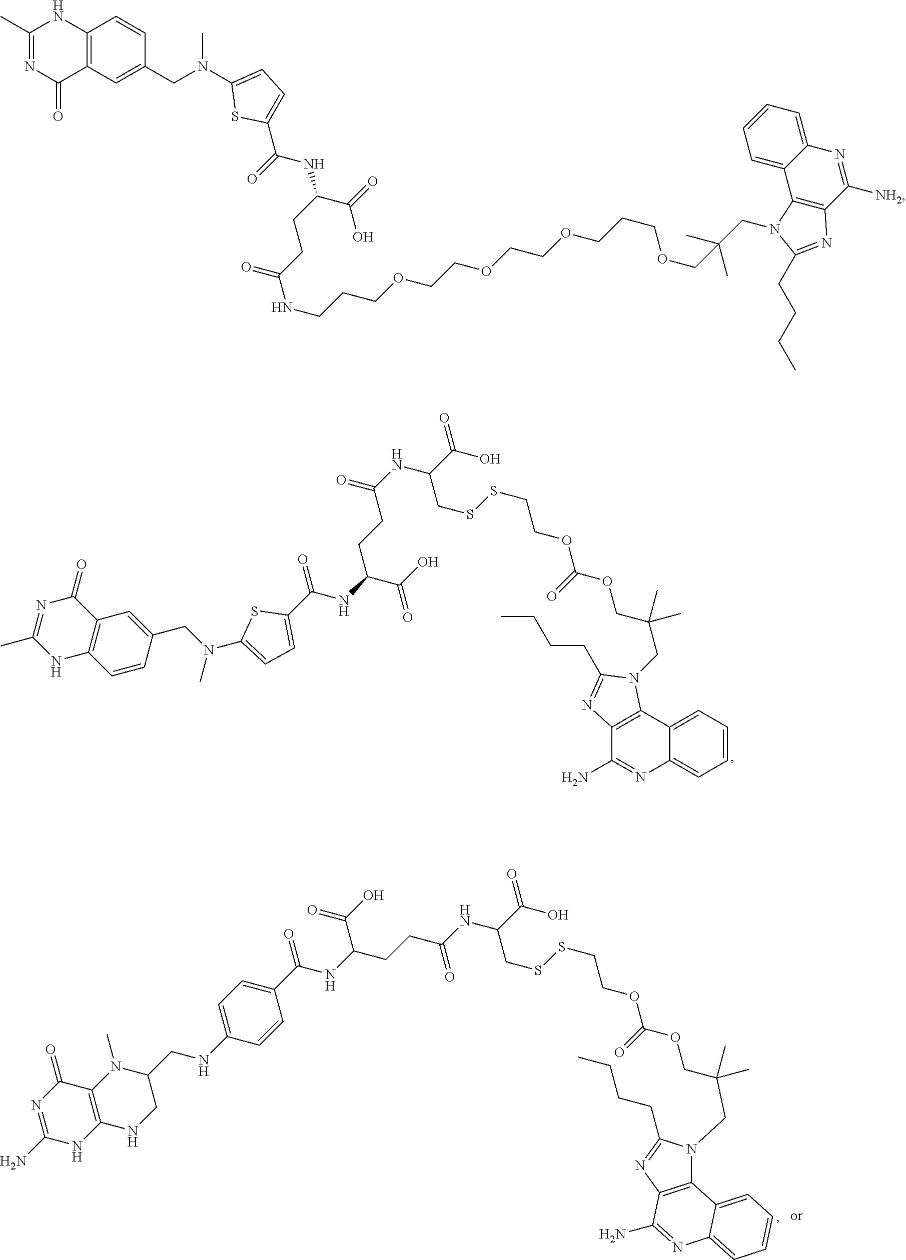 [SMILES: CCCCc1nc2c(N)nc3ccccc3c2n1CC(C)(C)COCCCOCCOCCOCCCNC(=O)CC[C@H](NC(=O)c1ccc(N(C)Cc2ccc3[nH]c(C)nc(=O)c3c2)s1)C(=O)O ; CCCCc1nc2c(N)nc3ccccc3c2n1CC(C)(C)COC(=O)OCCSSCC(NC(=O)CC[C@H](NC(=O)c1ccc(N(C)Cc2ccc3[nH]c(C)nc(=O)c3c2)s1)C(=O)O)C(=O)O ; "," ; CCCCc1nc2c(N)nc3ccccc3c2n1CC(C)(C)COC(=O)OCCSSCC(NC(=O)CCC(NC(=O)c1ccc(NCC2CNc3[nH]c(N)nc(=O)c3N2C)cc1)C(=O)O)C(=O)O ; "," ; "or"]

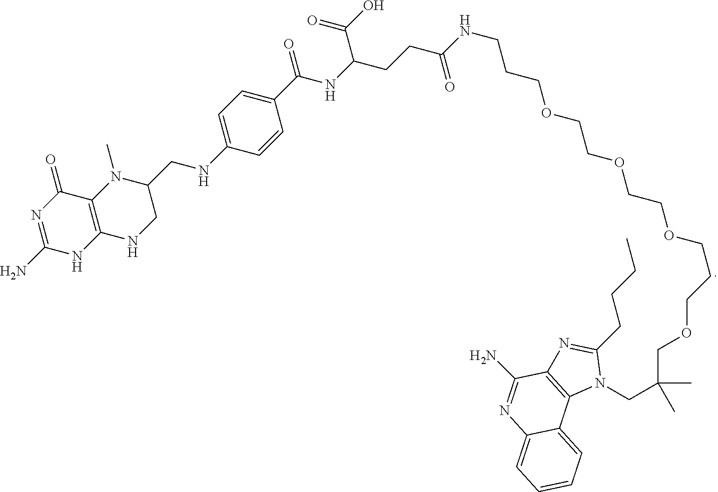 [SMILES: CCCCc1nc2c(N)nc3ccccc3c2n1CC(C)(C)COCCCOCCOCCOCCCNC(=O)CCC(NC(=O)c1ccc(NCC2CNc3[nH]c(N)nc(=O)c3N2C)cc1)C(=O)O ; "."]

A compound hereof can have, or can comprise, the following structure:

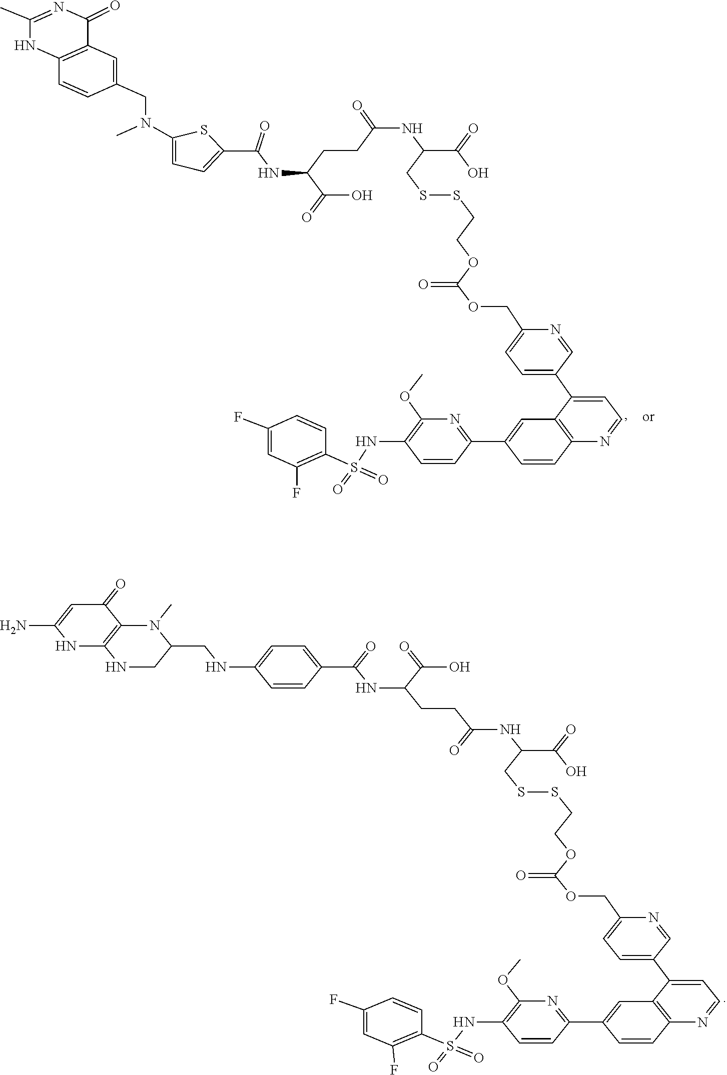 [SMILES: COc1nc(-c2ccc3nccc(-c4ccc(COC(=O)OCCSSCC(NC(=O)CC[C@H](NC(=O)c5ccc(N(C)Cc6ccc7[nH]c(C)nc(=O)c7c6)s5)C(=O)O)C(=O)O)nc4)c3c2)ccc1NS(=O)(=O)c1ccc(F)cc1F ; COc1nc(-c2ccc3nccc(-c4ccc(COC(=O)OCCSSCC(NC(=O)CCC(NC(=O)c5ccc(NCC6CNc7[nH]c(N)cc(=O)c7N6C)cc5)C(=O)O)C(=O)O)nc4)c3c2)ccc1NS(=O)(=O)c1ccc(F)cc1F]

A compound hereof can have, or can comprise, the following structure:

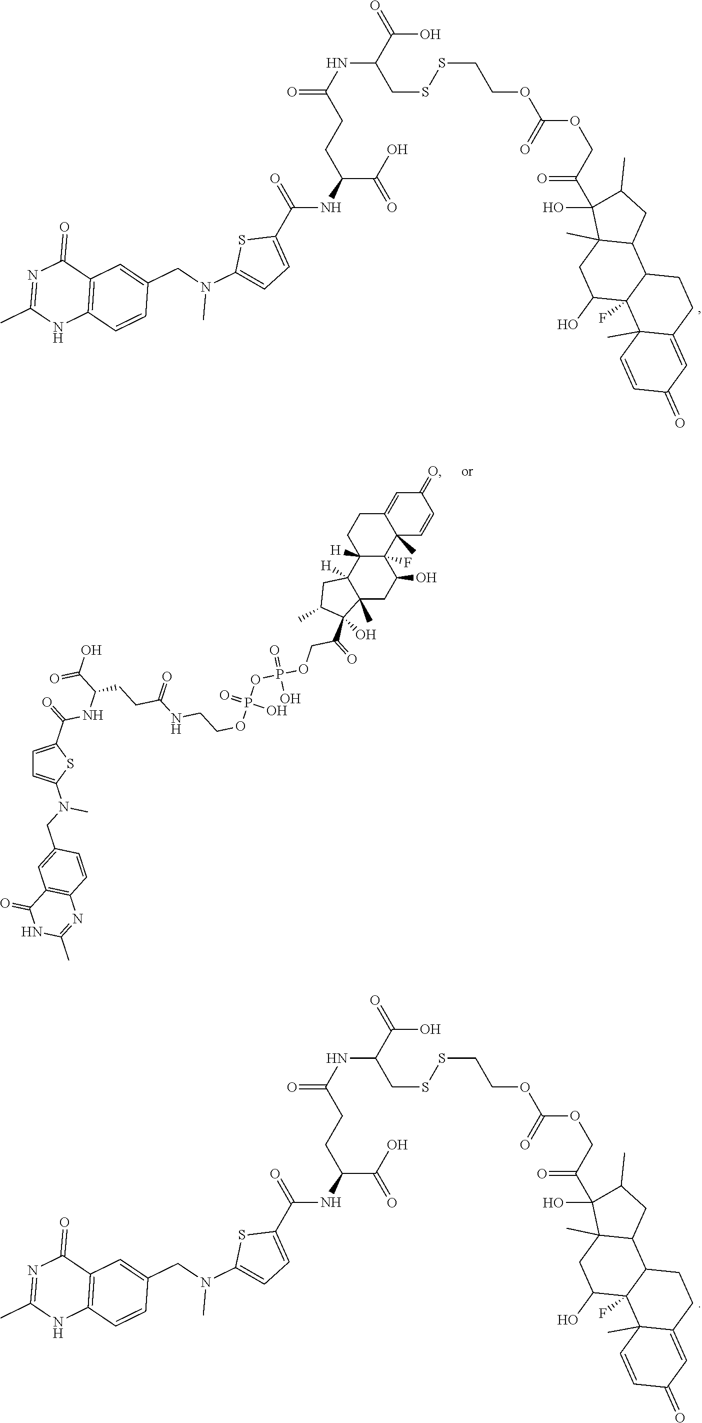 [SMILES: Cc1nc(=O)c2cc(CN(C)c3ccc(C(=O)N[C@@H](CCC(=O)NC(CSSCCOC(=O)OCC(=O)C4(O)C(C)CC5C6CCC7=CC(=O)C=CC7(C)[C@@]6(F)C(O)CC54C)C(=O)O)C(=O)O)s3)ccc2[nH]1 ; Cc1nc2ccc(CN(C)c3ccc(C(=O)N[C@@H](CCC(=O)NCCOP(=O)(O)OP(=O)(O)OCC(=O)[C@@]4(O)[C@H](C)C[C@H]5[C@@H]6CCC7=CC(=O)C=C[C@]7(C)[C@@]6(F)[C@@H](O)C[C@@]54C)C(=O)O)s3)cc2c(=O)[nH]1 ; Cc1nc(=O)c2cc(CN(C)c3ccc(C(=O)N[C@@H](CCC(=O)NC(CSSCCOC(=O)OCC(=O)C4(O)C(C)CC5C6CCC7=CC(=O)C=CC7(C)[C@@]6(F)C(O)CC54C)C(=O)O)C(=O)O)s3)ccc2[nH]1]

A compound hereof can have, or can comprise, the following structure:

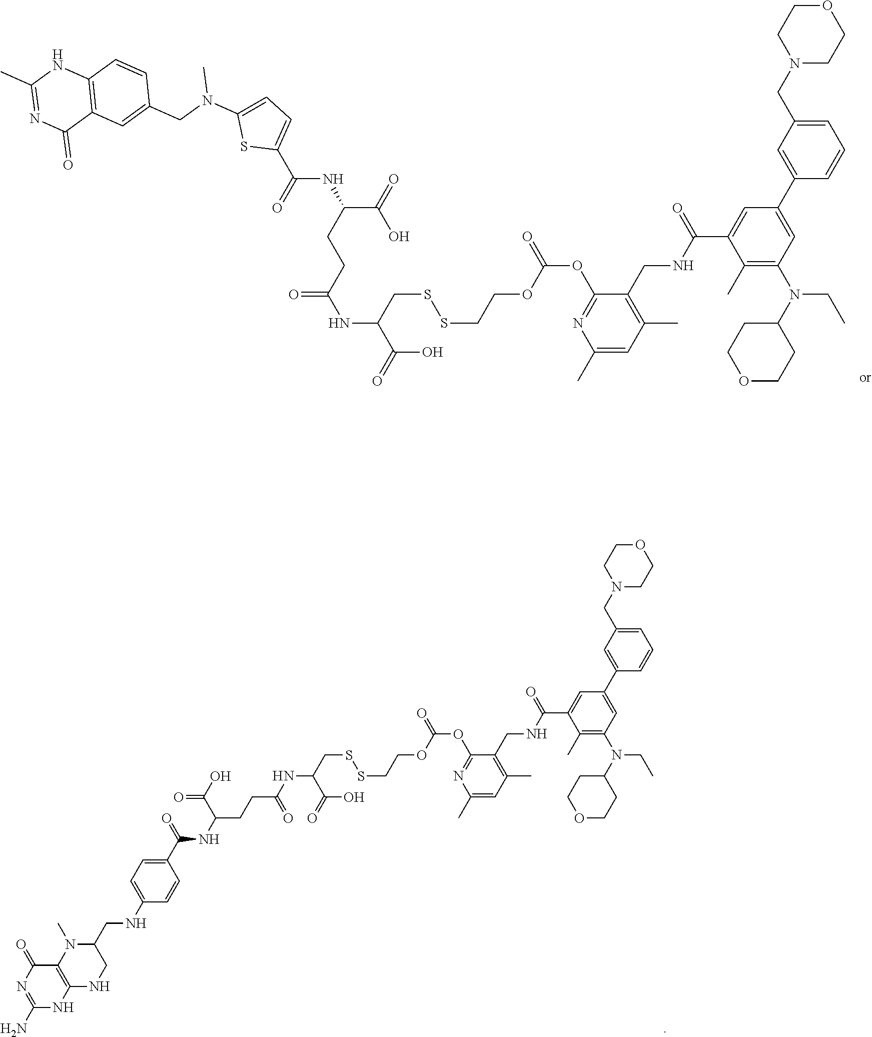

Examples of compounds comprising a TLR 7/8 agonist include, but are not limited to, the targeted, releasable raltitrexed-TLR7-TA compound having the structure:

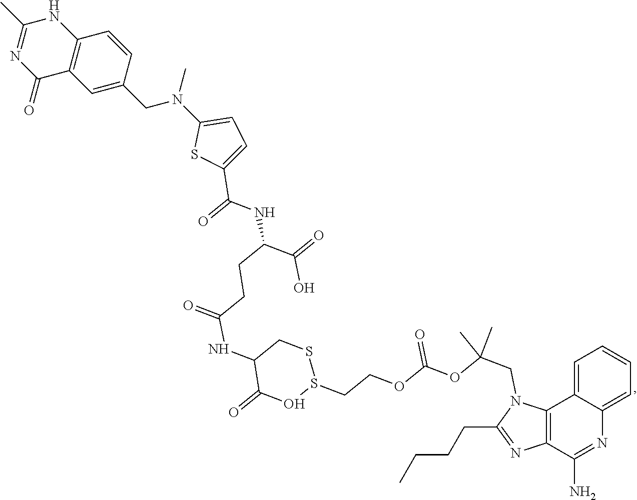

the targeted, non-releasable raltitrexed-TLR7-1 compound having the structure:

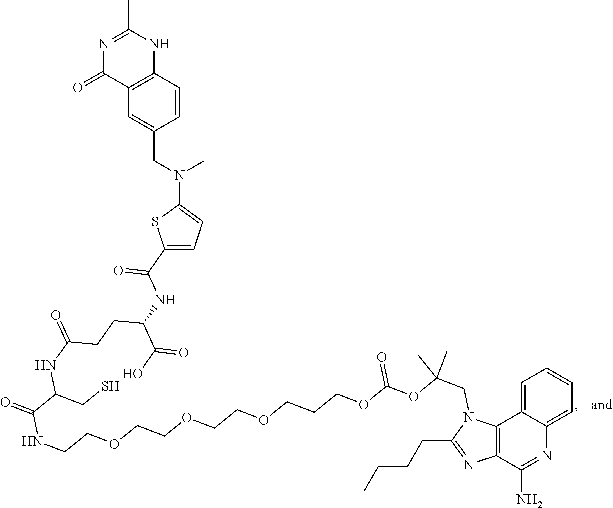 [SMILES: CCCCc1nc2c(N)nc3ccccc3c2n1CC(C)(C)OC(=O)OCCCOCCOCCOCCNC(=O)C(CS)NC(=O)CC[C@H](NC(=O)c1ccc(N(C)Cc2ccc3[nH]c(C)nc(=O)c3c2)s1)C(=O)O]

the targeted, non-releasable raltitrexed-TLR7-1A compound having the structure:

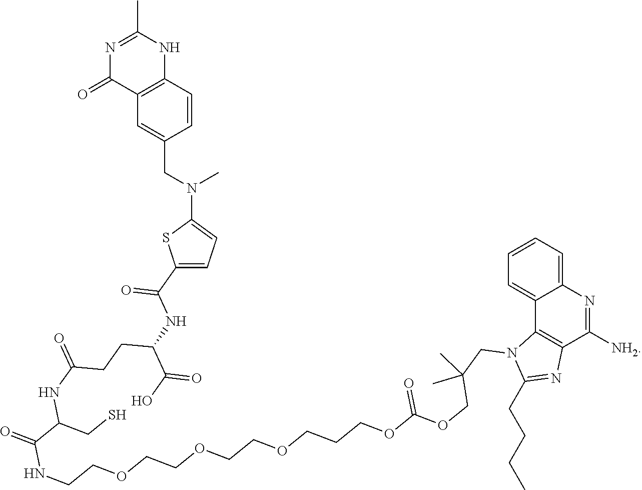 [SMILES: CCCCc1nc2c(N)nc3ccccc3c2n1CC(C)(C)COC(=O)OCCCOCCOCCOCCNC(=O)C(CS)NC(=O)CC[C@H](NC(=O)c1ccc(N(C)Cc2ccc3[nH]c(C)nc(=O)c3c2)s1)C(=O)O]

An example of a compound comprising an EZH2 antagonist includes but is not limited to, the targeted, releasable raltitrexed-EZH2 antagonist having the structure:

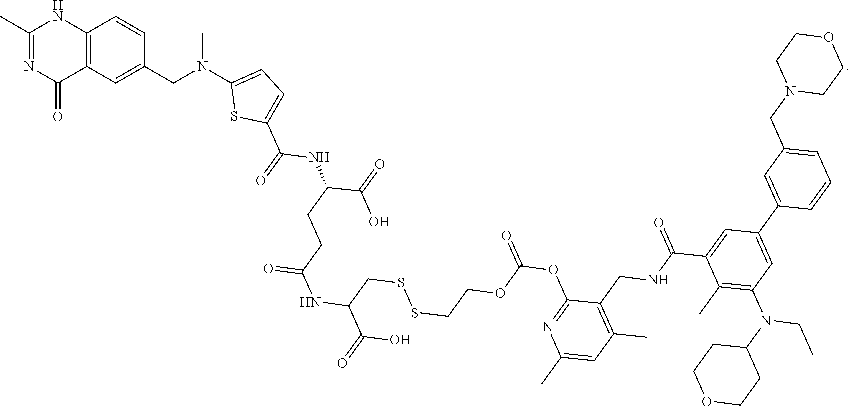 [SMILES: CCN(c1cc(-c2cccc(CN3CCOCC3)c2)cc(C(=O)NCc2c(C)cc(C)nc2OC(=O)OCCSSCC(NC(=O)CC[C@H](NC(=O)c2ccc(N(C)Cc3ccc4[nH]c(C)nc(=O)c4c3)s2)C(=O)O)C(=O)O)c1C)C1CCOCC1]

Any of the above compounds can further comprise a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the linker. The compound can further comprise an albumin binding group, e.g., an albumin binding group selected from the group consisting of

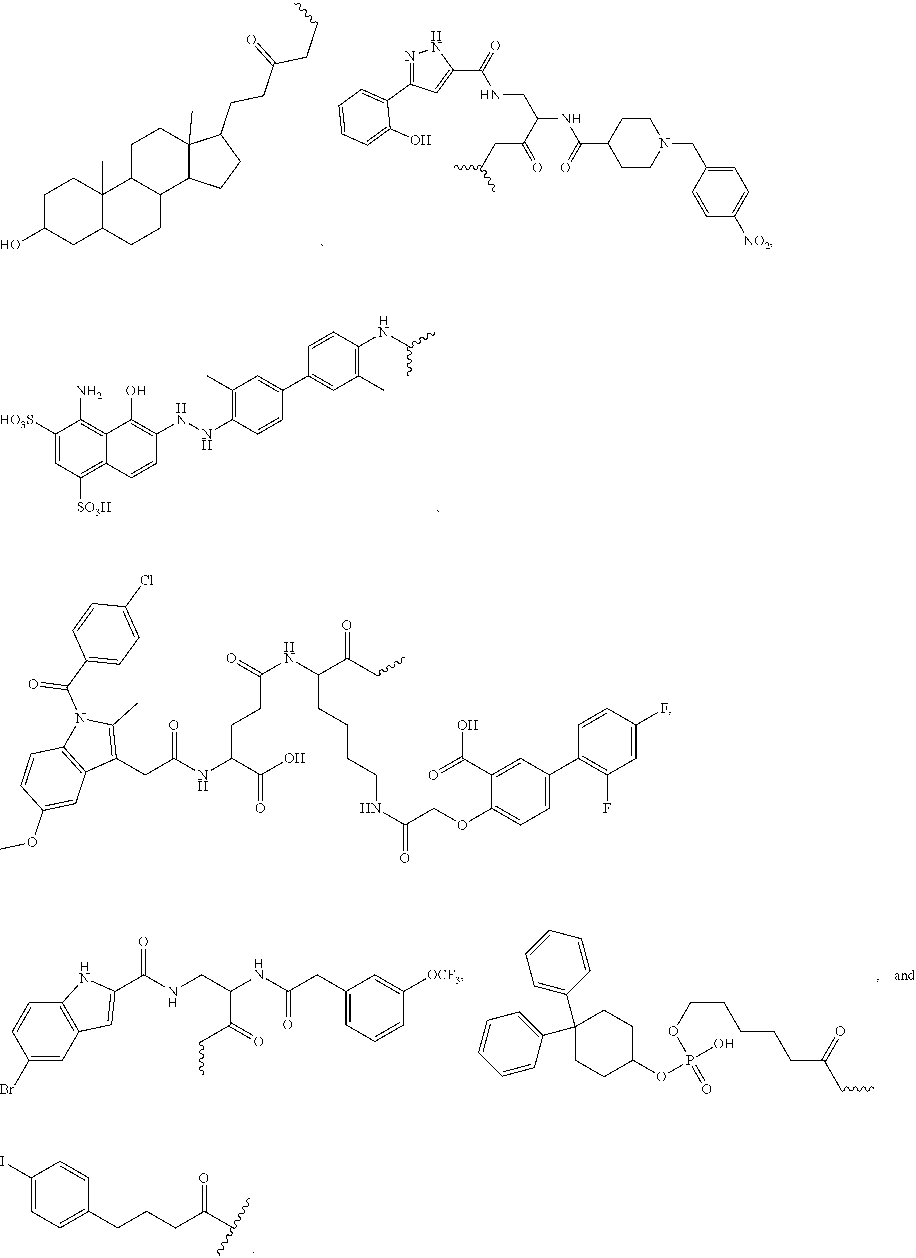

While structures shown above may be represented as flat, one of ordinary skill in the art will appreciate that the ligands and conjugates represented above include stereoisomers, i.e., ligands and conjugates with identical structures but different configurations or spatial arrangements. Stereoisomerism is often due to chirality or “handedness,” i.e., the presence of right-handed (R) and left-handed (L) forms of drugs, which are not superimposable mirror images (i.e., “enantiomers”).

Chiral conjugates (or conjugates comprising chiral ligands, for example) can be administered as mixtures or single enantiomers, particularly if there are important differences in their activity and pharmacokinetics to be taken into account. It is intended that the above structural representations encompass single enantiomers and mixtures thereof.

One of ordinary skill in the art will further appreciate that the above ligands and conjugates can be “deuterated,” meaning one or more hydrogen atoms can be replaced with deuterium. As deuterium and hydrogen have nearly the same physical properties, deuterium substitution is the smallest structural change that can be made. Replacement of hydrogen with deuterium can increase stability in the presence of other drugs, thereby reducing unwanted drug-drug interactions, and can significantly lower the rate of metabolism (due to the kinetic isotope effect). By lowering the rate of metabolism, half-life can be increased, toxic metabolite formation can be reduced, and the dosage amount and/or frequency can be decreased.

The above compounds can be synthesized using methods known in the art and exemplified herein. See, e.g., the Examples.

### Pharmaceutical Compositions

In view of the above, further provided is a pharmaceutical composition comprising any of the compounds herein. In certain embodiments, provided herein is a pharmaceutical composition comprising a compound described herein (e.g., a compound of Formula (I)) and one or more pharmaceutically acceptable excipients.

Provided is a compound of Formula (I):

T-L-E (I)

or a pharmaceutically acceptable salt thereof, wherein T is a radical of raltitrexed, 5-MTHF, an analog of raltitrexed, or an analog of 5-MTHF; L is a linker; and E is a radical of a therapeutic agent.

In certain embodiments, T has the structure of Formula (II):

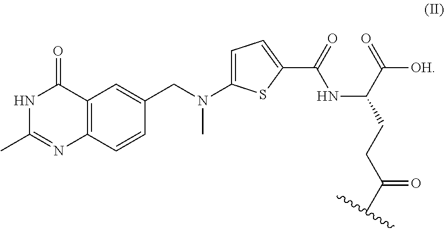 [SMILES: *C(=O)CC[C@H](NC(=O)c1ccc(N(C)Cc2ccc3nc(C)[nH]c(=O)c3c2)s1)C(=O)O]

In certain embodiments, T has the structure of Formula (III):

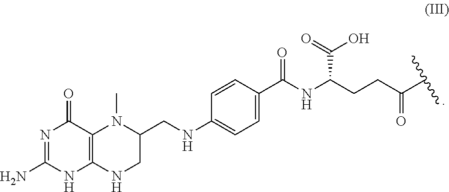 [SMILES: *C(=O)CC[C@H](NC(=O)c1ccc(NCC2CNc3[nH]c(N)nc(=O)c3N2C)cc1)C(=O)O]

The therapeutic agent can be selected from the group consisting of TLR7 agonist, a PI3K inhibitor, a steroid, a NLR2 agonist, a STING agonist, an EZH2 inhibitor, a NLRP3 inhibitor, a Caspase I inhibitor, a RLR agonist, an AIM2-like receptor agonist, and an agonist of RAGE.

The therapeutic agent can be a NLR2 agonist having the structure:

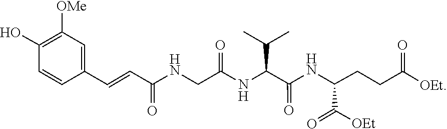 [SMILES: CCOC(=O)CC[C@@H](NC(=O)[C@@H](NC(=O)CNC(=O)/C=C/c1ccc(O)c(OC)c1)C(C)C)C(=O)OCC]

The therapeutic agent can be a STING agonist having the structure:

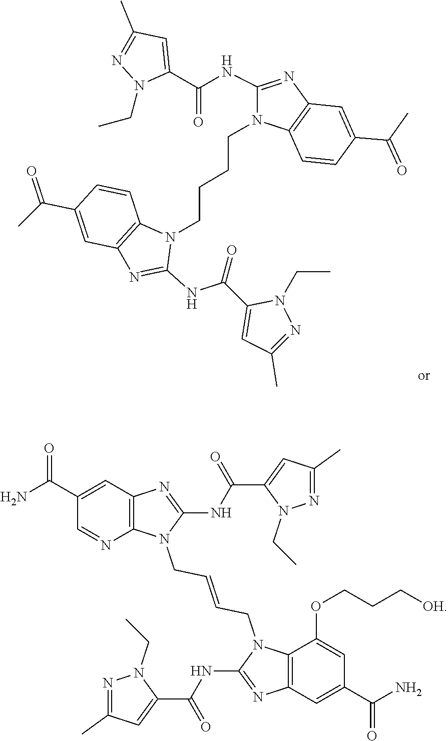 [SMILES: CCn1nc(C)cc1C(=O)Nc1nc2cc(C(C)=O)ccc2n1CCCCn1c(NC(=O)c2cc(C)nn2CC)nc2cc(C(C)=O)ccc21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO)c21]

In certain embodiments, the therapeutic agent is an EZH2 inhibitor. The EZH2 inhibitor can be

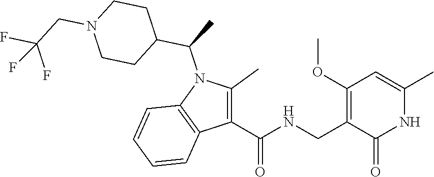 [SMILES: COc1cc(C)[nH]c(=O)c1CNC(=O)c1c(C)n([C@H](C)C2CCN(CC(F)(F)F)CC2)c2ccccc12]

or tazemetostat.

In certain embodiments, the therapeutic agent is a NLRP3 inhibitor having the structure:

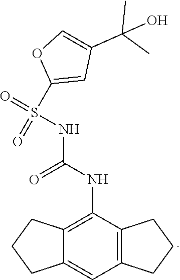 [SMILES: CC(C)(O)c1coc(S(=O)(=O)NC(=O)Nc2c3c(cc4c2CCC4)CCC3)c1]

In certain embodiments, the therapeutic agent is a Caspase I inhibitor having the structure:

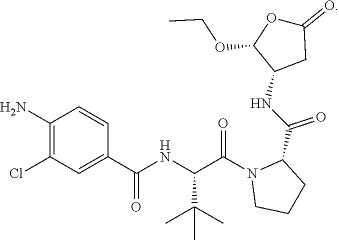 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C]

In certain embodiments, the therapeutic agent is a PI3 kinase inhibitor having the structure:

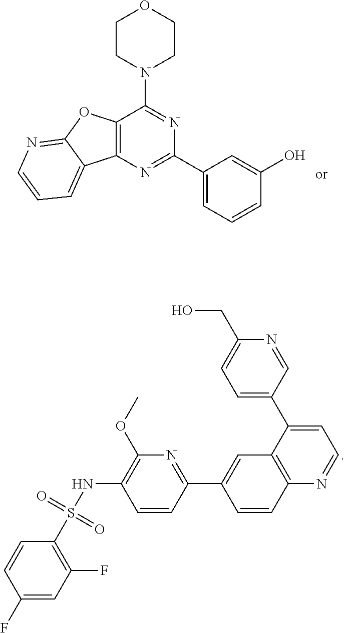 [SMILES: Oc1cccc(-c2nc(N3CCOCC3)c3oc4ncccc4c3n2)c1 ; COc1nc(-c2ccc3nccc(-c4ccc(CO)nc4)c3c2)ccc1NS(=O)(=O)c1ccc(F)cc1F]

In certain embodiments, the therapeutic agent is a RLR agonist having the structure:

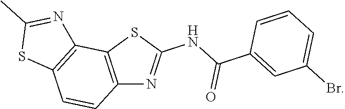 [SMILES: Cc1nc2c(ccc3nc(NC(=O)c4cccc(Br)c4)sc32)s1]

Also provided is a compound of the Formula (I):

T-L-E (I)

or a pharmaceutically acceptable salt thereof, wherein T is a radical of raltitrexed, 5-MTHF, an analog of raltitrexed, or an analog of 5-MTHF; L is a linker; and E is a radical of a TLR7 agonist represented by Formula (IV):

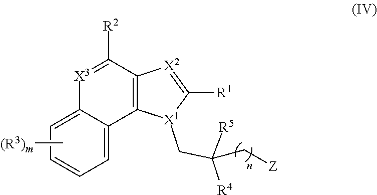

or a pharmaceutically acceptable salt thereof, wherein:
R¹, R³, R⁴, R⁵ are each independently a hydrogen (H), alkyl, alkoxyl, alkenyl, alkynyl, cycloalkyl, aryl halo, heteroaryl, —COR^{2x},

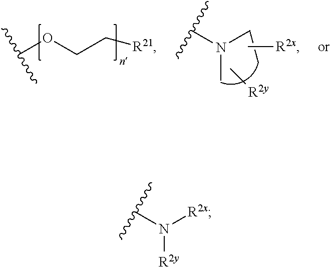

R² is a H, —OH, —NH₂, —NHR^{2x}, N₃, —NH—CH₂—NH₂, —CONH₂, —SO₂NH₂, —NH—CS—NH₂,

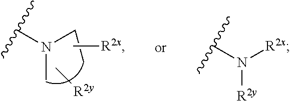

Z is a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂,

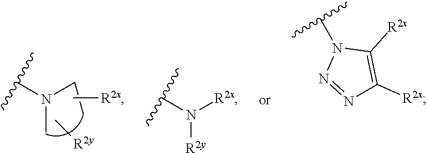

wherein:
   each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl; and
   each R^{2z} is independently selected from the group consisting of —NH₂, —NR^{2q}R^{2q′}, —O—R^{2q}, —SO—R^{2q} and —COR^{2q}; wherein each R^{2q} and R^{2q′} is independently alkyl or H,

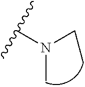 [SMILES: *N(C)CC]

is a 3-10 membered N-containing non-aromatic, mono- or bicyclic heterocycle,
R²¹ is H or alkyl,
n′ is 0-30; and
wherein in Formula (IV), each of X¹, X², X³ is independently CR^{q} or N, wherein each R^{q} is independently H, halogen, or optionally substituted alkyl,
   n is 0-30,
   m is 0-4; and
   when n is 0, Y is not H, —OH, or —O—R^{2x}.

E can be a radical of a compound represented by Formula (IVA):

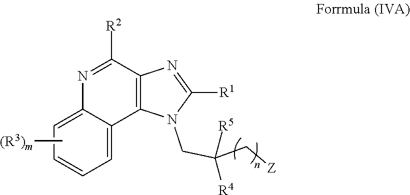

or a pharmaceutically acceptable salt thereof, wherein:
R¹ is an optionally substituted C₃-C₈ alkyl;
R² is H, —OR^{z}, —SO₂N(R^{Z})₂, —NR^{2x}R^{2y}, or N₃, wherein:
   R^{2x} and R^{2y} are each independently H, —N(R^{z})₂, —CON(R^{z})₂, —C(R^{z})₂—N(R^{z})₂, —CS—N(R^{z})₂, or optionally substituted alkyl,
   each R^{z} is independently hydrogen, halogen, or optionally substituted alkyl, or
   R^{2x} and R^{2y} are taken together to form an optionally substituted heterocycloalkyl;
Z is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂,
each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein the alkyl, alkoxy, heteroalkyl, cycloalkyl, or heterocycloalkyl is optionally substituted;
R⁴ and R⁵ are each independently alkyl, alkoxy, halogen, or cycloalkyl, wherein each of the alkyl, alkoxy, and cycloalkyl is optionally substituted;
n is 1-6; and
m is 0-4.

In certain embodiments, the compound of Formula (I) is represented by Formula (IVB) or Formula (IVC):

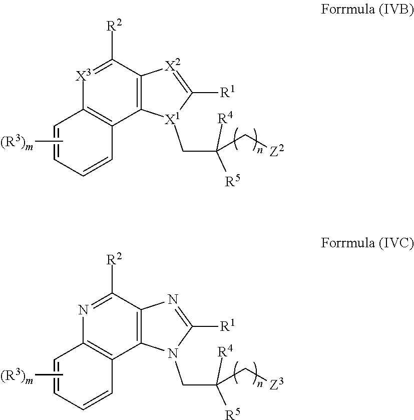

or a pharmaceutically acceptable salt thereof, wherein: each R¹ is independently an optionally substituted C₃-C₈ alkyl; each R² is independently H, —OR^{z}, —SO₂N(R^{z})₂, —NR^{2x}R^{2y}, or N₃; each R^{2x} and R^{2y} are independently H, —N(R^{z})₂, —CON(R^{z})₂, —C(R^{z})₂—N(R^{z})₂, —CS—N(R^{z})₂, or optionally substituted alkyl, each R^{Z} is independently H, halogen, or an optionally substituted alkyl, or R^{2x} and R^{2y} are taken together to form an optionally substituted heterocycloalkyl; each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein each of the alkyl, alkoxy, heteroalkyl, cycloalkyl, or heterocycloalkyl is optionally substituted; each R⁴ and R⁵ are independently alkyl, alkoxy, halogen, or cycloalkyl, wherein each of the alkyl, alkoxy, and cycloalkyl, is optionally substituted; n is 1-6; m is 0-4; each Z² and Z³ is independently a group of the formula T-L-, T-L-O—, T-L-O-alkyl-, T-L-S¹—, T-SO₂—NH—, T-L-NR^{a}R^{b}—, T-L-S(O)ₓ-alkyl-, T-L-CO—, T-L-aryl-, T-L-NH—CO—NH—, T-L-NH—O—, T-L-NH—NH—, T-L-NH—CS—NH, T-L-C(O)-alkyl-, or T-L-SO₂—; R^{a} and R^{b} are each independently H, halo, hydroxy, alkoxy, aryl, amino, acyl or C(O)R^{c}, wherein R^{c} is alkyl, aryl, oxy or alkoxy; S¹ is a spacer; x is 0-3; n is 1-3 and m is 0-4.

In other embodiments, the compound of Formula (I) is represented by Formula (IVB):

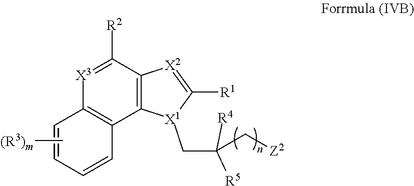

or a pharmaceutically acceptable salt thereof, wherein: R is an optionally substituted C₃-C₈ alkyl; R² is H, —OR^{z}, —SO₂N(R^{z})₂, —NR^{2x}R^{2y}, or N₃; R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl, each R^{z} is independently H, halogen, or an optionally substituted alkyl, or R^{2X} and R^{2y} are taken together to form an optionally substituted heterocycloalkyl; each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein each of the alkyl, alkoxy, heteroalkyl, cycloalkyl, or heterocycloalkyl is optionally substituted; R⁴ and R⁵ are each independently alkyl, alkoxy, halogen, or cycloalkyl, wherein each of the alkyl, alkoxy, and cycloalkyl, is optionally substituted; n is 1-6; m is 0-4; each of Z² is a group of the formula T-L-, T-L-O—, T-L-O-alkyl-, T-L-S¹—, T-SO₂—NH—, T-L-NR^{a}R^{b}—, T-L-S(O)ₓ-alkyl-, T-L-CO—, T-L-aryl-, T-L-NH—CO—NH—, T-L-NH—O—, T-L-NH—NH—, T-L-NH—CS—NH, T-L-C(O)-alkyl-, or T-L-SO₂—; R^{a} and R^{b} are each independently H, halo, hydroxy, alkoxy, aryl, amino, acyl or C(O)R^{c}, wherein R^{c} is alkyl, aryl, oxy or alkoxy; x is 0-3; n is 1-3; S¹ is a spacer; and m is 0-4.

In other embodiments, the compound of Formula (I) is represented by Formula (IVC):

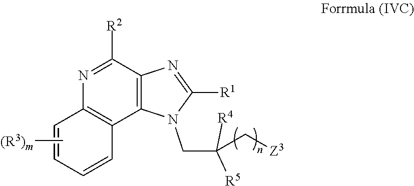

or a pharmaceutically acceptable salt thereof, wherein: R is an optionally substituted C₃-C₈ alkyl; R² is H, —OR^{z}, —SO₂N(R^{z})₂, —NR^{2x}R^{2y}, or N₃; R^{2x} and R^{2y} are each independently H, —N(R^{z}, —CON(R^{z})₂, —C(R^{z})₂—N(R^{z})₂, —CS—N(R^{z})₂, or optionally substituted alkyl, each R^{z} is independently H, halogen, or an optionally substituted alkyl, or R^{2x} and R^{2y} are taken together to form an optionally substituted heterocycloalkyl; each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein each of the alkyl, alkoxy, heteroalkyl, cycloalkyl, or heterocycloalkyl is optionally substituted; R⁴ and R⁵ are each independently alkyl, alkoxy, halogen, or cycloalkyl, wherein each of the alkyl, alkoxy, and cycloalkyl, is optionally substituted; n is 1-6; m is 0-4; each of Z³ is a group of the formula T-L-, T-L-O—, T-L-O-alkyl-, T-L-S¹—, T-SO₂—NH—, T-L-NR^{a}R^{b}—, T-L-S(O)ₓ-alkyl-, T-L-CO—, T-L-aryl-, T-L-NH—CO—NH—, T-L-NH—O—, T-L-NH—NH—, T-L-NH—CS—NH, T-L-C(O)-alkyl-, or T-L-SO₂—; R^{a} and R^{b} are each independently H, halo, hydroxy, alkoxy, aryl, amino, acyl or C(O)R^{c}, wherein R^{c} is alkyl, aryl, oxy or alkoxy; x is 0-3; S¹ is a spacer; n is 1-3 and m is 0-4.

In certain embodiments where the compound of Formula (I) is represented by Formula (IVB) or Formula (IVC), R¹ is a C₁-C₆ alkyl optionally substituted with 1-3 substituents, each substituent independently being halogen or C₁-C₆ alkoxy; R² is —NR^{2x}R^{2y}, where R^{2x} and R^{2y} are each independently a H or a C₁-C₆ alkyl; each R³ is independently a halogen, —CN, C₁-C₆ alkyl, C₁-C₆ heteroalkyl, C₃-C₇ cycloalkyl, C₁-C₆ alkoxy, amino, hydroxy, carboxyl, or thiol; R⁴ and R⁵ are each independently C₁-C₆ alkyl; each X¹, X², and X³ is N; each of Z² and Z³ is independently T-L- or T-L-O—; n is 1; and m is 0-4. In certain embodiments where the compound of Formula (I) is represented by Formula (IVB) or Formula (IVC), each of Z² and Z³ is T-L-O—.

In certain embodiments of Formula (IV) or Formula (IVA), Z can be a group of the formula T-L-, T-L-O—, T-L-O-alkyl-, T-L-S¹—, T-SO₂—NH—, T-L-NR^{a}R^{b}—, T-L-S(O)ₓ-alkyl-, T-L-CO—, T-L-aryl-, T-L-NH—CO—NH—, T-L-NH—O—, T-L-NH—NH—, T-L-NH—CS—NH, T-L-C(O)-alkyl-, or T-L-SO₂—, wherein:

R^{a} and R^{b} are each independently H, halo, hydroxy, alkoxy, aryl, amino, acyl or C(O)R^{c}, wherein R^{c} is alkyl, aryl, oxy or alkoxy; S¹ is a spacer; and x is 0-3.

In certain embodiments of Formula (IVA), R¹ is a C₁-C₆ alkyl optionally substituted with 1-3 substituents, each substituent independently being halogen or C₁-C₆ alkoxy; R² is —NR^{2x}R^{2y}, where R^{2x} and R^{2y} are each independently a H or a C₁-C₆ alkyl; each R³ is independently a halogen, —CN, C₁-C₆ alkyl, C₁-C₆ heteroalkyl, C₃-C₇ cycloalkyl, C₁-C₆ alkoxy, amino, hydroxy, carboxyl, or thiol; R⁴ and R⁵ are each independently be C₁-C₆ alkyl; each X¹, X², and X³ is N; Z is T-L- or T-L-O—; n is 1; and m is 0. Z can be T-L-O—.

R¹ can be optionally substituted C₃-C₆ alkyl. R¹ can be an optionally substituted acyclic C₃-C₆ alkyl. R² can be —NR^{2x}R^{2y}. R² can be —NH₂.

The compound of Formula (IVA) can be one of the formulae:

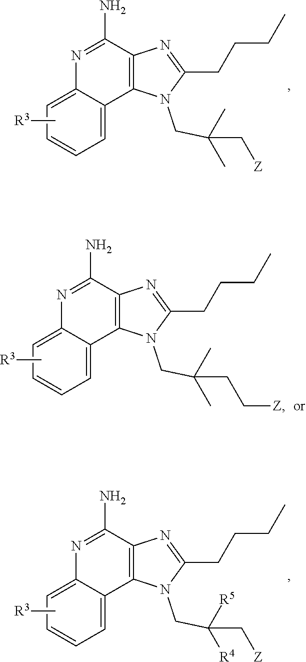

or a pharmaceutically acceptable salt thereof, wherein R³ is optionally absent. The compound of Formula (IV) can be one of the formulae:

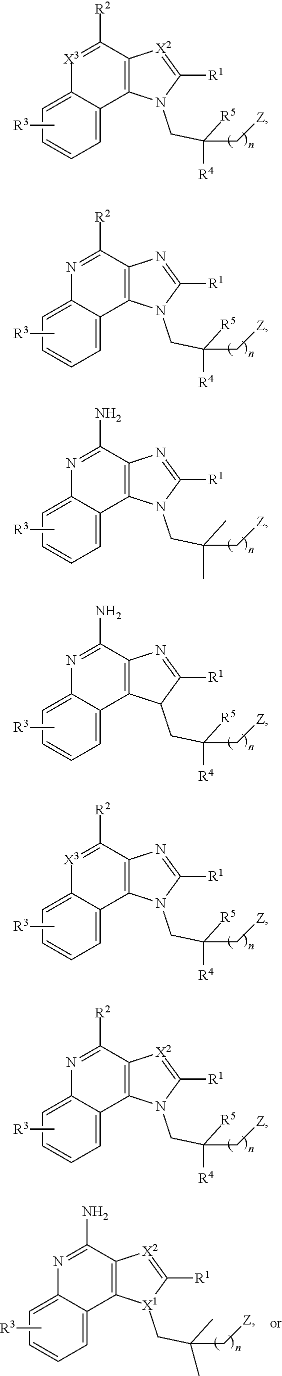

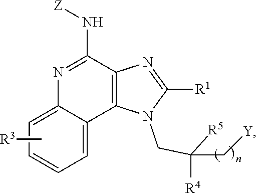

or a pharmaceutically acceptable salt of any of the foregoing formulae, wherein R³ is optionally absent.

R¹ can be a C₁-C₆ alkyl. R² can be —NH₂. R³ can be absent.

In certain embodiments. R¹ is a C₁-C₆ alkyl; R² is —NH₂; n is 1; and R³ is absent.

In certain embodiments, the compound of the Formula (I) is a compound represented by Formula (V):

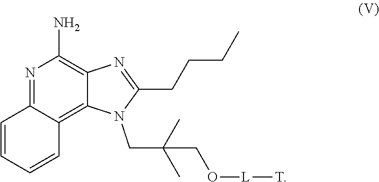

In certain embodiments, the radical of the TLR7 agonist (e.g., E) has the structure:

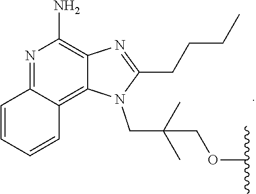

Further provided is a compound of the Formula (I):

T-L-E (I)

or a pharmaceutically acceptable salt thereof, wherein T is a radical of raltitrexed, 5-MTHF, an analog of raltitrexed, or an analog of 5-MTHF; L is a linker; and E is

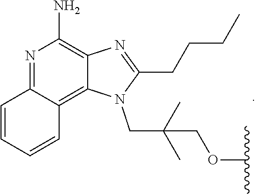

Still further provided is a compound of the Formula (I):

T-L-E (I)

or a pharmaceutically acceptable salt thereof, wherein T is a radical of raltitrexed, 5-MTHF, an analog of raltitrexed, or an analog of 5-MTHF; L is a linker; and E is a radical of the structure:

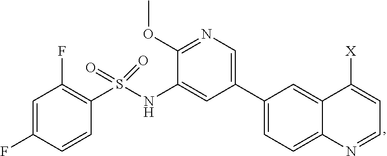

wherein X can be any of the following:

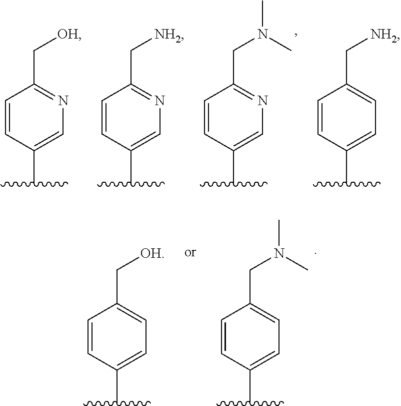

E can comprise a radical of the structure:

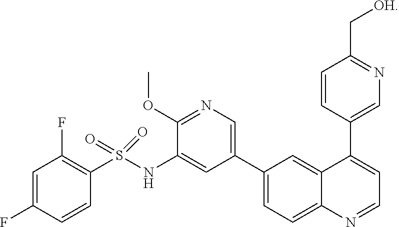

In certain embodiments, the compound is of Formula (I):

T-L-E (I)

or is a pharmaceutically acceptable salt thereof, wherein:
T is a radical of raltitrexed, 5-MTHF, an analog of raltitrexed, or an analog of 5-MTHF;
L is a linker; and
E is a radical of a corticosteroid.

The corticosteroid can be betamethasone, cortisone, cortivazol, difluprednate, hydrocortisone, prednisolone, methylprednisolone, prednisone, dexamethasone, hydrocortisone-17-valerate, budesonide, flumethazone, fluticasone propionate, fluorocortisone, fludrocortisone, paramethasone, eplerenone, or an ester of any of the foregoing.

L can be a releasable linker. L can be a non-releasable linker. L can comprise an optionally substituted heteroalkyl. In certain embodiments, the optionally substituted heteroalkyl is substituted with at least one substituent selected from the group consisting of alkyl, hydroxyl, acyl, polyethylene glycol (PEG), carboxylate, and halo. L can comprise a substituted heteroalkyl with at least one disulfide bond in the backbone thereof. L can comprise a peptide or a peptidoglycan with at least one disulfide bond in the backbone thereof. L can be a releasable linker that can be cleaved by enzymatic reaction, a reactive oxygen species (ROS), or reductive conditions. L can comprise the formula —NH—CH₂—CR⁶R⁷—S—S—CH₂—CH₂—O—CO—, wherein R⁶ and R⁷ are each, independently, H, alkyl, or heteroalkyl. L can be a group, or can comprise a group, of the formulae:

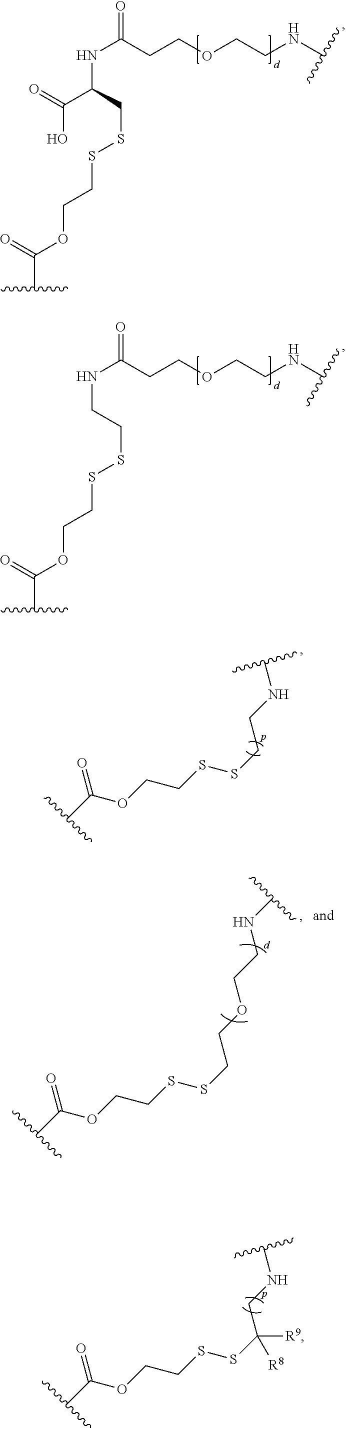

wherein p is an integer from 0 to 30; d is an integer from 1 to 40; and R⁸ and R⁹ are each, independently, H, alkyl, a heterocyclyl, a cycloalkyl, an aryl, or a heteroalkyl.

L can comprise one or more linker moieties, each of the one or more linker moieties independently selected from the group consisting of alkylene, heteroalkylene, —O— alkynylene, alkenylene, acyl, aryl, heteroaryl, amide, oxime, ether, ester, triazole, PEG, carboxylate, carbonate, carbamate, amino acid, peptide, and peptidoglycan. L can be, or can comprise, a peptide or a peptidoglycan. L can be, or can comprise, an amino acid. L can be, or can comprise, a PEG group. L can be, or can comprise, a polysaccharide. L can be, or can comprise, a group represented by the structure:

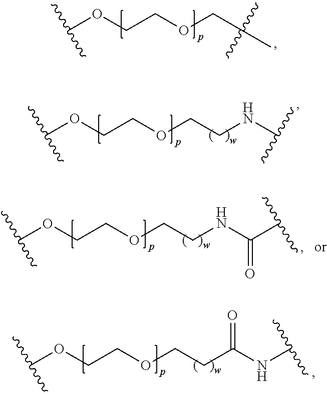

wherein w is 0-5 and p is 1-30. L can be, or can comprise, a linker moiety selected from the group consisting of:

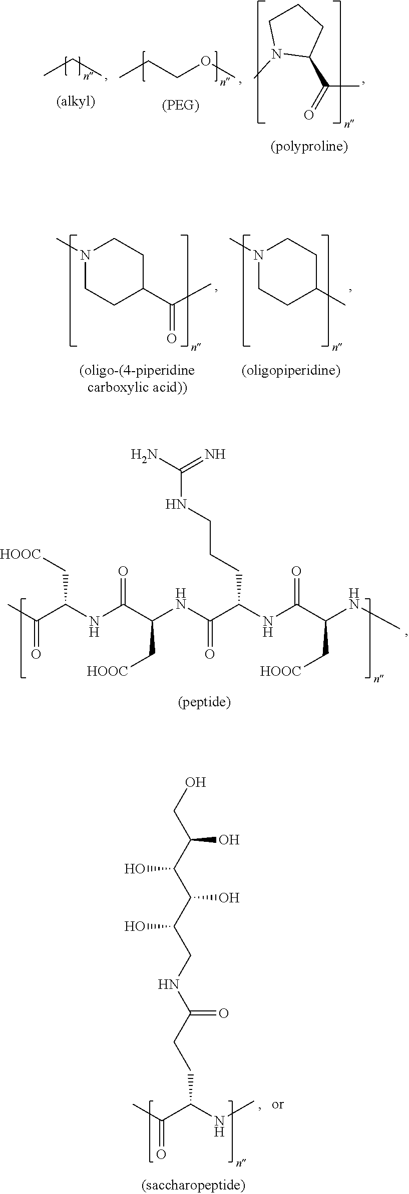

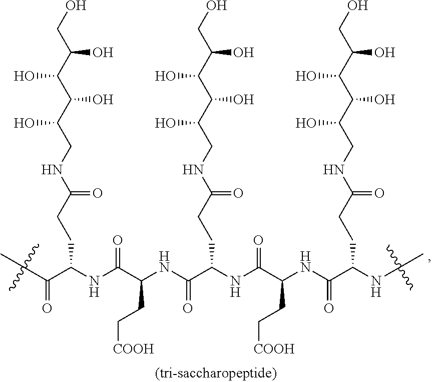

wherein n″ is 0-30. L can be a bivalent linker. L can be a trivalent linker.

Any of the above compounds can further comprise a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the linker.

The compound can further comprise an albumin binding group, e.g., an albumin binding group selected from the group consisting of:

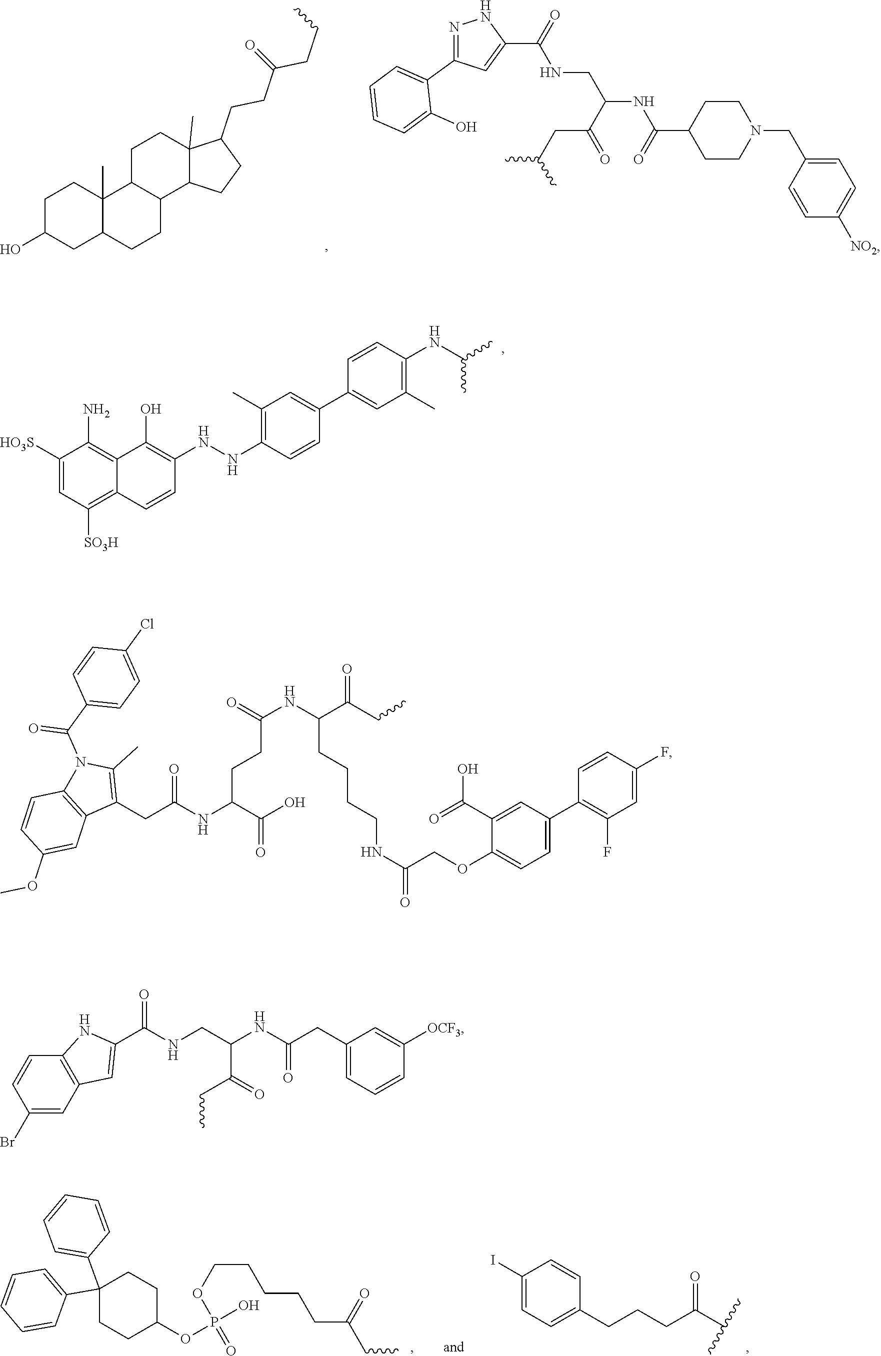

e.g., an albumin binding group selected from a group consisting of:

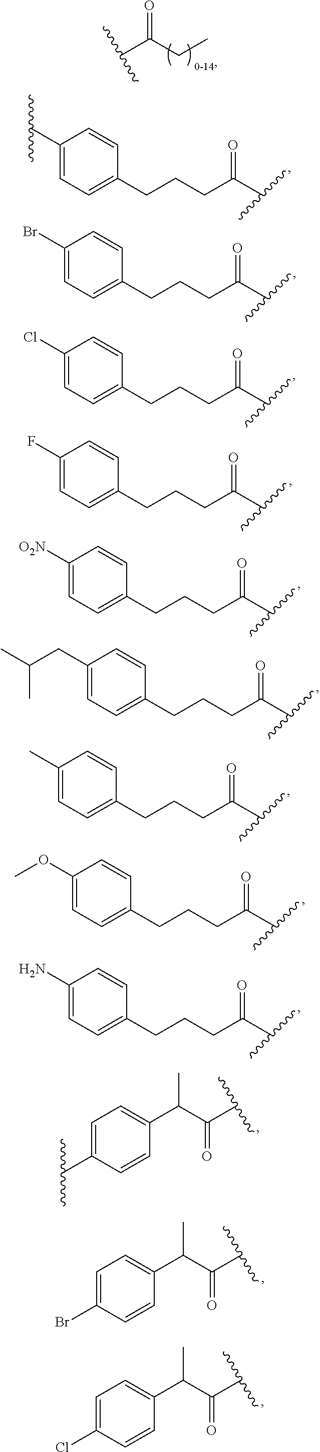

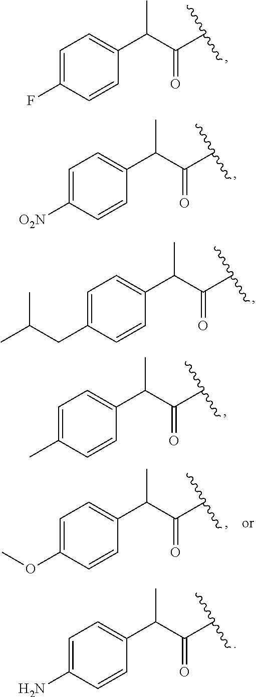

In certain embodiments, the compound comprises (e.g., consists of) one of the following structures:

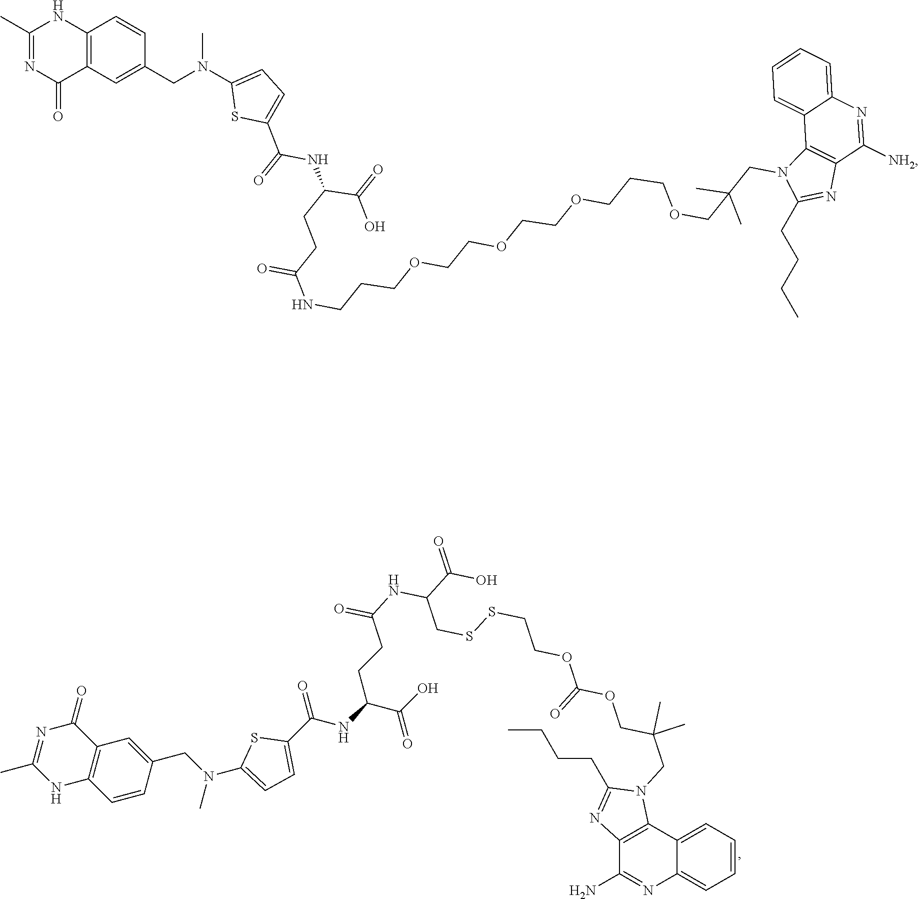 [SMILES: CCCCc1nc2c(N)nc3ccccc3c2n1CC(C)(C)COCCCOCCOCCOCCCNC(=O)CC[C@H](NC(=O)c1ccc(N(C)Cc2ccc3[nH]c(C)nc(=O)c3c2)s1)C(=O)O ; CCCCc1nc2c(N)nc3ccccc3c2n1CC(C)(C)COC(=O)OCCSSCC(NC(=O)CC[C@H](NC(=O)c1ccc(N(C)Cc2ccc3[nH]c(C)nc(=O)c3c2)s1)C(=O)O)C(=O)O]

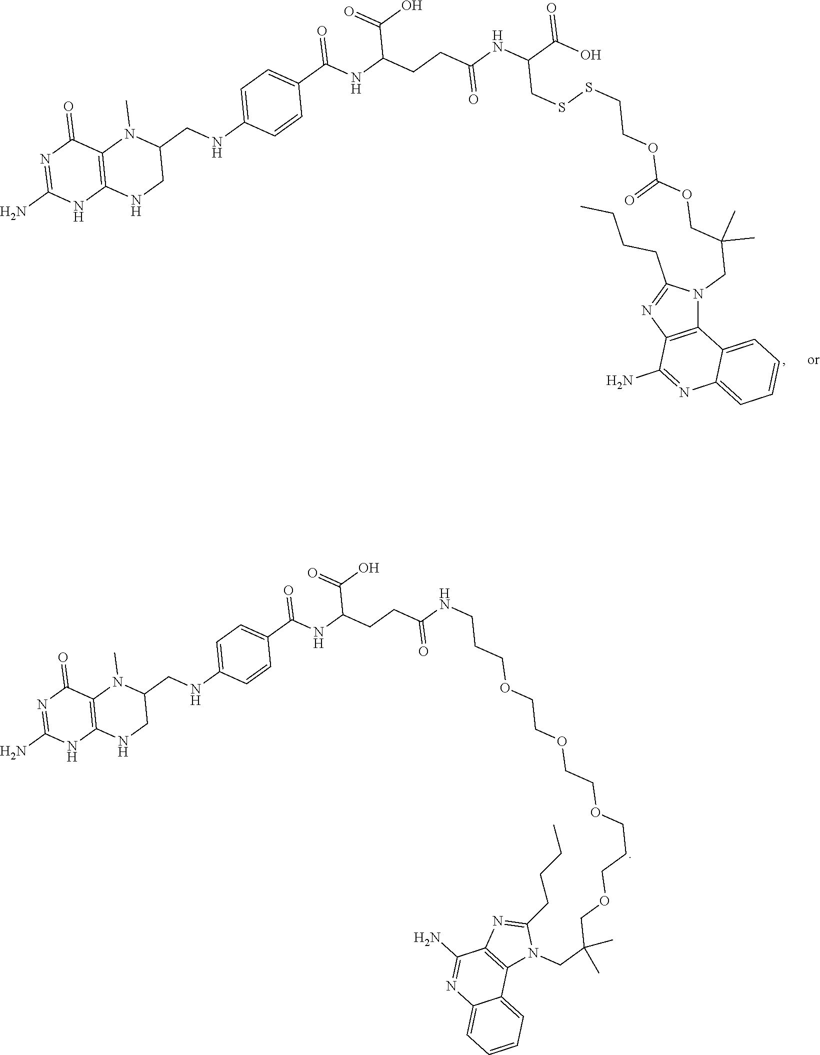

In certain embodiments, the compound comprises (e.g., consists of) one of the following structures:

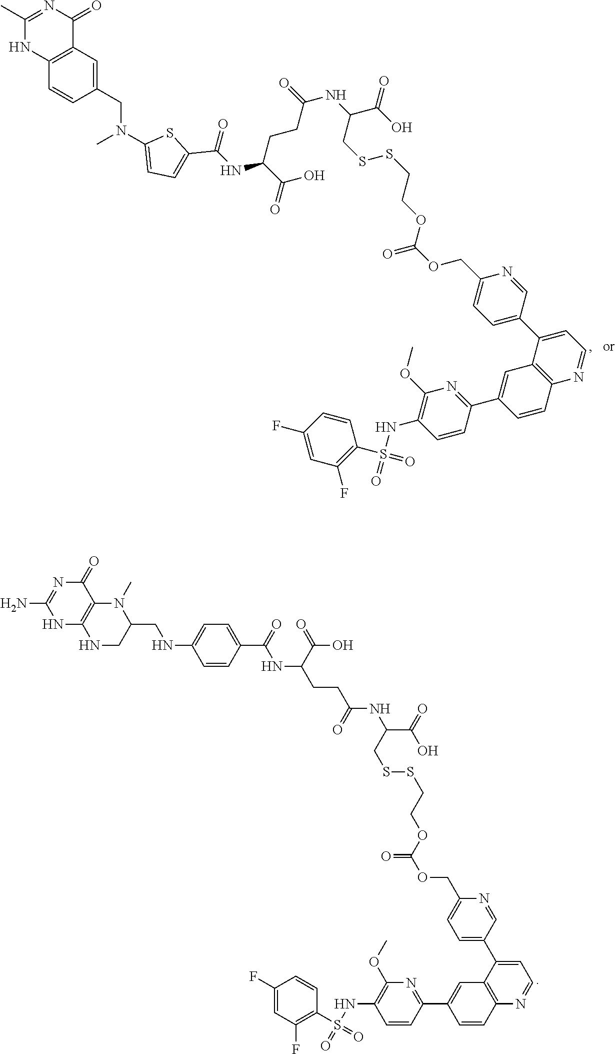 [SMILES: COc1nc(-c2ccc3nccc(-c4ccc(COC(=O)OCCSSCC(NC(=O)CC[C@H](NC(=O)c5ccc(N(C)Cc6ccc7[nH]c(C)nc(=O)c7c6)s5)C(=O)O)C(=O)O)nc4)c3c2)ccc1NS(=O)(=O)c1ccc(F)cc1F ; COc1nc(-c2ccc3nccc(-c4ccc(COC(=O)OCCSSCC(NC(=O)CCC(NC(=O)c5ccc(NCC6CNc7[nH]c(N)nc(=O)c7N6C)cc5)C(=O)O)C(=O)O)nc4)c3c2)ccc1NS(=O)(=O)c1ccc(F)cc1F]

In certain embodiments, the compound comprises (e.g., consists of) one of the following structures:

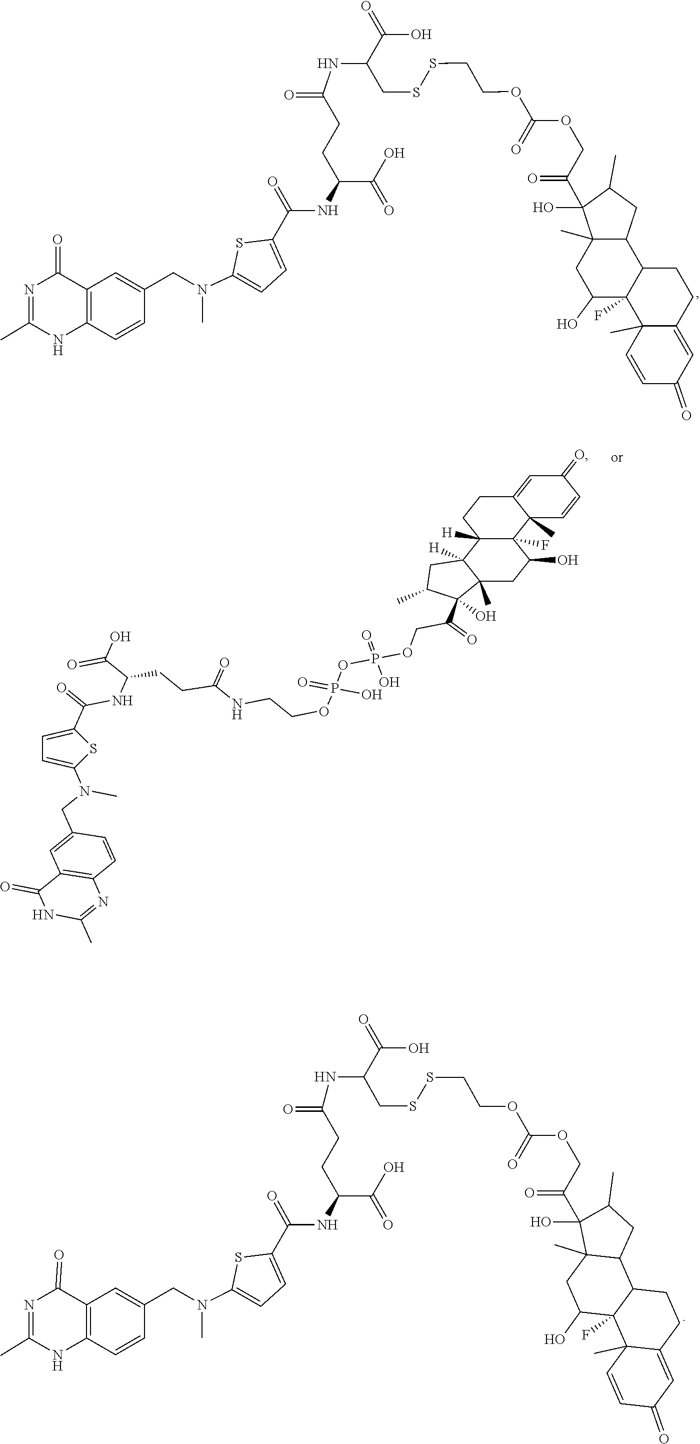 [SMILES: Cc1nc(=O)c2cc(CN(C)c3ccc(C(=O)N[C@@H](CCC(=O)NC(CSSCCOC(=O)OCC(=O)C4(O)C(C)CC5C6CCC7=CC(=O)C=CC7(C)[C@@]6(F)C(O)CC54C)C(=O)O)C(=O)O)s3)ccc2[nH]1 ; Cc1nc2ccc(CN(C)c3ccc(C(=O)N[C@@H](CCC(=O)NCCOP(=O)(O)OP(=O)(O)OCC(=O)[C@@]4(O)[C@H](C)C[C@H]5[C@@H]6CCC7=CC(=O)C=C[C@]7(C)[C@@]6(F)[C@@H](O)C[C@@]54C)C(=O)O)s3)cc2c(=O)[nH]1 ; Cc1nc(=O)c2cc(CN(C)c3ccc(C(=O)N[C@@H](CCC(=O)NC(CSSCCOC(=O)OCC(=O)C4(O)C(C)CC5C6CCC7=CC(=O)C=CC7(C)[C@@]6(F)C(O)CC54C)C(=O)O)C(=O)O)s3)ccc2[nH]1]

In certain embodiments, the compound comprises (e.g., consists of) one of the following structures:

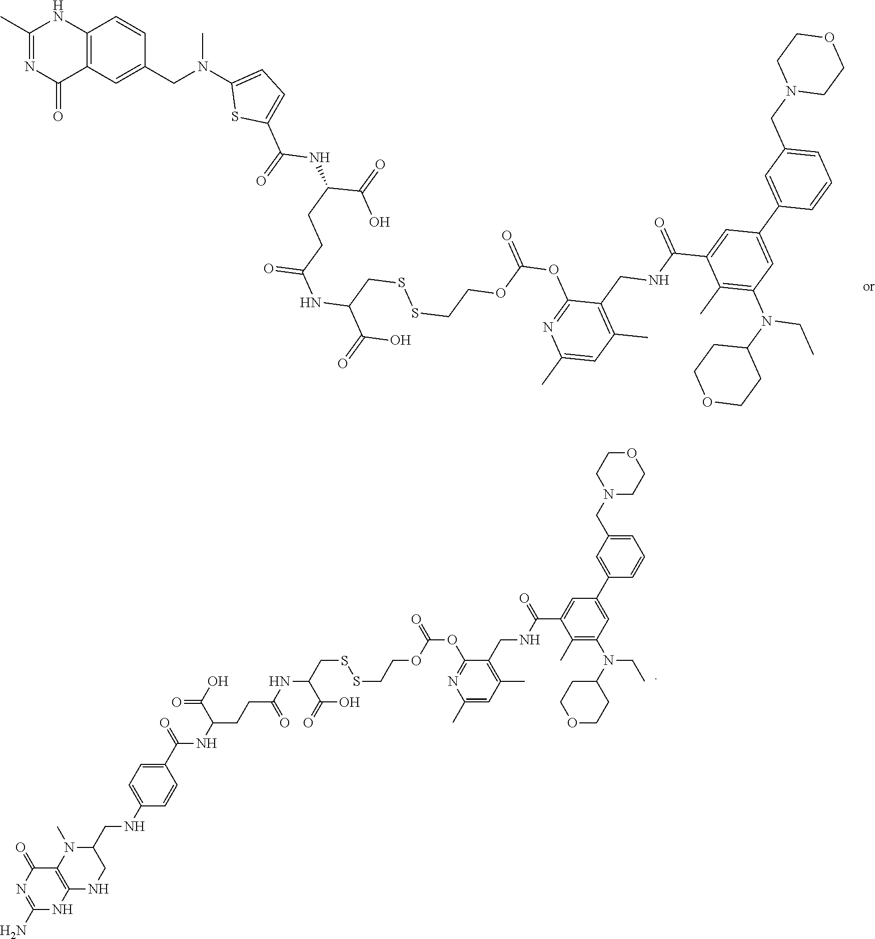 [SMILES: CCN(c1cc(-c2cccc(CN3CCOCC3)c2)cc(C(=O)NCc2c(C)cc(C)nc2OC(=O)OCCSSCC(NC(=O)CC[C@H](NC(=O)c2ccc(N(C)Cc3ccc4[nH]c(C)nc(=O)c4c3)s2)C(=O)O)C(=O)O)c1C)C1CCOCC1 ; CCN(c1cc(-c2cccc(CN3CCOCC3)c2)cc(C(=O)NCc2c(C)cc(C)nc2OC(=O)OCCSSCC(NC(=O)CCC(NC(=O)c2ccc(NCC3CNc4[nH]c(N)nc(=O)c4N3C)cc2)C(=O)O)C(=O)O)c1C)C1CCOCC1]

Any of the above compounds can further comprise a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the linker. The compound can further comprise an albumin binding group, e.g., an albumin binding group selected from the group consisting of:

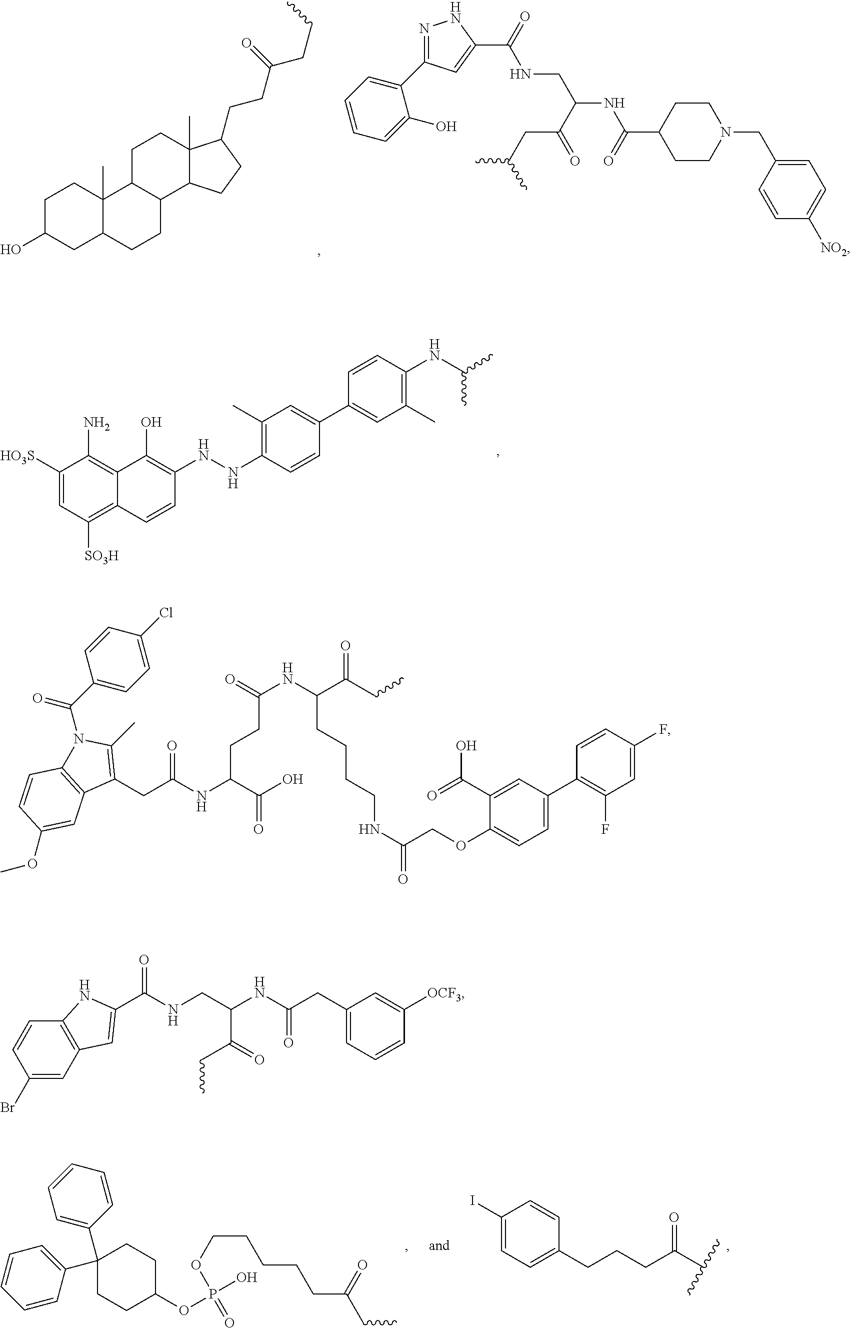

e.g., an albumin binding group selected from a group consisting of

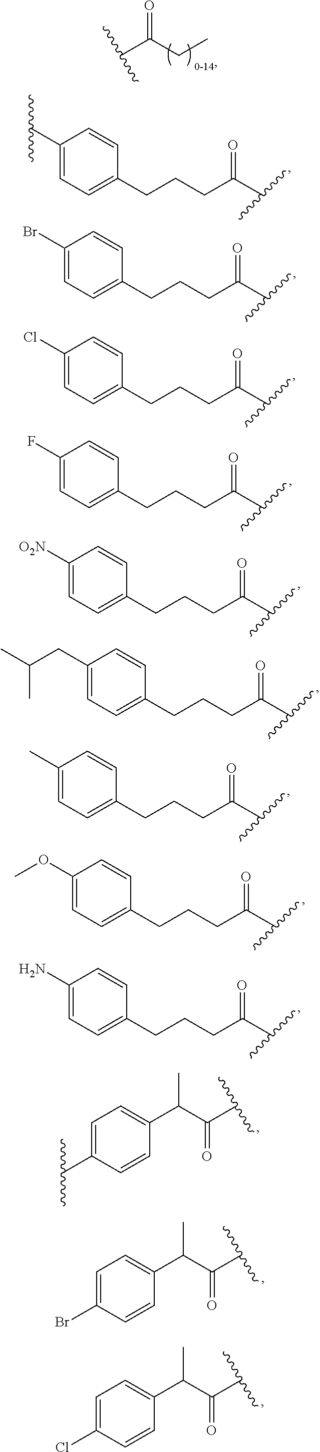

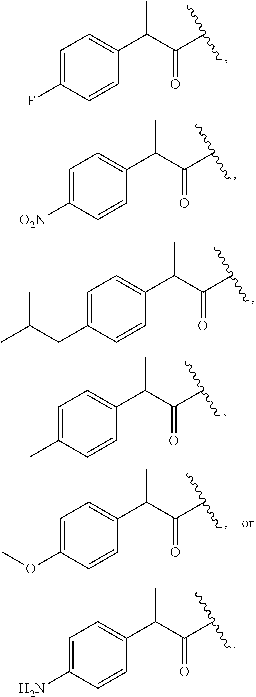

The pharmaceutical composition can further comprise a compound of formula F-L′-G or a pharmaceutically acceptable salt thereof, wherein F is a radical of folate or an analog thereof; L′ is a linker; and G is a radical of a sugar (e.g., glucosamine).

The pharmaceutical composition can further comprise a compound of formula F-L′-G or a pharmaceutically acceptable salt thereof, wherein F is a radical of folate or an analog thereof; L′ is a linker; and G is a radical of glucosamine.

A combination of pharmaceutical compositions is also provided. In certain embodiments, the combination comprises (i) a first pharmaceutical composition comprising any of the compounds herein (e.g., a compound of Formula (I)); and (ii) a second pharmaceutical composition comprising a compound of formula F-L′-G or a pharmaceutically acceptable salt thereof, wherein F is a radical of folate or an analog thereof; L′ is a linker; and G is a radical of glucosamine. The first and second pharmaceutical compositions can be administered by the same or different routes, such as simultaneously or sequentially in either order, and/or by the same or different dosing regimens. In certain embodiments, the first and second pharmaceutical compositions are administered sequentially, such as sequentially in either order. In certain embodiments, the first and second pharmaceutical compositions are administered contemporaneously, simultaneously, sequentially, or separately.

The pharmaceutical composition can comprise one or more pharmaceutically acceptable carriers, adjuvants, diluents, excipients, and/or vehicles (e.g., conventional nontoxic pharmaceutically acceptable carriers, adjuvants, and vehicles), and combinations thereof.

The pharmaceutical composition can be formulated, e.g., for a given route of administration, and manufactured in accordance with methods in the art and described, for example, in Remington, The Science and Practice of Pharmacy, 22^{nd} edition (2012). For example, and without limitation, the composition can be an injectable composition, such as a composition that can be injected subcutaneously.

The pharmaceutical composition can be administered to a mammalian host, such as a human patient, in a variety of forms adapted to the chosen route of administration. In certain embodiments, the pharmaceutical composition is formulated to be administered subcutaneously. In certain embodiments, the pharmaceutical composition is formulated to be administered orally. In certain embodiments, the pharmaceutical composition is formulated to be administered intramuscularly, intravenously, intraarterially, intraperitoneally, or as any other art-recognized route of parenteral administration.

In certain embodiments, the pharmaceutical composition is systemically administered in combination with a pharmaceutically acceptable vehicle. The percentages of the components of the compositions and preparations can vary and can be between about 1 to about 99% weight of the active ingredient(s) (e.g., the compound) and a binder, excipients, a disintegrating agent, a lubricant, and/or a sweetening agent (as are known in the art). The amount of active compound in such therapeutically useful compositions is such that an effective dosage level can be obtained.

Illustrative means of parenteral administration include needle (including microneedle) injectors, needle-free injectors and infusion techniques, as well as any other means of parenteral administration recognized in the art. Parenteral formulations are typically aqueous solutions, which can contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, can readily be accomplished using standard pharmaceutical techniques well-known to those skilled in the art.

The pharmaceutical dosage forms suitable for administration can include sterile aqueous solutions or dispersions or sterile powders comprising the active ingredients that are adapted for the extemporaneous preparation of sterile injectable or infusible solutions or dispersions, optionally encapsulated in liposomes, nanocrystals, or polymeric nanoparticles. In all cases, the ultimate dosage form should be sterile, fluid, and stable under the conditions of manufacture and storage. The liquid carrier or vehicle can be a solvent or liquid dispersion medium comprising, for example and without limitation, water, electrolytes, sugars, ethanol, a polyol (e.g., glycerol, propylene glycol, liquid polyethylene glycols, and the like), vegetable oils, nontoxic glyceryl esters, and/or suitable mixtures thereof. In at least one embodiment, the desired fluidity can be maintained by the formation of liposomes, by the maintenance of the required particle size in the case of dispersions or by the use of surfactants.

Sterile injectable solutions can be prepared by incorporating the pharmaceutical compositions in the required amount of the appropriate solvent with one or more of the other ingredients set forth above, as required, followed by filter sterilization. In the case of sterile powders for the preparation of sterile injectable solutions, vacuum-drying and freeze-drying techniques can be employed, which can yield a powder of the active ingredient plus any additional desired ingredient present in the previously sterile-filtered solutions.

The compounds and pharmaceutical compositions can be administered in unit dosage forms and/or compositions. As used herein, the term “administering” and its variants include all means of introducing the compound(s) and compositions described herein to the subject, including, without limitation, oral (p.o.), intravenous (i.v.), intramuscular (i.m.), subcutaneous (s.c.), transdermal, via inhalation (e.g., intranasal (i.n.)), buccally, intraocularly, sublingually, vaginally, rectally, and the like.

For methods described herein, the compound(s) and compositions can be administered in a single dose, or via a combination of multiple dosages, which can be administered by any suitable means, contemporaneously, simultaneously, sequentially, or separately. Where the dosages are administered in separate dosage forms, the number of dosages administered per day for each compound or composition can be the same or different. The compound and/or composition dosages can be administered via the same or different routes of administration. The compounds or compositions can be administered according to simultaneous or alternating regimens, at the same or different times during the course of the therapy, concurrently in divided or single forms.

The compound/composition can be administered more than once, such as daily (1-3 or more times per day; q.d. (once a day), b.i.d. (twice a day), t.i.d. (three times a day)), weekly (including 1-3 or more times on a given day), bi-weekly (including 1-3 or more times on a given day), monthly (including 1-3 or more times on a given day), or bimonthly (including 1-3 or more times on a given day). In each case it is understood that the effective amounts described herein correspond to the instance of administration, or alternatively to the total daily, weekly, month, or quarterly dose, as determined by the dosing protocol.

An effective amount of the compound or a pharmaceutical composition comprising the same can be determined in accordance with methods known in the art (e.g., animal models, human data, and human data for compounds that are used in a similar manner). The dosage/effective amount can be determined by taking into consideration several factors, including: the mode of administration, the potency of the compound, the specific disease or disorder involved, the response of the individual subject, the severity and/or details of the subject's present condition, the use of concomitant medication, the age, weight, and health of the subject, and other relevant circumstances. Additionally, pharmacogenomic (the effect of genotype on the pharmacokinetic, pharmacodynamic or efficacy profile of the antigen or composition) information about a particular patient may affect the dosage used.

Depending upon the route of administration, a wide range of permissible dosages are contemplated. For example, the effective amount of the compound and/or pharmaceutical composition can range from about 0.1 μg/kg/day, such as 0.5 μg/kg/day, 0.7 μg/kg/day, or 0.01 mg/kg/day up to about 1,000 mg/kg/day. Intravenous doses can be several orders of magnitude lower.

### Methods of Immunomodulating Tregs

Still further provided is a method of immunomodulating Tregs in a subject (e.g., in need thereof). In certain embodiments, the method comprises administering to the subject an effective amount of a first compound or a pharmaceutical composition comprising the first compound. Administration of an effective amount of a first compound or a pharmaceutical composition comprising the first compound can result in the Tregs being activated, inhibited (e.g., such as in the case of cancer), proliferated, or killed.

In certain embodiments, the subject has cancer; the E of the first compound is a radical of a TLR7 agonist, a PI3K inhibitor, a NLR2 agonist, a STING agonist, an EZH2 inhibitor, an NLRP3 inhibitor, a Caspase I inhibitor, or a RLR agonist; and administration the first compound or pharmaceutical composition comprising the first compound alters Tregs's promotion of tumor growth and metastasis and/or inhibition of anti-tumor immunity.

The cancer can be lung cancer, bone cancer, pancreatic cancer, skin cancer, cancer of the head, cancer of the neck, cutaneous melanoma, intraocular melanoma, uterine cancer, ovarian cancer, endometrial cancer, epithelial cancer, leiomyosarcoma, rectal cancer, stomach cancer, colon cancer, breast cancer, triple-negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, bladder cancer, gastric cancer, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, Hurthle cell thyroid cancer, or adenocarcinoma of the gastroesophageal junction. The cancer can be lung cancer, triple negative breast cancer, colon cancer, gastric cancer, bladder cancer, prostate cancer, or pancreatic cancer.

In certain embodiments, the method (e.g., administration of an effective amount of the first compound or pharmaceutical composition comprising the first compound) does not induce unwanted inflammation in the subject.

In certain embodiments, the method comprises administering to the subject an effective amount of a first compound or a pharmaceutical composition comprising the first compound, wherein E of the first compound is a radical of a TLR agonist (e.g., a TLR7 agonist), a PI3K inhibitor, a NLR2 agonist, a STING agonist, an EZH2 inhibitor, an NLRP3 inhibitor, a Caspase I inhibitor, or a RLR agonist; and administration the first compound or pharmaceutical composition comprising the first compound alters Tregs's promotion of tumor growth and metastasis and/or inhibition of anti-tumor immunity. The method can further comprise administering a second compound of formula F-L′-G or a pharmaceutically acceptable salt thereof, or a pharmaceutical composition comprising the second compound, wherein F is a radical of folate or an analog thereof; L′ is a linker; and G is a radical of glucosamine. Administration of the second compound or pharmaceutical composition comprising the second compound can be performed simultaneously or sequentially with the first compound or pharmaceutical composition comprising the first compound in either order, by the same or different routes.

The method can further comprise administering an additional therapeutic agent (e.g., a third therapeutic agent), such as an anticancer agent. The anticancer agent can be a chemotherapeutic agent or a radiotherapeutic agent, for example. Administration of the additional therapeutic agent can be performed simultaneously or sequentially with the first compound or pharmaceutical composition comprising the first compound and/or the second compound or pharmaceutical composition comprising the second compound in any order, by the same or different routes.

In another embodiment of the method, the subject has a fibrotic disease or disorder, and the method comprises administering an effective amount of a compound, in which E is a radical of a TLR7 agonist, a PI3K inhibitor, a NLR2 agonist, a STING agonist, an EZH2 inhibitor, an NLRP3 inhibitor, a Caspase I inhibitor, or an RLR agonist, or a pharmaceutical composition comprising the same. Administration of an effective amount of the first compound or pharmaceutical composition comprising the first compound can alter Tregs' promotion of tumor growth and metastasis and/or inhibit anti-cancer immunity in the subject. The fibrotic disease or disorder can be arthrofibrosis, autoimmune pancreatitis, bladder fibrosis, chronic kidney disease, chronic wounds, Crohns's disease, desmoid tumor, Dupuytren's contracture, endometrial fibroids, fibromatosis, graft-versus-host disease, heart fibrosis, keloids, liver fibrosis, mediastinal fibrosis, myelofibrosis, nephrogenic systemic fibrosis, Peyronie's disease, pulmonary fibrosis, retroperitoneal cavity fibrosis, scleroderma or systemic sclerosis, or skin fibrosis. The fibrotic disease or disorder can be pulmonary fibrosis, liver fibrosis, scleroderma, myelofibrosis, Crohn's disease, or chronic kidney disease. The pulmonary fibrosis can be idiopathic pulmonary fibrosis (IPF). The liver fibrosis can be NASH or cirrhosis.

In certain embodiments of the method, the subject has an inflammatory disease, and the method comprises administering an effective amount of a first compound or a pharmaceutical composition comprising the first compound, in which E is a radical of a steroid. The inflammatory disease can be Crohn's disease, lupus, inflammatory bowel disease (IBS), Addison's disease, Grave's disease, Sjogren's syndrome, celiac disease, Hashimoto's thyroiditis, myasthenia gravis, autoimmune vasculitis, reactive arthritis, psoriatic arthritis, pernicious anemia, ulcerative colitis, rheumatoid arthritis, type 1 diabetes, organ transplant rejection, multiple sclerosis, graft vs. host disease (GVHD), fatty liver disease, asthma, osteoporosis, sarcoidosis, ischemia-reperfusion injury, prosthesis osteolysis, glomerulonephritis, scleroderma, psoriasis, autoimmune myocarditis, spinal cord injury, central nervous system inflammation, viral infection, influenza, coronavirus infection, cytokine storm syndrome bone damage, inflammatory brain disease, or atherosclerosis.

The method can further comprise administering the second compound of formula F-L′-G or a pharmaceutically acceptable salt thereof, wherein F is a radical of folate or an analog thereof; L′ is a linker; and G is a radical of glucosamine, wherein administering of the second compound or the pharmaceutical composition comprising the second compound can be simultaneously or sequentially with the first compound or pharmaceutical composition comprising the first compound in either order, by the same or different routes.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present compounds, compositions, and methods are defined by the below claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### Methods of Treatment

Further provided herein is a method of treating cancer in a subject in need thereof, comprising administering to the subject a therapeutically effective amount of a compound described herein (e.g., a compound of the formula (I) or a pharmaceutically-acceptable salt thereof).

The cancer can be lung cancer, bone cancer, pancreatic cancer, skin cancer, cancer of the head, cancer of the neck, cutaneous melanoma, intraocular melanoma, uterine cancer, ovarian cancer, endometrial cancer, epithelial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, bladder cancer, gastric cancer, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, Hurthle cell thyroid cancer, or adenocarcinoma of the gastroesophageal junction. The cancer can be lung cancer, triple negative breast cancer, colon cancer, gastric cancer, bladder cancer, prostate cancer, or pancreatic cancer.

Further provided herein is a method of treating a fibrotic disease or disorder in a subject in need thereof, comprising administering to the subject a therapeutically effective amount of a compound described herein (e.g., a compound of the formula (I) or a pharmaceutically acceptable salt thereof).

The fibrotic disease or disorder can be arthrofibrosis, autoimmune pancreatitis, bladder fibrosis, chronic kidney disease, chronic wounds, Crohns's disease, desmoid tumor, Dupuytren's contracture, endometrial fibroids, fibromatosis, graft-versus-host disease, heart fibrosis, keloids, liver fibrosis, mediastinal fibrosis, myelofibrosis, nephrogenic systemic fibrosis, Peyronie's disease, pulmonary fibrosis, retroperitoneal cavity fibrosis, scleroderma or systemic sclerosis, or skin fibrosis. The fibrotic disease or disorder is pulmonary fibrosis, liver fibrosis, scleroderma, myelofibrosis, Crohn's disease, or chronic kidney disease. The pulmonary fibrosis can be idiopathic pulmonary fibrosis (IPF). The liver fibrosis can be NASH or cirrhosis.

Further provided herein is a method of treating an inflammatory disease in a subject in need thereof, comprising administering to the subject a therapeutically effective amount of a compound described herein (e.g., a compound of the formula (I) or a pharmaceutically acceptable salt thereof).

### Additional Methods of Use

A method of binding a compound of Formula (I′) or a pharmaceutically acceptable salt thereof to a receptor in a cell (e.g., in a subject, e.g., in a subject in need thereof) is also provided. The method can comprise binding a compound of the Formula (I′) or a pharmaceutically acceptable salt thereof to a receptor in a cell (e.g., in a subject, e.g., in a subject in need thereof), wherein Formula (I′) has the structure:

T′-L-E (I′)

wherein T′ is a targeting moiety (e.g., a radical of raltitrexed, 5-MTHF, an analog of raltitrexed, or an analog of 5-MTHF, a compound of Formula I-VII, Formula II-VII, Formula III-VII, Formula IV-VII, Formula V-VII, Formula VI-VII, or Formula VII-VII, or a compound of Table I, II, or III, as described herein; L is a linker; and E is a radical of a therapeutic agent (e.g., a radical of a therapeutic agent described herein); comprising contacting the cell with the compound. In some embodiments, T can bind to a receptor of a cell. In some embodiments, T can bind to a pattern recognition receptor in a cell. In some embodiments, T can bind to an immune cell receptor. In some embodiments, T selectively binds to a folate receptor. In some embodiments, T selectively binds to FRβ. In some embodiments, T selectively binds to FRδ. In some embodiments, T binds to FRδ with a higher affinity than T binds to FRβ. In some embodiments, the cell is a macrophage. In some embodiments, the cell is a tumor-associated macrophage. In some embodiments, the cell is a tumor-associated macrophage. In some embodiments, the cell is an M1-macrophage. In some embodiments, the cell is an M2-macrophage.

A method of binding a compound of the Formula (I′) or a pharmaceutically acceptable salt thereof to a receptor in a subject (e.g., in a subject in need thereof) is also provided, wherein Formula (I′) has the formula:

T′-L-E (I′)

wherein T′ is a targeting moiety (e.g., a radical of raltitrexed, 5-MTHF, an analog of raltitrexed, or an analog of 5-MTHF, a compound of Formula I-VII, Formula II-VII, Formula III-VII, Formula IV-VII, Formula V-VII, Formula VI-VII, or Formula VII-VII, or a compound of Table I, II, or III, as described herein; L is a linker; and E is a radical of a therapeutic agent (e.g., a radical of a therapeutic agent described herein); wherein the method comprises contacting the cell with the compound. In some embodiments, T binds to a receptor of a cell. In some embodiments, T binds to a pattern recognition receptor in a cell. In some embodiments, T binds to an immune cell receptor. In some embodiments, T selectively binds to a folate receptor. In some embodiments, T selectively binds to FRβ. In some embodiments, T selectively binds to FRδ. In some embodiments, T binds to FRδ with a higher affinity than T binds to FRβ. In some embodiments, the subject has cancer (e.g., a cancer described herein). In some embodiments, the subject has a fibrotic disease or disorder (e.g., a fibrotic disease or disorder described herein). In some embodiments, the subject has an inflammatory disease (e.g., an inflammatory disease described herein).

### Certain Definitions

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range. The term “substantially” can allow for a degree of variability in a value or range, for example, within 90%, within 95%, 99%, 99.5%, 99.9%, 99.99%, or at least about 99.999% or more of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “patient” and “subject” are used interchangeably and include a human patient, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, or a whale. The patient to be treated is preferably a mammal, in particular a human being.

### EXAMPLES

The following examples serve to illustrate the present disclosure. The examples are not intended to limit the scope of the claimed invention in any way.

### Example 1

### High-Affinity Binding of Raltitrexed to Folate Receptor δ (FR δ) in Silico

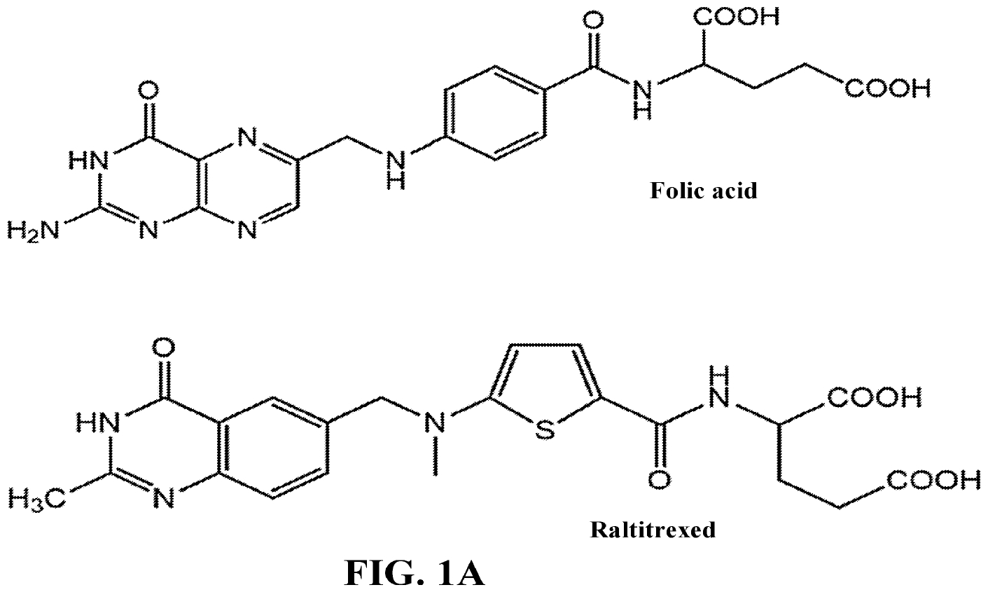

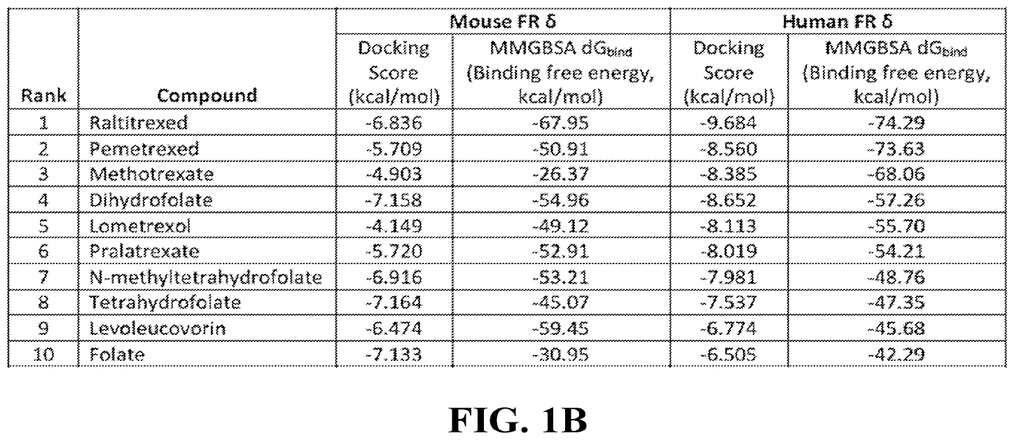

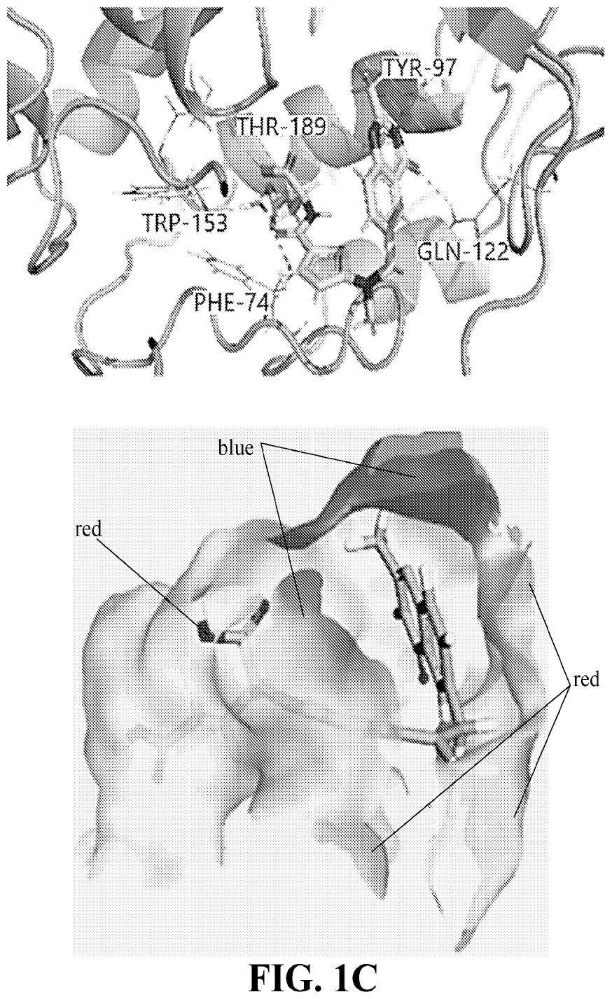

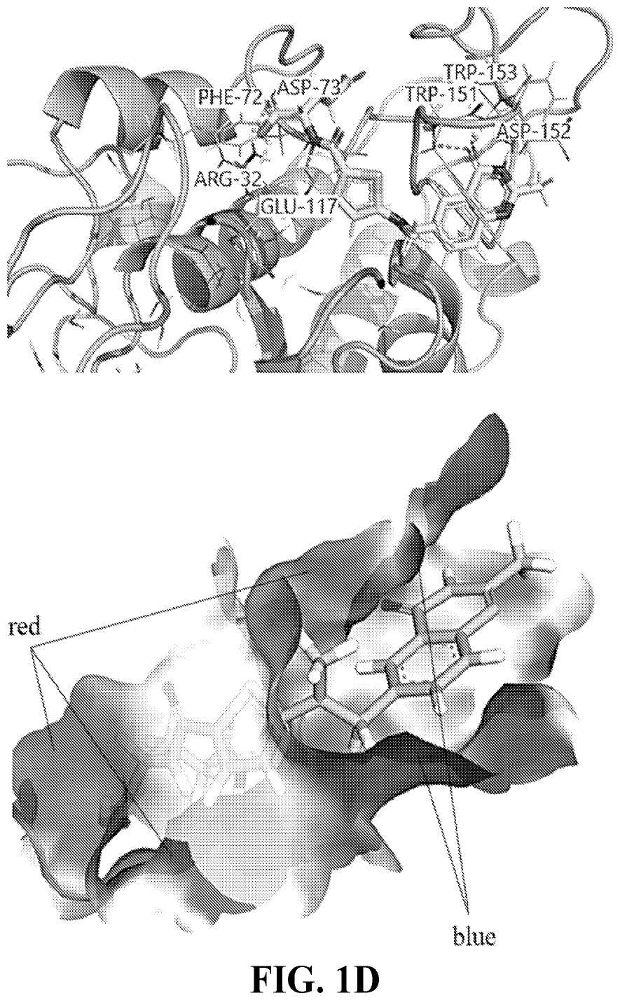

Virtual binding studies of raltitrexed to FR δ (previously known as JUNO protein (PDB:5F4E)) were conducted. These studies showed relatively high binding affinity to FR δ in silico. Folic acid was shown to have about 1,000-fold lower binding affinity to FR δ than that for folate receptor α (FR α) and folate receptor β (FR β). FIG. 1A shows the structures of raltitrexed and folic acid. FIG. 1B shows the predicted relative binding free energies (MMGBSA dGbind) and docking scores (XP GScore) for binding of folic acid analogs to human (PDB: 5F4Q) and mouse (PDB:5JYJ) FRδ. FIG. 1C shows the binding poses of raltitrexed on human FRδ. FIG. 1D shows the binding poses of raltitrexed on mouse FRδ. Ribbon diagrams display the ligand-protein interactions while surface topographies reveal the electrostatic potential maps and binding orientations of the ligand in the FRδ binding cavity. Blue represents the positively charged regions and red represents the negatively charged regions of the binding site.

### Example 2

### Raltitrexed Targets Tregs In Vivo Selectively Through FRδ

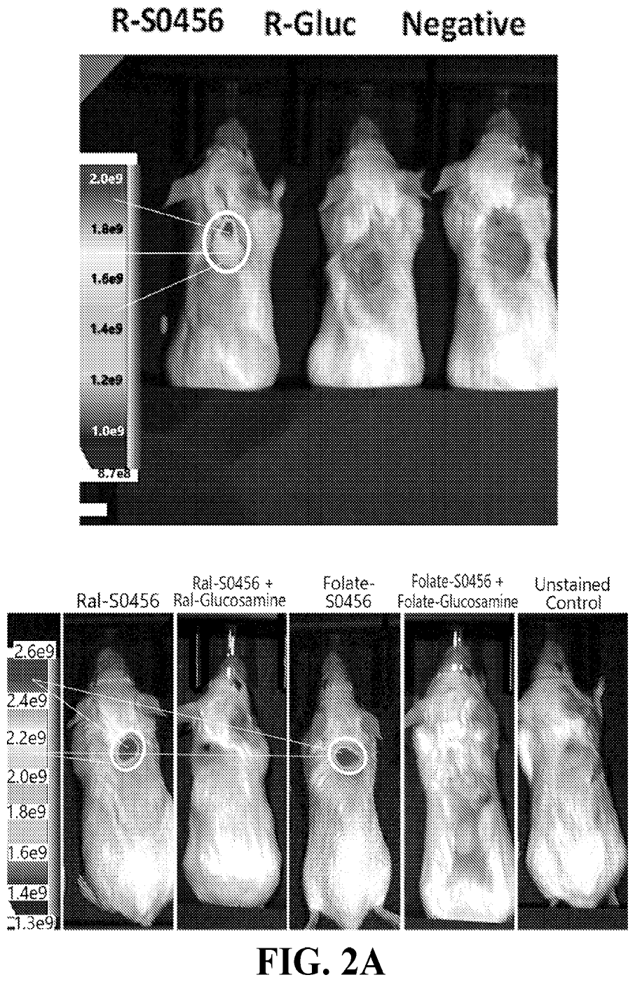

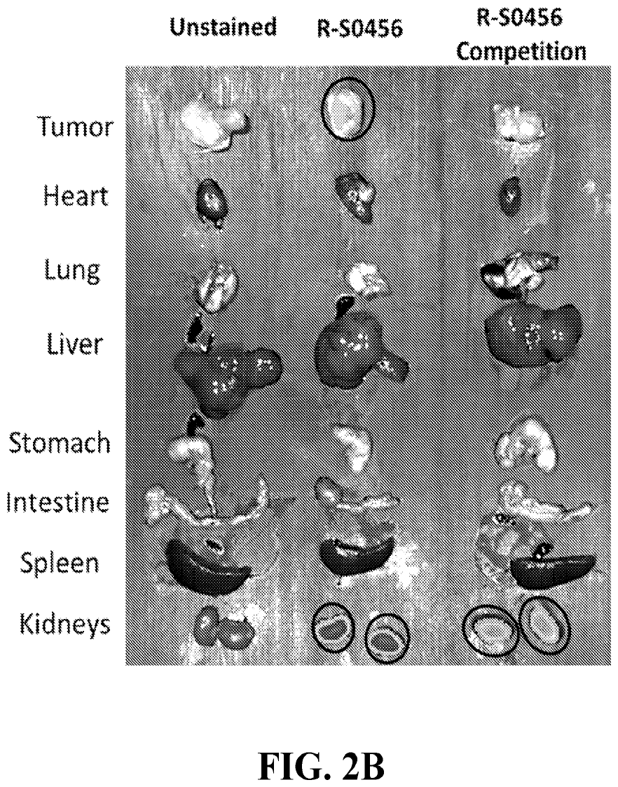

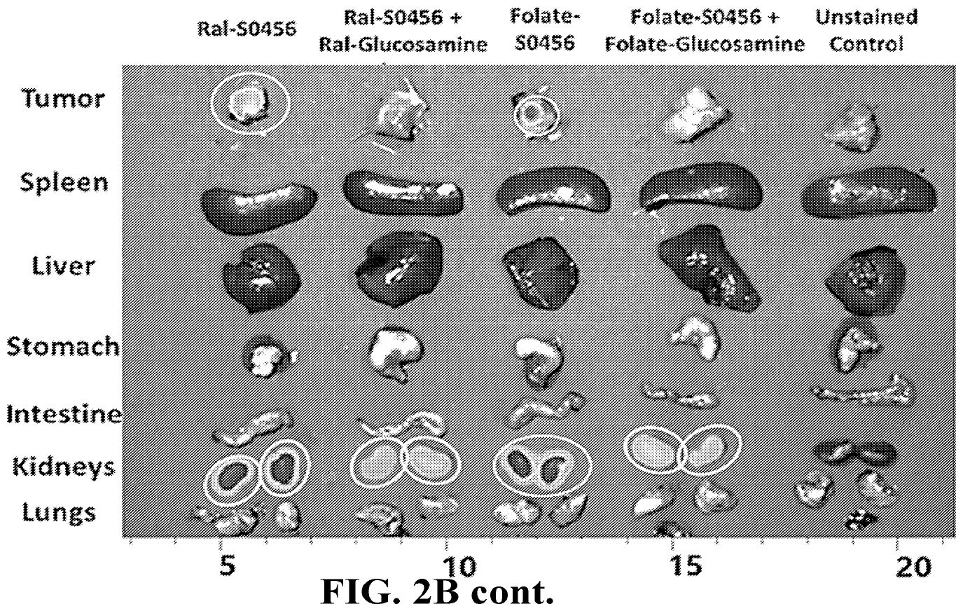

Female balb/c mice and C57BL/6 FRδ knockout mice were implanted with 250,000 cells of 4T1 breast cancer cell line and MB49 bladder cancer cell line (folate receptor-negative cell lines) subcutaneously, respectively. When the tumor volume reached ˜400 mm³, mice were tail vein-injected with 10 nmol of raltitrexed-S0456 (Ral-S056) (left mouse), 10 nmol of raltitrexed-S0456 with 200× competition with raltitrexed-glucosamine (middle mouse in top image of FIG. 2A and second from left mouse in bottom image of FIG. 2A)), 10 nmol of folate-S0456 with 200× competition with Folate-Glucosamine (second from right mouse in bottom image of FIG. 2A), or vehicle as a control (right mouse), and 10 nmol of folate-S0456 (middle mouse in bottom image of FIG. 2A) as another control. Images were taken 4 hours after injection.

FIG. 2A shows imaging of tumor-bearing mice taken four hours post-injection. Thereafter, the tumor and the other major organs were excised and imaged alone (FIG. 2B, which shows imaging of most of the major organs taken from the treated mice). The glucosamine conjugates served as competing ligands, compared to negative control.

After digesting the tumor and the spleen, a comprehensive flow cytometric analysis of most immune cells was performed

FIG. 3A shows flow cytometry scatter plot results for live cells, and FIG. 3B shows flow cytometry scatter plot results for cells labeled with anti-CD45 antibody. FIG. 3C shows flow cytometry scatter plot results for cells labeled with anti-CD4 and anti-CD25 antibodies. FIG. 3D shows flow cytometry scatter plot results for cells labeled with anti-CD127 and anti-FR δ antibodies. FIG. 3E shows the uptake of Ral-S0356 by CD45+CD4+CD25+CD127+ FRδ+ Tregs isolated from murine tumor and spleen, with or without 200× Ral-Glucosamine competition, compared to folate-S0456 and the unstained control. FIG. 3F shows a comparison of the uptake of Ral-S0456 and Folate-S0456 by different white blood cell populations in the tumors. FIG. 3G Shows no binding of Ral-S0456 or Folate-S0456 to CD45− cells conjugate. FIG. 3H shows no binding of Ral-S0456 or Folate-S0456 to CD45+CD8+ cytotoxic T cells. FIG. 3I shows no binding of Ral-S0456 or Folate-S0456 to CD45+CD4+CD25−FRδ− cells, whereas Ral-S0456, but not Folate-S0456, shows binding to CD45+CD4+CD25−FRδ+ memory T cells. FIG. 3J shows some binding of Ral-S0456, but significantly higher binding of Folate-S0456, to CD45+CD11b+F4/80 macrophages. FIG. 3K shows flow cytometry data demonstrating that Ral-S0456 accumulates in tumor Tregs of wild type but not FRδ knockout mice, demonstrating that Ral-S0456 uptake is FRδ-receptor mediated.

In sum, raltitrexed-S0456 showed selective uptake in activate, tumor Tregs, without any uptake in resting, spleen Tregs. The analysis also indicated no uptake of folate-S0456 by Tregs. Competing raltitrexed-S0456 with 200×raltitrexed-glucosamine did not show any signal, which supports the binding was receptor-mediated. Finally, the FRδ knockout mice showed diminished uptake of raltitrexed-S0456 compared to wild type mice.

Accordingly, raltitrexed, but not folate, targeted the activated Tregs in TME but not the resting Tregs in spleen. Also, the data supports this uptake is FRδ receptor-mediated.

### Example 3

### Synthesis of Raltitrexed-S0456

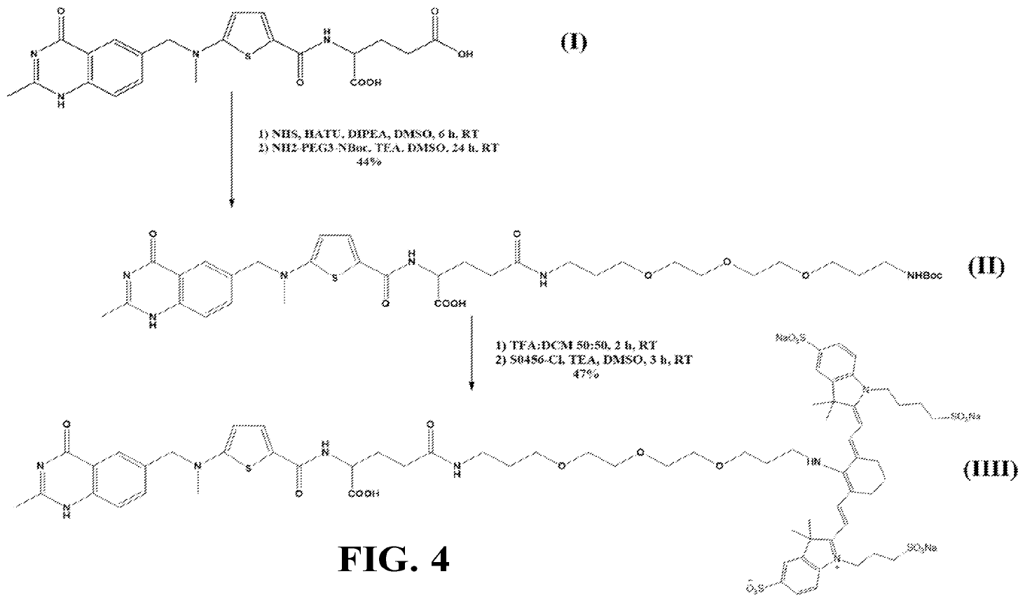

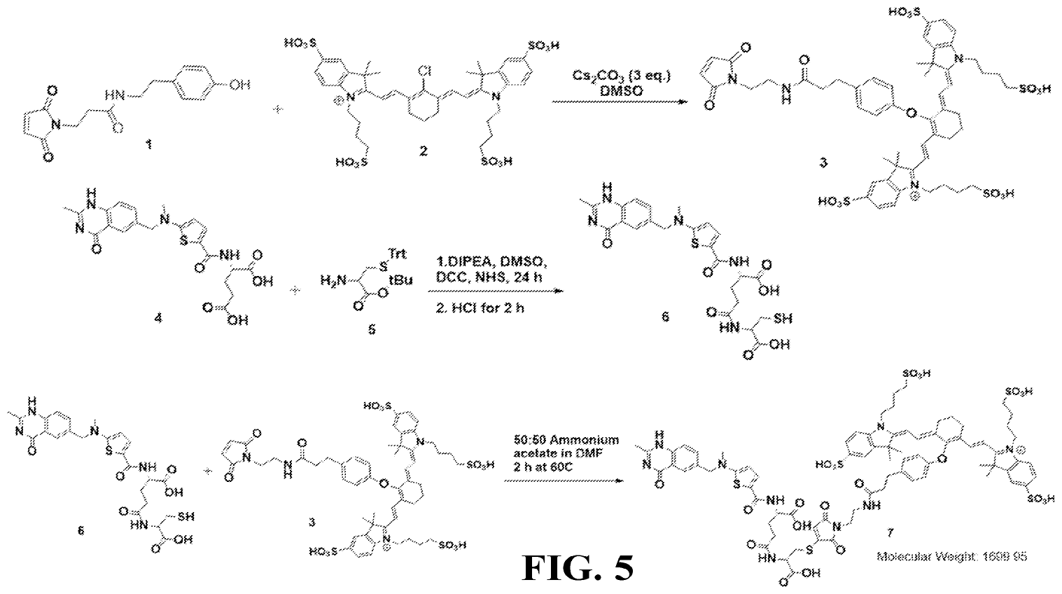

FIGS. 4 and 5 show synthetic schemes for producing raltitrexed-S0456 that were utilized in the binding studies shown above.

Raltitrexed (I) (0.109 mmol, 1 eq) was dissolved in dimethyl sulfoxide (DMSO) (150 μL). N,N′-Diisopropylethylamine (DIPEA) (0.33 mmol, 3 eq) and hexafluorophosphate Azabenzotriazole Tetramethyl Uronium (HATU) (0.23 mmol, 2 eq) were added, and a brown color resulted. The solution was stirred for 15 minutes and, thereafter, N-hydroxy-succinamide (NHS) (0.109 mmol, 1 eq) dissolved in DMSO (250 μL) was added to the reaction mixture and the reaction mixture was stirred for 6 hours. The product was precipitated with an excess amount of ether (15 mL×3) and centrifuged, thereby removing all impurities and forming a sticky, brownish, oily material, which was used in the next step without further purification.

Next, NH₂-PEG-NBoc (75 μL) was dissolved in DMSO (150 μL) with triethylamine (0.359 mmol), and the reaction mixture was stirred for 15 minutes, after which the crude from previous step (dissolved in 150 μL DMSO) was added. The reaction mixture was stirred overnight. The reaction was purified with high-performance liquid chromatography (HPLC) and the % yield was 44% (30 mg of Compound (II)).

Compound (II) (0.039 mmol) was dissolved in 500 μL of 50:50 2,2,2-trifluoroacetic acid (TFA):dichloromethane (DCM), and the reaction mixture was stirred for 2 hours. Thereafter, the solvent was evaporated and further washed with DCM (1 mL×3). Then, the deprotected product was dissolved in DMSO (100 μL) with triethylamine (TEA) (0.25 mmol), and the reaction mixture was stirred for 15 minutes, after which S0456-Cl near-infrared (NIR) dye (0.078 mmol, 2 eq) dissolved in DMSO (100 μL) was added, and the reaction mixture was stirred for 3 hours. The final product (III) (M.Wt. 1,512 g/mol) was purified using HPLC, with a % yield of 25% (15 mg) final product, (III)).

### Example 4

### Synthesis of TLR7-1A Agonist

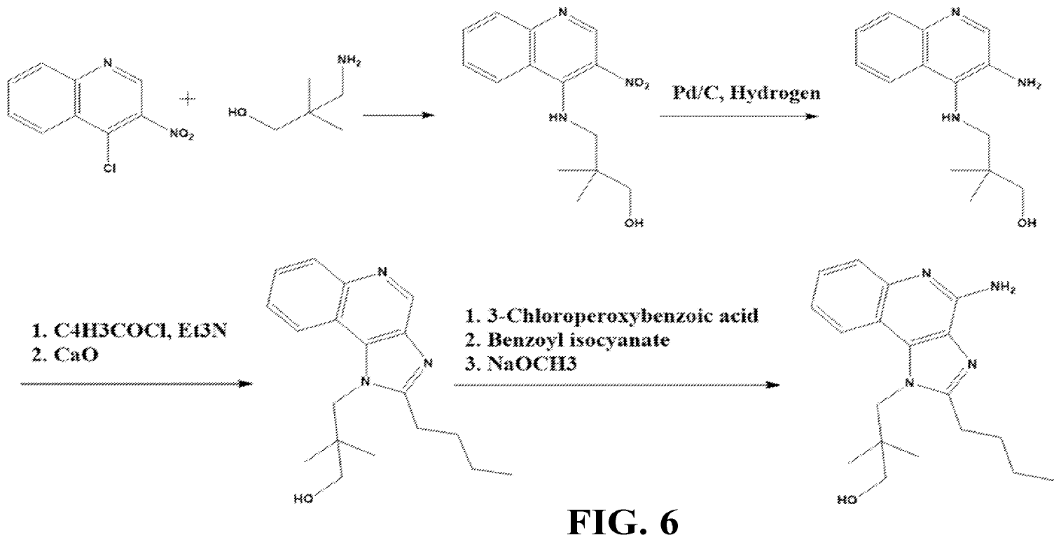

FIG. 6 shows the synthetic scheme of the TLR7-1A agonist that was used in the therapeutic evaluation and targeting studies herein.

3-Amino-2,2-dimethyl-1-propanol (2.4 mmol) was added to the solution of 4-chloro-3-nitroquinoline (1.2 mmol) and triethylamine (Et3N) (3 mmol) in a 4:1 mixture of toluene and 2-propanol. The mixture was heated to 70° C. for 0.5 hour until a solid started precipitating. The reaction mixture was then cooled, filtered, and washed with toluene/2-propanol (7:3), ether, and cold water. The residue was dried at 80° C. to obtain 2,2-dimethyl-3-((3-nitroquinolin-4-yl)amino)propan-1-ol. LCMS: [M+H]+m/z=275.31.

2,2-dimethyl-3-((3-nitroquinolin-4-yl)amino)propan-1-ol (1.72 mmol) was dissolved in methanol and hydrogenated over Pd/C as a catalyst with a hydrogen balloon for 4 hours. The solution was then filtered using celite, followed by evaporation of the solvent under reduced pressure to afford 3-((3-aminoquinolin-4-yl)amino)-2,2-dimethylpropan-1-ol. LCMS: [M+H]+m/z=245.33.

To a solution of 3-((3-aminoquinolin-4-yl)amino)-2,2-dimethylpropan-1-ol (0.43 mmol) in anhydrous tetrahydrofuran (THF), triethylamine was added (0.65 mmol) and valeryl chloride (0.52 mmol). The reaction mixture was then stirred for 6-8 hours, followed by the removal of the solvent under vacuum. The residue was dissolved in ethyl acetate (EtOAc), washed with water and brine, and then dried over Na₂SO4 to obtain the intermediate amide compound. This was dissolved in MeOH, followed by the addition of calcium oxide, and was heated in a microwave at 110° C. for 1 hour. The solvent was then removed and the residue was purified using column chromatography (9% MeOH/dichloromethane) to obtain 3-(2-butyl-1H-imidazo[4,5-c]quinolin-1-yl)-2,2-dimethylpropan-1-ol (58 mg). LCMS: [M+H]+m/z=311.43.

To a solution of 3-(2-butyl-1H-imidazo[4,5-c]quinolin-1-yl)-2,2-dimethylpropan-1-ol (0.186 mmol) in a solvent mixture of MeOH:dichloromethane:chloroform (0.1:1:1) was added 3-chloroperoxybenzoic acid (0.49 mmol), and the solution was refluxed at 45-50° C. for 40 minutes. The solvent was then removed, and the residue was purified using column chromatography (20% MeOH/dichloromethane) to obtain the oxide derivative (55 mg). This was then dissolved in anhydrous dichloromethane, followed by the addition of benzoyl isocyanate (0.26 mmol), and heated at 45° C. for 15 minutes. The solvent was then removed under vacuum, and the residue was dissolved in anhydrous MeOH, followed by the addition of excess sodium methoxide. The reaction mixture was then heated at 80° C. for one hour. The solvent was removed under vacuum, and the residue was purified using column chromatography (11% MeOH/dichloromethane) to obtain 3-(4-amino-2-butyl-1H-imidazo[4,5-c]quinolin-1-yl)-2,2-dimethylpropan-1-ol. LCMS: [M+H]+m/z=313.44

### Example 5

### TLR7-1A Agonist Inhibits the Immunosuppressive Characteristics of Murine Tregs Ex Vivo

Murine CD45+CD4+CD25+ Tregs and CD45+CD4+CD25− effector T cells were isolated from healthy mice using a StemCell mouse Treg Isolation Kit (StemCell Technologies, Cambridge, MA). The cells were used in two different assays.

First, Tregs that were either pre-treated with 10 nM TLR7-1A agonist for 3 hours or left untreated were co-cultured with CFSE-labeled effector T cells at 1:4 ratio, with or without CD3/CD28 activation beads. Incubation proceeded for 4 days before flow cytometry analysis.

In the second assay, Tregs that were pre-treated with 10 nM TLR7-1A agonist for 3 hours or left untreated were co-cultured with effector T cells at 1:4 ratio, with or without CD3/CD28 activation beads. Incubation proceeded for 48 hours before the supernatant was analyzed with enzyme-linked immunoassay (ELISA) for interleukin-10 (IL-10) and transforming growth factor beta (TGF-β) release.

Tregs inhibited CD4+ T cells proliferation. However, the TLR7-1A agonist reversed the suppressive activity, resulting in restoration of CD4+ cells proliferation. Additionally, TLR7-1A reversal of Tregs' suppressive activity was evident with the reduced release of IL-10 and TGF-Beta immunosuppressive cytokines.

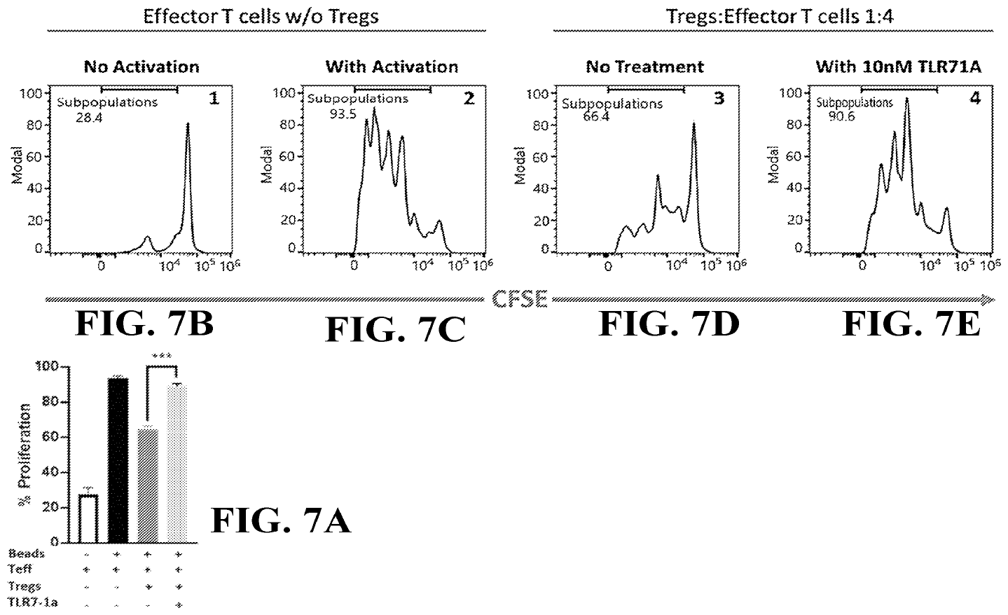

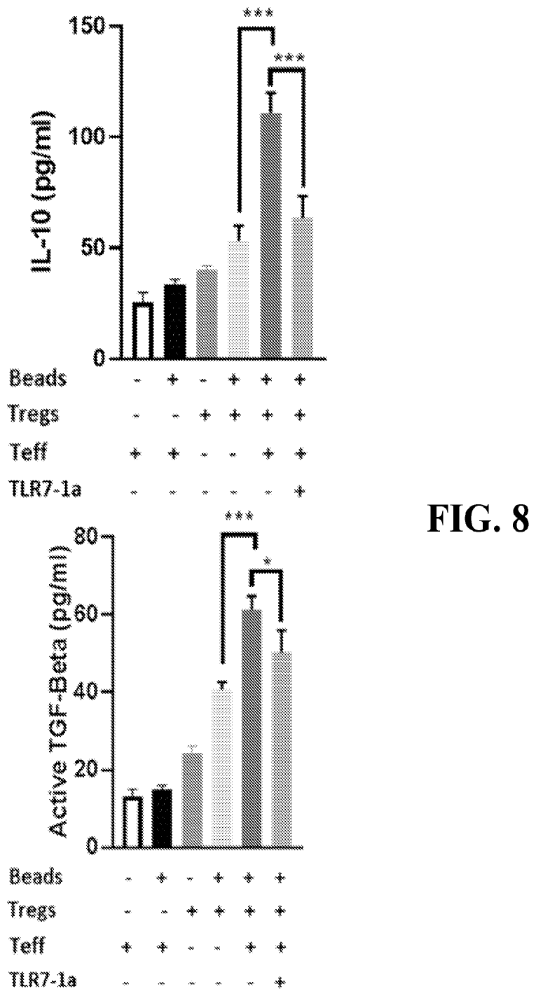

Representative data are shown below in FIGS. 7A-7E and FIG. 8. FIG. 7A shows the parent CFSE-labeled CD4+CD25− population without co-cultured Tregs, treatment, or CD3/CD28 beads activation. FIG. 7B Shows divided CFSE-labeled CD4+CD25− population upon activation, without co-cultured Tregs or treatment. FIG. 7C Shows the effect of Tregs on the division of CFSE-labeled CD4+CD25− effector T cells when co-cultured at 1:4 ratio. FIG. 7D shows the effect of pre-treating Tregs with 10 nM TLR7-1A before co-culturing with CFSE-labeled CD4+CD25− cells at 1:4 ratio. FIG. 7E shows a bar graph representation of the data, and FIG. 8 shows the immunomodulatory effect of TLR7-1A on murine CD45+CD4+CD25+ Tregs in vitro, where Tregs were pre-treated with TLR7-1A for 3 hours before being co-cultured with murine CD45+CD4+CD25− effector T cells for 48 hours, after which supernatant is analyzed by enzyme-linked immunoassay (ELISA) for IL-10 and TGF-Beta release.

Accordingly, the TLR7-1A agonist may be a good candidate to target Tregs in vivo.

### Example 6

### Synthesis of Raltitrexed-TLR7-1A Agonist Releasable Conjugate (Compound 11)

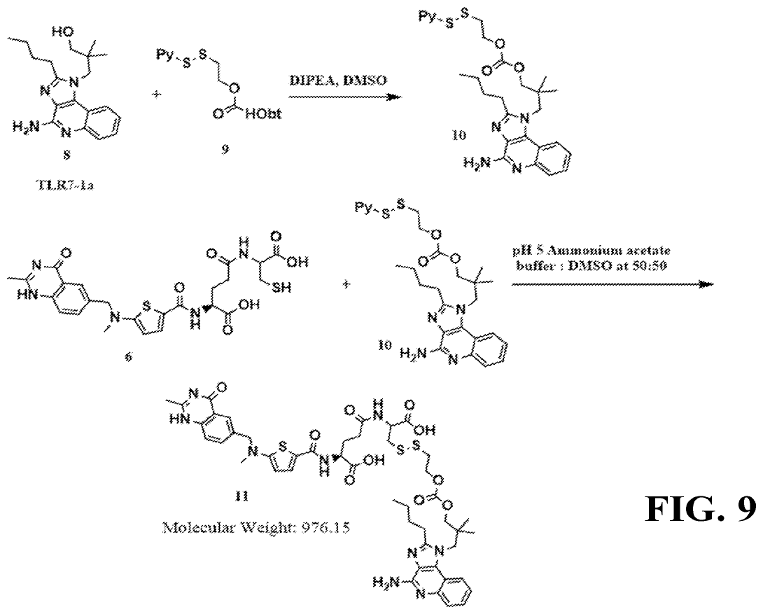

FIG. 9 shows a synthetic scheme of Ral-TLR7-1A agonist that was used in the in vivo targeting studies hereof.

To synthesize compound 10, DIPEA (100 ul) and 1H-benzo[d][1,2,3]triazol-1-yl (2-(pyridin-2-yldisulfaneyl)ethyl) carbonate 9 (0.3 mmol) were added to a solution of TLR7-1A (0.1 mmol) (see Example 4) in DMSO, and stirred for 12 hours. The crude reaction mixture was then purified by HPLC using 5-95% water in acetonitrile mobile phase, and the combined purified fractions were lyophilized to yield product 10 as a colorless crystalline solid (Yield ˜33%).

To synthesize compound 11, compound 10 (0.3 mmol) was added to a solution of raltitrexed-cysteine compound 6 (0.1 mmol) in 1:1 mixture of aqueous ammonium acetate (pH −5) and dimethylformamide (DMF) and stirred for 12 hours. After the starting material was completely consumed, the crude reaction mixture was purified by ultra-high-performance liquid chromatography (UHPLC) using 5-95% water in acetonitrile mobile phase. The combined purified fractions were lyophilized to yield raltitrexed-TLR7-TA conjugate (compound 11) as a colorless solid (yield ˜63%). The product is confirmed by liquid chromatography-mass spectrometry (LCMS) (M.Wt. 976.15 g/mol).

### Example 7

Tumor Growth Inhibition of 4T1 Breast Cancer Model in BALB c Mice In Vivo, with Ral-TLR7-1A Agonist Releasable Conjugate (Compound 11)

8-10-week-old female BALB/c mice were implanted with 50,000 cells of 4T1 breast cancer cell line (folate receptor-negative cell line) subcutaneously. When the tumor size reached ˜50 mm³, treatment was started with raltitrexed-TLR7-TA agonist 10 nmol daily dose, raltitrexed 10 nmol daily dose, raltitrexed-TLR7-TA agonist 10 nmol plus 200× raltitrexed-glucosamine competing ligands daily dose, or phosphate-buffered saline (PBS) only, for 5 days/week. Tumors were measured every other day and mice were sacrificed when some of the untreated mice tumor reached ˜1500 mm³. At the end of study, tumors were digested, a single cell suspension was obtained, and cells were analyzed on flow cytometry to obtain a comprehensive immune cells analysis.

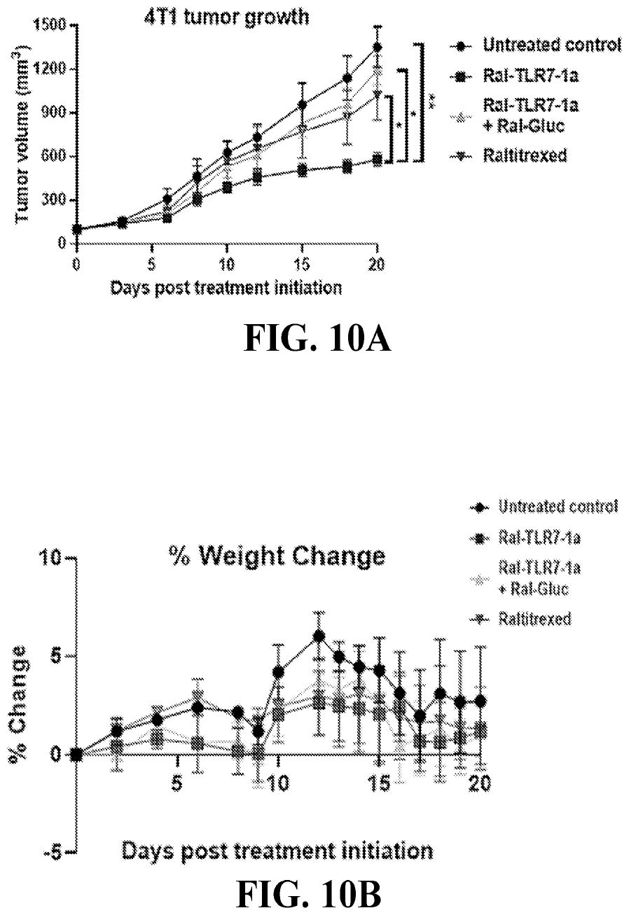

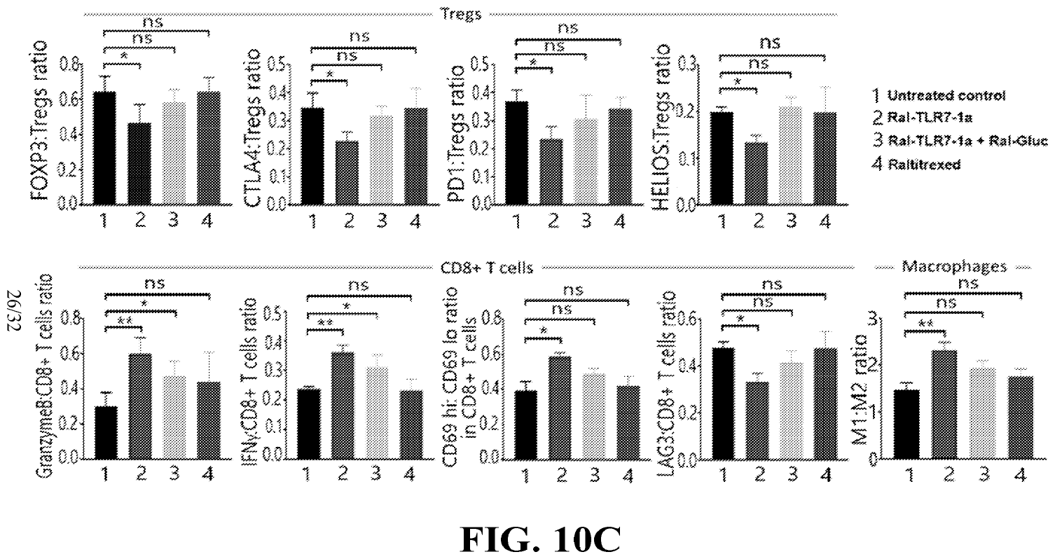

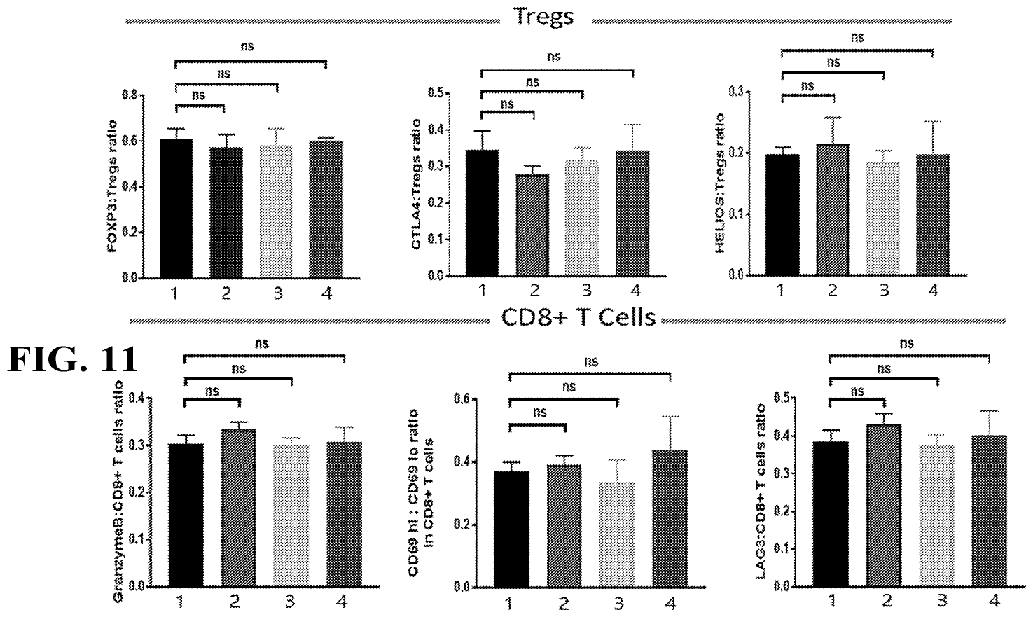

Tumor growth was significantly reduced in ral-TLR7-TA agonist (as shown in FIG. 10A, which shows the 4T1 breast tumor volume/mm³ vs. days post-initiation of treatment compared to untreated control). Body weight, which generally serves as a reflection of toxicity, did not change significantly over the course of the treatment (FIG. 10B). Finally, the analysis of Tregs, CD8⁺ cytotoxic T cells and macrophages isolated from tumors showed shifting in their characteristics to more pro-inflammatory phenotypes, whereas the same cells isolated from spleen showed no change (as shown in FIG. 10C, where the relevant phenotypic markers are listed on each y-axis and the treatment regimen is indicated on each x-axis). FIG. 11 shows the evaluation of the effect of ral-TLR7-TA on the phenotypic markers of splenic Tregs and CD8⁺ cytotoxic T cells isolated from the tumor-bearing mice of FIG. 10A.

Accordingly, raltitrexed-TLR7-1A agonist releasable conjugate therapy showed promising results in inhibiting tumor growth by targeting Tregs and reprogramming the tumor immune environment but not Tregs present in healthy tissues.

### Example 8

Tumor Growth Inhibition of CT26 Colorectal Cancer Model in BALB/c Mice In Vivo, with Ral-TLR7-1A Agonist Releasable Conjugate (Compound 11)

8-10-week-old female BALB/c mice were implanted with 50,000 cells of CT26 colorectal cancer cell line (folate receptor-negative cell line) subcutaneously. When the tumor size reached ˜50 mm³, treatment was started with raltitrexed-TLR7-1A agonist at 10 nmol daily dose or PBS only, for 5 days/week. Tumors were measured every other day and mice were sacrificed when some of the untreated mice tumor reached ˜1500 mm³.

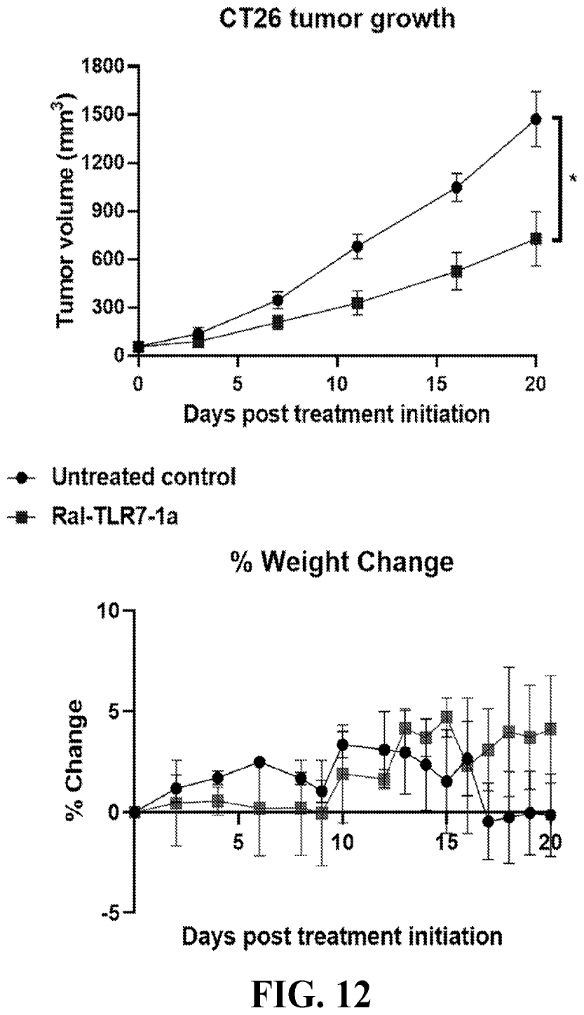

Tumor growth was significantly reduced in the group treated with ral-TLR71A agonist (FIG. 12). Body weight did not significantly change over the course of the treatment.

Accordingly, ral-TLR7-1A agonist releasable conjugate therapy is effective in inhibiting tumor growth in yet another tumor model, which supports that this treatment approach may be universally applicable to multiple tumor models.

### Example 9

Tumor Growth Inhibition of MB49 Bladder Cancer Model in FRβ Knockout C57BL 6 Mice In Vivo, with Ral-TLR7-1A Agonist Releasable Conjugate (Compound 11)

8-10-week old female FRβ knockout C57BL/6 mice were implanted with 50,000 cells of MB49 bladder cancer cell line (folate receptor-negative cell line) subcutaneously. When tumor size reached ˜50 mm³, treatment was started with raltitrexed-TLR7-1A agonist 10 nmol daily dose or PBS only, for 5 days/week. Tumors were measured every other day, and mice were sacrificed when some of the untreated mice tumor reached ˜1500 mm³. Phenotypic markers of Tregs and CD8⁺ cytotoxic T cells were assessed.

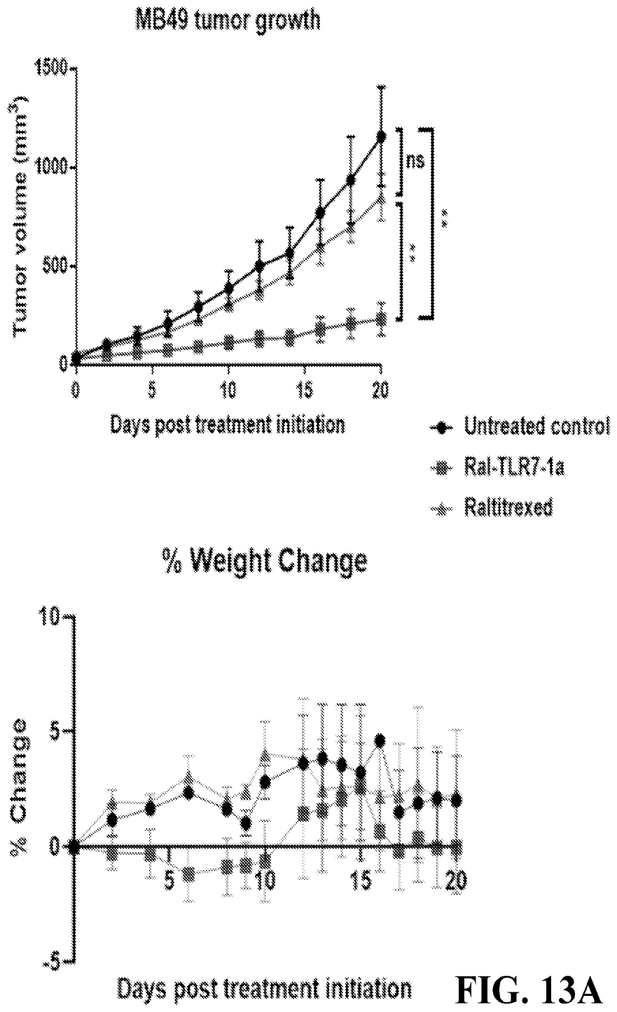

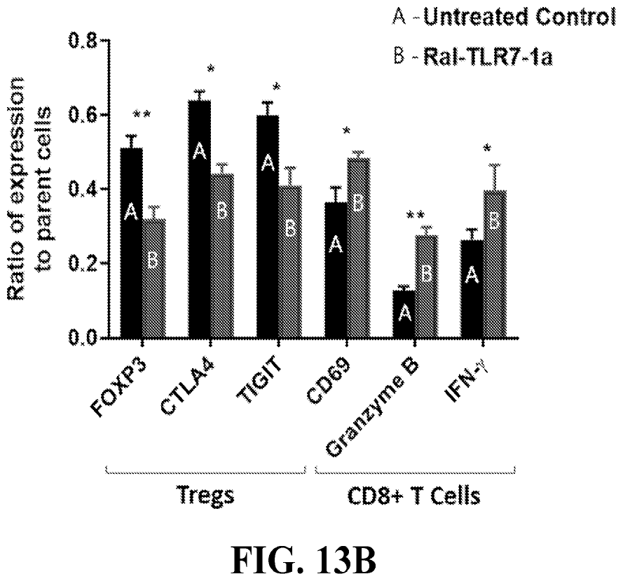

As shown in FIG. 13A, tumor growth was significantly reduced in the group treated with ral-TLR7-1A agonist. Body weight did not change significantly over the course of the treatment. The analysis of Tregs and CD8⁺ cytotoxic T cells showed shifting in their characteristics to more pro-inflammatory phenotypes (FIG. 13B).

Raltitrexed-TLR7-1A agonist releasable conjugate therapy inhibited tumor growth despite not targeting any macrophages that normally express FRβ, and the result obtained was solely due to targeting and reprogramming Tregs.

### Example 10

### Synthesis of Raltitrexed-Dexamethasone Releasable Conjugate (Compound 19)

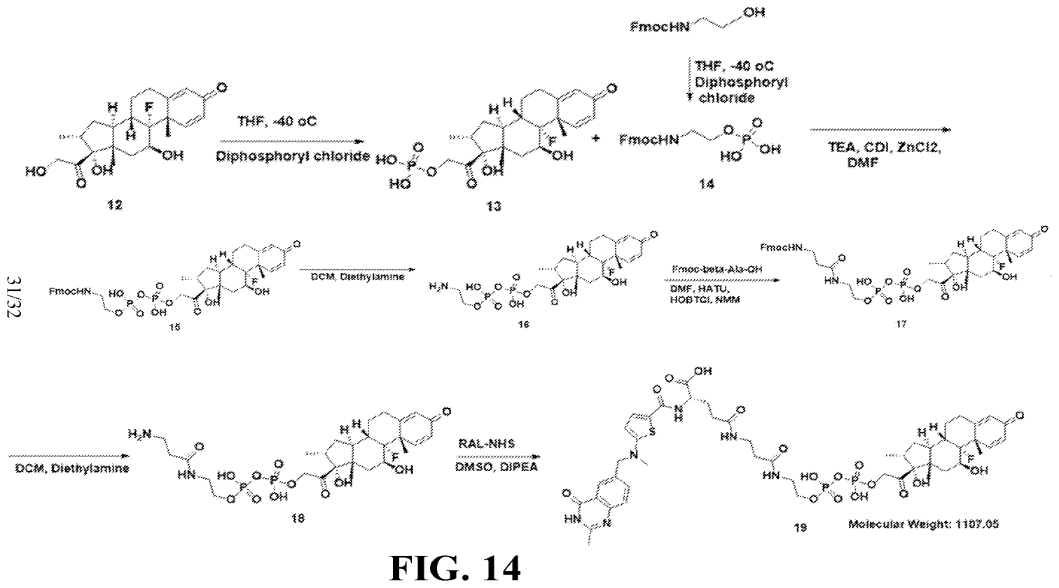

FIG. 14 shows the synthetic scheme of ral-dexamethasone (compound 19) that was used in the in vivo targeting studies described herein.

To synthesize compound 13, dexamethasone (500 mg) was added to a 50 mL round bottom flask followed by anhydrous THF (2.5 mL). Then, the reaction was cooled to −40° C. with a dry ice/acetonitrile cold bath. 0.53 mL (3 eq) of diphosphoryl chloride was added and stirred at −40° C. for 1 hour. The reaction was quenched with water and titrated to pH ˜8 with saturated aqueous sodium bicarbonate solution. The solution was made acidic pH ˜2 using a 1 N HCl solution and extracted several times with ethyl acetate. The combined organic phase was dried over anhydrous Na2SO₄ then removed under the vacuum and used in the next step without any further purification (yield=0.51 g).

To synthesize compound 15, (9H-fluoren-9-yl)methyl(2-Hydroxyethyl)carbamate (500 mg) was added to a 50 mL round bottom flask followed by 3.4 mL anhydrous THF. Then, the reaction was cooled to −40° C. with a dry ice/acetonitrile cold bath. 0.6 mL of diphosphoryl chloride was added and stirred at −40° C. for 1 hour. The reaction was quenched with water and titrated to pH ˜8 with saturated aqueous sodium bicarbonate solution. The solution was made acidic pH ˜2 using a 1 N HCl solution and extracted several times with ethyl acetate. The combined organic phase was dried over anhydrous Na2SO₄ then de vacuo and used in the next step without any further purification (yield=0.64 g).

Triethylamine (0.16 mL) and CDI (0.45 g) were then added to a stirred solution of 395 mg of Fmoc-phosphate in DMF (3 mL). The resulting solution was stirred at room temperature for 30 minutes. To this mixture Dexamethasone21-phosphate (500 mg) and ZnCl2 (1.18 g) were added, and the mixture was allowed to stir at room temperature overnight. The reaction was diluted with 1 N HCl and extracted several times with ethyl acetate. The combined organic layers were concentrated, and reverse phase column chromatography using combi flash (yield=255 mg).

To synthesize compound 16, diethylamine (1 mL) was added to a stirred solution of Fmoc-pyro-Dex (255 mg) in DCM (4 mL), and the resulting mixture was stirred at room temperature for 2 hours. The DCM/DEA mixture was removed under vacuum (yield=185 mg).

To synthesize compound 17, a solution of 95 mg of Fmoc-β-Ala-acid was activated with 85 mg of HATU, 85 mg of HOBtCl, 11 mL DMF and 0.3 mL N-methylmorpholine (NMM). This mixture was then added to Fmoc deprotected pyro-Dex residue (185 mg) and stirred for 3 hours for completion of the reaction monitored by LCMS. The reaction mixture was purified by reverse phase column chromatography using combi flash (yield=120 mg).

To synthesize compound 18, diethylamine (1 mL) was added to a stirred solution of FmocNH-β-Ala-pyro-Dex (120 mg) in DCM (4 mL). The resulting mixture was stirred at room temperature for 2 hours. The DCM/DEA mixture was removed under vacuum (yield=80 mg).

To synthesize compound 19, raltitrexed (100 mg), N-hydroxyl-succinimide (31.3 mg), and dicyclohexylcarbodiimide (46.7 mg) were dissolved in dry DMSO (10 mL). The reaction mixture was stirred, in the dark, at room temperature for 15 hours. The NHS-raltitrexed was precipitated by the addition of ethyl acetate, filtered, and washed with ethyl acetate once and anhydrous diethyl ether thrice. The NHS-raltitrexed was dried under a desiccator and used immediately. Fmoc-deprotected compound (15 mg) was dissolved in DMSO (1 mL) then added NHS-raltitrexed and DIPEA. The reaction was monitored by LCMS and upon completion was purified by RP-HPLC and verified by LCMS (yield=3.5 mg).

### Example 11

Tumor Growth Inhibition and Enhancement of 4T1 Breast Cancer Model in BALB/c Mice In Vivo, with Ral-TLR71A Agonist and Ral-Dexamethasone Releasable Conjugates (Compounds 11 and 19, Respectively)

8-10-week-old female BALB/c mice were implanted with 50,000 cells of 4T1 breast cancer cell line subcutaneously. When tumor size reached ˜50 mm³, treatment was started with either ral-TLR7-TA agonist (compound 11) or ral-dexamethasone (compound 19) at 10 nmoles daily dose or PBS only, for 5 days/week. Tumors were measured every other day and mice were sacrificed when some of the untreated mice tumor reached ˜1500 mm³. Phenotypic makers of Tregs and CD8⁺ cytotoxic T cells were assessed.

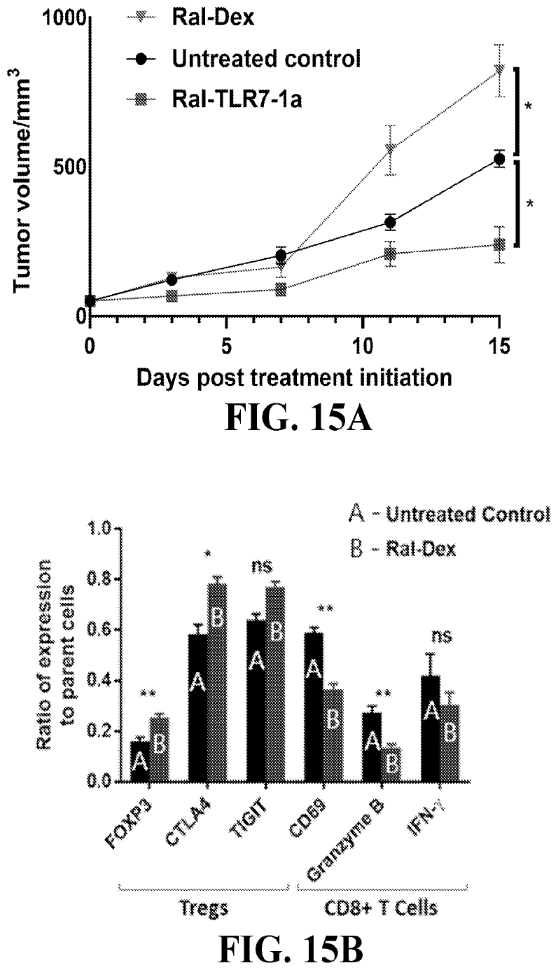

Tumor growth was significantly reduced in the ral-TLR7-1A agonist treatment group and enhanced in the ral-dexamethasone treatment groups (FIG. 15A). The analysis of Tregs and CD8⁺ cytotoxic T cells in the ral-dexamethasone treatment group showed significant enhancement of the immunosuppressive Treg phenotypes and less cytotoxic characteristics of CD8⁺ T cells (FIG. 15B).

In sum, raltitrexed-dexamethasone releasable conjugate therapy enhanced tumor growth due to the enhancement of Tregs' immunosuppressive capacity.

1. A compound of the formula (I):
T-L-E (I)
or a pharmaceutically acceptable salt thereof, wherein:
T is a radical of raltitrexed, 5-methyltetrahydrofolate (5-MTHF), an analog of raltitrexed, or an analog of 5-MTHF;
L is a linker; and
E is a radical of a therapeutic agent.

2. The compound of claim 1, wherein T has the structure of Formula (II):

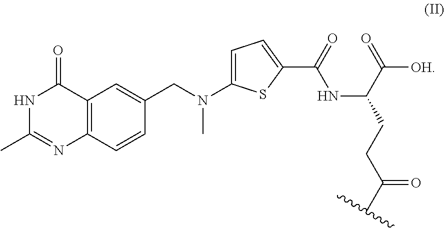 [SMILES: *C(=O)CC[C@H](NC(=O)c1ccc(N(C)Cc2ccc3nc(C)[nH]c(=O)c3c2)s1)C(=O)O]

3. The compound of claim 1, wherein T has the structure of Formula (III):

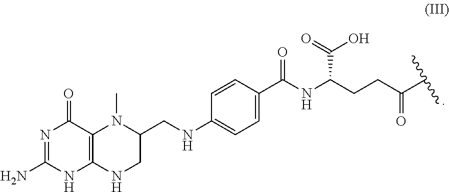 [SMILES: *C(=O)CC[C@H](NC(=O)c1ccc(NCC2CNc3[nH]c(N)nc(=O)c3N2C)cc1)C(=O)O]

4. The compound of any one of claims 1-3, wherein the therapeutic agent is selected from the group consisting of a toll-like receptor 7 (TLR7) agonist, a phosphoinositide 3-kinase (PI3k) inhibitor, a steroid, a nucleotide-binding and oligomerization domain (NOD)-like receptor 2 (NLR2) agonist, a stimulatory of interferon gene (STING) agonist, an enhancer of zeste homolog 2 (EZH2) inhibitor, a NOD-, LRR- and pyrin domain-containing protein 3 (NLRP3) inhibitor, a Caspase I inhibitor, a retinoic acid-inducible gene I (RIG-I)-like receptor (RLR) agonist, an absent in melanoma 2 (AIM2)-like receptor agonist, and an agonist of a receptor for advanced glycation end products (RAGE).

5. The compound of claim 1, wherein the therapeutic agent is a NLR2 agonist having the structure:

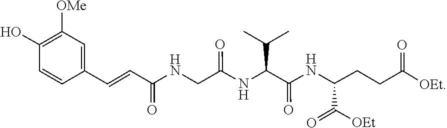 [SMILES: CCOC(=O)CC[C@@H](NC(=O)[C@@H](NC(=O)CNC(=O)/C=C/c1ccc(O)c(OC)c1)C(C)C)C(=O)OCC]

6. The compound of claim 1, wherein the therapeutic agent is a STING agonist having the structure:

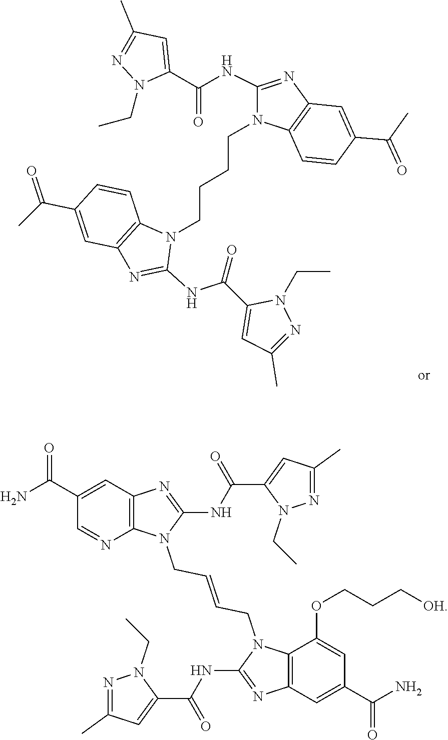 [SMILES: CCn1nc(C)cc1C(=O)Nc1nc2cc(C(C)=O)ccc2n1CCCCn1c(NC(=O)c2cc(C)nn2CC)nc2cc(C(C)=O)ccc21 ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO)c21]

7. The compound of claim 1, wherein the therapeutic agent is an EZH2 inhibitor.

8. The compound of claim 7, wherein the EZH2 inhibitor is 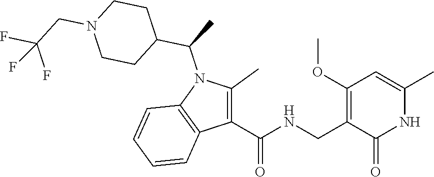 [SMILES: COc1cc(C)[nH]c(=O)c1CNC(=O)c1c(C)n([C@H](C)C2CCN(CC(F)(F)F)CC2)c2ccccc12]
or tazemetostat.

9. The compound of claim 1, wherein the therapeutic agent is a NLRP3 inhibitor having the structure:

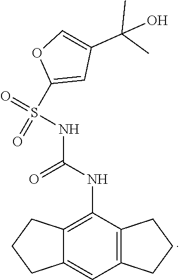 [SMILES: CC(C)(O)c1coc(S(=O)(=O)NC(=O)Nc2c3c(cc4c2CCC4)CCC3)c1]

10. The compound of claim 1, wherein the therapeutic agent is a Caspase I inhibitor having the structure:

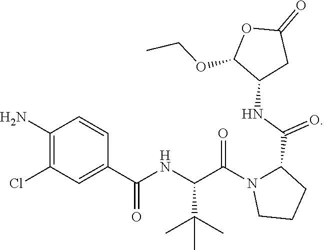 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C]

11. The compound of claim 1, wherein the therapeutic agent is a PI3 kinase inhibitor having the structure:

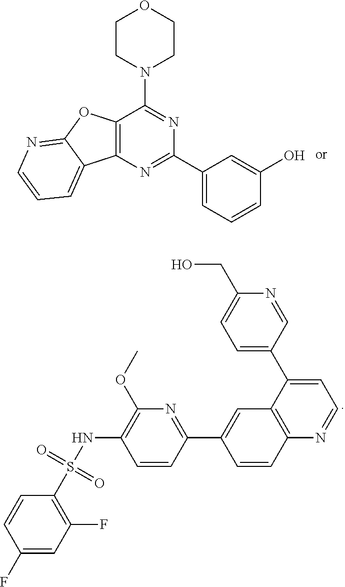

12. The compound of claim 1, wherein the therapeutic agent is a RLR agonist having the structure:

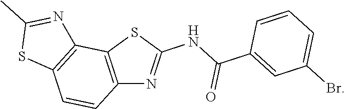 [SMILES: Cc1nc2c(ccc3nc(NC(=O)c4cccc(Br)c4)sc32)s1]

13. A compound of the formula (I):
T-L-E (I)
or a pharmaceutically acceptable salt thereof, wherein:
T is a radical of raltitrexed, 5-MTHF, an analog of raltitrexed, or an analog of 5-MTHF;
L is a linker; and
E is a radical of a TLR7 agonist represented by Formula (IV): 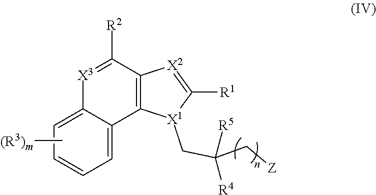
or a pharmaceutically acceptable salt thereof, wherein:
R¹, R³, R⁴, R⁵ are each independently H, alkyl, alkoxyl, alkenyl, alkynyl, cycloalkyl, aryl halo, heteroaryl, —COR^{2x}, 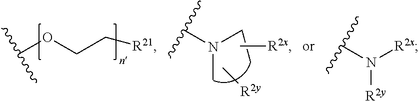
R² is a H, —OH, —NH₂, -NHR^{2x}, N₃, —NH—CH₂—NH₂, —CONH₂, —SO₂NH₂, —NH—CS—NH₂, 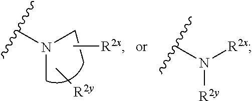
Z is a H, —OH, —NH₂, —NHR^{2x}, —O—R^{2x}, —SO—R^{2x}, —SH, —SO₃H, —N₃, —CHO, —COOH, —CONH₂, —COSH, —COR^{2x}, —SO₂NH₂, alkenyl, alkynyl, alkoxyl, —NH—CH₂—NH₂, —CONH₂, —SO₂NH₂, —NH—CS—NH₂, 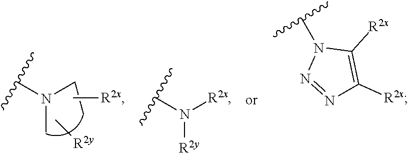
wherein:
each of R^{2x} and R^{2y} is independently selected from the group consisting of H, —OH, —CH₂—OH, —NH₂, —CH₂—NH₂, —COOMe, —COOH, —CONH₂, —COCH₃, alkyl, alkenyl, alkynyl, alicyclic, aryl, biaryl, and heteroaryl,
each R^{2z} is independently selected from the group consisting of —NH₂, —N^{2q}R^{2q′}—O—R^{2q}, —SO—R^{2q}, and —COR^{2q}; wherein each R^{2q} and R^{2q′} is independently alkyl or H, 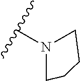 [SMILES: *N1CCCC1]
is a 3-10 membered N-containing non-aromatic, mono- or bicyclic heterocycle;
R²¹ is H or alkyl, and
n′ is 0-30; and
wherein, in Formula (IV):
each of X¹, X², X³ is independently CR^{q} or N, wherein each R^{q} is independently H, halogen, or an optionally substituted alkyl;
n is 0-30;
m is 0-4; and
when n is 0, Y is not H, —OH, or —O—R^{2x}.

14. The compound of claim 13, wherein E is a radical of a compound represented by Formula (IVA): 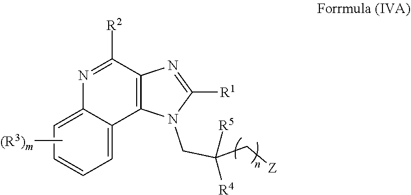
or a pharmaceutically acceptable salt thereof, wherein:
R¹ is an optionally substituted C₃-C₈ alkyl;
R² is H, —OR^{z}, —SO₂N(R^{z})₂, —NR^{2x}R^{2y}, or N₃;
Z is H, —OR^{z}, —NR^{2x}R^{2y}, —SR^{z}, —SOR^{z}, —SO₃R^{z}, —N₃, —COR^{z}, —COOR^{z}, —CON(R^{z})₂, —COSR^{z}, —SO₂N(R^{z})₂, or —CON(R^{z})₂, wherein:
R^{2x} and R^{2y} are each independently hydrogen, —N(R^{z})₂, —CON(R^{z})₂, —C(R^{z})₂—N(R^{z})₂, —CS—N(R^{z})₂, or optionally substituted alkyl,
each R^{z} is independently H, halogen, or an optionally substituted alkyl, or R^{2x} and R^{2y} are taken together to form an optionally substituted heterocycloalkyl;
each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein each of the alkyl, alkoxy, heteroalkyl, cycloalkyl, or heterocycloalkyl is optionally substituted;
R⁴ and R⁵ are each independently alkyl, alkoxy, halogen, or cycloalkyl, wherein each of the alkyl, alkoxy, and cycloalkyl, is optionally substituted;
n is 1-6; and
m is 0-4.

15. The compound of claim 13 or 14, wherein the compound of Formula (I) is represented by Formula (IVB) or Formula (IVC): 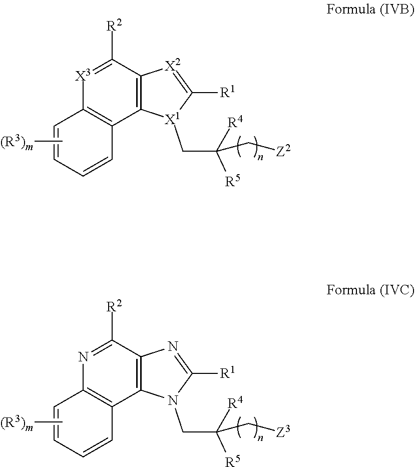
or a pharmaceutically acceptable salt thereof, wherein:
each R¹ is independently an optionally substituted C₃-C₈ alkyl;
each R² is independently H, —OR^{z}, —SO₂N(R^{z})₂, —NR^{2x}R^{2y}, or N₃;
each R^{2x} and R^{2y} are independently hydrogen, —N(R^{z})₂, —CON(R^{z})₂, —C(R^{z})₂—N(R^{z})₂, —CS—N(R^{z})₂, or optionally substituted alkyl,
each R^{z} is independently H, halogen, or an optionally substituted alkyl, or R^{2x} and R^{2y} are taken together to form an optionally substituted heterocycloalkyl;
each R³ is independently halogen, —N₃, —CN, —NO₂, —COR^{z}, —COOR^{z}, —CON(R^{z})₂, —COSR^{z}, —SO₂N(R^{z})₂, or —CON(R^{z})₂, alkyl, heteroalkyl, cycloalkyl, heterocycloalkyl, alkoxy, amino, hydroxy or thiol, wherein each of the alkyl, alkoxy, heteroalkyl, cycloalkyl, or heterocycloalkyl is optionally substituted;
each R⁴ and R⁵ are independently alkyl, alkoxy, halogen, or cycloalkyl, wherein each of the alkyl, alkoxy, and cycloalkyl, is optionally substituted;
n is 1-6;
m is 0-4;
each Z² and Z³ is independently a group of the formula T-L-, T-L-O—, T-L-O-alkyl-, T-L-S¹—, T-SO₂—NH—, T-L-NR^{a}R^{b}—, T-L-S(O)ₓ-alkyl-, T-L-CO—, T-L-aryl-, T-L-NH—CO—NH—, T-L-NH—O—, T-L-NH—NH—, T-L-NH-L-CS—NH, T-L-C(O)-alkyl-, or T-L-SO₂—;
R^{a} and R^{b} are each independently H, halo, hydroxy, alkoxy, aryl, amino, acyl or C(O)R^{c}, wherein R^{c} is alkyl, aryl, oxy or alkoxy;
S¹ is a spacer;
x is 0-3;
n is 1-3 and m is 0-4.

16. The compound of claim 15, wherein:
R¹ is a C₁-C₆ alkyl optionally substituted with 1-3 substituents, each substituent independently being halogen or C₁-C₆ alkoxy;
R² is —NR^{2x}R^{2y}, where R^{2x} and R^{2y} are each independently a H or a C₁-C₆ alkyl;
each R³ is independently a halogen, —CN, C₁-C₆ alkyl, C₁-C₆ heteroalkyl, C₃-C₇ cycloalkyl, C₁-C₆ alkoxy, amino, hydroxy, carboxyl, or thiol;
R⁴ and R⁵ are each independently C₁-C₆ alkyl;
each X¹, X², and X³ is N;
each of Z² and Z³ is independently T-L- or T-L-O—;
n is 1; and
m is 0-4.

17. The compound of claim 15, wherein each of Z² and Z³ is T-L-O—.

18. The compound of claim 13 or 14, wherein R¹ is optionally substituted C₃-C₆ alkyl.

19. The compound of claim 13 or 14, wherein R¹ is an optionally substituted acyclic C₃-C₆ alkyl.

20. The compound of claim 13 or 14, wherein R² is —NR^{2x}R^{2y}.

21. The compound of claim 13 or 14, wherein R² is —NH₂.

22. The compound of claim 14, wherein the compound of Formula (IVA) is one of the formulae: 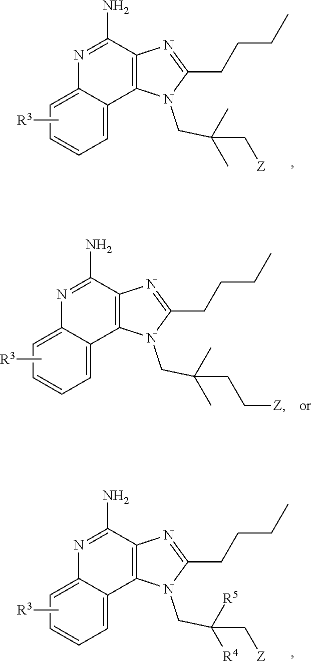
or a pharmaceutically acceptable salt thereof, wherein R³ is optionally absent.

23. The compound of claim 13, wherein the compound of Formula (IV) is one of the formulae: 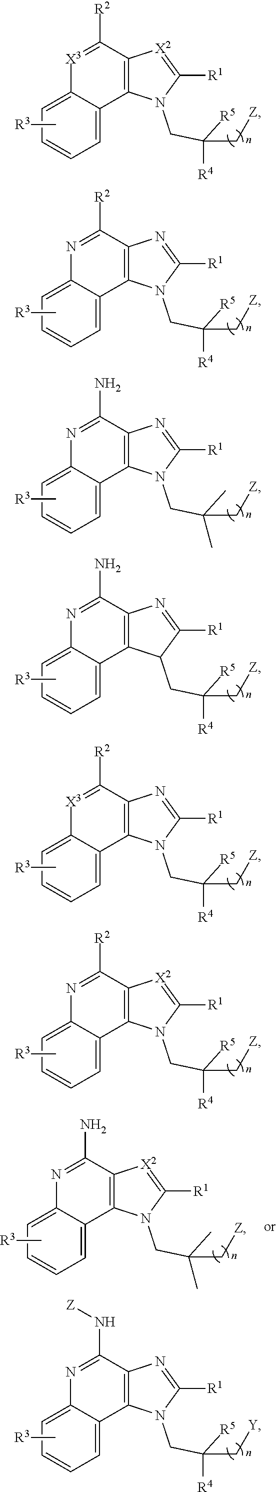
or a pharmaceutically acceptable salt thereof, wherein R³ is optionally absent.

24. The compound of claim 23, wherein R¹ is a C₁-C₆ alkyl.

25. The compound of claim 23 or 24, wherein R² is —NH₂.

26. The compound of claim 23 or 24, wherein R³ is absent.

27. The compound of claim 23 or 24, wherein R² is —NH₂ and R³ is absent.

28. The compound of claim 23, wherein R¹ is a C₁-C₆ alkyl, R² is —NH₂, n is 1, and R³ is absent.

29. The compound of claim 28, wherein the compound of Formula (I) is a compound represented by Formula (V):

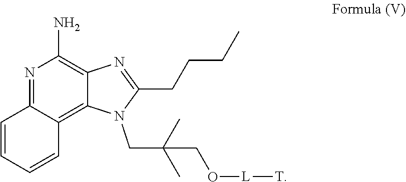

30. A compound of the Formula (I):
T-L-E (I)
or a pharmaceutically acceptable salt thereof, wherein:
T is a radical of raltitrexed, 5-MTHF, an analog of raltitrexed, or an analog of 5-MTHF;
L is a linker; and
E has the structure:

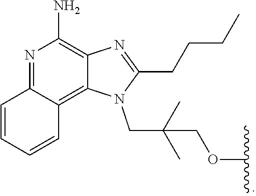 [SMILES: *OCC(C)(C)Cn1c(CCCC)nc2c(N)nc3ccccc3c21]

31. A compound of the Formula (I):
T-L-E (I)
or a pharmaceutically acceptable salt thereof, wherein:
T is a radical of raltitrexed, 5-MTHF, an analog of raltitrexed, or an analog of 5-MTHF;
L is a linker; and
E is a radical of the structure: 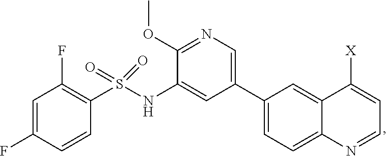
wherein X is any of the following structures:

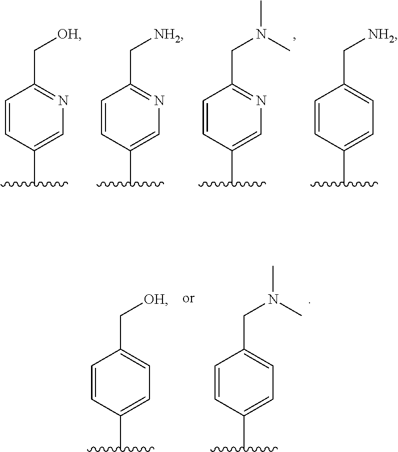

32. The compound of claim 31, wherein E comprises a radical of the structure:

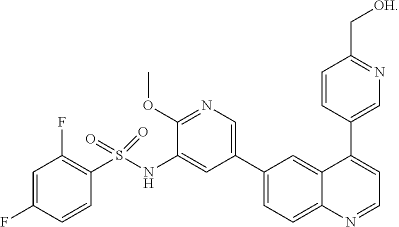

33. A compound of the Formula (I):
T-L-E (I)
or a pharmaceutically acceptable salt thereof, wherein:
T is a radical of raltitrexed, 5-MTHF, an analog of raltitrexed, or an analog of 5-MTHF;
L is a linker; and
E is a radical of a corticosteroid.

34. The compound of claim 33, wherein the corticosteroid is betamethasone, cortisone, cortivazol, difluprednate, hydrocortisone, prednisolone, methylprednisolone, prednisone, dexamethasone, hydrocortisone-17-valerate, budesonide, flumethazone, fluticasone propionate, fluorocortisone, fludrocortisone, paramethasone, eplerenone, or an ester of any of the foregoing.

35. The compound of any one of claims 1-3, 5-14, 22-24, and 28-34, wherein L is a releasable linker.

36. The compound of any one of claims 1-3, 5-14, 22-24, and 28-34, wherein L is a non-releasable linker.

37. The compound of claim 1, wherein L comprises an optionally substituted heteroalkyl.

38. The compound of claim 37, wherein the optionally substituted heteroalkyl is substituted with at least one substituent selected from the group consisting of alkyl, hydroxyl, acyl, polyethylene glycol (PEG), carboxylate, and halo.

39. The compound of any one of claims 1-3, 5-14, 22-24, and 28-34, 37, and 38, wherein L comprises a substituted heteroalkyl with at least one disulfide bond in the backbone thereof.

40. The compound of any one of claims 1-3, 5-14, 22-24, and 28-34, 37, and 38, wherein L comprises a peptide or a peptidoglycan with at least one disulfide bond in the backbone thereof.

41. The compound of any one of claims 1-3, 5-14, 22-24, and 28-34, 37, and 38, wherein L is a releasable linker that can be cleaved by enzymatic reaction, a reactive oxygen species (ROS), or reductive conditions.

42. The compound of any one of claims 1-3, 5-14, 22-24, and 28-34, 37, and 38, wherein L comprises the formula —NH—CH₂—CR⁶R⁷—S—S—CH₂—CH₂—O—CO—, wherein R⁶ and R⁷ are each, independently, H, alkyl, or heteroalkyl.

43. The compound of any one of claims 1-3, 5-14, 22-24, and 28-34, 37, and 38, wherein L is a group or comprises a group of the formulae: 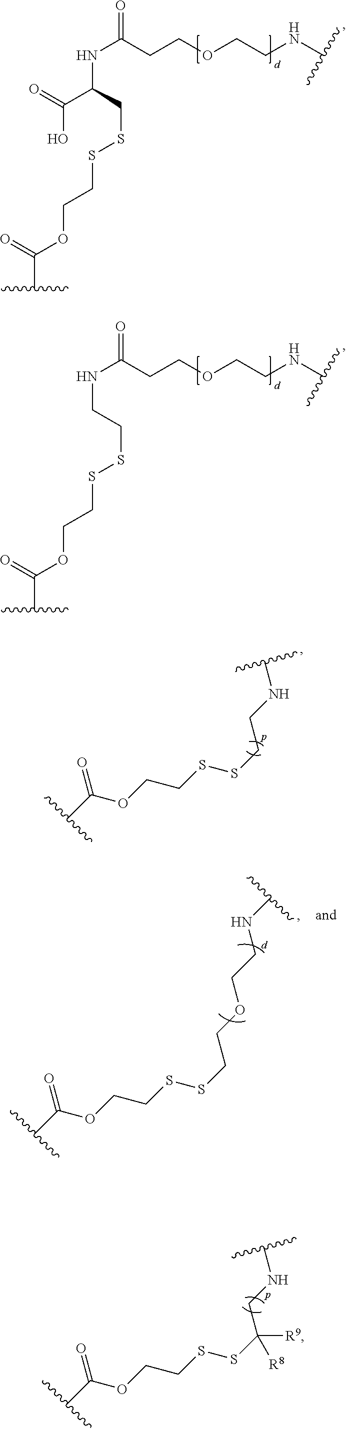
wherein:
p is an integer from 0 to 30;
d is an integer from 1 to 40; and
R⁸ and R⁹ are each, independently, H, alkyl, a heterocyclyl, a cycloalkyl, an aryl, or a heteroalkyl.

44. The compound of claim 1, wherein L comprises one or more linker moieties, each of the one or more linker moieties independently selected from the group consisting of alkylene, heteroalkylene, —O— alkynylene, alkenylene, acyl, aryl, heteroaryl, amide, oxime, ether, ester, triazole, PEG, carboxylate, carbonate, carbamate, amino acid, peptide, and peptidoglycan.

45. The compound of claim 44, wherein L is or comprises a peptide or a peptidoglycan.

46. The compound of any one of claims 1-3, 5-14, 22-24, and 28-34, 37, 38, 44 and 45, wherein L is or comprises an amino acid.

47. The compound of any one of claims 1-3, 5-14, 22-24, and 28-34, 37, 38, 44 and 45, wherein L is or comprises a PEG group.

48. The compound of any one of claims 1-3, 5-14, 22-24, and 28-34, 37, 38, 44 and 45, wherein L is or comprises a polysaccharide.

49. The compound of any one of claims 1-3, 5-14, 22-24, and 28-34, 37, 38, 44 and 45, wherein L is or comprises a group represented by the structure: 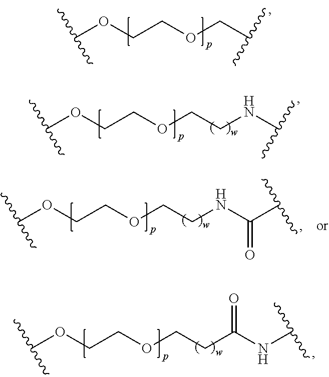
wherein w is 0-5 and p is 1-30.

50. The compound of any one of claims 1-3, 5-14, 22-24, and 28-34, 37, 38, 44 and 45, wherein L is or comprises a linker moiety selected from the group consisting of: 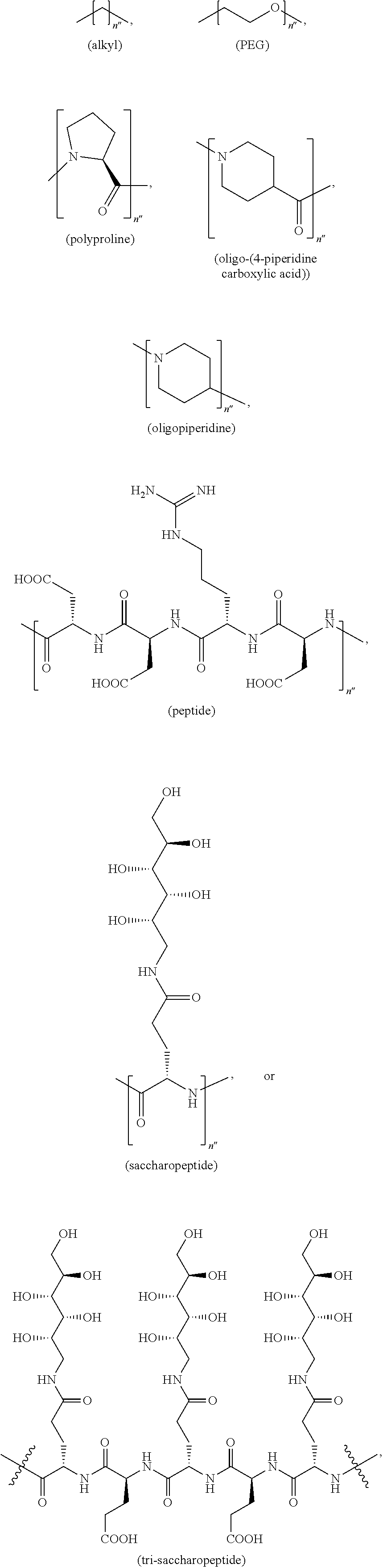
wherein n″ is 0-30.

51. The compound of any one of claims 1-3, 5-14, 22-24, and 28-34, 37, 38, 44 and 45, wherein L is a bivalent linker or a trivalent linker.

52. The compound of claim 1, further comprising a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the linker.

53. The compound of claim 52, wherein the compound further comprises an albumin binding group selected from the group consisting of

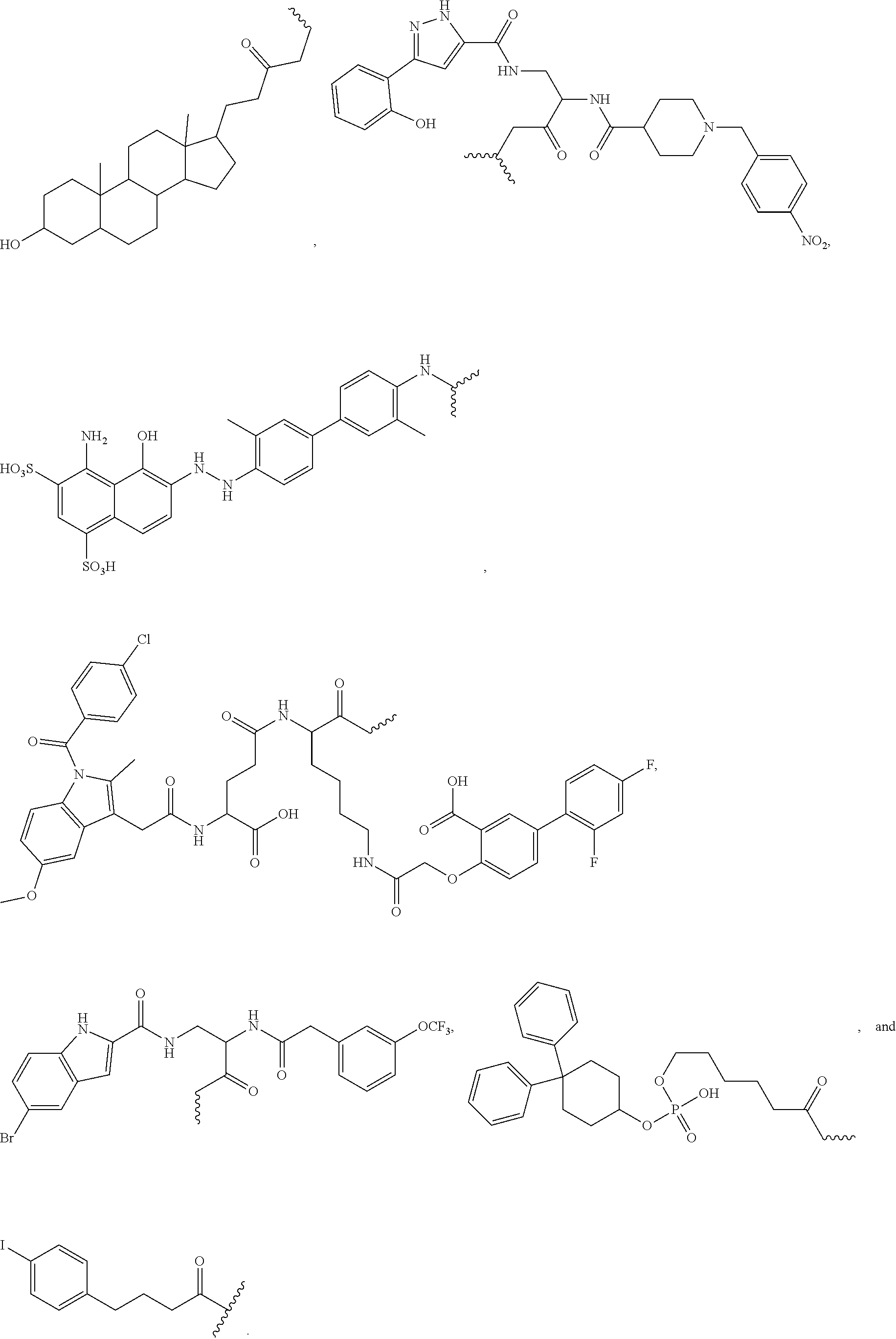

54. A compound comprising one of the following structures:

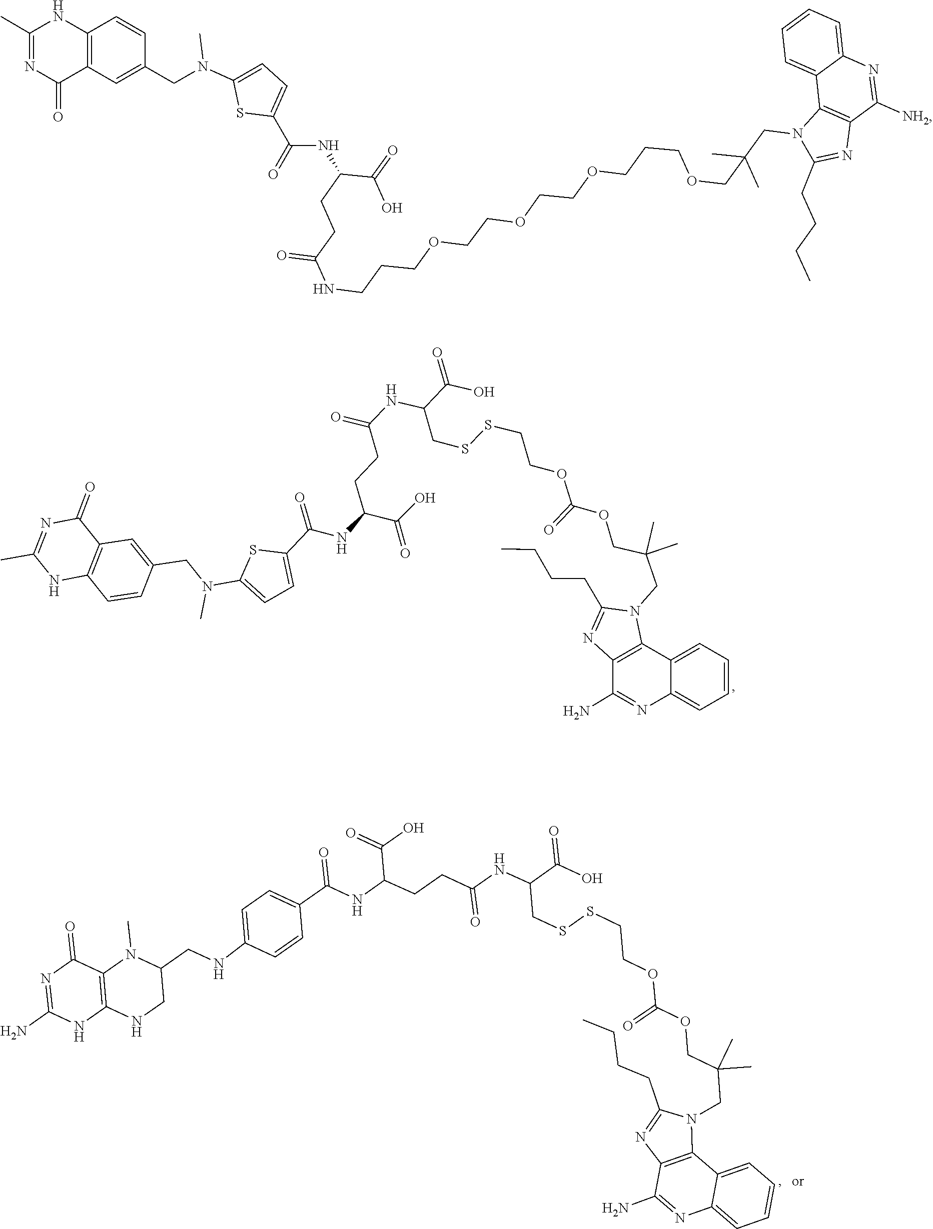 [SMILES: CCCCc1nc2c(N)nc3ccccc3c2n1CC(C)(C)COCCCOCCOCCOCCCNC(=O)CC[C@H](NC(=O)c1ccc(N(C)Cc2ccc3[nH]c(C)nc(=O)c3c2)s1)C(=O)O ; CCCCc1nc2c(N)nc3ccccc3c2n1CC(C)(C)COC(=O)OCCSSCC(NC(=O)CC[C@H](NC(=O)c1ccc(N(C)Cc2ccc3[nH]c(C)nc(=O)c3c2)s1)C(=O)O)C(=O)O ; CCCCc1nc2c(N)nc3ccccc3c2n1CC(C)(C)COC(=O)OCCSSCC(NC(=O)CCC(NC(=O)c1ccc(NCC2CNc3[nH]c(N)nc(=O)c3N2C)cc1)C(=O)O)C(=O)O]

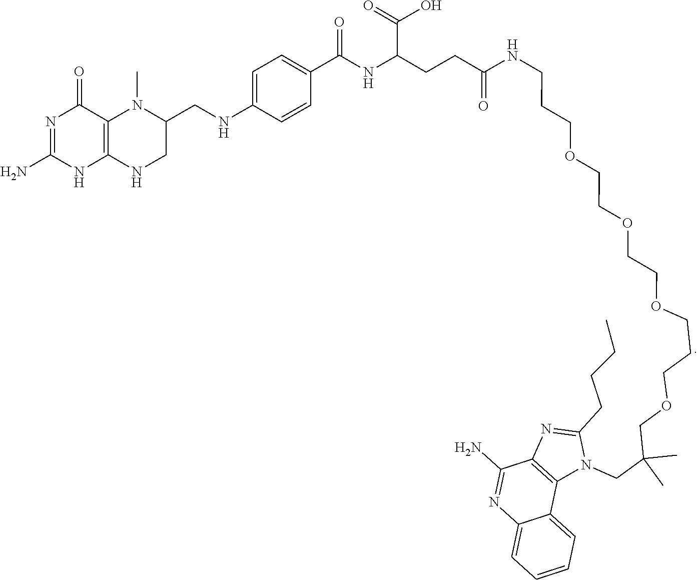 [SMILES: CCCCc1nc2c(N)nc3ccccc3c2n1CC(C)(C)COCCCOCCOCCOCCCNC(=O)CCC(NC(=O)c1ccc(NCC2CNc3[nH]c(N)nc(=O)c3N2C)cc1)C(=O)O]

55. A compound comprising one of the following structures:

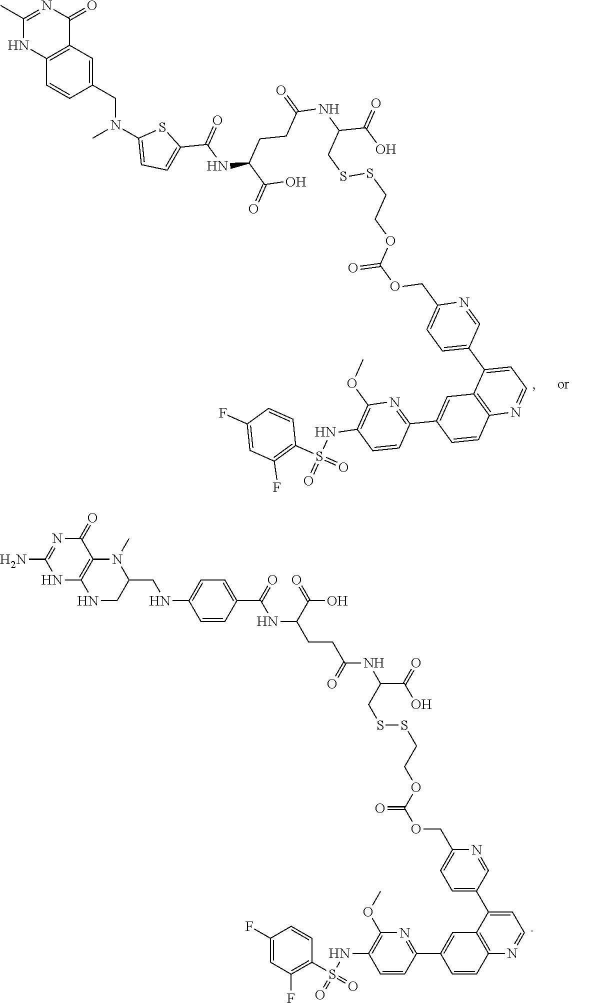 [SMILES: COc1nc(-c2ccc3nccc(-c4ccc(COC(=O)OCCSSCC(NC(=O)CC[C@H](NC(=O)c5ccc(N(C)Cc6ccc7[nH]c(C)nc(=O)c7c6)s5)C(=O)O)C(=O)O)nc4)c3c2)ccc1NS(=O)(=O)c1ccc(F)cc1F ; COc1nc(-c2ccc3nccc(-c4ccc(COC(=O)OCCSSCC(NC(=O)CCC(NC(=O)c5ccc(NCC6CNc7[nH]c(N)nc(=O)c7N6C)cc5)C(=O)O)C(=O)O)nc4)c3c2)ccc1NS(=O)(=O)c1ccc(F)cc1F]

56. A compound comprising one of the following structures:

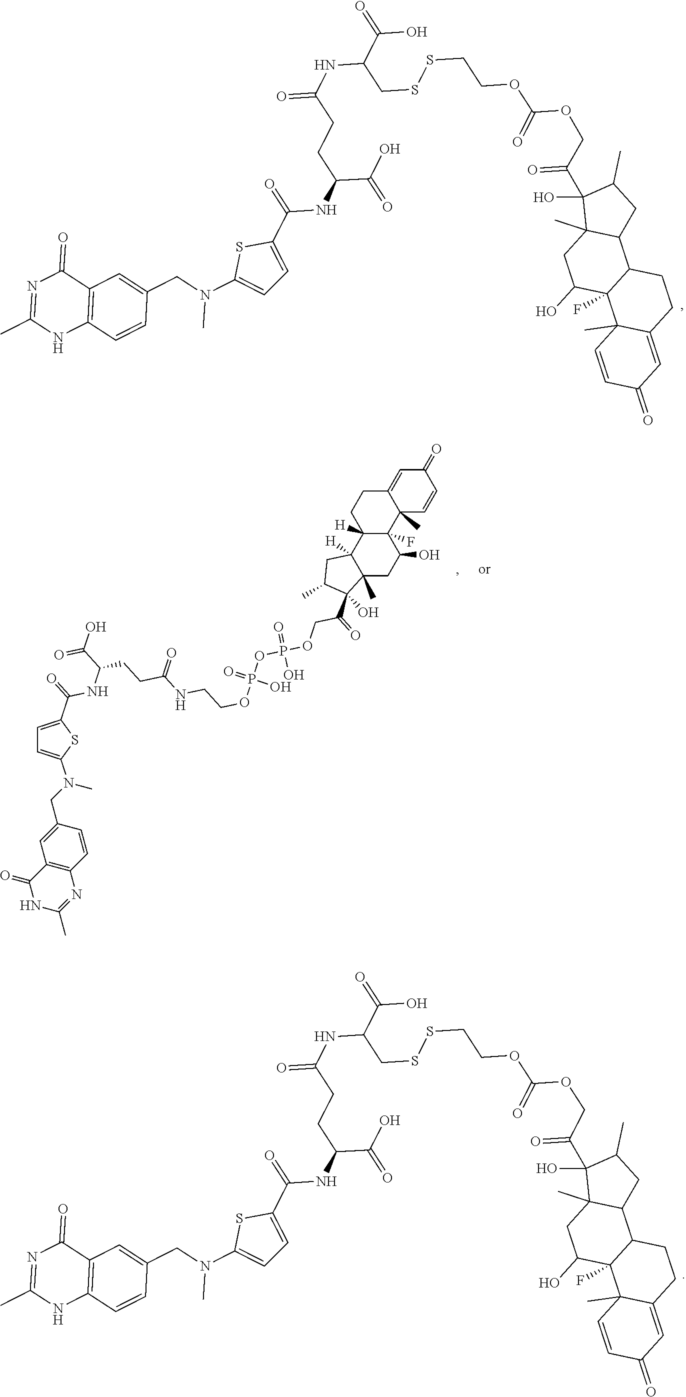 [SMILES: Cc1nc(=O)c2cc(CN(C)c3ccc(C(=O)N[C@@H](CCC(=O)NC(CSSCCOC(=O)OCC(=O)C4(O)C(C)CC5C6CCC7=CC(=O)C=CC7(C)[C@@]6(F)C(O)CC54C)C(=O)O)C(=O)O)s3)ccc2[nH]1 ; Cc1nc2ccc(CN(C)c3ccc(C(=O)N[C@@H](CCC(=O)NCCOP(=O)(O)OP(=O)(O)OCC(=O)[C@@]4(O)[C@H](C)C[C@H]5[C@@H]6CCC7=CC(=O)C=C[C@]7(C)[C@@]6(F)[C@@H](O)C[C@@]54C)C(=O)O)s3)cc2c(=O)[nH]1 ; Cc1nc(=O)c2cc(CN(C)c3ccc(C(=O)N[C@@H](CCC(=O)NC(CSSCCOC(=O)OCC(=O)C4(O)C(C)CC5C6CCC7=CC(=O)C=CC7(C)[C@@]6(F)C(O)CC54C)C(=O)O)C(=O)O)s3)ccc2[nH]1]

57. A compound comprising one of the following structures:

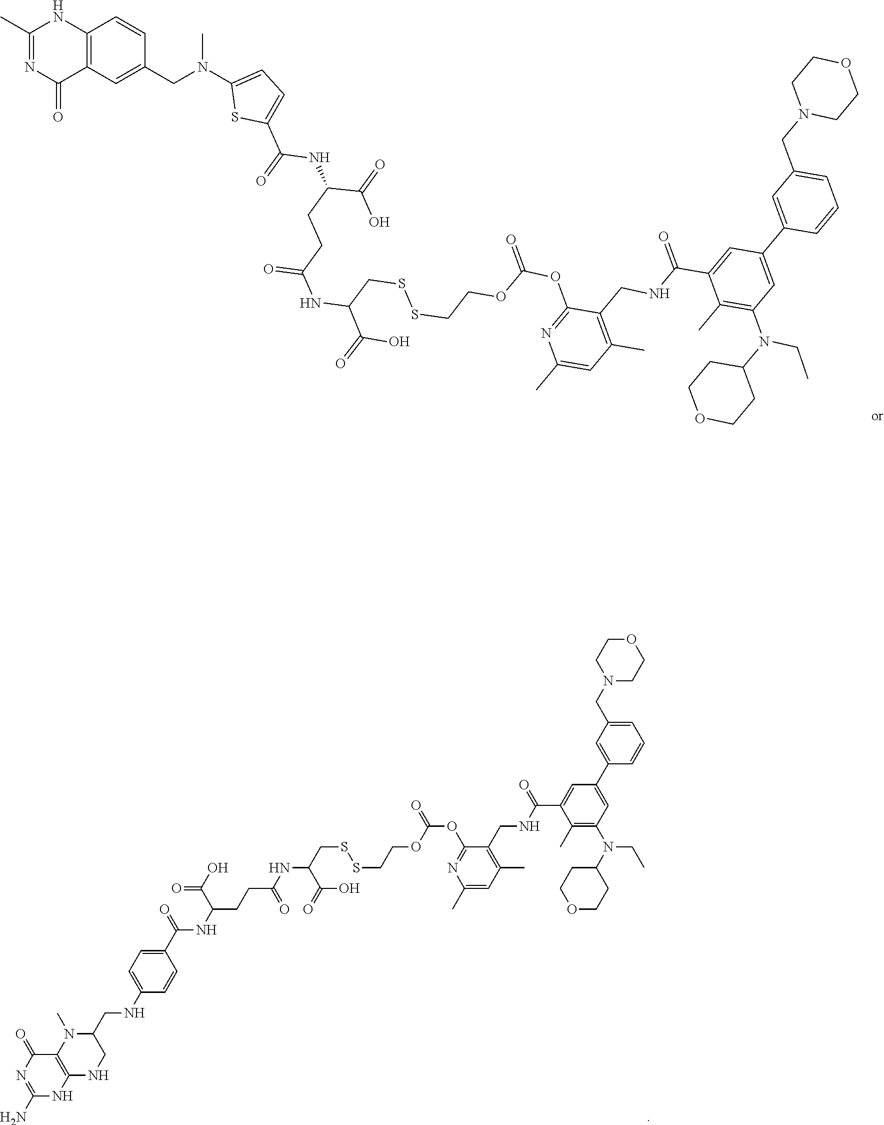 [SMILES: CCN(c1cc(-c2cccc(CN3CCOCC3)c2)cc(C(=O)NCc2c(C)cc(C)nc2OC(=O)OCCSSCC(NC(=O)CC[C@H](NC(=O)c2ccc(N(C)Cc3ccc4[nH]c(C)nc(=O)c4c3)s2)C(=O)O)C(=O)O)c1C)C1CCOCC1 ; CCN(c1cc(-c2cccc(CN3CCOCC3)c2)cc(C(=O)NCc2c(C)cc(C)nc2OC(=O)OCCSSCC(NC(=O)CCC(NC(=O)c2ccc(NCC3CNc4[nH]c(N)nc(=O)c4N3C)cc2)C(=O)O)C(=O)O)c1C)C1CCOCC1]

58. The compound of any one of claims 54-57, further comprising a radical of a PEG group, a peptide group, a glycopeptide group, a saccharide group, or an albumin-binding group, wherein the radical of the PEG group, the peptide group, the glycopeptide group, the saccharide group, or the albumin-binding group is attached to the linker.

59. The compound of any one of claims 54-57, further comprising an albumin binding group selected from the group consisting of:

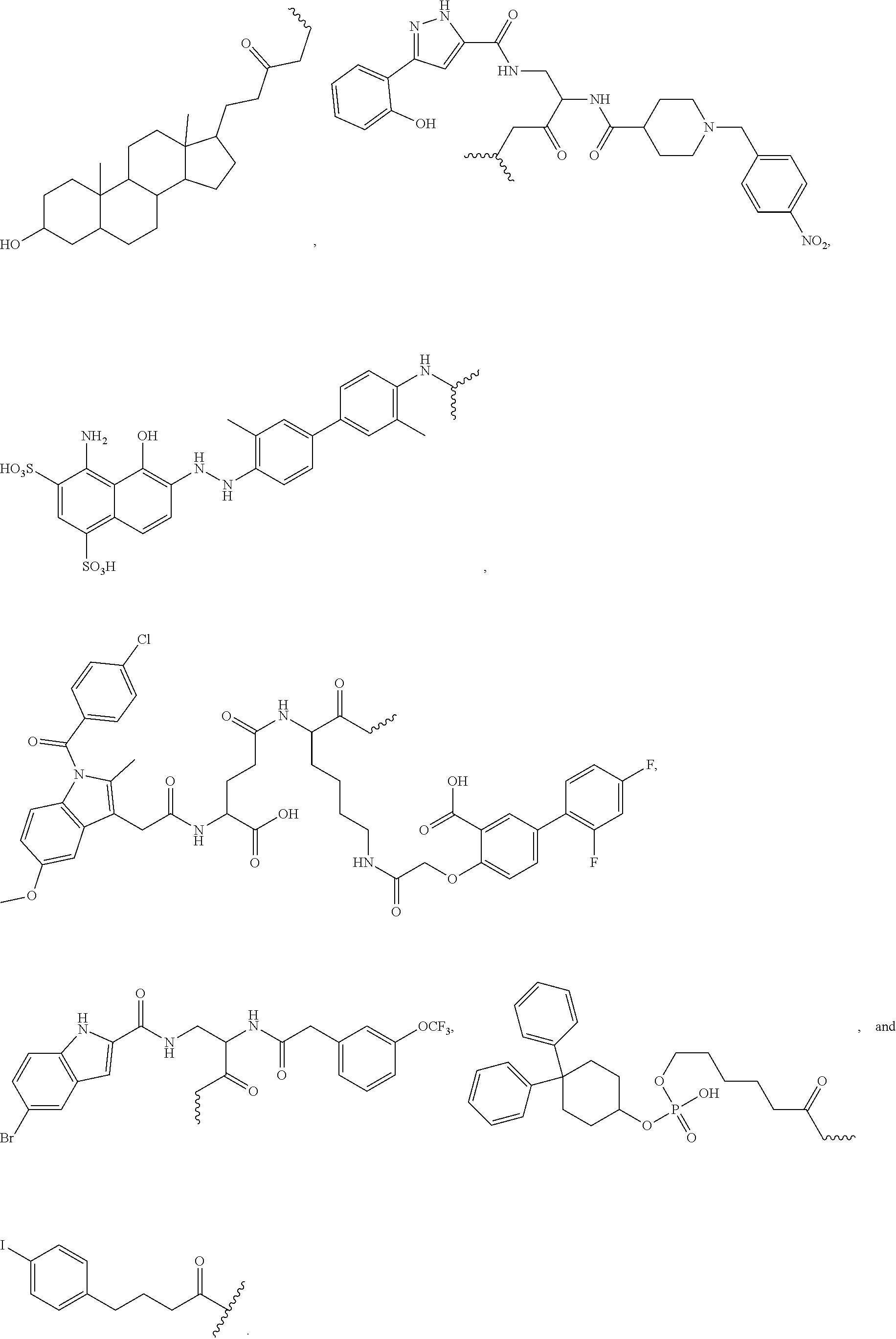

60. A pharmaceutical composition comprising a compound of any one of claims 1-3, 5-14, 22-24, and 28-34, 37, 38, 44 and 45, and a pharmaceutically acceptable carrier or excipient.

61. The pharmaceutical composition of claim 60, which further comprises a compound of formula:
F-L′-G
or a pharmaceutically acceptable salt thereof, wherein:
F is a radical of folate or an analog thereof,
L′ is a linker; and
G is a radical of glucosamine.

62. A combination of pharmaceutical compositions comprising:
(i) a pharmaceutical composition of claim 60; and
(ii) a pharmaceutical composition comprising a compound of formula:
F-L′-G
or a pharmaceutically acceptable salt thereof, wherein:
F is a radical of folate or an analog thereof;
L′ is a linker; and
G is a radical of glucosamine,
wherein (i) and (ii) can be administered to a subject by the same or different routes.

63. A method of immunomodulating regulatory T cells (Tregs) in a subject comprising administering to the subject an effective amount of a first compound of any one of claims 1-57 or a first pharmaceutical composition of claim 60.

64. The method of claim 63, further comprising administering to the subject a second compound of formula:
F-L′-G
or a pharmaceutically acceptable salt thereof, wherein:
F is a radical of folate or an analog thereof;
L′ is a linker; and
G is a radical of glucosamine;
wherein administering the second compound can be performed simultaneously or sequentially with administering the first compound or the first pharmaceutical composition in either order, and by the same or different routes.

65. The method of claim 63, wherein:
the subject has cancer; and
wherein:
E of the first compound or the first pharmaceutical composition is a radical of a TLR7 agonist, a PI3K inhibitor, a NLR2 agonist, a STING agonist, an EZH2 inhibitor, an NLRP3 inhibitor, a Caspase I inhibitor, or an RLR agonist; and
administration of an affective amount of the first compound or first pharmaceutical composition alters Tregs' promotion of tumor growth and metastasis and/or inhibition of anti-tumor immunity in the subject.

66. The method of claim 65, further comprising administering to the subject a third therapeutic agent.

67. The method of claim 66, wherein the third therapeutic agent is an anti-cancer agent such as a chemotherapeutic agent or a radiotherapeutic agent.

68. The method of claim 63, wherein the subject has a fibrotic disease or disorder, and the E of the first compound or the first pharmaceutical composition is a radical of a therapeutic agent selected from the group consisting of a TLR7 agonist, a PI3K inhibitor, a steroid, a NLR2 agonist, a STING agonist, an EZH2 inhibitor, a NLRP3 inhibitor, a Caspase I inhibitor, and a RLR agonist.

69. The method of claim 63, wherein the subject has an inflammatory disease, and the E of the first compound or the first pharmaceutical composition is a radical of a steroid.

70. The method of claim 68 or 69, further comprising administering a second compound of formula:
F-L′-G
or a pharmaceutically acceptable salt thereof, wherein:
F is a radical of folate or an analog thereof;
L′ is a linker; and
G is a radical of glucosamine.

71. A method of treating cancer in a subject, comprising administering to the subject a therapeutically effective amount of the compound of any one of claims 1-3, 5-14, 22-24, and 28-34, 37, 38, 44 and 45, or a pharmaceutically acceptable salt thereof.

72. A method of treating a fibrotic disease or disorder in a subject, comprising administering to the subject a therapeutically effective amount of the compound of any one of claims 1-3, 5-14, 22-24, and 28-34, 37, 38, 44 and 45, or a pharmaceutically acceptable salt thereof.

73. A method of treating an inflammatory disease in a subject, comprising administering to the subject a therapeutically effective amount of the compound of any one of claims 1-3, 5-14, 22-24, and 28-34, 37, 38, 44 and 45, or a pharmaceutically acceptable salt thereof.
